SEQUENCE LISTING (240 sequences)

SEQ ID NO: 1 - DNA, 14067 bp, Escherichia coli
  misc_feature (1)..(14067): Transposon Tn7 (E. coli), Genbank Accession Number BM_NC_002525, extracted from NC002525, comprising left and right ends of Tn7, plus tnsABCDE in the right half, and int12, dhfr1, sat, aadA, ybeA, ybfA, ybfB, and ybgA in the left half of the transposon.
tgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttaactaatctagctaacctgccgctcagcttagtacgatcctatatccaactgatcataatcctttcatcagtatcccaaattaaatccccctcctagactgcctcaccaacactttacagtgatcccgtatttccggacaggtgattgatttttttcgcttgtacaggtgttaagccacagttatcttgatgtggacattatgcattgtaatagcccattaacatgcgctaagagcataaattaatatacagtaacaagcattacgaggtgctaatgtttggaaagcagcctcttccaactacaataataagttaactatcctccacccacaaataattacaaatgacccctttccgctataactccgatctcaccagcggctcgctacaaacccgagagtgtcgcattattactggcttgttactccaagagcttgatgaagctgcctgggataaagccatgtataaggaaaatgtgcttcagaaacgcacgcaatctacggtaagacgtatttcttcagcgcttagaaaacgcctagaacacctaagctctgacttttgggcttttgcgtttttatgttaggcattgtctggcaactggaggaagtgtaatgaccatgaatggtcactgtgattgacagtatcttgcttaaacttatctgaacaatggtagaactggctcatatttatcccctctatgattgtttatctttttcaccataaaaagtatcaggggctactcagggctacataaagatatttatctacttttactgattgataagtagcatcagtccatccgcaggactggtggtgccggcaaaatgctgacccaacacatgcgtatagatttgcgtggtcttaacatcgttatgccctaagagttcttgcacagtgcgaatatcacgccccgcttgtaatagatgcgtagcaaacgagtgacgaaatgtatgacaagtgacacgcttgctaacgatgcctgctttttgtacggctgccttcaatgcctttcgcgcaacggagtcatgcagatgatggcggcataatttgccgttatacgggtggttgcagagcgtgctggagggaaagacaaacatccacgccgcttgtcgataagcagaagggtatttgtgatctaaagcaaaaggcagcgatggccctacgccttgtaagttgtcgtcttgctgaataagccgcgcttgctcaatgagttattttattgctgggattaggcgcgtgggcagtaggctgtttctgcttttcccacccttaccgtcatgcacagtgatgcagccattatcaaaatcaaaatctttaacccgcaaacgcaagcattcattaatgcgcaaacctgcaccatacagcagcgtaaaaataacttggttgcgagtatccataacctgcaaaatgcgttgcacttcatttgcagagataacagagggtagccgtctaggcttgcttgcagggatataatcaatatcgcccaacggctgttgtaaaaacctgttgtacaaaaaagctagggcatttaaagcgattttctgcgtgtttatggctacatgtctgctgtttgctaagctggataaaaacagcctgacctcttcactgcccatggtctgaggatgacgttttttgtgaaacagaataaaacgcttaatccagtgcaggtaagttttttcagttttcagcgcataacctttttgtcgcatatccgtgcgtatagaatttaaaaatggactgttagacataaaacgctccttgtcttgcaactgtctgcctatacagcctattctagctgggatttaaaaaagtgcctgttttttacgcctagagatgcttgtttaccggtagagttttaatttaatgctaaataaattaaaatgttatgagttctttgggtgagataatgtgcatcgtgcaagcaggatagacggcatgcacgatttgtaataacagagtgtcttgtatttttaaagaaagtctatttaatacaagtgattatattaattaacggtaagcatcagcgggtgacaaaacgagcatgcttactaataaaatgttaacctctgaggaagaattgtgaaactatcactaatggtagctatatcgaagaatggagttatcgggaatggccctgatattccatggagtgccaaaggtgaacagctcctgtttaaagctattacctataaccaatggctgttggttggacgcaagacttttgaatcaatgggagcattacccaaccgaaagtatgcggtcgtaacacgttcaagttttacatctgacaatgagaacgtattgatctttccatcaattaaagatgctttaaccaacctaaagaaaataacggatcatgtcattgtttcaggtggtggggagatatacaaaagcctgatcgatcaagtagatacactacatatatctacaatagacatcgagccggaaggtgatgtttactttcctgaaatccccagcaattttaggccagtttttacccaagacttcgcctctaacataaattatagttaccaaatctggcaaaagggttaacaagtggcagcaacggattcgcaaacctgtcacgccttttgtaccaaaagccgcgccaggtttgcgatccgctgtgccaggcgttaggcgtcatatgaagatttcggtgatccctgagcaggtggcggaaacattggatgctgagaaccatttcattgttcgtgaagtgttcgatgtgcacctatccgaccaaggctttgaactatctaccagaagtgtgagcccctaccggaaggattacatctcggatgatgactctgatgaagactctgcttgctatggcgcattcatcgaccaagagcttgtcgggaagattgaactcaactcaacatggaacgatctagcctctatcgaacacattgttgtgtcgcacacgcaccgaggcaaaggagtcgcgcacagtctcatcgaatttgcgaaaaagtgggcactaagcagacagctccttggcatacgattagagacacaaacgaacaatgtacctgcctgcaatttgtacgcaaaatgtggctttactctcggcggcattgacctgttcacgtataaaactagacctcaagtctcgaacgaaacagcgatgtactggtactggttctcgggagcacaggatgacgcctaacaattcattcaagccgacaccgcttcgcggcgcggcttaattcaggagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctaggcaggcttatcttggacaagaagatcgcttggcctcgcgcgcagatcagttggaagaatttgttcactacgtgaaaggcgagatcaccaaggtagtcggcaaataatgtctaacaattcgttcaagccgacgccgcttcgcggcgcggcttaactcaagcgttagagagctggggaagactatgcgcgatctgttgaaggtggttctaagcctcgtacttgcgatggcatcggggcaggcacttgctgacctgccaattgttttagtggatgaagctcgtcttccctatgactactccccatccaactacgacatttctccaagcaactacgacaactccataagcaattacgacaatagtccatcaaattacgacaactctgagagcaactacgataatagttcatccaattacgacaatagtcgcaacggaaatcgtaggcttatatatagcgcaaatgggtctcgcactttcgccggctactacgtcattgccaacaatgggacaacgaacttcttttccacatctggcaaaaggatgttctacaccccaaaaggggggcgcggcgtctatggcggcaaagatgggagcttctgcggggcattggtcgtcataaatggccaattttcgcttgccctgacagataacggcctgaagatcatgtatctaagcaactagcctgctctctaataaaatgttaggcctcaacatctagtcgcaagctgaggggaaccactagtgtcatacgaacctccaagagacggttacacaaacgggtacattgttgatgtcatgtatgacaatcgcccaagtaagtatccagctgtgttcagaacgtacgtccgaattccagactatgtcgaactagataacgcggaggagctggccgacgcaatcattcgagaggttgagttaaaggaggcatggctcacaagggtcttggggaactcagccgatcctgacgcttctgtgggcaagtgggaccggcgcctagcgcagtgcggtggtattgatgagatgtcaagtggggtgcaggcaacatacttcagtccaaacggtcagctggtcggcttagtggcatacattgatgccgaatggatggatgaatagctctgacgttacgattcaggtgagacaaccagatgggcattgcgtacaagctcagttgccaccgatgtggctacgagtctgatcttctttacctaggccaaggtatggcgatgcttcctgagcaaatagtcggaacgtgcacatgctgcgacaatctaacgacgatttctataaatgaaagctacggagtatgctcactttgtggggctaaggatcgtgtgatatttacaagctatcagcatgaggcaaggccaagaaatccctgctatctagagagaagtttcaaatatcaatgcccaagatgccatgccttcagtatggagcctccggatctaccggagatgctttgggactaatgctccgctttgcctcgggtatccgttcgacctatgtttaagaggaaacgcacgtgtctgccgccaaagctcttgcctatgcactttgtgacaagttggaaagcaaaaaagttgatgaagtcgtacgaaatcttgtttctagcggctggaatattagggaagtggcctgggaagcttcgtcctttgatcatgaaatgccctactttgacacggggcgggacttcgatattggatttcctgacacgctgtcatccaagattgagtttttgaccgcgaatggatcaatcgtccaagcacgtgctcgcctgcacttcaagcaggctctaatttttgccaaggcgtccaaacatgtcgccgcgctcaagaactcattggattactactatggatcgtctgtaatcccccctcaaaaccacggaggctatttgtagaaaattccattaatatgaactcaaggtgttccctacggacgaatcagcgatgaaggtggtgtacctggctatccaggcggcgaccaagaaatggacgatgccgatccgcaactggaagccggccatgaaccgttttatgatcgaatttggtgaccgtttaaatggccacctttaactgaccggcaaaagcacttacacaaaagggtttacaggctctttcagtcattcctttaatgcgtaaattgctctctaaccactctctcagcccacccttccatatcctctctcgatacttgcagctcccccaacccctttggatgattcacccctctatgcccgtcttgtccatataattgatcaaacaaactatttggccaattcaatgagctcttcaccactccacgccgtatcttttcaaaatcattccgccatgtttcgctatcaacgccactcaacactttggtagaaagcattttcaccccatccgatgcatccacctctagcaatacaaattcactgccattacgttgcaccccaacggctttaatcactcttggtgagccatctttacacagatgtaatcggctacgcccaacttttggcaaaaccaaggtttcttcaaaaagcacccgacatgcaaatttagtttttagaattgaaagtagctcatcaaaagcagcaaatcgattagcaaaaatagagttgtaattggttgcatcctgcttcccacccacatctgccgctgctaggacaccacctaagtgtggctcatcagtactaaccaaattactattttcacttgttgttgcttcttctttttctaaaatagtttggctactttttcttgccctgcgagatttttcaattcgactaggtctactaaaacttatccatgtcggctcagcctctatgactcgtgtgtctgtgtctatattggctgtttcctcatcgtccaactgtagatgctcatcatcaacaactgactctgttgaaaccgctatgtgctgcgtactaccatcacctgcctccttttcctgaaaagaggcatggctaatagctgttgtgctaggaagcatagcgttgatttctaaacctactatttcctcaactaaataatccttatcctcgttagaagaacgtccttttacatgtaatttccaaccctgcatgggcggagggtcaaagctaaagcaccagctttcaacgccgttcttaatttctctattttgttgatagtgcctaaaaatactttcatacgaatccataacatcttgatccgaaaacaaccaaaccaaaagctgcactacggccgactgctctaacgcccctttaggaaacgatgaactgggtaagatccttatctctaaatgatctcgctcaacctcatactgaacgtcgaactcttgctgtaaagcggtactgctcaaacagcttcgacagaaataggagttaattaaaaataatgaccgagctagctctaactgtgggatgtgataacgagtcttgctatatggtgtttcagcttcgtaaataaatgaactctgcgctccatccttgttgcgtacggaagcaaaggacgagagattaggaaaatcgattattttggctcgttgaagatccgatgcttcaaactcaatcacacgatccgccttatttatttgctttgttgtactgttaaccaccttagctcgacttaataaaggcaaatgagtaaatcgagtccatttccgttcttgcattggattaaaccaaacaaaaatacgccactccttatgacccgagttacggaataaatgaccaatatgtacaacctgcacattgtcattaaatgtagctagcctaaccactcactcttccccataaaccatttccaagaatctttgtgcttcctcagttatccgctctttagataacgttgcacttcttaataatcgccagcgcctaagctcaacatcttcctgtttaagcttaataaaagcttggctaatccgtctaatttgataatcttccacactctctgagtaacgctttaagcaaagcgctaccaaaggcagtttctgtagattttttgcaagagaggttccgttaggagtttgctttaacagccagctcgatgtcgctcttgggtgatcaaggctactatctagacgctttatgattcttaatagttgtcgtacagcaattcgatctctttggttccaatcaactctaggggcgggtgccagacgctcttgttgatgctgttgattccagtgaactagccaatccctgtcatgtcggtaaagccaagcgtatagcaccccaccctctaaagactgtcttgccgccttaattccttggtatttatgcactagttgctgccagtctttatgcttaacagataaatcttcagagttaggttgcacagactggctaacaggtcttgtcgttatagagtgctcagtaagagcacttgcctgctgtagcgcttctataaccgttagttttggcaataaggcttgccacacaatactatgctgtaaataactaaaggcttttctatgcttacggaatatacttttcagccaacacgtgtccttgttctctgccaactttaaatccagtttctctagtgcctcatcactaaaagtctgcctcactctctccgccaccaagtcatgacgaatgtgcttgcttttggttagccctagatcctgcgctaagcgctgataaaacagcgtccactgctcaaggcttggggaaagctcttgcgctcgtggagcatctaacagaggggctatataagcagctagtgctgttaattgagatagggagtctttggggtagtctgaaagcagctcagtatgacccaaagcccaaaattgatgtcggtgatcatctacagctctatcaaagaagactaaagcaccgtgttttggacaatatggcaaagcgggcaaataccaatctcgttgccaaaaggcttccccatacctatttagctgaagagcaacgcaatcagggcagtatctaaagcggttatcgctcttaactctagaagcagcgactcctagcattaaatgcaccgcaccttgcgcttggtactccattaaccgaatagcttcgtctcggcgctccttgcctacaaacggagcatataaagggaataaggtatgctcataaataagctgctgaacagcgtaacgtcctgtttgatgtagatgtcttgctatcacacctaaatgcgagggcagacctaaggtagcgaccaccttgcggttgccatacacctcatccaacagctgcttaggactaacaatcccttgataaacgcctgcccgtgcaatagtgctataaatcagctcattcgagtacggaacaggaaagtttctcatggctaacctgcttgtttaaataagctcgccatatccactattacccctttcccttttaaccgttcatgcatggttttttcaggttggcgttgtgaatagatataacgtaaatccgtatcaggcaagctatcccagtcgctgggcttgactacctttatagccgaaacctttttactcttggagggcttttctagttctgacactaccgtttcgccttccatcaaccactgcaaaacaagaggcagtaacttttgtcttgtcatcgttggattctggctaaacgcttttttaatagtgggaattaacaggcttgaatcgtaatcctctttcagcatcagatataaatgacgctgatcttcggtatctaactcttgaagggctttttcttctggtgtttgttcttgtatcgctgcgatatctagctgaagttggattaaccgtttatcaatctcgggaacgactagatcagaataacgagcaatgcgttctgggatacccgagcgtaatgcctctagcatggggtgcacaggctttaactcatcttgatacacttgccgcaataaaccagcggtaatacgctcattgcctaaagctagcgcacggagctgagcgagtacaaaaagttttactacaatgtccatcactccttggcttagctcataccacacatcacggacctcatccgataacagcgcatctttgcgttgtaaaagctgtaattgccaaagattatccgtaaaagcgatccactcttgattgggctttccacgttgcgtttgttgtataggatcccagaatatagctccaaaccctgcccctctacgtgcagaccgcaaatcagcctcaaaaatctctcgtgctttaggggtaccaatcaacatcactggtacgccaataatattcaccatcgtcacaaaaaagttcagcatctcttgagatccacccgaacgagagcggcttaaatgctgaatttcatcaataaccaacaaccctaaagcatgtgcattggctatttgcgacatcaaagccaacatggtttctataccatgacgttttaagccataacgacgctcatagttcgagcccaaggctcgatccaacgctctgaaaaaattcaagcagatttcttttagcgaaccattatgcgagcagtctattttcaaatacaccacctgctctacattgagttcacgatggtaaatcacctgaggatacgtggctagaatacgatgaagagaggtcgtcttcccactaccagaacaaccaattaacaataagctttgtgccgtagatcgtgcctcctcaaagcgaaatgtctccaactctcccgtttgaacacgctcataaccattttgtaaatgcttttgtaaatctcctgttttaggatttctgcctacgtagccacctcgaatcatgaccgaaatacgctcacttagtagcaaatgcgtacctaatggctgaaaatagtcatctggaatacgacaaatggtatgagctctgataacacgggacttttgcaagtcagaggaagtaagctgtaaagaggatttcagtgatgcagcactattcactgactcttgtaatggtggtaaggcctcgataaaaggattatcacgataagcctctacccccgtatcacgataaactgcttgaatccgggtagcactcatgactcatccttttctggtggatcctgaaataattcaggcacgtatgtgggtaggctgtaatcttcttgatcatccgcttccactgcgttgaaaggaataactttagcctcatcacctgaagagcttggcttcaaatgctccgcacgttttttacgctccgaggtcacggcttcttttttattagttttaatctgcttaatgcgtgttgatttgggctcagtagtactgggcgttaacttattcgctttctgaatggtttgctgaataaacgcctcaagctccctgcgtttagttaactcatcctgcttcgcattggctttattgtgtttttcttgtgcttgtatatcccaaacctcccaaaatgagagacctttaaactgccgactacgttccgtcagattacagcgccaaaatacacggctgccaacttgcggaaacaaataaatcgtatcaaccagcactgggtcataagccgcttctaaatgttgaggtctagctatatcagtgctccgctgcaaccaaccctcacgcagaatctctgaccccgagtaatacaaaccccacaaattaacgccaaatgaagaaatagagacctttcggcgaggcagtaacgctactcgcaactgctcttgctccacagcccttaaactacctgtacgatgctgcataccccattgccatagctggacaggaatagacggtaaatctgtaggaaaatcagcatctcgatcgtatttatccatcaccagatggttatttctgaataagatcgtacgcaaaataatttgtgtgaactcaaataccgacagagatgcatctaacctatagtctgtttcaccatggcttttgatccgactgccctctacaatgccaggtgcaaaggacttaaactcggcttgtagtgttctaaaagtgctttccactatgcctttagcatcgccacgtctaggtggagcactttccactcgcacattaaaactagaaactaaggcttcgacctgatgactcattaattcgccacggtccgctagcaacacatctggcaaacctacacacggccagtctgagctactaatctcaatatcatgctgggcacaaatggccgttttgtcagagcaagcatttacaaaagcctgcatcgccaccacataagacggattttcaaagccgatataaaagcccgtgatcatccgactaaacacatcaatcacaatgtaaagcgttggtcttcctatgattttttggcgatcatgatgatccactaaataaatatcagcaatcgtggcatcaatctcataacgactcccagggcctaacgcctgagaagtggctgtactacttaagggtcgtacgtcttttttatatacccctgctttaactctagactttaagcgctgagctttagggtattctcgatcataaaaataacgaaactgacgtagtgttgggtaatcctcttggggaatgcgaggaaaatactgagcaaacaagtccacaaatcgtctataggcaacggtggtctttgtacctttttgatttaacaggtgcttttctatggtcaacctaaaaaggcgttcaatctcgggcgttaccttggttccttcacccttaccatattctctggctcggccaatctttgctgttcctgtcgctgaacgtctttcccctggtgcaccgctgtttttgtagtcaggaattaatgcattaggcgtttgaccacgctgccagtaacggcgtaacaacttataaactgtagccttagtaaccttatgttcttggaccacatgctcaacgagttcgcttctgactttagggtcgaaacgatccttactattaataataggaagaatttttcgataatcctcctcgcgcttctggaagctgacagaatctacagacggctcttctaaatcaagatgtacataagggtcatcagttctaactaagcggccttcatctaaatactgcattagcaacaactcagccctagcttgtggaactcctttatcagcgcttatttgcatccagacaacttggccatcctctatagccaaaatgcgatacggatcattatcaaatagcacaacctcattaatttgccacatagcgcaactcctccatattcactacttggctaatacagagatctgcacacttatttgccctgaaagacttataaatattgaacttaataaaaccatttgcggttaaggctcgtatctcactcaatgttttgcctaactccaaatcataagcaatatcaacctgcttacagacattgataatgttttcatctcctttttcttgcaggatatgggccaatggggatagttgtgctaaaagctccgcagaaacttcttctgttttcactgaataaagccattcaatattttcttttactacgggatttatttctttatcagtaaaaatgaaccaaggaatttgcttttgctgccaatagcgacgctctagttctagtttttctaaggtacgctcgtcttgtaaggctgctgcaggtttgacttgaatagcaaactgctcaaaaggaccatctttgcagtccactaaaaaatcagtagacataacctgatctacaccacgaataacaggatgcttaataccactatctattgcaatctgcctggtatcactaggtaataaggggaactgctcgcgtatatctagcacgctgctctcccactcaagactgagaaaaacagcaagctctaagtcagatagcaaatgatggactcgtcccgtcttatgagaataaatacggtgggaacgacctgaagaaggaacttcttgtactgttagccatggaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccaca

SEQ ID NO: 2 - DNA, 61 bp, Escherichia coli
  misc_feature (1)..(61): Sequences extending from -2, -1, 0, +1 +2, and +3 to +58 of the attachment site for Tn7 near the E. coli glmS gene, where positions -2 to +2 are duplicated as 5-bp sequences at both ends of a Tn7 element after transposition into this sequence.
cccgcttacgcagggcatccatttattactcaaccgtaaccgattttgccaggttacgcgg

SEQ ID NO: 3 - DNA, 13 bp, Artificial Sequence
  Junction of 5-bp duplication near Tn7L inserted between positions -2 to +2 of attTn7 near 3′ end of E. coli glmS gene.
  misc_feature (1)..(13): Junction of 5- duplication when Tn7 inserts betweenn positions -2 to +2 of attTn7L near 3′ end of E. coli glmS gene.
  misc_feature (1)..(13): Junction of 5-bp duplication when Tn7 inserts betweenn positions -2 to +2 of attTn7L near 3′ end of E. coli glmS gene.
cccgctgtgggcg

SEQ ID NO: 4 - DNA, 69 bp, Artificial Sequence
  Junction of 5-bp duplication near Tn7R inserted between positions -2 to +2 of attTn7 near 3′ end of E. coli glmS gene.
  misc_feature (1)..(69): Junction of 5-bp duplication when Tn7 inserts betweenn positions -2 to +2 of attTn7R near 3′ end of E. coli glmS gene.
cgcccacacccgcttacgcagggcatccatttattactcaaccgtaaccgattttgccaggttacgcgg

SEQ ID NO: 5 - DNA, 549 bp, Artificial Sequence
  Synthetic lacZ-alpha-mini-attTn7 sequence.
atgaccatgattacgccaagctcggaattaaccctcactaaagggaacaaaagctggagctccaccgcggtggcggccgctctagaactagtggatcccccgggctgcaggaattcacataacaggaagaaaaatgccccgcttacgcagggcatccatttattactcaaccgtaaccgattttgccaggttacgcggctgtcgacctcgagggggggcccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcttcgaagacgctcgacagcgacacacttgcatcggatgcagcccggttaacgtgccggcacggcctgggtaaccaggt

SEQ ID NO: 6 - DNA, 266 bp, Artificial Sequence
  Synthetic truncated lacZalpha-mini-attTn7
atgaccatgattacgaattcacataacaggaagaaaaatgccccgcttacgcagggcatccatttattactcaaccgtaaccgattttgccaggttacgcggctgtcgacttcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggtaa

SEQ ID NO: 7 - DNA, 76 bp, Artificial Sequence
  Sequences From the Tat/ScaI site to the BaeGI/ Bme1508I at the 3′ end of the Type I cat gene, adding SrfI and XmaI sites.
  CDS (1)..(33): Polypeptide sequence encoded at carboxy terminal region of Type I CAT protein, represented by QYCDEWQGGA*
cagtactgcgatgagtggcagggcggggcgtaatttttttaaggcagttattggtgcccgggcttaaacgcctggt

SEQ ID NO: 8 - protein, 10 aa, Artificial Sequence
  Synthetic Construct
QYCDEWQGGA

SEQ ID NO: 9 - DNA, 76 bp, Artificial Sequence
  Sequences From the Tat/ScaI site to the BaeGI/ Bme1508I at the 3′ end of the Type I cat gene, adding SrfI and XmaI sites, changing the GAT to a TAA stop codon.
cagtactgctaagagtggcagggcggggcgtaatttttttaaggcagttattggtgcccgggcttaaacgcctggt

SEQ ID NO: 10 - DNA, 76 bp, Artificial Sequence
  Sequences From the Tat/ScaI site to the BaeGI/ Bme1508I at the 3′ end of the Type I cat gene, adding SrfI and XmaI sites, changing the GAT to a TGA, stop codon.
cagtactgctgagagtggcagggcggggcgtaatttttttaaggcagttattggtgcccgggcttaaacgcctggt

SEQ ID NO: 11 - DNA, 76 bp, Artificial Sequence
  Sequences From the Tat/ScaI site to the BaeGI/ Bme1508I at the 3′ end of the Type I cat gene, adding SrfI and XmaI sites, changing the GAT to a TAG stop codon.
cagtactgctaggagtggcagggcggggcgtaatttttttaaggcagttattggtgcccgggcttaaacgcctggt

SEQ ID NO: 12 - DNA, 100 bp, Artificial Sequence
  3′ end of the Type I cat gene, adding SrfI and XmaI sites, before changing the GAT to a TAA, TGA, or TAG stop codon, and adding an overlapping mini-attTn7 site
  misc_feature (1)..(100): Sequences From the TatI/ScaI site to the BaeGI/ Bme1508I at the 3′ end of the Type I cat gene, adding SrfI and XmaI sites, before changing the GAT to a TAA, TGA, or TAG stop codon, and adding an mini-attTn7 site overlapping with the stop codon
cagtactgcgatcttacgcagggcatccatttattactcaaccgtaaccgattttgccaggttacgcggctgtcgacgtgcccgggcttaaacgcctggt

SEQ ID NO: 13 - DNA, 100 bp, Artificial Sequence
  Sequences From the Tat/ScaI site to the BaeGI/ Bme1508I at the 3′ end of the Type I cat gene, adding SrfI and XmaI sites, changing the GAT to a TAA stop codon, and adding an overlapping mini-attTn7 site.
  misc_feature (1)..(100): Sequences From the TatI/ScaI site to the BaeGI/ Bme1508I at the 3′ end of the Type I cat gene, adding SrfI and XmaI sites, changing the GAT to a TAA stop codon, and adding an mini-attTn7 site overlapping with the stop codon
cagtactgctaacttacgcagggcatccatttattactcaaccgtaaccgattttgccaggttacgcggctgtcgacgtgcccgggcttaaacgcctggt

SEQ ID NO: 14 - DNA, 100 bp, Artificial Sequence
  Sequences From the Tat/ScaI site to the BaeGI/ Bme1508I at the 3′ end of the Type I cat gene, adding SrfI and XmaI sites, changing the GAT to a TGA, stop codon, and adding an overlapping mini-attTn7 site.
  misc_feature (1)..(100): Sequences From the TatI/ScaI site to the BaeGI/ Bme1508I at the 3′ end of the Type I cat gene, adding SrfI and XmaI sites, changing the GAT to a TGA  stop codon, and adding an mini-attTn7 site overlapping with the stop codon
cagtactgctgacttacgcagggcatccatttattactcaaccgtaaccgattttgccaggttacgcggctgtcgacgtgcccgggcttaaacgcctggt

SEQ ID NO: 15 - DNA, 100 bp, Artificial Sequence
  Sequences From the Tat/ScaI site to the BaeGI/ Bme1508I at the 3′ end of the Type I cat gene, adding SrfI and XmaI sites, changing the GAT to a TAG stop codon, and adding an overlapping mini-attTn7 site.
  misc_feature (1)..(100): Sequences From the TatI/ScaI site to the BaeGI/ Bme1508I at the 3′ end of the Type I cat gene, adding SrfI and XmaI sites, changing the GAT to a TAA, TGA, or TAG stop codon, and adding an mini-attTn7 site overlapping with the stop codon
cagtactgctagcttacgcagggcatccatttattactcaaccgtaaccgattttgccaggttacgcggctgtcgacgtgcccgggcttaaacgcctggt

SEQ ID NO: 16 - DNA, 93 bp, Artificial Sequence
  Sequences From the TatI/ScaI site to the BaeGI/ Bme1508I at the 3′ end of Type I cat gene, adding SrfI and XmaI sites, changing the TGC to a TAA, TGA, or TAG stop codon, and the GAT to a TAA stop codon, adding mini-attTn7 overlapping with the first stop codon
  misc_feature (1)..(93): Sequences From the TatI/ScaI site to the BaeGI/ Bme1508I at the 3′ end of Type I cat gene, adding SrfI and XmaI sites, changing the TGC to a TAA, TGA, or TAG stop codon, and the GAT to a TAA stop codon, adding mini-attTn7 overlapping with the first stop codon
cagtactgcgatagggcatccatttattactcaaccgtaaccgattttgccaggttacgcggctgtcgacgtgcccgggcttaaacgcctggt

SEQ ID NO: 17 - DNA, 93 bp, Artificial Sequence
  Sequences From the TatI/ScaI site to the BaeGI/ Bme1508I at the 3′ end of Type I cat gene, adding SrfI and XmaI sites, changing the TGC to a TAA stop codon, and the GAT to a TAA stop codon, adding mini-attTn7 overlapping with the first stop codon
  misc_feature (1)..(93): Sequences From the TatI/ScaI site to the BaeGI/ Bme1508I at the 3′ end of Type I cat gene, adding SrfI and XmaI sites, changing the TGC to a TAA stop codon, and the GAT to a TAA stop codon, adding mini-attTn7 overlapping with the first stop codon
cagtactaataaagggcatccatttattactcaaccgtaaccgattttgccaggttacgcggctgtcgacgtgcccgggcttaaacgcctggt

SEQ ID NO: 18 - DNA, 93 bp, Artificial Sequence
  Sequences From the Tat/ScaI site to the BaeGI/ Bme1508I at the 3′ end of the Type I cat gene, adding SrfI and XmaI sites, changing the GAT to a TAG stop codon, and adding a long mini-attTn7 site.
  misc_feature (1)..(93): Sequences From the TatI/ScaI site to the BaeGI/ Bme1508I at the 3′ end of Type I cat gene, adding SrfI and XmaI sites, changing the TGC to a TGA stop codon, and the GAT to a TAA stop codon, adding mini-attTn7 overlapping with the first stop codon
cagtactgataaagggcatccatttattactcaaccgtaaccgattttgccaggttacgcggctgtcgacgtgcccgggcttaaacgcctggt

SEQ ID NO: 19 - DNA, 93 bp, Artificial Sequence
  Sequences From the Tat/ScaI site to the BaeGI/ Bme1508I at the 3′ end of the Type I cat gene, adding SrfI and XmaI sites, changing the GAT to a TGA stop codon, and adding a long mini-attTn7 site.
  misc_feature (1)..(93): Sequences From the TatI/ScaI site to the BaeGI/ Bme1508I at the 3′ end of Type I cat gene, adding SrfI and XmaI sites, changing the TGC to a TAG stop codon, and the GAT to a TAA stop codon, adding mini-attTn7 overlapping with the first stop codon
cagtactagtaaagggcatccatttattactcaaccgtaaccgattttgccaggttacgcggctgtcgacgtgcccgggcttaaacgcctggt

SEQ ID NO: 20 - DNA, 39 bp, Artificial Sequence
  Sequences at the 3′ end of a Type I cat gene after transposition of a mini-Tn7 into an over overlapping mini-attTn7 site.
  CDS (3)..(38): Polypeptide sequences 3′ end of a Type I cat gene after transposition of a mini-Tn7 into an over overlapping mini-attTn7 site.
gctttattaatcggctacaagagttgcgttatgtgggcg

SEQ ID NO: 21 - protein, 12 aa, Artificial Sequence
  Synthetic Construct
FINRLQELRYVG

SEQ ID NO: 22 - DNA, 22 bp, Artificial Sequence
  Synthetic sequence
  misc_feature (1)..(22): 3′ end of Tn7R after transposition an over overlapping mini-attTn7 site
cgcccacatgttattacgcagg

SEQ ID NO: 23 - DNA, 67 bp, Artificial Sequence
  3′ end of Type I cat gene to mimic insertion of Tn7L replacing stop codon for Cys codon
  misc_feature (1)..(67): Sequences at the 3′ end of a Type I cat gene that mimic Tn7L at the junction of mini-Tn7 replacing a stop codon for a Cys codon in an overlapping mini-attTn7 site
  CDS (1)..(24): Polypeptide sequence that mimics insertion of the Tn7L replacing the stop codon for a Cys codon, restoring activity to the encoded CAT fusion protein
cagtactgtgggcggacaaaatagggggcgtaatttttttaaggatcctattggtgcccgggctgcc

SEQ ID NO: 24 - protein, 7 aa, Artificial Sequence
  Synthetic Construct
QYCGRTK

SEQ ID NO: 25 - DNA, 180 bp, Escherichia coli
  CDS (1)..(180): 5′ end of E. coli lacZ gene nucleotides 1-180
atgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgc

SEQ ID NO: 26 - protein, 60 aa, Escherichia coli
MTMITDSLAVVLQRRDWENPGVTQLNRLAAHPPFASWRNSEEARTDRPSQQLRSLNGEWR

SEQ ID NO: 27 - DNA, 57 bp, Escherichia coli
  CDS (1)..(57): 5′ end of lacZ delta M15 gene of E. coli enoding amino acids 1-11 and 42 -49
atgaccatgattacggattcactggccgtcgtggcccgcaccgatcgcccttcccaa

SEQ ID NO: 28 - protein, 19 aa, Escherichia coli
MTMITDSLAVVARTDRPSQ

SEQ ID NO: 29 - DNA, 360 bp, Artificial Sequence
  LacZ alpha gene with MCS region pUC19 from positions 1-360
  CDS (1)..(324): LacZ alpha gene with MCS region pUC19 from positions 1-360
atgaccatgattacgccaagcttgcatgcctgcaggtcgactctagaggatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgac

SEQ ID NO: 30 - protein, 107 aa, Artificial Sequence
  Synthetic Construct
MTMITPSLHACRSTLEDPRVPSSNSLAVVLQRRDWENPGVTQLNRLAAHPPFASWRNSEEARTDRPSQQLRSLNGEWRLMRYFLLTHLCGISHRIWCTLSTICSDAA

SEQ ID NO: 31 - DNA, 260 bp, Escherichia coli
  CDS (1)..(186): Sequences from 1-260 of the lacZ gene, but polypetide sequence diverges areound nucleotide 186 compared to those in pUC19
atgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgt

SEQ ID NO: 32 - protein, 62 aa, Escherichia coli
MTMITDSLAVVLQRRDWENPGVTQLNRLAAHPPFASWRNSEEARTDRPSQQLRSLNGEWRFA

SEQ ID NO: 33 - DNA, 120 bp, Artificial Sequence
  PvuII to KasI sites of LacZ alpha gene pUC18 or pUC19
  CDS (1)..(120)
acccaacttaatcgccttgcagcacatccccctttcgccagctggcgtaatagcgaagaggcccgcaccgatcgcccttcccaacagttgcgcagcctgaatggcgaatggcgcctgatg

SEQ ID NO: 34 - protein, 40 aa, Artificial Sequence
  Synthetic Construct
TQLNRLAAHPPFASWRNSEEARTDRPSQQLRSLNGEWRLM

SEQ ID NO: 35 - DNA, 120 bp, Artificial Sequence
  PvuII to KasI sites of LacZ alpha gene pUC18 or pUC19 with synthetic oligonucleotides comprising two TAA stop codons near codons encoding NS
  CDS (1)..(54): PvuII to KasI sites of LacZ alpha gene pUC18 or pUC19 with synthetic oligonucleotides comprising two TAA stop codons near codons encoding NS
acccaacttaatcgccttgcagcacatccccctttcgccagctggcgttaataagaagaggcccgcaccgatcgcccttcccaacagttgcgcagcctgaatggcgaatggcgcctgatg

SEQ ID NO: 36 - protein, 16 aa, Artificial Sequence
  Synthetic Construct
TQLNRLAAHPPFASWR

SEQ ID NO: 37 - DNA, 120 bp, Artificial Sequence
  PvuII to KasI sites of LacZ alpha gene pUC18 or pUC19 with synthetic oligonucleotides comprising two TAA stop codons near codons encoding SE
  CDS (1)..(57)
acccaacttaatcgccttgcagcacatccccctttcgccagctggcgtaattaataagaggcccgcaccgatcgcccttcccaacagttgcgcagcctgaatggcgaatggcgcctgatg

SEQ ID NO: 38 - protein, 17 aa, Artificial Sequence
  Synthetic Construct
TQLNRLAAHPPFASWRN

SEQ ID NO: 39 - DNA, 120 bp, Artificial Sequence
  PvuII to KasI sites of LacZ alpha gene pUC18 or pUC19 with synthetic oligonucleotides comprising two TAA stop codons near codons encoding EE
  CDS (1)..(60): PvuII to KasI sites of LacZ alpha gene pUC18 or pUC19 with synthetic oligonucleotides comprising two TAA stop codons near codons encoding EE
acccaacttaatcgccttgcagcacatccccctttcgccagctggcgtaatagctaataagcccgcaccgatcgcccttcccaacagttgcgcagcctgaatggcgaatggcgcctgatg

SEQ ID NO: 40 - protein, 18 aa, Artificial Sequence
  Synthetic Construct
TQLNRLAAHPPFASWRNS

SEQ ID NO: 41 - DNA, 120 bp, Artificial Sequence
  PvuII to KasI sites of LacZ alpha gene pUC18 or pUC19 with synthetic oligonucleotides comprising two TAA stop codons near codons encoding EA
  CDS (1)..(63): PvuII to KasI sites of LacZ alpha gene pUC18 or pUC19 with synthetic oligonucleotides comprising two TAA stop codons near codons encoding EA
acccaacttaatcgccttgcagcacatccccctttcgccagctggcgtaatagcgaataataacgcaccgatcgcccttcccaacagttgcgcagcctgaatggcgaatggcgcctgatg

SEQ ID NO: 42 - protein, 19 aa, Artificial Sequence
  Synthetic Construct
TQLNRLAAHPPFASWRNSE

SEQ ID NO: 43 - DNA, 120 bp, Artificial Sequence
  PvuII to KasI sites of LacZ alpha gene pUC18 or pUC19 with synthetic oligonucleotides comprising two TAA stop codons near codons encoding AR
  CDS (1)..(66): PvuII to KasI sites of LacZ alpha gene pUC18 or pUC19 with synthetic oligonucleotides comprising two TAA stop codons near codons encoding AR
acccaacttaatcgccttgcagcacatccccctttcgccagctggcgtaatagcgaagagtaataaaccgatcgcccttcccaacagttgcgcagcctgaatggcgaatggcgcctgatg

SEQ ID NO: 44 - protein, 20 aa, Artificial Sequence
  Synthetic Construct
TQLNRLAAHPPFASWRNSEE

SEQ ID NO: 45 - DNA, 84 bp, Artificial Sequence
  PvuII to KasI sites of LacZ alpha gene pUC18 or pUC19
  CDS (1)..(84): PvuII to KasI sites of LacZ alpha gene pUC18 or pUC19
gccagctggcgtaatagcgaagaggcccgcaccgatcgcccttcccaacagttgcgcagcctgaatggcgaatggcgcctgatg

SEQ ID NO: 46 - protein, 28 aa, Artificial Sequence
  Synthetic Construct
ASWRNSEEARTDRPSQQLRSLNGEWRLM

SEQ ID NO: 47 - DNA, 84 bp, Artificial Sequence
  PvuII to KasI sites of LacZ alpha gene pUC18 or pUC19 with stop codons replacing SE codons
  misc_feature (1)..(84): PvuII to KasI sites of LacZ alpha gene pUC18 or pUC19 with stop codons replacing SE codons
gccagctggcgttaataagaagaggcccgcaccgatcgcccttcccaacagttgcgcagcctgaatggcgaatggcgcctgatg

SEQ ID NO: 48 - DNA, 84 bp, Artificial Sequence
  PvuII to KasI sites of LacZ alpha gene pUC18 or pUC19 with stop codons replacing NS codons
  misc_feature (1)..(84): PvuII to KasI sites of LacZ alpha gene pUC18 or pUC19 with stop codons replacing NS codons
gccagctggcgtaattaataagaggcccgcaccgatcgcccttcccaacagttgcgcagcctgaatggcgaatggcgcctgatg

SEQ ID NO: 49 - DNA, 84 bp, Artificial Sequence
  PvuII to KasI sites of LacZ alpha gene pUC18 or pUC19 with stop codons replacing EE codons
gccagctggcgtaatagctaataagcccgcaccgatcgcccttcccaacagttgcgcagcctgaatggcgaatggcgcctgatg

SEQ ID NO: 50 - DNA, 84 bp, Artificial Sequence
  PvuII to KasI sites of LacZ alpha gene pUC18 or pUC19 with stop codons replacing EA codons
  misc_feature (1)..(84): PvuII to KasI sites of LacZ alpha gene pUC18 or pUC19 with stop codons replacing EE codons
gccagctggcgtaatagcgaataataacgcaccgatcgcccttcccaacagttgcgcagcctgaatggcgaatggcgcctgatg

SEQ ID NO: 51 - DNA, 84 bp, Artificial Sequence
  PvuII to KasI sites of LacZ alpha gene pUC18 or pUC19 with stop codons replacing AR codons
  misc_feature (1)..(84): PvuII to KasI sites of LacZ alpha gene pUC18 or pUC19 with stop codons replacing EE codons
gccagctggcgtaatagcgaagagtaataaaccgatcgcccttcccaacagttgcgcagcctgaatggcgaatggcgcctgatg

SEQ ID NO: 52 - DNA, 85 bp, Artificial Sequence
  Synthetic mini-attTn7 from -2 to +2 with nunknown nucleotides at the insertion site, followed by +3 to +58, then Synthetic SalI, KasI and other restriction sites
  misc_feature (1)..(5): The 5 “n” residues from positions 1-5 each represent ANY nucleotde residue in the attachment site for Tn7 (attTn7) that are part of a 5-bp sequence that is duplicated at both ends of Tn7 after transposition.
  misc_feature (6)..(85): A mini-attTn7 target site from +3 to + 58 followed by synthetic SalI, KasI and other restriction sites
nnnnnttacgcagggcatccatttattactcaaccgtaaccgattttgccaggttacgcggctgtcgacgtgcccgggcggcgcc

SEQ ID NO: 53 - DNA, 43 bp, Artificial Sequence
  Sequences near double stop codons replacing EA codons in lacZalpha peptide after transposition of a mini-Tn7 into an overlapping mini-attTn7 site
  misc_feature (1)..(43): Seqiemces from -2 to  +41 of a mini-attTn7, including zero position between -2 and +2
aagagttacgcagggcatccatttattactcaaccgtaaccga

SEQ ID NO: 54 - DNA, 14 bp, Artificial Sequence
  Junction near target site after transptotion into TAA stop codon reading frame +1
agcgaatgtgggcg

SEQ ID NO: 55 - DNA, 15 bp, Artificial Sequence
  Junction near target site after transptotion into TAA stop codon reading frame +1
agcgaattgtgggcg

SEQ ID NO: 56 - DNA, 16 bp, Artificial Sequence
  Junction near target site after transptotion into TAA stop codon reading frame +3
agcgaatatgtgggcg

SEQ ID NO: 57 - DNA, 381 bp, Artificial Sequence
  pUC18 lacZalpha region containing an EcoRI-SalI fragment from  bMON14272 comprising a mini-attTn7 fragment
  CDS (1)..(381): Chimareic fusion protein comprising lacZalpha fragment with insertion of EcoRI-SalI fragment comprising a synthetic mini-attTn7 fragment
atgaccatgattacgaattcacataacaggaagaaaaatgccccgcttacgcagggcatccatttattactcaaccgtaaccgattttgccaggttacgcggctgtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatag

SEQ ID NO: 58 - protein, 126 aa, Artificial Sequence
  Synthetic Construct
MTMITNSHNRKKNAPLTQGIHLLLNRNRFCQVTRLSTCRHASLALAVVLQRRDWENPGVTQLNRLAAHPPFASWRNSEEARTDRPSQQLRSLNGEWRLMRYFLLTHLCGISHRIWCTLSTICSDAA

SEQ ID NO: 59 - DNA, 60 bp, Artificial Sequence
  Sequences near the unique PstI site in the beta lactamase gene of pACYC177
  CDS (1)..(60): Sequences near unique PstI site in pACYC177
ccaaacgacgagcgtgacaccacgatgcctgcagcaatggcaacaacgttgcgcaaacta

SEQ ID NO: 60 - protein, 20 aa, Artificial Sequence
  Synthetic Construct
PNDERDTTMPAAMATTLRKL

SEQ ID NO: 61 - DNA, 60 bp, Artificial Sequence
  paCYC177 with unique PistI site mutated to EagI site
  misc_feature (1)..(60): Sequences near unique PstI site in pACYC177 mutated to EagI site
ccaaacgacgagcgtgacaccacgatgccggccgcaatggcaacaacgttgcgcaaacta

SEQ ID NO: 62 - DNA, 60 bp, Artificial Sequence
  pACYC177 with unique PstIsite mutated  to unique PvuII site
  misc_feature (1)..(60): Sequences near unique PstI site mutated to unique PvuII site
ccaaacgacgagcgtgacaccacgatgccagctgcaatggcaacaacgttgcgcaaacta

SEQ ID NO: 63 - DNA, 60 bp, Artificial Sequence
  pACYC177 with PstI site near the 3′ end of the NPT-II gene that don′t change the amino acids “LQ” encoded by the wild-type gene
  CDS (1)..(48): Sequences near 3′ end of the NPT-II gene encoding a protein conferring resitance to kanamycin
gatatgaataaattgcagtttcatttgatgctcgatgagtttttctaatcagaattggtt

SEQ ID NO: 64 - protein, 15 aa, Artificial Sequence
  Synthetic Construct
DMNKLQFHLMLDEFF

SEQ ID NO: 65 - DNA, 60 bp, Artificial Sequence; identical to SEQ ID NO: 63
  pACYC177 with PstI site near 3′ end of NPT-II gene that do not change the amino acids encoded at that position
  CDS (1)..(48): Sequences near 3′ end of pACYC177 with a new PstI site that don′t change amino acids “LQ” encoded at that postion in the NPT-II gene

SEQ ID NO: 66 - protein, 15 aa, Artificial Sequence
  Synthetic Construct
DMNKLQFHLMLDEFF

SEQ ID NO: 67 - DNA, 51 bp, Artificial Sequence
  pKM2 3′ end of NPT-II gene
  CDS (1)..(21): pKM2 3′ end of NPT-II gene
cttcttgacgagttcttctgagcgggactctggggttcgaaatgaccacca

SEQ ID NO: 68 - protein, 6 aa, Artificial Sequence
  Synthetic Construct
LLDEFF

SEQ ID NO: 69 - DNA, 27 bp, Artificial Sequence
  pKM243 3′ end of NPT-II gene
  CDS (1)..(27): pKM243 3′ end of NPT-II gene
cttcttgacgagttcttccaagcttaa

SEQ ID NO: 70 - protein, 8 aa, Artificial Sequence
  Synthetic Construct
LLDEFFQA

SEQ ID NO: 71 - DNA, 18 bp, Artificial Sequence
  pKM243/1 3′ end of NPT-II gene
  CDS (1)..(18): pKM243/1 3′ end of NPT-II gene
cttcttgacgagttcttc

SEQ ID NO: 72 - protein, 6 aa, Artificial Sequence
  Synthetic Construct
LLDEFF

SEQ ID NO: 73 - DNA, 51 bp, Artificial Sequence
  pKM143-1 3′ end of NPT-II gene
  CDS (1)..(51): pKM243-1 3′ end pf NPT-II gene
cttcttgacgagttcttcccaagctttaatgcggtagtttatcacagttaa

SEQ ID NO: 74 - protein, 16 aa, Artificial Sequence
  Synthetic Construct
LLDEFFPSFNAVVYHS

SEQ ID NO: 75 - DNA, 43 bp, Artificial Sequence
  pACYC177 3′ end of NPT-II gene
  CDS (1)..(21): pACYC177 3′ end of NPT-II gene
atgctcgatgagtttttctaatcagaattggttaattggttgt

SEQ ID NO: 76 - protein, 6 aa, Artificial Sequence
  Synthetic Construct
MLDEFF

SEQ ID NO: 77 - DNA, 43 bp, Artificial Sequence
  pACYC177-QA 3′ end of NPT-II gene
  CDS (1)..(27): pACYC177-QA 3′ end of NPT-II gene
atgctcgatgagtttttccaagcttaattggttaattggttgt

SEQ ID NO: 78 - protein, 8 aa, Artificial Sequence
  Synthetic Construct
MLDEFFQA

SEQ ID NO: 79 - DNA, 43 bp, Artificial Sequence
  pACYC177-PS 3′ end of NPT-II gene
  CDS (1)..(27): pACYC177-PS 3′ end of NPT-II gene
atgctcgatgagtttttcccaagctaattggttaattggttgt

SEQ ID NO: 80 - protein, 8 aa, Artificial Sequence
  Synthetic Construct
MLDEFFPS

SEQ ID NO: 81 - DNA, 51 bp, Artificial Sequence
  pACYC177-PSFNAVVYHS 3′ end of NPT-II gene
  CDS (1)..(51): pACYC177-PSFNAVVYHS 3′ end of NPT-II gene
atgctcgatgagtttttcccaagctttaatgcggtagtttatcacagttaa

SEQ ID NO: 82 - protein, 16 aa, Artificial Sequence
  Synthetic Construct
MLDEFFPSFNAVVYHS

SEQ ID NO: 83 - DNA, 43 bp, Artificial Sequence
  pACYC177-Q** with two TAA stop codons after Q codon
  CDS (1)..(27): pACYC177-Q** with two stop codons after a Q codon
atgctcgatgagtttttccaataataattggttaattggttgt

SEQ ID NO: 84 - protein, 7 aa, Artificial Sequence
  Synthetic Construct
MLDEFFQ

SEQ ID NO: 85 - DNA, 43 bp, Artificial Sequence
  pACYC177-P** with two TAA stop codons after a P codon
  CDS (1)..(27): pACYC177-PS with two TAA stop codons after a P codon
atgctcgatgagtttttcccataataattggttaattggttgt

SEQ ID NO: 86 - protein, 7 aa, Artificial Sequence
  Synthetic Construct
MLDEFFP

SEQ ID NO: 87 - DNA, 50 bp, Artificial Sequence
  pACYC177 3′ end of beta-lactamase gene
  CDS (2)..(28): pACYC177 3′ end of beta lactamase gene
aggtgcctcactgattaagcattggtaactgtcagaccaagtttactcat

SEQ ID NO: 88 - protein, 8 aa, Artificial Sequence
  Synthetic Construct
GASLIKHW

SEQ ID NO: 89 - DNA, 50 bp, Artificial Sequence
  pACYC177-K*** with two TAA stop codons befoe the normal TAA stop codon
  CDS (2)..(25): pACYC177-K*** with two TAA stop codons befoe the normal TAA stop codon
aggtgcctcactgattaagtaataataactgtcagaccaagtttactcat

SEQ ID NO: 90 - protein, 6 aa, Artificial Sequence
  Synthetic Construct
GASLIK

SEQ ID NO: 91 - DNA, 50 bp, Artificial Sequence
  pACYC177-KH**
  CDS (2)..(28): pACYC177-KH** with two stop codons after KH, one replacing “essential Tryptophan (W) codon
aggtgcctcactgattaagcattaataactgtcagaccaagtttactcat

SEQ ID NO: 92 - protein, 7 aa, Artificial Sequence
  Synthetic Construct
GASLIKH

SEQ ID NO: 93 - DNA, 50 bp, Artificial Sequence
  pACYC177-KWH**
  CDS (2)..(31): pACYC177-KHW** with two stop codons at site of normal TAA stop codon
aggtgcctcactgattaagcattggtaataatcagaccaagtttactcat

SEQ ID NO: 94 - protein, 8 aa, Artificial Sequence
  Synthetic Construct
GASLIKHW

SEQ ID NO: 95 - DNA, 11 bp, Artificial Sequence
  pACYC177-AAG
aagtgtgggcg

SEQ ID NO: 96 - DNA, 12 bp, Artificial Sequence
  pACYC177-AAGT
aagttgtgggcg

SEQ ID NO: 97 - DNA, 13 bp, Artificial Sequence
  pACYC177-AAGTA
aagtatgtgggcg

SEQ ID NO: 98 - DNA, 14 bp, Artificial Sequence
  pACYC177-AAGCAT
aagcattgtgggcg

SEQ ID NO: 99 - DNA, 15 bp, Artificial Sequence
  pACYC177-AAGCATTT
aagcatttgtgggcg

SEQ ID NO: 100 - DNA, 16 bp, Artificial Sequence
  pACYC177-AAGCATTA
aagcattatgtgggcg

SEQ ID NO: 101 - DNA, 17 bp, Artificial Sequence
  pACYC177-AAGCATTGG
aagcattggtgtgggcg

SEQ ID NO: 102 - DNA, 18 bp, Artificial Sequence
  pACYC1770AAGCATTGGT
aagcattggttgtgggcg

SEQ ID NO: 103 - DNA, 19 bp, Artificial Sequence
  pACYC177-AAGCATTGGTA
aagcattggtatgtgggcg

SEQ ID NO: 104 - DNA, 141 bp, Artificial Sequence
  pACUC177-PstI-BglI spanning junction between alpha and omega fragments of beta-lactamase
  CDS (1)..(141): pACUC177-PstI-BglI spanning junction between alpha and omega fragments of beta-lactamase
atgcctgcagcaatggcaacaacgttgcgcaaactattaactggcgaactacttactctagcttcccggcaacaattaatagactggatggaggcggataaagttgcaggaccacttctgcgctcggcccttccggctggc

SEQ ID NO: 105 - protein, 47 aa, Artificial Sequence
  Synthetic Construct
MPAAMATTLRKLLTGELLTLASRQQLIDWMEADKVAGPLLRSALPAG

SEQ ID NO: 106 - DNA, 105 bp, Artificial Sequence
  pACYC177-Pst-AseI with linker
  CDS (1)..(105): pACYC177-PstI-AseI with synthetic linker at junction of alpha and omega fragments of beta lactamase
atgcctgcagcaatggcaacaacgttgcgcaaactattaactggcgaaacgaattcgcggccgcgtcgacgtctacttactctagcttcccggcaacaattaata

SEQ ID NO: 107 - protein, 35 aa, Artificial Sequence
  Synthetic Construct
MPAAMATTLRKLLTGETNSRPRRRLLTLASRQQLI

SEQ ID NO: 108 - DNA, 180 bp, Artificial Sequence
  pACYC177-bla-alpha-omega-mini-attTn7
  CDS (1)..(180): pACYC177-bla-alpha-omega-mini-attTn7 with mini-attTn7 at the junction of the alpha and omega peptides of beta-lactamase
atgcctgcagcaatggcaacaacgttgcgcaaactattaactggcgaaacgaattcacataacaggaagaaaaatgccccgcttacgcagggcatccatttattactcaaccgtaaccgattttgccaggttacgcgggtcgaccgtctacttactctagcttcccggcaacaattaata

SEQ ID NO: 109 - protein, 60 aa, Artificial Sequence
  Synthetic Construct
MPAAMATTLRKLLTGETNSHNRKKNAPLTQGIHLLLNRNRFCQVTRVDRLLTLASRQQLI

SEQ ID NO: 110 - protein, 401 aa, Escherichia coli
  DOMAIN (181)..(207): Interdomain loop in Tn10 tetracycline resistance protein ETKNTRDNTDTEVGVETQSNSVYITLF
MNSSTKIALVITLLDAMGIGLIMPVLPTLLREFIASEDIANHFGVLLALYALMQVIFAPWLGKMSDRFGRRPVLLLSLIGASLDYLLLAFSSALWMLYLGRLLSGITGATGAVAASVIADTTSASQRVKWFGWLGASFGLGLIAGPIIGGFAGEISPHSPFFIAALLNIVTFLVVMFWFRETKNTRDNTDTEVGVETQSNSVYITLFKTMPILLIIYFSAQLIGQIPATVWVLFTENRFGWNSMMVGFSLAGLGLLHSVFQAFVAGRIATKWGEKTAVLLGFIADSSAFAFLAFISEGWLVFPVLILLAGGGIALPALQGVMSIQTKSHQQGALQGLLVSLTNATGVIGPLLFAVIYNHSLPIWDGWIWIIGLAFYCIIILLSMTFMLTPQAQGSKQETSA

SEQ ID NO: 111 - protein, 396 aa, Shigella flexneri
  DOMAIN (183)..(209): Interdomain loop in pACYC184 tetraccyline gene indrectly derived from pSC101 isolated from Shigella flexneri ESHKGERRPMPLRAFNPVSSFRWARGM
MKSNNALIVILGTVTLDAVGIGLVMPVLPGLLRDIVHSDSIASHYGVLLALYALMQFLCAPVLGALSDRFGRRPVLLASLLGATIDYAIMATTPVLWILYAGRIVAGITGATGAVAGAYIADITDGEDRARHFGLMSACFGVGMVAGPVAGGLLGAISLHAPFLAAAVLNGLNLLLGCFLMQESHKGERRPMPLRAFNPVSSFRWARGMTIVAALMTVFFIMQLVGQVPAALWVIFGEDRFRWSATMIGLSLAVFGILHALAQAFVTGPATKRFGEKQAIIAGMAADALGYVLLAFATRGWMAFPIMILLASGGIGMPALQAMLSRQVDDDHQGQLQGSLAALTSLTSIIGPLIVTAIYAASASTWNGLAWIVGAALYLVCLPALRRGAWSRATST

SEQ ID NO: 112 - DNA, 210 bp, Artificial Sequence
  pACYC184 reverse complement spanning Tet Iinterdomain Loop
  CDS (1)..(210): Sequence from the reverse complement of pACYC184 flanking the interdomain loop of the tetracycline resistance protein
tccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacag

SEQ ID NO: 113 - protein, 70 aa, Artificial Sequence
  Synthetic Construct
SLHAPFLAAAVLNGLNLLLGCFLMQESHKGERRPMPLRAFNPVSSFRWARGMTIVAALMTVFFIMQLVGQ

SEQ ID NO: 114 - DNA, 297 bp, Artificial Sequence
  pACYC184 reverse complement Tet-mini-attTn7
  CDS (1)..(297): pACYC184 reverse complement Tet-mini-attTn7, with synthetic mini-attTn7 inserted near SalI site in the sequences encoding the interdomain linker of the tetracycline resistance protein
tccttgcatgcaccattccttgcggcggcggtgctcaacggcctcaacctactactgggctgcttcctaatgcaggagtcgcataagggagagaattcacataacaggaagaaaaatgccccgcttacgcagggcatccatttattactcaaccgtaaccgattttgccaggttacgcggcgtcgaccgatgcccttgagagccttcaacccagtcagctccttccggtgggcgcggggcatgactatcgtcgccgcacttatgactgtcttctttatcatgcaactcgtaggacag

SEQ ID NO: 115 - protein, 99 aa, Artificial Sequence
  Synthetic Construct
SLHAPFLAAAVLNGLNLLLGCFLMQESHKGENSHNRKKNAPLTQGIHLLLNRNRFCQVTRRRPMPLRAFNPVSSFRWARGMTIVAALMTVFFIMQLVGQ

SEQ ID NO: 116 - DNA, 95 bp, Artificial Sequence
  An EcoRI-SalI fragment comrpising a synthetic mini-attTn7
  misc_feature (1)..(95): EcoRI-SalI fragment comprising a synthetic mini-attTn7
gaattcacataacaggaagaaaaatgccccgcttacgcagggcatccatttattactcaaccgtaaccgattttgccaggttacgcggcgtcgac

SEQ ID NO: 117 - DNA, 22 bp, Artificial Sequence
  Syntheitc Not-PspOMI linker
  misc_feature (1)..(22): Synthetic linker comprising NotI and PspOMI sites GC′GGCC,GC nnnnnnnn G′GGCC,C
  misc_feature (9)..(16): N repreasents any nucleotide
gcggccgcnnnnnnnngggccc

SEQ ID NO: 118 - DNA, 37 bp, Artificial Sequence
  Synthetic Linker with NotI-scar-PspOMI sites
  misc_feature (1)..(37): Synthetic linker with NotI-scar-PspOMI sites GC′GGCC,GC nnnnnnnn GGGCCGC nnnnnnnn G′GGCC,C
  misc_feature (9)..(16): N repreasents any nucleotide
  misc_feature (24)..(31): N repreasents any nucleotide
gcggccgcnnnnnnnngggccgcnnnnnnnngggccc

SEQ ID NO: 119 - DNA, 22 bp, Artificial Sequence
  PspOMI-NotI linker
  misc_feature (1)..(22): Synthetic PspOMI-NotI linker
gggcccnnnnnnnngcggccgc

SEQ ID NO: 120 - DNA, 37 bp, Artificial Sequence
  Synthetic PspOMI-scar-NotI linker
  misc_feature (1)..(37): Synthetic PspOMI-scar--NotI linker
gggcccnnnnnnnngcggcccnnnnnnnngcggccgc

SEQ ID NO: 121 - DNA, 24 bp, Artificial Sequence
  Syntheitc AbsI-SgrDI linker
  misc_feature (1)..(24): Synthetic AbsI-SgrDI linker
cctcgaggnnnnnnnncgtcgacg

SEQ ID NO: 122 - DNA, 40 bp, Artificial Sequence
  Synthetic AbsI-scar-SgrDI linker
  misc_feature (1)..(40): Synthetic AbsI-scar-SgrDI linker
cctcgaggnnnnnnnncgtcgaggnnnnnnnncgtcgacg

SEQ ID NO: 123 - DNA, 24 bp, Artificial Sequence
  Synthetic SgrDI-AbsI linker
  misc_feature (1)..(24): Synthetic SgrDI-AbsI linker
cgtcgacgnnnnnnnncctcgagg

SEQ ID NO: 124 - DNA, 40 bp, Artificial Sequence
  Synthetic SgrDI-scar-AbsI linker
  misc_feature (1)..(40): Synthetic SgrDI-scar-AbsI linker
cgtcgacgnnnnnnnncctcgacgnnnnnnnncctcgagg

SEQ ID NO: 125 - DNA, 24 bp, Artificial Sequence
  Synthetic MauBI-AscI linker
  misc_feature (1)..(24): Synthetic MauBI-AscI linker
cgcgcgcgnnnnnnnnggcgcgcc

SEQ ID NO: 126 - DNA, 40 bp, Artificial Sequence
  Synthetic MauBI-scar-AscI linker
  misc_feature (1)..(40): Synthetic MauBI-scar-AscI linker
cgcgcgcgnnnnnnnnggcgcgcgnnnnnnnnggcgcgcc

SEQ ID NO: 127 - DNA, 24 bp, Artificial Sequence
  Synthetic AscI-MauBI linker
  misc_feature (1)..(24): Synthetic AscI-MauBI linker
ggcgcgccnnnnnnnncgcgcgcg

SEQ ID NO: 128 - DNA, 40 bp, Artificial Sequence
  Synthetic AscI-scar-MauBI linker
  misc_feature (1)..(40): Synthetic AscI-scar-MauBI linker
ggcgcgccnnnnnnnncgcgcgccnnnnnnnncgcgcgcg

SEQ ID NO: 129 - DNA, 24 bp, Artificial Sequence
  Synthetic MauBI-AbsI linker
  misc_feature (1)..(24): Synthetic MauBI-AbsI linker
cgcgcgcgnnnnnnnncctcgagg

SEQ ID NO: 130 - DNA, 24 bp, Artificial Sequence
  Syntheitc MauBI-SgrDI linker
  misc_feature (1)..(24): Syntheitc MauBI-SgrDI linker
cgcgcgcgnnnnnnnncgtcgacg

SEQ ID NO: 131 - DNA, 24 bp, Artificial Sequence
  Synthetic AscI-AbsI linker
  misc_feature (1)..(24): Synthetic AscI-AbsI linker
ggcgcgccnnnnnnnncctcgagg

SEQ ID NO: 132 - DNA, 24 bp, Artificial Sequence
  Synthteit AscI-SgrDI linker
  misc_feature (1)..(24): Synthteit AscI-SgrDI linker
ggcgcgccnnnnnnnncgtcgacg

SEQ ID NO: 133 - DNA, 24 bp, Artificial Sequence
  Synthetic AbsI-MauBI linker
  misc_feature (1)..(24): Synthetic AbsI-MauBI linker
cctcgaggnnnnnnnncgcgcgcg

SEQ ID NO: 134 - DNA, 24 bp, Artificial Sequence
  Synthetic AbsI-AscI linker
  misc_feature (1)..(24): Synthetic AbsI-AscI linker
cctcgaggnnnnnnnnggcgcgcc

SEQ ID NO: 135 - DNA, 24 bp, Artificial Sequence
  Synthetic SgrDI-MauBI linker
  misc_feature (1)..(24): Synthetic SgrDI-MauBI linker
cgtcgacgnnnnnnnncgcgcgcg

SEQ ID NO: 136 - DNA, 24 bp, Artificial Sequence
  Synhtetic SgrDI-AscI linker
  misc_feature (1)..(24): Syntheitc SgrDI-AscI linker
cgtcgacgnnnnnnnnggcgcgcc

SEQ ID NO: 137 - DNA, 24 bp, Artificial Sequence
  Synthetic MauBI-PacI-AbsI linker
  misc_feature (1)..(24): Synthetic MauBI-PacI-AbsI linker
cgcgcgcgttaattaacctcgagg

SEQ ID NO: 138 - DNA, 24 bp, Artificial Sequence
  Syntheitc MauBI-PacI-SgrDI linker
  misc_feature (1)..(24): Syntheitc MauBI-PacI-SgrDI linker
cgcgcgcgttaattaacgtcgacg

SEQ ID NO: 139 - DNA, 24 bp, Artificial Sequence
  Synthetic AscI-PacI-AbsI linker
  misc_feature (1)..(24): Synthetic AscI-PacI-AbsI linker
ggcgcgccttaattaacctcgagg

SEQ ID NO: 140 - DNA, 24 bp, Artificial Sequence
  Synthetic AscI-PacI-SgrDI linker
  misc_feature (1)..(24): Synthetic AscI-PacI-SgrDI linker
ggcgcgccttaattaacgtcgacg

SEQ ID NO: 141 - DNA, 24 bp, Artificial Sequence
  Synthetic AbsI-PacI-MauBI linker
  misc_feature (1)..(24): Synthetic AbsI-PacI-MauBI linker
cctcgaggttaattaacgcgcgcg

SEQ ID NO: 142 - DNA, 24 bp, Artificial Sequence
  Synthetic AbsI-PacI-AscI linker
  misc_feature (1)..(24): Synthetic AbsI-PacI-AscI linker
cctcgaggttaattaaggcgcgcc

SEQ ID NO: 143 - DNA, 24 bp, Artificial Sequence
  Synthetic SgrDI-PacI-MauBI linker
  misc_feature (1)..(24): Synthetic SgrDI-PacI-MauBI linker
cgtcgacgttaattaacgcgcgcg

SEQ ID NO: 144 - DNA, 24 bp, Artificial Sequence
  Synthetic SgrDI-PacI-AscI linker
  misc_feature (1)..(24): SgrDI-PacI-AscI linker
cgtcgacgttaattaaggcgcgcc

SEQ ID NO: 145 - DNA, 54 bp, Artificial Sequence
  Synthetic MauBI-PacI-AbsI-AvrII-SgrDI-PacI-AscI linker
  misc_feature (1)..(54): Synthetic MauBI-PacI-AbsI-AvrII-SgrDI-PacI-AscI linker
cgcgcgcgttaattaacctcgaggcctaggcgtcgacgttaattaaggcgcgcc

SEQ ID NO: 146 - DNA, 54 bp, Artificial Sequence
  MauBI-PacI-SgrDI-AvrII-AbsI-PacI-AscI linker
  misc_feature (1)..(54): MauBI-PacI-SgrDI-AvrII-AbsI-PacI-AscI linker
cgcgcgcgttaattaacgtcgacgcctaggcctcgaggttaattaaggcgcgcc

SEQ ID NO: 147 - DNA, 54 bp, Artificial Sequence
  Synthetic AscI-PacI-AbsI-AvrII-SgrDI-PacI-MauBI linker
  misc_feature (1)..(54): Synthetic AscI-PacI-AbsI-AvrII-SgrDI-PacI-MauBI linker
ggcgcgccttaattaacctcgaggcctaggcgtcgacgttaattaacgcgcgcg

SEQ ID NO: 148 - DNA, 54 bp, Artificial Sequence
  Synthetic AscI-PacI-SgrDI-AvrII-AbsI-PacI-MauBI linker
  misc_feature (1)..(54): Synthetic AscI-PacI-SgrDI-AvrII-AbsI-PacI-MauBI linker
ggcgcgccttaattaacgtcgacgcctaggcctcgaggttaattaacgcgcgcg

SEQ ID NO: 149 - DNA, 54 bp, Artificial Sequence
  Synthetic AbsI-PacI-MauBI-AvrII-AscI-PacI-SgrDI linker
  misc_feature (1)..(54): Synthetic AbsI-PacI-MauBI-AvrII-AscI-PacI-SgrDI linker
cctcgaggttaattaacgcgcgcgcctaggggcgcgccttaattaacgtcgacg

SEQ ID NO: 150 - DNA, 54 bp, Artificial Sequence
  AbsI-PacI-AscI-AvrII-MauBI-PacI-SgrDI linker
  misc_feature (1)..(54): Synthetic AbsI-PacI-AscI-AvrII-MauBI-PacI-SgrDI linker
cctcgaggttaattaaggcgcgcccctaggcgcgcgcgttaattaacgtcgacg

SEQ ID NO: 151 - DNA, 54 bp, Artificial Sequence
  Synthetic SgrDI-PacI-MauBI-AvrII-AscI-PacI-AbsI linker
  misc_feature (1)..(54): Synthetic SgrDI-PacI-MauBI-AvrII-AscI-PacI-AbsI linker
cgtcgacgttaattaacgcgcgcgcctaggggcgcgccttaattaacctcgagg

SEQ ID NO: 152 - DNA, 54 bp, Artificial Sequence
  Synthetic SgrDI-PacI-AscI-AvrII-MauBI-PacI-AbsI linker
  misc_feature (1)..(54): Synthetic SgrDI-PacI-AscI-AvrII-MauBI-PacI-AbsI linker
cgtcgacgttaattaaggcgcgcccctaggcgcgcgcgttaattaacctcgagg

SEQ ID NO: 153 - DNA, 24 bp, Artificial Sequence
  Syntheitc MauBI-PacI-AscI linker
  misc_feature (1)..(24): Syntheitc MauBI-PacI-AscI linker
cgcgcgcgttaattaaggcgcgcc

SEQ ID NO: 154 - DNA, 24 bp, Artificial Sequence
  Synthetic AscI-PacI-MauBI linker
  misc_feature (1)..(24): Synthetic AscI-PacI-MauBI linker
ggcgcgccttaattaacgcgcgcg

SEQ ID NO: 155 - DNA, 24 bp, Artificial Sequence
  Synteitc AscI-PacI-SgrDI linker
  misc_feature (1)..(24): Synthetic AscI-PacI-SgrDI linker
cctcgaggttaattaacgtcgacg

SEQ ID NO: 156 - DNA, 24 bp, Artificial Sequence
  Synthetic SgrDI-PacI-AbsI linker
  misc_feature (1)..(24): Syntehitc SgrDI-PacI-AbsI linker
cgtcgacgttaattaacctcgagg

SEQ ID NO: 157 - DNA, 2007 bp, Artificial Sequence
  pTwist-Kan-MC vector
cgcgcgcgttaattaacctcgaggcctaggcgtcgacgttaattaaggcgcgccaggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaagtctgacgctctattcaacaaagccgccgtccatgggtagggggcttcaaatcgtccccccatacgatataagttgttactagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattgatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacct

SEQ ID NO: 158 - DNA, 2159 bp, Artificial Sequence
  pTwist-Kan-MC vector with MauBI-PacI-AbsI-AvrII-SgrDI-PacI-AscI polylinker
cgcgcgcgttaattaacctcgaggcctaggcgtcgacgttaattaaggcgcgccaggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaagtctgacgctctattcaacaaagccgccgtccatgggtagggggcttcaaatcgtccgctctgccagtgttacaaccaattaacaaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttcccccgccgctctagaactagtggatccaaataaaacgaaaggctcagtcgaaagactgggcctttcgttttatctgttgtttgtcgcattatacgagacgtccaggttgggatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacct

SEQ ID NO: 159 - DNA, 876 bp, Artificial Sequence
  Synthetic DNA
  CDS (163)..(822): cat gene from pACYC184
cgcgcgcgttaattaacctcgaggcctccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccgggccgtcgacgttaattaaggcgcgcc

SEQ ID NO: 160 - protein, 219 aa, Artificial Sequence
  Synthetic Construct
MEKKITGYTTVDISQWHRKEHFEAFQSVAQCTYNQTVQLDITAFLKTVKKNKHKFYPAFIHILARLMNAHPEFRMAMKDGELVIWDSVHPCYTVFHEQTETFSSLWSEYHDDFRQFLHIYSQDVACYGENLAYFPKGFIENMFFVSANPWVSFTSFDLNVANMDNFFAPVFTMGKYYTQGDKVLMPLAIQVHHAVCDGFHVGRMLNELQQYCDEWQGGA

SEQ ID NO: 161 - DNA, 876 bp, Artificial Sequence
  Synthetic DNA
  CDS (163)..(801): cat gene from pACYC184 with one TAA stop codon
cgcgcgcgttaattaacctcgaggcctccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgctaagagtggcagggcggggcgtaatttttttaaggcagttattggtgcccgggccgtcgacgttaattaaggcgcgcc

SEQ ID NO: 162 - protein, 212 aa, Artificial Sequence
  Synthetic Construct
MEKKITGYTTVDISQWHRKEHFEAFQSVAQCTYNQTVQLDITAFLKTVKKNKHKFYPAFIHILARLMNAHPEFRMAMKDGELVIWDSVHPCYTVFHEQTETFSSLWSEYHDDFRQFLHIYSQDVACYGENLAYFPKGFIENMFFVSANPWVSFTSFDLNVANMDNFFAPVFTMGKYYTQGDKVLMPLAIQVHHAVCDGFHVGRMLNELQQYC

SEQ ID NO: 163 - DNA, 876 bp, Artificial Sequence
  Synthetic DNA
  CDS (163)..(801): cat gene from pACYC184 with two TAA stop codons
cgcgcgcgttaattaacctcgaggcctccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactaataagagtggcagggcggggcgtaatttttttaaggcagttattggtgcccgggccgtcgacgttaattaaggcgcgcc

SEQ ID NO: 164 - protein, 211 aa, Artificial Sequence
  Synthetic Construct
MEKKITGYTTVDISQWHRKEHFEAFQSVAQCTYNQTVQLDITAFLKTVKKNKHKFYPAFIHILARLMNAHPEFRMAMKDGELVIWDSVHPCYTVFHEQTETFSSLWSEYHDDFRQFLHIYSQDVACYGENLAYFPKGFIENMFFVSANPWVSFTSFDLNVANMDNFFAPVFTMGKYYTQGDKVLMPLAIQVHHAVCDGFHVGRMLNELQQY

SEQ ID NO: 165 - DNA, 889 bp, Artificial Sequence
  Synthetic DNA
  CDS (163)..(801): cat gene from pACYC184 and two TAA stop codons followed by mini-attTn7 target site
cgcgcgcgttaattaacctcgaggcctccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactaataaagggcatccatttattactcaaccgtaaccgattttgccaggttacgcggctgggtgcccgggccgtcgacgttaattaaggcgcgcc

SEQ ID NO: 166 - protein, 211 aa, Artificial Sequence; identical to SEQ ID NO: 164
  Synthetic Construct

SEQ ID NO: 167 - DNA, 896 bp, Artificial Sequence
  Synthetic DNA
  CDS (163)..(813): gene fusion comprising cat gene from pACYC194 fused to reading frame 1 from end of Tn7L
cgcgcgcgttaattaacctcgaggcctccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgtgggcggacaaaatagttgggaactgggaggggtggaaatggagtttttaaggattatttagggggtgcccgggccgtcgacgttaattaaggcgcgcc

SEQ ID NO: 168 - protein, 216 aa, Artificial Sequence
  Synthetic Construct
MEKKITGYTTVDISQWHRKEHFEAFQSVAQCTYNQTVQLDITAFLKTVKKNKHKFYPAFIHILARLMNAHPEFRMAMKDGELVIWDSVHPCYTVFHEQTETFSSLWSEYHDDFRQFLHIYSQDVACYGENLAYFPKGFIENMFFVSANPWVSFTSFDLNVANMDNFFAPVFTMGKYYTQGDKVLMPLAIQVHHAVCDGFHVGRMLNELQQYCGRTK

SEQ ID NO: 169 - DNA, 897 bp, Artificial Sequence
  Synthetic DNA
  CDS (163)..(849): gene fusion comprising cat gene from pACYC194 fused to reading frame 2 from end of Tn7L
cgcgcgcgttaattaacctcgaggcctccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtacttgtgggcggacaaaatagttgggaactgggaggggtggaaatggagtttttaaggattatttagggggtgcccgggccgtcgacgttaattaaggcgcgcc

SEQ ID NO: 170 - protein, 228 aa, Artificial Sequence
  Synthetic Construct
MEKKITGYTTVDISQWHRKEHFEAFQSVAQCTYNQTVQLDITAFLKTVKKNKHKFYPAFIHILARLMNAHPEFRMAMKDGELVIWDSVHPCYTVFHEQTETFSSLWSEYHDDFRQFLHIYSQDVACYGENLAYFPKGFIENMFFVSANPWVSFTSFDLNVANMDNFFAPVFTMGKYYTQGDKVLMPLAIQVHHAVCDGFHVGRMLNELQQYLWADKIVGNWEGWKWSF

SEQ ID NO: 171 - DNA, 898 bp, Artificial Sequence
  Synthetic DNA
  CDS (163)..(822): gene fusion comprising cat gene from pACYC194 fused to reading frame 3 from end of Tn7L
cgcgcgcgttaattaacctcgaggcctccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactatgtgggcggacaaaatagttgggaactgggaggggtggaaatggagtttttaaggattatttagggggtgcccgggccgtcgacgttaattaaggcgcgcc

SEQ ID NO: 172 - protein, 220 aa, Artificial Sequence
  Synthetic Construct
MEKKITGYTTVDISQWHRKEHFEAFQSVAQCTYNQTVQLDITAFLKTVKKNKHKFYPAFIHILARLMNAHPEFRMAMKDGELVIWDSVHPCYTVFHEQTETFSSLWSEYHDDFRQFLHIYSQDVACYGENLAYFPKGFIENMFFVSANPWVSFTSFDLNVANMDNFFAPVFTMGKYYTQGDKVLMPLAIQVHHAVCDGFHVGRMLNELQQYYVGGQNSWE

SEQ ID NO: 173 - DNA, 1953 bp, Artificial Sequence
  pTwist-Chlor-MC cloining vector
aggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaagtctgacgctctattcaacaaagccgccgtccatgggtagggggcttcaaatcgtccccccatacgatataagttgttactagtgcttggattctcaccaataaaaaacgcccggcggcaaccgagcgttctgaacaaatccagatggagttctgaggtcattactggatctatcaacaggagtccaagcgagctcgatatcaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacgaaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattgatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacct

SEQ ID NO: 174 - DNA, 2007 bp, Artificial Sequence; identical to SEQ ID NO: 157
  pTwist+Chlor+MC vectior with MauBI-PacI-AbsI-AvrII-SgrDI-PacI-AscI polylinker

SEQ ID NO: 175 - DNA, 1028 bp, Artificial Sequence
  Synthetic DNA
  CDS (138)..(971): gene fusion comprising kanamycin gene from pACYC177 extended to also encode CGRTK and one stop codon
cgcgcgcgttaattaacctcgaggcctaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgaattcttctgtgggcggacaaaatagtaactagttaattggttgtaacactggcagagcaggcgtcgacgttaattaaggcgcgcc

SEQ ID NO: 176 - protein, 276 aa, Artificial Sequence
  Synthetic Construct
MSHIQRETSCSRPRLNSNMDADLYGYKWARDNVGQSGATIYRLYGKPDAPELFLKHGKGSVANDVTDEMVRLNWLTEFMPLPTIKHFIRTPDDAWLLTTAIPGKTAFQVLEEYPDSGENIVDALAVFLRRLHSIPVCNCPFNSDRVFRLAQAQSRMNNGLVDASDFDDERNGWPVEQVWKEMHKLLPFSPDSVVTHGDFSLDNLIFDEGKLIGCIDVGRVGIADRYQDLAILWNCLGEFSPSLQKRLFQKYGIDNPDMNKLQFHLMLDEFFCGRTK

SEQ ID NO: 177 - DNA, 1040 bp, Artificial Sequence
  Synthetic DNA
  CDS (138)..(983): gene fusion comprising kanamycin gene from pACYC177 extended to also encode PSFNAVVYHS and one stop codon
cgcgcgcgttaattaacctcgaggcctaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgaattcttcccaagctttaatgcggtagtttatcacagttaactagttaattggttgtaacactggcagagcaggcgtcgacgttaattaaggcgcgcc

SEQ ID NO: 178 - protein, 281 aa, Artificial Sequence
  Synthetic Construct
MSHIQRETSCSRPRLNSNMDADLYGYKWARDNVGQSGATIYRLYGKPDAPELFLKHGKGSVANDVTDEMVRLNWLTEFMPLPTIKHFIRTPDDAWLLTTAIPGKTAFQVLEEYPDSGENIVDALAVFLRRLHSIPVCNCPFNSDRVFRLAQAQSRMNNGLVDASDFDDERNGWPVEQVWKEMHKLLPFSPDSVVTHGDFSLDNLIFDEGKLIGCIDVGRVGIADRYQDLAILWNCLGEFSPSLQKRLFQKYGIDNPDMNKLQFHLMLDEFFPSFNAVVYHS

SEQ ID NO: 179 - DNA, 1016 bp, Artificial Sequence
  Synthetic DNA
  CDS (138)..(959): gene fusion comprising kanamycin gene from pACYC177 extended to also encode PS and one stop codon
cgcgcgcgttaattaacctcgaggcctaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgaattcttcccaagctaactagttaattggttgtaacactggcagagcaggcgtcgacgttaattaaggcgcgcc

SEQ ID NO: 180 - protein, 273 aa, Artificial Sequence
  Synthetic Construct
MSHIQRETSCSRPRLNSNMDADLYGYKWARDNVGQSGATIYRLYGKPDAPELFLKHGKGSVANDVTDEMVRLNWLTEFMPLPTIKHFIRTPDDAWLLTTAIPGKTAFQVLEEYPDSGENIVDALAVFLRRLHSIPVCNCPFNSDRVFRLAQAQSRMNNGLVDASDFDDERNGWPVEQVWKEMHKLLPFSPDSVVTHGDFSLDNLIFDEGKLIGCIDVGRVGIADRYQDLAILWNCLGEFSPSLQKRLFQKYGIDNPDMNKLQFHLMLDEFFPS

SEQ ID NO: 181 - DNA, 1074 bp, Artificial Sequence
  Synthetic DNA
  CDS (138)..(968): gene fusion comprising kanamycin gene from pACYC177 extended to also encode CGRTK and one stop codon followed by partial Tn7L
cgcgcgcgttaattaacctcgaggcctaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgaattcttctgtgggcggacaaaatagttgggaactgggaggggtggaaatggagtttttaaggattatttagggactagttaattggttgtaacactggcagagcaggcgtcgacgttaattaaggcgcgcc

SEQ ID NO: 182 - protein, 276 aa, Artificial Sequence; identical to SEQ ID NO: 176
  Synthetic Construct

SEQ ID NO: 183 - DNA, 1075 bp, Artificial Sequence
  Synthetic DNA
  CDS (138)..(1004): gene fusion comprising kanamycin gene from pACYC177 extended to also encode LWADKIVGNWEGWKWSF and one stop codon followed by partial Tn7L in reading frame 2
cgcgcgcgttaattaacctcgaggcctaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgaattcttcctgtgggcggacaaaatagttgggaactgggaggggtggaaatggagtttttaaggattatttagggactagttaattggttgtaacactggcagagcaggcgtcgacgttaattaaggcgcgcc

SEQ ID NO: 184 - protein, 288 aa, Artificial Sequence
  Synthetic Construct
MSHIQRETSCSRPRLNSNMDADLYGYKWARDNVGQSGATIYRLYGKPDAPELFLKHGKGSVANDVTDEMVRLNWLTEFMPLPTIKHFIRTPDDAWLLTTAIPGKTAFQVLEEYPDSGENIVDALAVFLRRLHSIPVCNCPFNSDRVFRLAQAQSRMNNGLVDASDFDDERNGWPVEQVWKEMHKLLPFSPDSVVTHGDFSLDNLIFDEGKLIGCIDVGRVGIADRYQDLAILWNCLGEFSPSLQKRLFQKYGIDNPDMNKLQFHLMLDEFFLWADKIVGNWEGWKWSF

SEQ ID NO: 185 - DNA, 1076 bp, Artificial Sequence
  Synthetic DNA
  CDS (138)..(1007): gene fusion comprising kanamycin gene from pACYC177 extended to also encode PVGSQNSWELGGVEMEFLRII and one stop codon in reading frame 3
cgcgcgcgttaattaacctcgaggcctaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgaattcttccctgtgggcggacaaaatagttgggaactgggaggggtggaaatggagtttttaaggattatttagggactagttaattggttgtaacactggcagagcaggcgtcgacgttaattaaggcgcgcc

SEQ ID NO: 186 - protein, 290 aa, Artificial Sequence
  Synthetic Construct
MSHIQRETSCSRPRLNSNMDADLYGYKWARDNVGQSGATIYRLYGKPDAPELFLKHGKGSVANDVTDEMVRLNWLTEFMPLPTIKHFIRTPDDAWLLTTAIPGKTAFQVLEEYPDSGENIVDALAVFLRRLHSIPVCNCPFNSDRVFRLAQAQSRMNNGLVDASDFDDERNGWPVEQVWKEMHKLLPFSPDSVVTHGDFSLDNLIFDEGKLIGCIDVGRVGIADRYQDLAILWNCLGEFSPSLQKRLFQKYGIDNPDMNKLQFHLMLDEFFPVGGQNSWELGGVEMEFLR

SEQ ID NO: 187 - DNA, 1069 bp, Artificial Sequence
  Synthetic DNA
  CDS (138)..(959): gene fusion comprising kanamycin gene from pACYC177 extended to also encode PS and one stop codon and overlapping mini-attTn7 site
cgcgcgcgttaattaacctcgaggcctaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgaattcttcccaagctaagcatccatttattactcaaccgtaaccgattttgccaggttacgcggctgtaactagttaattggttgtaacactggcagagcaggcgtcgacgttaattaaggcgcgcc

SEQ ID NO: 188 - protein, 273 aa, Artificial Sequence; identical to SEQ ID NO: 180
  Synthetic Construct

SEQ ID NO: 189 - DNA, 1016 bp, Artificial Sequence
  Synthetic DNA
  CDS (138)..(959): gene fusion comprising kanamycin gene from pACYC177 extended to also encode PS and one stop codon
cgcgcgcgttaattaacctcgaggcctaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttcccaagctaattggttaattggttgtaacactggcagagcaggcgtcgacgttaattaaggcgcgcc

SEQ ID NO: 190 - protein, 273 aa, Artificial Sequence; identical to SEQ ID NO: 180
  Synthetic Construct

SEQ ID NO: 191 - DNA, 1016 bp, Artificial Sequence
  Synthetic DNA
  CDS (138)..(953): Unaltered kanamycn gene from pACYC177 and one TAA stop codon
cgcgcgcgttaattaacctcgaggcctaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcaggcgtcgacgttaattaaggcgcgcc

SEQ ID NO: 192 - protein, 271 aa, Artificial Sequence
  Synthetic Construct
MSHIQRETSCSRPRLNSNMDADLYGYKWARDNVGQSGATIYRLYGKPDAPELFLKHGKGSVANDVTDEMVRLNWLTEFMPLPTIKHFIRTPDDAWLLTTAIPGKTAFQVLEEYPDSGENIVDALAVFLRRLHSIPVCNCPFNSDRVFRLAQAQSRMNNGLVDASDFDDERNGWPVEQVWKEMHKLLPFSPDSVVTHGDFSLDNLIFDEGKLIGCIDVGRVGIADRYQDLAILWNCLGEFSPSLQKRLFQKYGIDNPDMNKLQFHLMLDEFF

SEQ ID NO: 193 - DNA, 837 bp, Artificial Sequence
  Synthetic DNA
  CDS (262)..(804): lacZalpha gene comprising mini-attTn7 target site
cgcgcgcgttaattaacctcgaggcctcctgcaggaattaagtcttcgaaccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctcggaattaaccctcactaaagggaacaaaagctggagctccaccgcggtggcggccgctctagaactagtggatcccccgggctgcaggaattcacataacaggaagaaaaatgccccgcttacgcagggcatccatttattactcaaccgtaaccgattttgccaggttacgcggctgtcgacctcgagggggggcccggtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcttcgaagacgctcgacagcgacacacttgcatcggatgcagcccggttaacgtgccggcacggcctgggtaaccaggtaggcgtcgacgttaattaaggcgcgcc

SEQ ID NO: 194 - protein, 180 aa, Artificial Sequence
  Synthetic Construct
MTMITPSSELTLTKGNKSWSSTAVAAALELVDPPGCRNSHNRKKNAPLTQGIHLLLNRNRFCQVTRLSTSRGGPVPNSPYSESYYARSLAVVLQRRDWENPGVTQLNRLAAHPPFASWRNSEEARTDRPSQQLRSLNGEWDAPCSGALSAAGVVVTRFEDARQRHTCIGCSPVNVPARPG

SEQ ID NO: 195 - DNA, 687 bp, Artificial Sequence
  Synthetic DNA
  CDS (262)..(654): lacZalpha gene comprising micro-attTn7 target site
cgcgcgcgttaattaacctcgaggcctcctgcaggaattaagtcttcgaaccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcacataacaggaagaaaaatgccccgcttacgcagggcatccatttattactcaaccgtaaccgattttgccaggttacgcggctgtcgacttcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcttcgaagacgctcgacagcgacacacttgcatcggatgcagcccggttaacgtgccggcacggcctgggtaaccaggtaggcgtcgacgttaattaaggcgcgcc

SEQ ID NO: 196 - protein, 130 aa, Artificial Sequence
  Synthetic Construct
MTMITNSHNRKKNAPLTQGIHLLLNRNRFCQVTRLSTSLAVVLQRRDWENPGVTQLNRLAAHPPFASWRNSEEARTDRPSQQLRSLNGEWDAPCSGALSAAGVVVTRFEDARQRHTCIGCSPVNVPARPG

SEQ ID NO: 197 - DNA, 2221 bp, Artificial Sequence
  pTwist-Amp-HC cloning vector
aggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtcctcgtgataccaattcggagcctgcttttttgtacaaacttgttgataatggcaattcaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagagccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccagatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacct

SEQ ID NO: 198 - DNA, 2275 bp, Artificial Sequence
  pTwist+Amp+HC with MauBI-AbsI-AvrII-SgrDI-AscI polylinker
cgcgcgcgttaattaacctcgaggcctaggcgtcgacgttaattaaggcgcgccaggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtcctcgtgataccaattcggagcctgcttttttgtacaaacttgttgataatggcaattcaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagagccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccagatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacct

SEQ ID NO: 199 - DNA, 546 bp, Artificial Sequence
  Synthetic DNA
  misc_feature (27)..(521): new mini-Tn7 with Tn7L - MauBI-PacI-AbsI-AvrII(PstI)SacII-SgrDI-PacI-AscI polylinker-Tn7R
acataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcgcgcgcgttaattaacctcgaggcctaggcctgcaggccgcggcgtcgacgttaattaaggcgcgccagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccattta

SEQ ID NO: 200 - DNA, 986 bp, Artificial Sequence
  Synthetic DNA
  misc_feature (27)..(961): new mini-Tn7 with lacZalpha gene from pUC18 between Tn7L and Tn7R
  CDS (438)..(680): lacZalpha gene ending with two TAA stop codons with internal MCS region derived from pUC18 bwtween Tn7L and Tn7R in a new mini-Tn7 sequence
acataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcgcgcgcgttaattaacctcgaggcctccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatcctctagagtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctttaataaaggcgtcgacgttaattaaggcgcgccagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccattta

SEQ ID NO: 201 - protein, 79 aa, Artificial Sequence
  Synthetic Construct
MTMITNSSSVPGDPLESTCRHASLALAVVLQRRDWENPGVTQLNRLAAHPPFASWRNSEEARTDRPSQQLRSLNGEWRL

SEQ ID NO: 202 - DNA, 1028 bp, Artificial Sequence; identical to SEQ ID NO: 175
  Synthetic DNA

SEQ ID NO: 203 - DNA, 1028 bp, Artificial Sequence; identical to SEQ ID NO: 175
  Synthetic DNA

SEQ ID NO: 204 - DNA, 1040 bp, Artificial Sequence; identical to SEQ ID NO: 177
  Synthetic DNA

SEQ ID NO: 205 - DNA, 1069 bp, Artificial Sequence; identical to SEQ ID NO: 187
  Synthetic DNA

SEQ ID NO: 206 - DNA, 1074 bp, Artificial Sequence; identical to SEQ ID NO: 181
  Synthetic DNA

SEQ ID NO: 207 - DNA, 1075 bp, Artificial Sequence; identical to SEQ ID NO: 183
  Synthetic DNA

SEQ ID NO: 208 - DNA, 1076 bp, Artificial Sequence; identical to SEQ ID NO: 185
  Synthetic DNA

SEQ ID NO: 209 - DNA, 1016 bp, Artificial Sequence; identical to SEQ ID NO: 189
  Synthetic DNA

SEQ ID NO: 210 - DNA, 1016 bp, Artificial Sequence; identical to SEQ ID NO: 191
  Synthetic DNA

SEQ ID NO: 211 - DNA, 876 bp, Artificial Sequence; identical to SEQ ID NO: 159
  Synthetic DNA

SEQ ID NO: 212 - DNA, 876 bp, Artificial Sequence; identical to SEQ ID NO: 161
  Synthetic DNA

SEQ ID NO: 213 - DNA, 876 bp, Artificial Sequence; identical to SEQ ID NO: 163
  Synthetic DNA

SEQ ID NO: 214 - DNA, 889 bp, Artificial Sequence; identical to SEQ ID NO: 165
  Synthetic DNA

SEQ ID NO: 215 - DNA, 896 bp, Artificial Sequence; identical to SEQ ID NO: 167
  Synthetic DNA

SEQ ID NO: 216 - DNA, 897 bp, Artificial Sequence; identical to SEQ ID NO: 169
  Synthetic DNA

SEQ ID NO: 217 - DNA, 898 bp, Artificial Sequence; identical to SEQ ID NO: 171
  Synthetic DNA

SEQ ID NO: 218 - DNA, 687 bp, Artificial Sequence; identical to SEQ ID NO: 195
  Synthetic DNA

SEQ ID NO: 219 - DNA, 837 bp, Artificial Sequence; identical to SEQ ID NO: 193
  Synthetic DNA

SEQ ID NO: 220 - DNA, 1016 bp, Artificial Sequence
  Synthetic DNA
cgcgcgcgttaattaacctcgaggcctaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgaattcttctaatcagaattggttaattggttgtaacactggcagagcaggcgtcgacgttaattaaggcgcgcc

SEQ ID NO: 221 - DNA, 1025 bp, Artificial Sequence
  Synthetic DNA
cgcgcgcgttaattaacctcgaggcctaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagttttgtgggcggacaaaatagtaattggttaattggttgtaacactggcagagcaggcgtcgacgttaattaaggcgcgcc

SEQ ID NO: 222 - DNA, 1016 bp, Artificial Sequence
  Synthetic DNA
cgcgcgcgttaattaacctcgaggcctaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagctgttctaatcagaattggttaattggttgtaacactggcagagcaggcgtcgacgttaattaaggcgcgcc

SEQ ID NO: 223 - DNA, 1016 bp, Artificial Sequence
  Synthetic DNA
cgcgcgcgttaattaacctcgaggcctaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagctgttcccaagctaattggttaattggttgtaacactggcagagcaggcgtcgacgttaattaaggcgcgcc

SEQ ID NO: 224 - DNA, 1021 bp, Artificial Sequence
  Synthetic DNA
cgcgcgcgttaattaacctcgaggcctaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagctgtttaattaacaataaagttggttaattggttgtaacactggcagagcaggcgtcgacgttaattaaggcgcgcc

SEQ ID NO: 225 - DNA, 1022 bp, Artificial Sequence
  Synthetic DNA
cgcgcgcgttaattaacctcgaggcctaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagctgttttaattaacaataaagttggttaattggttgtaacactggcagagcaggcgtcgacgttaattaaggcgcgcc

SEQ ID NO: 226 - DNA, 1022 bp, Artificial Sequence
  Synthetic DNA
cgcgcgcgttaattaacctcgaggcctaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagctgttctagataacaataaagttggttaattggttgtaacactggcagagcaggcgtcgacgttaattaaggcgcgcc

SEQ ID NO: 227 - DNA, 1064 bp, Artificial Sequence
  Synthetic DNA
cgcgcgcgttaattaacctcgaggcctaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagctgttcccaagctaaaatttattactcaaccgtaaccgattttgccaggttacgcggctgtaattggttaattggttgtaacactggcagagcaggcgtcgacgttaattaaggcgcgcc

SEQ ID NO: 228 - DNA, 1188 bp, Artificial Sequence
  Synthetic DNA
cgcgcgcgttaattaacctcgaggcctcctgcaggaattaagtcttcgaaccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagctgttccaagcttaaaatttattactcaaccgtaaccgattttgccaggttacgcggctgtaattggttaattggttgtaacactggcagagcaggcgtcgacgttaattaaggcgcgcc

SEQ ID NO: 229 - DNA, 1188 bp, Artificial Sequence
  Synthetic DNA
cgcgcgcgttaattaacctcgaggcctcctgcaggaattaagtcttcgaaccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagctgttcccaagctaaaatttattactcaaccgtaaccgattttgccaggttacgcggctgtaattggttaattggttgtaacactggcagagcaggcgtcgacgttaattaaggcgcgcc

SEQ ID NO: 230 - DNA, 965 bp, Artificial Sequence
  Synthetic DNA
cgcgcgcgttaattaacctcgaggcctccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactaataaagggcatccatttattactcaaccgtaaccgattttgccaggttacgcggctgggtgcccgggccgtcgacgttaattaaggcgcgcc

SEQ ID NO: 231 - DNA, 965 bp, Artificial Sequence
  Synthetic DNA
cgcgcgcgttaattaacctcgaggcctccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgctaaagggcatccatttattactcaaccgtaaccgattttgccaggttacgcggctgggtgcccgggccgtcgacgttaattaaggcgcgcc

SEQ ID NO: 232 - DNA, 965 bp, Artificial Sequence; identical to SEQ ID NO: 231
  Synthetic DNA

SEQ ID NO: 233 - DNA, 1016 bp, Artificial Sequence
  Synthetic DNA
cgcgcgcgttaattaacctcgaggcctccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgtcttattcaaagcaaggcatcgcacaagtaatgaagacgaaataccatatggaaggcagtgtcaatggccatgaattcacgatcgaaggtgtaggaactggaaacccttacgaaggcacacagatgtccgaattagtgatcaccaagcctgcaggaaaaccccttccattctcctttgacattctgtcaacagtctttcagtatggaaacaggtgcttcacaaagtaccctgaaggaatgactgactatttcaagcaagcattcccagatggaatgtcatttgaaaggtcatttctatatgaggatggaggagttgctacagccagctggaacattcgtcttgagagagattgcttcatccacaaatccatctatcatggcgttaactttcccgctgatggacccgtaatgaaaaagaagaccattggctgggataaagccttcgaaaaaatgactgtgtccaaagacgttttaagaggtgatgtgactgagtttcttatgctcgaaggaggtggttaccacagctgccagtttcactccacttacaaaccagagaagccggttacactgcccccgaatcatgtcgtggaacatcacattgtgaggactgaccttggccaaactgcaaaaggcttcacagtcaagctggaagaacatgctgcggctcatgttaaccctttgaaggttcaataaaggatttattactcaaccgtaaccgattttgccaggttacgcggctgggtgcccgggccgtcgacgttaattaaggcgcgcc

SEQ ID NO: 234 - DNA, 1822 bp, Artificial Sequence
  Synthetic DNA
acataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcgcgcgcgttaattaacctcgaggcctccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatcctctagagtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctttaataaaggcgtcgacgttaattaacctcgaggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgctcttctaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggcgtcgacgttaattaaggcgcgccagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccattta

SEQ ID NO: 235 - DNA, 1822 bp, Artificial Sequence
  Synthetic DNA
acataacaggaagaaaaatgccccgctgttttaattaaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcgcgcgcgttaattaacctcgaggcctccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatcctctagagtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctttaataaaggcgtcgacgttaattaacctcgaggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgctcttctaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggcgtcgacgttaattaaggcgcgccagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccattta

SEQ ID NO: 236 - DNA, 1822 bp, Artificial Sequence
  Synthetic DNA
acataacaggaagaaaaatgccccgctgtttaattaaaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcgcgcgcgttaattaacctcgaggcctccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatcctctagagtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctttaataaaggcgtcgacgttaattaacctcgaggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgctcttctaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggcgtcgacgttaattaaggcgcgccagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccattta

SEQ ID NO: 237 - DNA, 1822 bp, Artificial Sequence
  Synthetic DNA
acataacaggaagaaaaatgccccgctgttctagataaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcgcgcgcgttaattaacctcgaggcctccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatcctctagagtcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctttaataaaggcgtcgacgttaattaacctcgaggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgctcttctaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggcgtcgacgttaattaaggcgcgccagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccattta

SEQ ID NO: 238 - DNA, 1868 bp, Artificial Sequence
  Artificial Sequence
acataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcgcgcgcgttaattaacctcgaggccttggcgggatcgttgtatatttcttgacaccttttcggcatcgccctaaaattcggcgtcctcatattgtgtgaggacgttttattacgtgtttacgaagcaaaagctaaaaccaggagctatttaatggcaacagttaaccagctggtacgcaaaccacgtgctcgcaaagttgcgaaaagcaacgtgcctgcgctggaagcatgcccgcaaaaacgtggcgtatgtactcgtgtatatactaccactcctaaaaaaccgaactccgcgctgcgtaaagtatgccgtgttcgtctgactaacggtttcgaagtgacttcctacatcggtggtgaaggtcacaacctgcaggagcactccgtgatcctgatccgtggcggtcgtgttaaagacctcccgggtgttcgttaccacaccgtacgtggtgcgcttgactgctccggcgttaaagaccgtaagcaggctcgttccaagtatggcgtgaagcgtcctaaggcttaaaggcgtcgacgttaattaacctcgaggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgctcttctaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggcgtcgacgttaattaaggcgcgccagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccattta

SEQ ID NO: 239 - DNA, 2278 bp, Artificial Sequence
  Synthetic DNA
acataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttcggatctcgcgcgcgttaattaacctcgaggcctccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgtcttattcaaagcaaggcatcgcacaagtaatgaagacgaaataccatatggaaggcagtgtcaatggccatgaattcacgatcgaaggtgtaggaactggaaacccttacgaaggcacacagatgtccgaattagtgatcaccaagcctgcaggaaaaccccttccattctcctttgacattctgtcaacagtctttcagtatggaaacaggtgcttcacaaagtaccctgaaggaatgactgactatttcaagcaagcattcccagatggaatgtcatttgaaaggtcatttctatatgaggatggaggagttgctacagccagctggaacattcgtcttgagagagattgcttcatccacaaatccatctatcatggcgttaactttcccgctgatggacccgtaatgaaaaagaagaccattggctgggataaagccttcgaaaaaatgactgtgtccaaagacgttttaagaggtgatgtgactgagtttcttatgctcgaaggaggtggttaccacagctgccagtttcactccacttacaaaccagagaagccggttacactgcccccgaatcatgtcgtggaacatcacattgtgaggactgaccttggccaaactgcaaaaggcttcacagtcaagctggaagaacatgctgcggctcatgttaaccctttgaaggttcaataataaaggcgtcgacgttaattaacctcgaggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgctcttctaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaggtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggcgtcgacgttaattaaggcgcgccagttgttcggtaaattgtcacaacgccgcgaatatagtctttaccatgcccttggccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacacccgcttacgcagggcatccattta

SEQ ID NO: 240 - DNA, 240 bp, Artificial Sequence
  Synthetic Sequence
  misc_feature (1)..(193): Partial sequence of cat gene before junction with 5′ end of Tn7L
  Unsure (192)..(192)
  misc_feature (194)..(240): 5′ end of Tn7L
  Unsure (194)..(194)
  Unsure (197)..(197)
  Unsure (199)..(201)
  Unsure (203)..(203)
caatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtncngtngnnngncaaaatagttgggaactgggaggggtggaaatggagt

(19) United States
(12) Patent Application Publication
(10) Pub. No.: US 2022/0081692 A1
(43) Pub. Date: Mar. 17, 2022

(54) Combinatorial Assembly of Composite Arrays of Site-Specific Synthetic Transposons Inserted Into Sequences Comprising Novel Target Sites in Modular Prokaryotic and Eukaryotic Vectors

(71) Applicant: Verne A. Luckow, Chesterfield, MO (US)

(72) Inventor: Verne A. Luckow, Chesterfield, MO (US)

(21) Appl. No.: 17/013,546

(22) Filed: Sep. 5, 2020

(51) Int. Cl. C12N 15/62; C12N 9/10; C12N 15/70; C12N 15/86

(52) U.S. Cl. CPC C12N 15/62; C12N 9/1033; C12N 15/70; C12N 2710/14041; C12Y 203/01028; C07K 2319/00; C12N 15/86

(57) ABSTRACT

The design, assembly, and use of novel sequences comprising targeting and insertion sites for site-specific bacterial transposons are disclosed. One aspect relates to a nucleotide sequence comprising an attachment site for a site-specific transposon operably-linked to a screenable or selectable marker sequence, wherein said marker sequence encodes one or more active or inactive polypeptides capable of conferring a screenable or selectable phenotype upon a cell comprising the marker sequence, wherein insertion of the site-specific transposon into the attachment site changes the phenotype of a cell comprising the screenable or selectable marker sequence. High and low copy number vectors comprising the sequences, designated synthemids, including plasmids capable of propagating in bacteria, and shuttle vectors, capable of propagating in bacteria and a eukaryotic host cell or two types of bacteria by means of distinct replicons, are also disclosed. Related aspects include the design and assembly of synthetic insect and mammalian virus shuttle vectors, including shuttle vectors comprising segments of a double-stranded DNA virus, such as a baculovirus, which propagates in insect cells, or a herpesvirus, an adenovirus, or a pox virus, which propagate in mammalian cells. Other aspects relate to use of modified vectors to express polypeptides for use as therapeutic drug products, as vaccines, or as components of cell or gene therapy vector systems, and in model and crop plant cells, tissues, and whole plants to facilitate the basic and applied studies leading to improved food products, and as tools advancing the interests of institutions involved in industrial and environmental biotechnology.

### CROSS REFERENCE TO RELATED APPLICATIONS

This application claims priority to and the benefit of US 63-001,614 filed 2020 Mar. 30 U.S. Provisional Application No. U.S. 63/001,614, filed Mar. 30, 2020, U.S. Provisional Application No. 62/906,003, filed Sep. 25, 2019, and U.S. Provisional Application No. 62/896,494, filed Sep. 5, 2019, the entire contents of which are incorporated by reference in their entirety.

### INCORPORATION-BY-REFERENCE OF A SEQUENCE LISTING

The sequence listing contained in the file “950_951_012_US_01_Sequence_Listing_2020_09_05_ST25.txt”, created on 2020 Sep. 5, modified on 2020 Sep. 5, file size 301,133 bytes, and any original and amended sequence listings for “950_951_011_US_01_Sequence_Listing_2020_03_30_ST25.txt”, created on 2020 Mar. 30, modified on 2020 Mar. 30, file size 239,095 bytes, U.S. 62/906,003, filed Sep. 25, 2019, and U.S. 62/896,494, filed Sep. 5, 2019, are incorporated by reference in their entirety herein.

### FIELD OF THE INVENTION

The design, assembly, and use of novel sequences comprising targeting and insertion sites for site-specific bacterial transposons are disclosed.

A major aspect of the invention relates to a nucleotide sequence comprising a target site for a site-specific transposon, wherein said target site comprises a target sequence comprising a transcriptionally or translationally fused marker sequence encoding a selectable marker sequence or a screenable marker sequence operably-linked to a sequence comprising a specific target sequence for recognition and insertion of a site-specific transposon or a site-specific recombinase, wherein said fused marker sequence encodes an inactive or an active polypeptide capable of conferring a selectable or screenable phenotype upon a cell comprising the fused marker sequence, wherein insertion of the site-specific transposon into the target sequence to create a composite target sequence changes the phenotype of a cell comprising the composite screenable or selectable marker sequence compared to a cell comprising just the selectable or screenable marker sequence.

Another major aspect of the invention relates to a method of screening or selecting for transposition of a site-specific transposon into a nucleotide sequence comprising an attachment site for a site-specific transposon operably-linked to a screenable or selectable marker sequence, comprising the steps of (i) introducing into a bacterial cell a target vector comprising a marker sequence that encodes one or more active or inactive polypeptides capable of conferring a screenable or selectable phenotype upon a cell comprising the marker sequence, wherein insertion of the site-specific transposon into the attachment site to create a composite marker sequence changes the phenotype of a cell comprising the screenable or selectable marker sequence; (ii) introducing into said cell comprising said target vector, a donor vector comprising sequences capable of transposing the wild type or a variant form of the site-specific transposon, and optionally a helper vector comprising sequences encoding one or more transposase gene products; (iii) culturing and optionally plating bacteria comprising the target vector, and optionally donor and helper vectors, (iv) screening or selecting for bacterial colonies where transposition of the site-specific transposon into the attachment site on the target vector to create a composite marker sequence changes the phenotype of the bacterial cell harboring the target vector.

Related aspects include the combinatorial assembly of ordered composite arrays of site-specific synthetic transposons inserted into sequences comprising novel target sites in stable locations on modular prokaryotic and eukaryotic vectors.

Other aspects relate to vectors comprising high or low copy number replicons comprising target or composite target sequences, designated synthemids, including plasmids capable of propagating in bacteria, and shuttle vectors, capable of propagating in bacteria and a eukaryotic host cell or two types of bacteria by means of distinct replicons.

Related aspects include the design and assembly of synthetic insect and mammalian virus shuttle vectors, including shuttle vectors comprising one or more segments of a double-stranded DNA virus, such as a baculovirus, which propagates in insect cells, or a herpesvirus, an adenovirus, or a pox virus, which propagate in mammalian cells. Other aspects of the invention relate to use of modified vectors to express polypeptides for use as therapeutic drug products, as vaccines, or as components of cell or gene therapy vector systems.

Related aspects also include the design and assembly of shuttle vectors for use in plant cell-based expression systems, and shuttle vectors for use in industrial or environmental biotechnology applications, such as vectors comprising a replicon that can facilitate propagation in unicellular or filamentous fungal cells, and vectors that can propagate in non-enteric bacteria, such as those associated with soil, aquatic, and extreme environments, are also disclosed.

### BACKGROUND OF THE INVENTION

The design and assembly of nucleic acids comprising one or more genetic elements in a desired order typically requires a variety of techniques, including cloning of one or more isolated DNA sequences into vectors which propagate in bacteria, sequencing of the cloned inserts, introduction of the vector into an appropriate host cell, and expression of polypeptides under the control of a promoter operably-linked to the inserted sequences. Structural and functional analysis of the expressed polypeptides advances research, and often leading to the development and commercialization of products intended for use as food or drug products, including transgenic plant materials, therapeutic drug products, vaccines, components of gene therapy vector systems, and as tools advancing the interests of institutions involved in industrial and environmental biotechnology.

Structural and functional analysis also requires the analysis of variants, obtained through mutagenesis of vectors comprising nucleotide sequences of interest, such as one or more substitutions, insertions, and deletions, or combinations thereof, at specific locations or scattered along many locations of the primary sequence of the sequence of interest. Substitutions in the nucleotide sequence may change a codon from one encoding an amino acid, to a stop codon, terminating translation from the corresponding mRNA, or change the codon to encode a different amino acid, which may affect the structural and functional properties of the expressed variant polypeptide. Insertions or deletions in the nucleotide sequence may affect the reading frame of the mRNA leading to expression of shorter or longer polypeptides often having reduced or no activity, or in some cases, retaining or enhancing activity, compared to an unaltered parent molecule. Gene fusions may comprise several genetic elements, typically regulatory sequences from one or several types of genes, operably-linked to a sequence encoding a polypeptide of interest. Protein fusions may comprise structural and functional domains of two or more polypeptides, such that the resulting molecule has new, perhaps desirable or even surprising properties, compared to domains located on separate parent molecules. Analysis of deletion and insertion variants, may facilitate the identification of amino acid residues that are involved in the catalytic activity of an enzyme, or the binding of a polypeptide to other structural molecules within or outside of a cell. Demonstrating that specific regions or residues along the primary sequence of a polypeptide are critical, compared to those that are more tolerant of alterations, greatly facilitates the development of strategies to facilitate expression of polypeptides having enhanced or reduced activity useful in basic and applied research, including structural analysis of polypeptides crystalized with substrates, cofactors, or binding domains of other large molecules.

### Cloning Techniques

A wide variety of techniques have been used to facilitate the cloning of segments of DNA comprising one or more genetic elements into a vector that can propagate in commonly-used laboratory strains of bacteria, such as Escherichia coli, and often other types of prokaryotic or eukaryotic host cells. Key features of traditional and more modern cloning techniques, such as BioBrick Assembly, 3A Assembly, Gibson Assembly, Infusion Cloning, Iterative Capped Assembly, Golden Gate Assembly, TOPO-TA cloning, and Overlap Extension PCR techniques, are summarized below.

Traditional sequential methods of cloning, often rely on Type II restriction endonucleases that cut double-stranded DNA (dsDNA) within a specific palindromic recognition sequence, that yield blunt ends, or sticky ends with 5′ or 3′ overhangs. Plasmid vectors comprising an intact replicon and one or more selectable marker are digested with one or more restriction enzymes and combined with a composition comprising an insert, typically a Gene of Interest (GOI) that was digested with compatible restriction enzymes to create compatible blunt ends or complementary sticky ends. T4 DNA ligase is used to create a circular vector containing the GOI, which is transformed into competent bacterial cells. Colonies of bacteria grown on selectable or screenable media are recovered, purified, and cultured, allowing recovery of plasmid DNA that can be analyzed by restriction fragment mapping, gene amplification techniques, or DNA sequencing methods to confirm that a desired insert was cloned. While over 500 types of restriction enzymes, these methods are often quite laborious and require knowledge of the number and relative locations of recognition sites for the enzymes used to digest the vector and the source of the cloned insert.

BioBrick Assembly methods rely on the standardization of cloning sites in vectors and sequences flanking genetic elements of interest, permitting the sequential assembly of complementary parts, into devices, having a defined function, and systems, comprising a set of devices that perform high level tasks [Knight, T. (2005). Idempotent Vector Design for Standard Assembly of BioBricks. MIT Synthetic Biology Working Group]. Assembly standard 10, relies on the use of synthetic sequences, called prefixes and suffixes, which flank each part cloned into a base vector. In one scheme, the prefix sequence comprises sites for EcoRI and XbaI, while the suffix sequence comprises sequences for SpeI and PstI. A vector comprising a first device of interest is digested with EcoRI and SpeI, and a second vector comprising a second device and a replicon and selectable marker is digested with EcoRI and XbaI. Samples from both digests are mixed and ligated together, to form a larger vector comprising two devices with a “scar” site formed by the ligation of the compatible XbaI and SpeI sticky ends, that is not recognized by either restriction enzyme. The two contiguous devices in the larger product vector can be released from digestion with EcoRI and SpeI, or retained in a vector digested with EcoRI and XbaI that are used in subsequent reactions to assemble vectors comprising three or more parts, which may function as devices or systems. Other variations include use of compatible prefixes comprising recognition sites for EcoRI and BglII and suffixes comprising recognition sites for BamHI and XhoI sites, and prefixes and suffixes that also contain recognition sites for AgeI and NgoMIV, respectively.

Three Antibiotic (3A) Assembly extends the BioBrick theme, and relies on three sets of plasmids each conferring resistance to different antibiotic resistance markers (A, B, and C). Digestion of plasmid A with EcoRI and SalI releases a first insert, while digestion of plasmid B, with XbaI and PstI releases a second insert, and digestion of plasmid C, retains the vector backbone comprising a replicon and the gene conferring resistance to antibiotic C. Samples from all three digests are mixed and ligated, transformed into bacteria, and plated on media containing antibiotic C. The resulting plasmid should contain contiguous first and second inserts with an internal scar, flanked by a prefix containing recognition sites for EcoRI and XbaI sites, and a suffix containing recognition sites for SpeI and PstI.

Gibson Assembly methods of cloning require several steps involving linearization of a vector or of inserts by digestion with restriction enzymes or by amplification of DNA segments using polymerase chain reaction (PCR) techniques, followed by treatment with a 3′-5′ exonuclease to generate complementary, overlapping ends that are annealed and extended by a DNA polymerase, and sealed by DNA ligase to produce a single, contiguous linear or circular strand of DNA. [Gibson et al, “Complete chemical synthesis, assembly, and cloning of a Mycoplasma genitalium genome.” Science, 319:1215-20, 2008] [Gibson et al, “Enzymatic assembly of DNA molecules up to several hundred kilobases.” Nat Meth, 6:343-5, 2009]. Overlapping segments should be unique, ranging from 15 to 80 nucleotides, and incapable of making secondary structures. This method, which requires careful experimental designs, is rapid and seamless (not producing any scars), but produces fragments that are not readily interchangeable with other parts, unless the flanking ends are designed to contain BioBrick-like prefix and suffix sequences. Up to six dsDNA fragments can be assembled in a single reaction. Larger, contiguous regions may require the coupling of segments prepared from several Gibson Assembly reactions.

In-Fusion™ PCR Cloning, developed by Clontech, is an efficient, ligation-independent method of cloning a linearized insert with a linearized vector, where the flanking ends contain 15 to 20 bp homologous overlapping segments. A proprietary In-Fusion enzyme mix is added, generating single-stranded 5′ overhangs at the termini of the insert and the linearized vector, incubated, and the non-covalently joined molecules are transformed into competent bacterial cells, which generate stable molecules. The enzyme mix contains a vaccinia virus DNA polymerase that has a 3′ to 5′ proofreading exonuclease that can degrade the ends of dsDNA to generate ssDNA tails. [Bird, L. E., Rada, H., Flanagan, J., Diprose, J. M., Gilbert, R. J. C. and Owens, R. J. (2014). Application of In-Fusion™ cloning for the parallel construction of E. coli expression vectors. Methods Mol. Biol. Clifton N.J. 1116: 209-234; Zhu, B., Cai, G., Hall, E. O. and Freeman, G. J. (2007). In-fusion assembly: seamless engineering of multidomain fusion proteins, modular vectors, and mutations. BioTechniques 43: 354-359; In-Fusion® HD Cloning Kit User Manual].

Golden Gate Assembly is a method of preparing vectors comprising multiple DNA parts in the presence of Type IIS restriction enzymes and T4 DNA ligase in a single step reaction. [C. Engler, R. Kandzia, and S. Marillonnet, “A one pot, one step, precision cloning method with high throughput capability.,” PLoS One, 3(11): p. e3647, January 2008.] Type IIS enzymes cut outside their recognition sequences, to produce DNA fragments that have sticky ends or overhangs that can be designed to be complementary to sticky ends generated by other Type II or IIS restriction enzymes. BsaI, for example, recognizes a 6 bp sequence and generates 4 base 5′ sticky end (GGTCTCN′NNNN,). A mixture of inserts prepared from several vectors cleaved by different enzymes is ligated to a recipient vector encoding a different antibiotic resistance marker digested with a type IIS enzyme, and the combined mixture treated with T4 DNA ligase to generate a vector comprising one or more inserts in a pre-determined order and orientation. The inserts and vectors are designed to place the Type IIS recognition site distal to the endonuclease cleavage site, so that the recognition sites are removed from the assembled vector comprising the inserts. The assembled vector cannot be digested again with the same Type IIS restriction enzymes.

Iterative Capped Assembly is similar to the Golden Gate method of assembling DNA fragments, requiring use of oligonucleotide monomers comprising sequences for Type IIS restriction enzymes that cleave dsDNAs outside of their recognition sites. Segments of DNA are bound to a solid substrate, and extended sequentially. The reactions require use of a complex set of oligonucleotides called The Initiator, The Terminator, and the Cap. Capping oligonucleotides which contain hairpins at one end, block incompletely extended chains, greatly increasing the frequency of full-length final products released from the solid substrate. [Adrian W. Briggs, Xavier Rios, Raj Chari, Luhan Yang, Feng Zhang, Prashant Mali and George M. Church (2012) Iterative capped assembly: rapid and scalable synthesis of repeat-module DNA such as TAL effectors from individual monomers. Nucleic Acids Research, 2012, Vol. 40, No. 15 e117 doi:10.1093/nar/gks624]. This method, while designed for assembly of modular, repetitive sequences, requires the introduction of sticky ends through end-extension PCR methods, is often more difficult to use than Gibson or Golden Gate methods of assembling non-repetitive sequences.

TOPO-TA Cloning is a method developed by Thermo Fisher that relies on Vaccinia virus DNA Topoisomerase I to provide quick, one step cloning of a Taq DNA polymerase-amplified PCR fragment into a plasmid vector. [Thermo Fisher (2015) TOPO Cloning Technology Brochure; Sigma Aldrich (2015) Topoisomerase I from Vaccinia Virus. Datasheet]. Taq polymerase adds a single adenosine (A) residue to the 3′ ends of amplified fragments, creating a mononucleotide overhang. A linearized TOPO vector having a single deoxythymidine (T) residue each of its 3′ ends is bound to the topoisomerase through a 3′ phosphate of the cleaved strand, permitting annealing of the insert to the vector, followed by ligation and release of the bound enzyme. This method is based on an earlier approach called TA cloning, relying on ligation of Taq-amplified inserts into linearized ddT-tailed vectors [Holton, T. A., Graham, M. W. (1991). A simple and efficient method for direct cloning of PCR products using ddT-tailed vectors. Nucleic Acids Research, 19(5): 1156.] While TOP-TA method is quick, only a limited number of linearized vectors are commercially available, and vectors comprising the insert in either orientations may be recovered.

Overlap Extension PCR is a two-step method requiring amplification and purification of an insert comprising flanking 5′ and 3′ ends that are homologous to segments in a cloning vector in the presence of a high fidelity thermostable DNA polymerase, followed by amplification of the insert in the presence of the desired cloning vector. This method does not require use of restriction enzymes or DNA ligase, and can be used to for site directed mutagenesis or insertion of short segments of DNA into specific positions within the cloning vector. [A. Urban, “A rapid and efficient method for site-directed mutagenesis using one-step overlap extension PCR.” Nucleic Acids Res., 25(11): 2227-2228, June 1997; M. I. Bryksin A., “Overlap extension PCR cloning: a simple and reliable way to create recombinant plasmids.” Biotechniques, 29(6): 997-1003, 2012].

### Mutagenesis Techniques

The ability to recognize changes in the phenotype of a microorganism, plant, or animal, and trace their origins to specific locations on heritable molecules, were remarkable achievements in the first half of the 20^{th} century. Systematic examination of changes induced by physical, chemical, and biological agents, led to the development of modern molecular genetics having applications that transformed the fields of therapeutic drug development, diagnostics, gene therapy systems, modified crop plants, environmental biology, and industrial microbiology. These and other fields, now encompassed by the term synthetic biology, rely heavily on mutagenic methods to facilitate the generation and analysis of structural and functional variants of genetic elements in nucleic acids comprising cis-acting regulatory sequences operably linked to sequences encoding polypeptides or sequences encoding other types of trans-acting regulatory and structural molecules.

A wide variety of techniques have been used to induce mutations in heritable genetic materials, primarily DNA. Agents of artificial mutations generally fall into two classes, physical and chemical mutagens. Biologic agents include viruses and transposons, which insert DNA sequences into regulatory regions or coding sequences of a gene, that often result in inactivation, or rarely, the formation of chimeric genes where the regulatory region of one gene is fused to the coding sequence of another, or the formation of genes encoding fusion proteins, where structural domains from one protein are fused in phase with structural domains of a second protein, that often do not retain their original functional properties.

Commonly used physical mutagens are based on radiation, as particles emitted from natural sources in the environment, or reactors, including X-rays, gamma rays, neutrons, beta particles, alpha particles, protons, and charged ions emitted from particle accelerators, each with different intensities, and half-lives, if emitted as a radiative isotope. The mutagenic effects are often the result of breakage of double-stranded DNA (dsDNA), often resulting in deletions or rearrangements of segments host chromosomes.

Chemical mutagens, which include alkylating agents, azides, hydroxylamine, some antibiotics, nitrous acid, acridines, and base analogues, generally induce single or clustered base mutations along the primary sequence of DNA. Alkylating agents, such as dimethyl sulfate (DMS), nitroso guanidines (NG), along with azide and hydroxylamine, react with bases producing alkylated forms, which may degrade to form an abasic site, which is mutagenic and recombinogenic, or subject to mispairing during DNA replication. Nitrous acid gives rise to transitions, where cytosine is replaced by uracil, which can pair with adenine instead of guanidine. Acridine orange intercalates between DNA bases, distorting the double helix, often resulting in insertions of an extra base on the opposite strand by DNA polymerase, leading to alterations in the reading frame of mRNA molecules transcribed from this region. Base analogues, such as 5 bromouracil (5-BU), 5-bromodeoxyuridine, maleic hydrazide, and 2 amino-purine (2AP), incorporate into DNA, replacing normal bases during replication, causing transitions (purine to purine, or pyrimidine to pyrimidine) and tautomerization (interconversion of guanine from its keto to enol form) which affect affecting pairing during strand displacement and polymerization.

Biological mutagens include mobile genetic elements, such as viruses and transposons, facilitated in some cases by plasmids that can collect and distribute genetic elements in a horizontal fashion from cell to cell. Some viruses integrate their genomes into the chromosomes of host cells in order to replicate, while others propagate as circular plasmids, or as episomes that can propagate as a plasmid that can also integrate into host chromosomes. In eukaryotes, an episome generally means a non-integrated extrachromosomal closed circular DNA molecule that can replicate in the nucleus, such as herpesviruses, adenoviruses, and polyomaviruses. Poxviruses, however, are episomes that replicate in the cytoplasm of infected cells. In prokaryotes, the bacteriophages lambda and Mu have been extensively studied as model systems to understand the relationships between the structure and function of a wide variety of genetic elements, primarily those relating to regulation of transcription and translation of genes encoding structural and regulatory molecules.

### Bacteriophages

Bacteriophages, which may contain single or double-stranded DNA or RNA that can range size from several kb to over 100 kb of nucleic acid, generally comprise replication genes, structural genes, and genes that facilitate recombination or insertion of the viral genome into random or specific locations in the chromosome of a host cell. Virulent bacteriophages can lyse the host bacteria and persist in the environment, while temperate bacteriophages have a quiescent non-lytic growth mode called lysogeny, which may be disrupted by environmental stimuli, such as DNA damaging agents or temperature changes, to provoke a switch to virulent replication, phage production, and cell lysis. Insertion and excision of temperate prophages into and out of chromosomes are often facilitated by homologous recombination events mediated by bacteriophage recombinases and preferred attachment sites on a host chromosome.

### Plasmids

Plasmids are collections of functional genetic elements comprising at least one stable, self-replicating replicon, with regulatory circuits that control its copy number, and genes that encode products for partitioning, that ensure stable inheritance of molecules during cell division. Replicons also contain genes that control incompatibility, generally preventing plasmids having the same replication mechanism to co-exist in the same cell.

Large, naturally occurring plasmids can be classified by their incompatibility group, with 26 groups recognized for the Enterobacteriaceae, 14 groups for the pseudomonads, and 18 groups for the Gram-positive staphylococci. Many synthetic high copy number cloning vectors such as the pUC series, pBR322, pET series, pGEX series, and ColE1 series are generally incompatible with each other, if they have origins of replication derived from ColE1, pMB1, or pBR322. Transforming a pUC-based plasmid into a cell comprising pBR322 and selecting for cells comprising the drug resistance marker carried on the pUC-based plasmid, but not the marker carried on pBR322 will recover cells containing the transformed plasmid. Low to medium copy number plasmids derived from R6K, pSC101, and the pACYC series (comprising a p15A replicon) are compatible with plasmids containing ColE1, pMB1, or pBR322-based replicons. Extremely low copy number conjugative plasmids having 1-2 copies per cell, such as the Fertility (F) plasmid (belonging to the IncFI group), or the Resistance (R) plasmid known as NR1/R100 (IncFII group), are compatible with each other, and all of the higher copy number plasmids noted above. Many synthetic vectors used to construct libraries of Bacterial Artificial Chromosomes (BACs), contain mini-F replicons that have contiguous sets of genetic elements responsible for replication, incompatibility, copy number control, and stability.

Plasmids can also be classified by general function, which are not mutually exclusive. Several classes are recognized: Fertility (F) plasmids contain many tra genes responsible for transfer of the plasmid, and occasionally additional DNA, from one cell to another through conjugation mediated by a pilus. Resistance (R) plasmids often contain many tra genes, plus one or more genes which confer resistance to antibiotics (e.g., chloramphenicol, kanamycin, tetracycline, ampicillin, sulfonamide, spectinomycin, streptomycin), heavy metals (e.g., mercury, silver, cadmium), or other types of toxic agents. Several clinically-relevant R plasmids confer resistance to over 12 different kinds of antibiotics. Col plasmids contain genes that encode bacteriocins (e.g., colicins, microcins, and tailocins) that can kill other bacteria. Degradative plasmids carry genes involved in the metabolysis of unusual organic compounds. Virulence plasmids carry genes which make a bacterium pathogenic under the right conditions. Plasmid-borne drug resistance, bacteriocin, degradation, or virulence genes, can become mobile when they are flanked by Insertion Sequences (IS elements), or become cargo sequences within a transposable element, that can be moved from one cell location to another, or from cell to cell by bacteriophages or conjugative transfer events.

### Transposons

Transposons comprise sequences that encode enzymes called transposases, and sometimes resolvases, that facilitate cut-and-paste transposition, or replicative transposition events. Transposons Tn5, Tn7, and Tn10, move by a non-replicative, cut-and-paste mechanism, leaving one copy on the target DNA site, while transposon Tn3, bacteriophage Mu, and many insertion sequences (IS elements), leave one copy on the donor and the target DNA sites. Many transposons integrate randomly in new locations on the host chromosome or a plasmid harbored by a cell, while a few, like Tn7 and related Tn7-like elements, are integrated at one or more preferred, neutral and defined target sites, typically near the end or within the intergenic region of a highly-conserved, essential host cell gene (e.g., glmS-like genes).

A wide variety of transposons have been used to randomly integrate transposons in bacteria [reviewed in Choi, K.-H. and Kim, K.-J. (2009) J. Microbiol. Biotechnol. 19(3): 217-228]. Bacteriophage Mu, has a replicative form of transposition, producing a 5 bp duplication at the target site, but requires host cell factors for transposition. Tn3 and Tn3-like transposons Tn817 and Tn4430 also have a replicative form of transposition, producing a 5 bp insertion at the target site. Tn5, has a cut-and-paste mechanism, producing a 9 bp duplication at its target site. Engineered forms of Tn5 and its transposase are often used for random mutagenesis of genes in vivo and in in vitro-based systems. Tn10 has a cut-and-paste mechanism, producing a 9 bp duplication at its unique 6 bp target site. Variants of the Tn7 transpose tnsC or tnsD gene products, have been used to generate random mutations, using a cut-and-paste mechanism, producing a 5 bp duplication at its target site.

The ability to randomly transpose cassettes of cargo genes into segments of a bacterial genome, or onto large plasmids propagated in bacteria, greatly facilitates the identification and characterization of essential and non-essential genes. Growth of cells comprising insertions into genes of interest, under specific physiological conditions, often suggests that the disrupted gene is not essential. Lack of growth, or inability to obtain insertions in a particular target segment, is often strong evidence that one or more genes in the targeted segment is essential. Amplification of DNA sequences using a pair of primers, one mapping within one end of the transposon, and the other mapping to a nearby gene of interest, can be used to rapidly identify the specific location of the transposon within the chromosome of a cell or plasmid that has been previously sequenced. Transposons allowing readthrough into either arm of a transposon to drive expression of a promoter-less reporter gene, to produce a gene fusion, have been used to determine the orientation and relative strength of promoters within the target DNA segment. Linker scanning mutagenesis methods have also been developed, where a transposon is randomly integrated into a target site, and a large part of the central core of the transposon removed, to produce random in-frame insertions of short peptides within the target gene.

A few transposons integrate into highly-selective conserved AT-rich target sequences. Insertion Sequence IS605, for example, integrates into the sequence TTAA or TTAAC. Tn916 and Tn1545, found in Gram positive bacteria, insert into a position harboring an A-rich sequence separated by 6 bp from a T-rich sequence, which may not be random enough, or specific enough, for many cell engineering applications.

A most remarkable transposon is Tn7, and Tn7-like elements found in diverse bacteria, that encode homologues of the Tn7 transposition proteins [Peters (2014)]; [Craig, Chapter 124 Transposition]. Tn7 is a 14 kb transposon that encodes resistance to trimethoprim (Tp^{R}) and streptomycin/spectinomycin (Sm^{R}/Spc^{R}) that was originally isolated from E. coli that had infected a calf several years after Tp was first used veterinary settings, and shown to be a mobilizable from an IncI antibiotic resistance plasmid, designated R483, to other plasmid replicons and a site in chromosome of E. coli K12 and in a C600 recA-deficient strain (Hedges et al, 1972; Barth et al, 1976).

The sequence of Tn7 has been determined (GenBank Locus Bm_Tn7, Accession Number BM_NC_002525) and shown to be 14,067 bp (SEQ ID NO: 1), encoding three drug resistance genes: dhfr1 encoding dihydrofolate reductase type I, sat encoding streptothricin acetyltransferase, and aadA encoding streptomycin 3′ adenyltransferase, which are located between positions +2,246 to +4,184. Four open reading frames encoding proteins of unknown function are located at positions +4,260 to +5,976. A gene called int12 located between +937 and +1,914, is described in the GenBank annotations as encoding a site-specific recombinase for integron cassettes, which is not translated beyond amino acid 178, unless a TAA codon is suppressed. The segment of DNA comprising the int12, dhfr1, sat, and aadA genes is called the variable region, and benefit the transposon or the bacterial host cell. Five genes designated tnsA, tnsB, tnsC, tnsD, and tnsE, encoding the TnsABCDE proteins or transposases, are located between positions +6,207 to +13,933, which are encoded on the opposite (−) strand, with tnsA starting near the right end of the transposon (Tn7R) and tnsE ending near the center of the transposon. The left and right arms of Tn7 (Tn7L and Tn7R) comprise sequences comprising a series of 22 bp tnsB binding sites, three in Tn7L extending in 150 bp from the left end of the transposon, and four tightly packed sites in Tn7R, extending in 90 bp from the right end of the transposon.

There are terminal repeats (TRs) located at both ends of the transposon:

(positions +1 to +13 of SEQ ID NO: 1)
5′-TGTGGGCGGACAA-3′

at the left end, and its exact complement

(positions +14,055 to 14,067 of SEQ ID NO: 1)
5′-TTGTCCGCCCACA-3′

at the right end.

Mutagenesis studies have also noted that the TGT and ACA sequences at the terminal left and right ends of these sequences are critical to the cut-and-paste reaction, and highly conserved in all Tn7-like transposons.

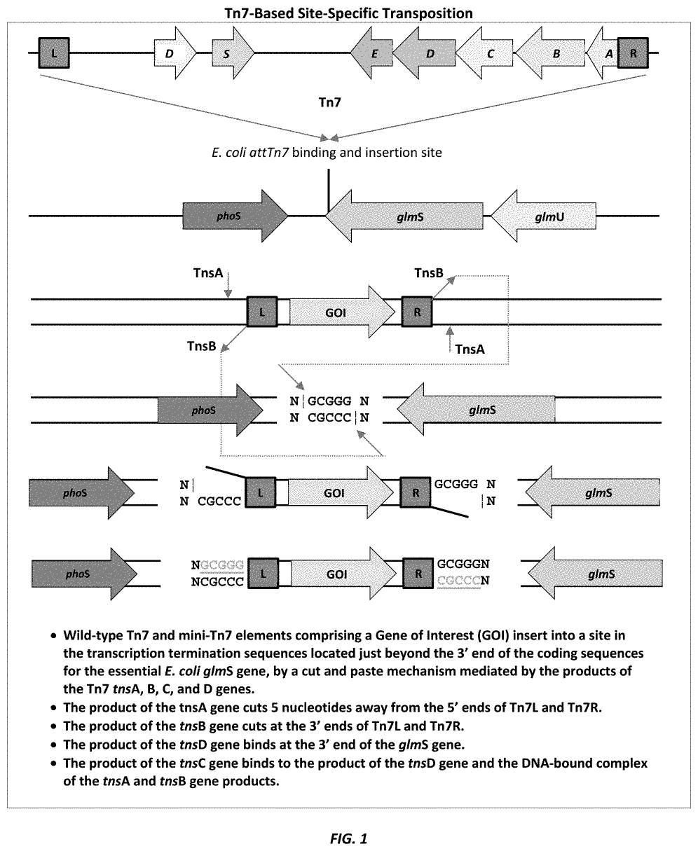

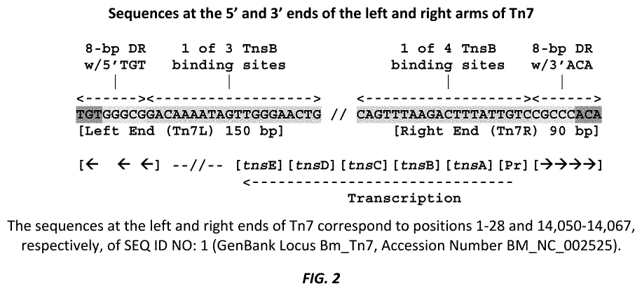

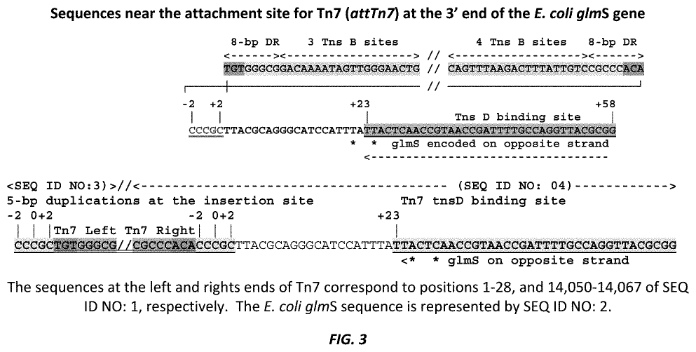

The relative locations and approximate sizes of key genetic elements are shown in FIG. 1, entitled “Tn7-Based Site-Specific Transposons”. FIG. 2 illustrates sequences extending in from the left and right ends of Tn7, designated Tn7L and Tn7R, respectively including the sequences of two of 7 TnsB binding sites and the 8-bp direct repeats (DRs) at both ends of the transposon. FIG. 3 illustrates sequences at the attachment site for Tn7 (attTn7) at the 3′ end of the E. coli glmS gene before and after transposition of a Tn7 element into the target sequence.

Tn7 can move from one location to another by two different pathways. One pathway favors insertion of Tn7 into a single site in the chromosome, called the attachment site, or attTn7, which favors vertical transmission of the transposon from a plasmid, to a daughter cell, while the other pathway, favors insertion of the transposon from the chromosome or other plasmids, into a conjugal plasmid, facilitating horizontal transmission into a new host cell. Site-specific transposition requires the trans-acting products of the tnsA, B, C, and D genes, plus the cis-acting sequences at the left and right ends of the transposon (the terminal repeat sequences, and the tnsB binding sites within Tn7L and Tn7R). Biased transposition, into replication forks on conjugal plasmids and a region in the chromosome where DNA replication terminates, requires the products of the tnsA, B, C, and E genes, plus the cis-acting sequences in Tn7L and Tn7R. In some model systems lacking conjugal plasmids, insertion of mini-Tn7 elements into other plasmids mediated by the products of the tnsA, B, C, and E genes may appear to be random.

The product of the tnsA gene (TnsA), which is 273 aa long, is responsible for cleaving DNA at the 5′ ends of the transposon. A catalytic domain is located in the N-terminal half of the protein, with a DNA binding domain, plus sites where the products of the tnsB and tnsC genes interact are located in the C-terminal half of the protein.

The product of the tnsB gene (TnsB), which is 702 aa long, is responsible for recognizing the left and right ends of the transposon, and allowing them to be paired in a process mediated by the product of the tnsA gene. It contains a catalytic domain near the center of the protein, and a short site for interaction with the product of the tnsA gene near the C-terminal end of the catalytic domain, and a short site for interaction with the product of the tnsC gene near the C-terminal end of the entire protein.

The product of the tnsC gene (TnsC), which is 555 aa long, has several functions. It plays a role in interacting with structural features of target DNA sequences, and has large segments involved in the interaction with product of the tnsD gene and with the product of the tnsA gene. A domain located in the center part of the molecule is involved in the binding and hydrolysis of ATP, which may play a role in target immunity, preventing transposition into segments of DNA comprising an existing copy of Tn7.

The product of the tnsD gene (TnsD), which is 508 aa long, is responsible for binding to the attTn7 target site. It has a conserved zinc finger domain, and a large segment in the first two-thirds of the protein involved in the binding to the product of the tnsC gene. Two host proteins, ACP, an acyl carrier protein, and L29, a component of the large ribosome also appear to play structural or regulatory roles in the insertions of Tn7 into the attTn7 site.

The product of the tnsE gene (TnsE), which is 538 aa long, is responsible for recognizing sites other than attTn7 as targets for insertion of the transposon. It is not a sequence-specific DNA binding protein, but appears to prefer binding to 3′ recessed ends of a replicating DNA structure and a sliding clamp processivity factor (β-clamp protein), encoded by the host dnaN gene. Double-stranded breaks in DNA, mediated by UV light and some chemical mutagens, stimulate DNA repair systems, allowing TnsE-mediated transposition events near replication-induced repair sites near the break. Two segments of the product of the tnsE gene, one near its N-terminus and one near its C-terminus, appear to be involved in binding to the product of the host dnaN gene.

The attachment site, attTn7, is present in the chromosomes of many types of bacteria in the transcriptional terminator of the glmUS operon, which encodes two proteins involved in cell wall biosynthesis [reviewed in Deboy and Craig (2000)]. The product of the glmU gene catalyzes two reactions in the synthesis of UDP-N-acetylglucosamine (UDP-GlcNAc), with the C-terminal domain catalyzing the transfer of an acetyl group from acetyl-CoA to N-acetyl-α-D-glucosamine-1-phosphate (GlcNAc-1-P), and the N-terminal domain catalyzing the transfer of uridine-5-monophosphate from UTP to produce diphosphate and UDP-N-acetyl-α-D-glucosamine. The product of the glmS gene (glutamine-fructose-6-phosphate transaminase (isomerizing)), catalyzes one of the first steps in hexosamine biosynthesis, converting D-fructose 6-phosphate and L-glutamine to D-glucosamine 6-phosphate and L-glutamate.

The nucleotide sequence of a 14.5 kb segment of E. coli DNA from chromosomal origin of replication, oriC, to start of the phoS gene (also called the pstS gene), which includes nine genes of the unc operon encoding subunits of ATPase and the glmS gene, was previously reported [Walker et al (1984)]. In this sequence, the second of two TAA stop codons ends at position +14,201, and the ATG start codon of the phoS gene, encoding a phosphate binding protein, is located at position +14,512, providing for an intergenic region of 310 (=14,511−14,202+1) nucleotides. The sequence of the phoS gene was also reported, including 270 nucleotides of the intergenic region between the end of the glmS gene and the start of the phoS gene [Magota et al, 1984].

Sequences near the 3′ end of the essential glmS gene, extending beyond two adjacent TAA stop codons into a hairpin loop in its transcriptional termination site that are important parts of the target for site-specific insertion of Tn7. The product of the tnsD gene, TnsD, recognizes a 35-bp segment at the 3′ end of the glmS gene, and insertion of the transposon occurs at a point that is about 25 bp away from the start of the TnsD binding site. The center nucleotide of a 5-bp sequence (from relative positions −2 to +2) that is duplicated on insertion, is designated position 0. The TnsD binding site is located in a segment spanning relative positions +23 to +58 in within the coding sequences of the glmS gene, as shown below.

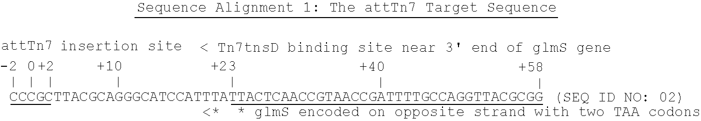

Sequences at the point of insertion are not important, compared to the highly conserved sequences within the 3′ end of the glmS gene [Gringauz et al (1988); Parks and Peters (2007)]. A U-rich stretch of sequences to left of the insertion site, from positions −10 to −6 (not shown), are at the 3′ end of the glmS mRNA, which contains a GC-rich region of dyad symmetry encompassing residues from positions −4 to +13.

Cut and paste transposition into the target site in the intergenic region generates a sequence with Tn7L proximal to the phoS gene, and Tn7R proximal to the glmS gene, flanked on either end by the 5-bp sequence of the insertion site, as shown below.

Sequence Alignment 2: 5-bp Duplications at the attTn7 Target Sequence

<SEQ ID NO: 03>//<------------------------------------- (SEQ ID NO: 04)------------>
5-bp duplications at the insertion site Tn7 tnsD binding site
−2 0+2 −2 0+2 +23 +58
| | |Tn7 Left Tn7 Right| | | | |

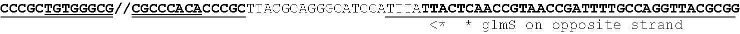

Mutagenesis experiments have demonstrated that changes to nucleotides from residues −2 to +13 do not alter the frequency of insertion into altered sites, suggesting that nucleotides required for attTn7 target activity are within residues +14 to +64. Three of six insertions into a synthetic segment comprising residues +7 to +64, had some wobble, with two having duplications of sequences from positions −1 to +3, one from positions +1 to +5, and the other three, as expected from positions −2 to +2. These results clearly demonstrate that the sequences immediately adjacent to the insertion point are irrelevant to attTn7 target activity [Gringauz et al (1988)].

These and many other observations on the structure and function of genes encoding transposition proteins that act on cis-acting sequences near the left and right ends of Tn7 and its attachment site, stimulated research into other mobile genetic elements capable of targeting specific sequences within the genome of a host cell, or on conjugal plasmids, allowing horizontal transmission of the element from one cell to another. Analysis of over 50 Tn7-like elements have revealed dynamic evolutionary relationships between sequences encoding transposition proteins, some highly conserved, others not, that insert in the same position and same orientation adjacent to a chromosomally-encoded glmS gene [Parks and Peters (2009)]. Diverse arrays of genes in the highly variable region in the left half of the transposon, often encode products with beneficial functions, that contribute to the survival of the host cell. Unlike Tn7, some Tn7-like elements are found in bacteria with multiple elements inserted in tandem near a specifically-defined DNA locus, creating “genomic islands” or clusters of related transposons comprising their highly divergent variable regions. Systematic analysis of these and other mobile genetic elements have greatly facilitated the development of vectors comprising expression cassettes encoding proteins of interest suitable for use in a wide variety of applications.

### Insect Cell-Based Baculovirus Shuttle Vector (Bacmid) Systems

One remarkably successful application of Tn7-mediated transposition of DNA cassettes into large plasmids propagated in E. coli, is the baculovirus shuttle vector (bacmid) system first described over 25 years ago [Luckow et al, 1993]. In this system, a viral shuttle vector was constructed comprising a contiguous segment of genetic elements, including a mini-F low copy number replicon, a gene conferring resistance to kanamycin, and a complex segment comprising a gene encoding the lacZ alpha peptide with an in-frame insertion comprising the attachment site for Tn7. The relative order of genetic elements in this segment is Kan, lacZalpha-mini-attTn7, and mini-F replicon, although these are functionally distinct, and could have been assembled in any order, and in different orientations with respect to each other. This segment, which is 8,579 bp, was inserted into the polyhedrin locus in the baculovirus Autographa californica Nuclear Polyhedrosis Virus (AcNPV) type E2, creating the shuttle vector, or bacmid designated bMON14272. This vector, which propagates in E. coli strain DH10B as a low copy number plasmid, is infectious when transfected into susceptible Lepidopteran insect cells, such as Spodoptera frugiperda Sf9 or Sf21 cells, or Trichoplusia ni cells. Infected cells typically release budded viruses about 24 hpi, but lyse after lyse after 72 hours.

A helper plasmid, designated pMON7124 comprising the right half of Tn7 cloned onto a derivative of pBR322, contains the Tn7R and the tnsABCDE genes encoding all five proteins needed for site-specific or random transposition of Tn7 into the chromosome or other plasmids within the cell [Barry, 1988]. When E. coli strain DH10B, harbors both the bacmid bMON14272, which confers resistance to Kanamycin, and the helper plasmid pMON7124, which confers resistance to Tetracycline, both plasmids co-exist because their replicons are in different incompatibility groups.

A donor plasmid, designated pMON14327, was constructed, that contains the left and right arms of Tn7 (Tn7L and Tn7R) flanking an internal region comprising a gene encoding resistance to gentamycin, along with the strong polyhedrin promoter (Ppolh) driving expression of a gene conceding β-glucuronidase, and a sequence comprising an SV40 poly(A) transcriptional terminator. The order of genetic elements is Tn7L, SV40 poly(A), β-gluc, Ppolh, GentR, and Tn7R, with the promoter and coding sequences for the gentamycin resistance gene oriented towards Tn7R, and the SV40 poly(A)-β-gluc-Ppolh segment oriented in the opposite strand, towards Tn7L. This plasmid derived through many steps, also contains an origin of replication from the cloning vector pUC8, and a gene encoding resistance to ampicillin (AmpR). The replicon in donor plasmid is incompatible with the replicon in the helper plasmid pMON7124, since they were both derived from replicons in the ColE1/pMB1/pBR322/pUC related series of cloning vectors.

When the donor plasmid pMON14327 was transformed into E. coli strain DH10B, harboring bMON14272 and pMON7124, and selecting for colonies on agar plates containing Gentamycin, Kanamycin, and Tetracycline, but not Ampicillin, in the presence of the inducer IPTG and a chromogenic substrate for β-galactosidase, a mixture of white and blue colonies was observed. White colonies were purified by restreaking a second time on the same type of agar plate, and plasmid DNA isolated, and characterized by restriction enzyme analysis. In all cases the plasmid DNA sample contained the bacmid bMON14272 with an insertion of the mini-Tn7 transposon derived from the donor plasmid, pMON14327, inserted into the attTn7 site within the lacZalpha gene, plus leftover (carrier) pMON7124 helper plasmid DNA.

When this mixture of DNA was transfected into Sf9 insect cells, budded viruses were produced, amplifying the infection, and the product of the β-glucuronidase gene expressed under the control of the polyhedrin promoter at very high levels. SDS-PAGE gels of cells infected with the virus vMON14272::Tn14327, derived from the “composite bacmid” bMON14272::Tn14327, had an abundant band corresponding to the expected size for the β-glucuronidase protein. Similar experiments were also carried out demonstrating high levels of expression of human leukotriene A₄ hydrolase, and a variant of human NMT.

One key advantage of this system at the time, was that it was possible to generate pure stocks of virus in 7-10 days, compared to 4 or more weeks using traditional methods of generating recombinant baculoviruses by homologous recombination between baculovirus DNA and a transfer vector in transfected insect cells, where the frequency of recombination was <1%, and requiring several additional plaque assays to confirm the their phenotype and to purify and amplify stocks of the desired recombinant viruses.

This system was patented and licensed by Monsanto to Gibco/BRL/Life Technologies, Inc., which was acquired by Invitrogen, Inc., and later by Thermo Fisher, Inc. The E. coli strain harboring both bMON14272 and pMON7124 is called DH10Bac®. Cloning kits containing a variety of components, including competent DH10Bac cells, and a variety of donor plasmids derived from pMON14327, called pFastBac vectors, and an instruction manual, were developed and sold by these vendors as part of the Bac-To-Bac® system, which are still available from Thermo Fisher. U.S. Pat. No. 5,348,886, which was filed in 1992, expired in 2012.

Three basic derivatives of the donor plasmid pMON14327 were designed and sold by Life Technologies, Inc. [Ciccarone et al (1997)]. The pFastBac1 vector has a large multiple cloning site inserted downstream from the strong polyhedrin promoter. The pFastBacHT vector is similar, but has an N-terminal 6×His tag for rapid affinity purification of recombinant fusion proteins, and a Tobacco Etch Virus (TEV) protease cleavage site allowing for removal of the histidine tag after purification. The pFastBacDual vector has the polyhedrin promoter and the strong p10 promoter for simultaneous expression of two proteins in insect cells. Dozens of derivatives of these and other min-Tn7-based donor vectors are now available from a wide variety of commercial, academic, and non-profit entity sources.

Despite continuous improvements in the design and use of donor vectors from 1993 to the present, very little development is evident from publicly available scientific, patent, or commercial product literature that highlight efforts to improve a key component of this system, the bacmid comprising the bacterial replicon, a drug resistance marker, and the target site for the site specific transposon, attTn7, which was inserted into a gene encoding the lacZalpha peptide. A large part of this may be due to the complexity of assembling the first two bacmids, designated bMON14271 and bMON14272, from 13 precursor plasmids or PCR fragments, and the assembly of the donor plasmid, pMON14327 from a different set of 13 precursor plasmids over a period of nearly two years, before they could be introduced into a cell to confirm that the mini-Tn7 sequence from the donor plasmid would transpose into the attachment site on the bacmid, and that the composite bacmid would express the gene of interest under the control of the polyhedrin promoter in at a high level in susceptible cultured insect cells. Manipulating large plasmids, such as a viral shuttle vector comprising two replicons, will continue to be a challenge, until easier methods of gene assembly, vector construction, gene insertion, and mutagenesis of genes of interest are developed and made available for use as research tools, and in the development of food and drug products, industrial processes, and in environmental research applications.

### Prokaryotic Cell Engineering

Tn7 is a widely-dispersed “cut and paste” bacterial transposon, capable of inserting at a very specific location within the chromosome, mediated by the products of the tnsA, B, C, and D genes, or at random locations on conjugal vectors by products of the tnsA, B, C, and E genes. It can also transpose into random locations in the chromosome or on a vector, by the products of the tnsA and B genes, plus a mutant “gain of function” product of the tnsC gene.

While procedures for engineering prokaryotic cells are fairly well established using a combination of donor, helper, and target vectors comprising sequences that include a mini-Tn7 element, genes encoding transposition proteins, and specific attachment sites, respectively, vectors and efficient procedures for modifying eukaryotic cells with Tn7-based elements, particularly mammalian, plant, and fungal cells, are lacking.

Engineering Tn7 to improve its ability to transpose into vectors harbored in eukaryotic cells, or directly into the chromosome will require vectors that have promoters that can drive expression of genes encoding specific transposon products. Each gene may need to be redesigned to reflect codon preferences for a specific host cell, and genes comprising one or more alterations, encoding protein variants, such as those enhancing the level of transposition (hyper-transposases) or the efficiency of insertion at a specific target site (altered specificity) located on a vector or in the host cell chromosome will also be generated and analyzed. Promoters and transcription termination signals may also need to be altered to function properly in a eukaryotic host cell.

The product of the tnsD gene binds to the 3′ end of the E. coli glmS gene, which facilitates the binding of the product of the tnsC gene that is also bound to the products of the tnsA and B genes bound to the 5′ and 3′ ends of Tn7. The Tn7 element inserts at a position that is about 25 bases away from the 5′ end of the TnsD binding site, producing a 5-bp duplication on both sides of the element. Human and yeast homologues of the E. coli glmS gene also bind the product of the tnsD gene, but at lower efficiencies, and while transposition of Tn7 into each of the two human homologues was demonstrated over 15 years ago, it was not demonstrated for the yeast homologue carried on a vector propagated in bacteria, or in a reconstituted system using purified bacterial proteins.

There do not appear to be any reports in the primary scientific literature disclosing experiments where sequences encoding the product of the tnsD gene were mutagenized, that were coupled to methods for the direct selection of variants that would have enhanced or altered specificities, to bind more favorably to sequences like the human or yeast homologues of the E. coli glmS gene, compared to the wild-type bacterial sequence. Our novel selection methods, can be used in directed evolution experiments to develop synthetic Tn7-based transposons that should efficiently insert transposons into the chromosome and shuttle vectors harbored in eukaryotic cells.

### Eukaryotic Cell Engineering

There is an emerging trend to use transposons to deliver large segments of DNA into cultured eukaryotic cells, including mammalian cells, supplanting decades of research involving use of viral vector delivery systems. Two which have emerged over the last decade, are the Sleeping Beauty (SB) transposon, derived from salmon, and the piggyBac (PB) transposon, derived from Trichoplusia ni, a caterpillar [Reviewed in Skipper et al (2013) J Biomedical Sci 20(1): 92]. Both are fairly simple, and capable of randomly transposing cassettes of sequences directly into chromosomes of eukaryotic cells, typically using two separate vectors that are co-transfected into a cell: a donor comprising the arms of the transposon that have inverted terminal repeats (ITRs) flanking an expression cassette, and a helper, comprising sequences encoding a transposase that can bind to the ITRs, allowing the donor cassette to be excised from the donor and randomly integrated elsewhere in the chromosome.

Eukaryotic transposons have several advantages over viral vector delivery systems:
Lower production costs, mostly related to production of plasmid DNA samples under GMP conditions compared to production, titering, and testing for replication-competent virus particles.
Lower biosafety requirements, using level 1 or 2 laboratory equipment and hoods.
Lower immunogenicity, due to absence of genetic materials that encode viral proteins, RNA molecules, or other regulatory DNA sequences that may give rise to immunological recognition of molecules associated with the background vector system.
Fairly large cargo capacity, of 12 kb for SB, without a significant loss in transposition efficiency.

Engineered SB and PB transposons face several obstacles as gene delivery systems, however, compared to viral vector systems.
Potential for remobilization and insertional mutagenesis, due to residual activity of the transposase already expressed by the helper vector that was lost from the cell, or expressed by a helper vector propagated as a plasmid, or with key sequences integrated elsewhere in the genome.
Potential for remobilization based on activities of homologous transposases encoded by other eukaryotic transposons.
Footprint mutagenesis, caused by the 3-5 bp sequences left behind when SB remobilizes to a new location, potentially altering reading frames of coding sequences now lacking the SB element.
The 5′ ITR of PB apparently has transcriptional activity that may interfere with nearby promoters.
The integration pattern of PB is similar to retroviral vectors, integrating mainly in transcriptional start sites and transcriptional units, raising concerns about the long-term safety of these vectors.
PB may integrate at locations other than target sites comprising expected TTAA sequences at a low frequency (2%).

The following tables compare key features of different gene editing systems, and key features of random and site-specific transposons, and the site-specificity and efficiency of different gene editing/gene Insertion systems.

TABLE 1
Key Features of ZFN, TALEN, CRISPR/Cas9 and Tn7 Gene Editing Systems*

| | ZFN | TALEN | CRISPR/Cas9 | Tn7 |
|---|---|---|---|---|
| Key | Site-specific cleavage | Site-specific | Ability to target specific | Efficient, reproducible |
| advantages | of dsDNA targeted by | cleavage of dsDNA | sequences complementary | insertion of large cargo DNA |
| | an engineered ZFN | targeted by an | to the guide RNA, where | segments into a specific site |
| | endonuclease | engineered TALEN | dsDNA cleavage events | located in a stable location on |
| | | endonuclease | take place, and repaired by | a target vector or in the host |
| | | | host cell gene products | cell chromosome of bacteria, |
| | | | | and eventually, eukaryotic |
| | | | | cells |
| Recognition | Zinc-finger protein | Tandem repeat of | Single-strand guide RNA | E. coli glmS gene and |
| site | | TALE protein | | homologues |
| Enzyme(s) | Fok1 nuclease | Fok1 nuclease | Cas9 nuclease | tnsABC+ D transposases |
| Target | Typically 9-18 bp/ | Typically 14-20 bp/ | Typically 20 bp guide | 44-bp tnsD product binding |
| sequence | ZFN monomer, 18-36 | TALEN monomer, | sequence + PAM sequence | site, with insertion 20 bp away |
| size | bp per ZFN pair | 28-40 bp/TALEN | | creating a 5-bp duplication |
| | | pair | | |
| Specificity | Tolerating a small | Tolerating a small | Tolerating positional/ | Highly specific binding by |
| | number of positional | number of positional | multiple consecutive | tnsD gene product |
| | mismatches | mismatches | mismatches | |
| Targeting | Difficult to target | 5′ targeted base must | Targeted site must precede | 3′ end of glmS gene is highly |
| limitations | non-G-rich sites | be a T for each | a PAM sequence | conserved in bacteria, with |
| | | TALEN monomer | | homologues in humans and |
| | | | | yeast |
| Difficulty | Requiring substantial | Requiring complex | Using standard cloning | Modifying E. coli systems to |
| of | protein engineering | molecular cloning | procedures and oligo | work in other bacteria should |
| engineering | | methods | synthesis | be easy, and feasible for |
| | | | | eukaryotic cells |
| Difficulty | Relatively easy as the | Difficult due to the | Moderate, as the | Components typically |
| of | small size of ZFN | large size of | commonly used SpCas9 is | delivered as target, helper, |
| delivering | expression elements is | functional | large and may cause | and donor vectors |
| | suitable for a variety | components | packaging problems for | |
| | of viral vectors | | viral vectors such as AAV, | |
| | | | but smaller orthologs exist | |

*ZFN: Zinc-finger nuclease;
TALEN: Transcription activator-like effector nuclease; and
CRISPR: Clustered regularly interspaced short palindromic repeat [Adapted from Li, H., Yang, Y., Hong, W., Huang, M., Wu, M., and Zhao, X. (2020) Signal Transduction and Targeted Therapy 5: 1].

TABLE 2
Key Features of Eukaryotic SB, PB, TcB, Leapin, and Prokaryotic Tn7 Cut and Paste Transposons*

| | Sleeping Beauty | piggyBac | Leap-in 1 and 2 | TcBuster | |
|---|---|---|---|---|---|
| | (SB) | (PB) | (L1 & L2) | (TcB) | Tn7 |
| Key | Fairly small | Fairly small | Fairly small | Fairly small | Efficient, reproducible insertion of |
| advantages | transposon | transposon | transposon | transposon | large cargo DNA segments into a |
| | integrates | integrates | integrates | integrates | specific target located in a stable |
| | randomly into | randomly into | randomly into | randomly into | location on a vector or in in the |
| | TA sequence | TTAA | TTAA, TTAA | NNNTANNN | chromosome of bacteria, and with |
| | | sequences, | sequences, no | sequences in | synthetic transposon and helper |
| | | no excision | excision footprint | GC-rich regions | systems, in eukaryotic cell |
| | | footprint | | | |
| Kingdom | Eukaryotic | Eukaryotic | Eukaryotic | Eukaryotic | Prokaryotic |
| Superfamily | Tc1/mariner | piggyBac | piggyBac | hAT | Tn7 |
| Original | Reconstructed | AcNPV | Leap-In 1 | Consensus | E. coli Incl plasmid R483 |
| Source | by reverse | baculovirus | (Xenopus | sequence derived | |
| | evolution of | propagated in | tropicalis) | from the flour | |
| | consensus from | Trichoplusia ni | Leap-In 1 | beetle Tribolium | |
| | 8 Salmonid | 368 cabbage | (Bombyx mori) | castaneum | |
| | species | looper cells | | | |
| Original size | 1.6 kb | 2,475 bp | N/A | 2,489 bp | 14,067 bp |
| Flanking | 230-bp long IRs | Identical 13-bp | Nearly identical | 328 bp L end and | ~150-bp Tn7L and ~90-bp Tn7R. |
| Regions | | TIRs and | 16 bp ITR (L1) | 145 bp R end | containing 8 bp DIRs adjacent to |
| | | asymmetric | Identical 16-bp | containing 18-bp | 5-bp duplications |
| | | 19-bp IRs, | ITR (L2) | TIRs | |
| | | ~311 bp 5′ end, | | | |
| | | ~235 bp 3′ end | | | |
| Transposase | 360 (SBase) | 594 (PBase) | 589 (L1) requiring | 639 (TcBase) | 273 (TnsA) |
| length (aa), | | PB 23% to L1 | NLS fused to | | 702 (TnsB) 555 (TnsC) 508 (TnsD) |
| homology | | PB 36% to L2 | transposase, | | 538 (TnsE) |
| (%) | | | 610 (L2) | | |
| | | | L1 22% to L2 | | |
| Integration | Random, in | Random, in | Random, 80-90% | Random, in | Site-specific (tnsABC + D), |
| preference | AT-rich regions | AT-rich | transcriptionally- | GC-rich regions, | or Random (tnsABC + E) |
| | (31-39% into | regions, | active gene rich | Transcriptional | |
| | genes) | Transcriptional | genomic segments | units | |
| | | units (47-67% | | | |
| | | into genes) | | | |
| Recognition, | TA | TTAA | TTAA | NNNTANNN | 5-bp staggered cut ~25 bp from 3′ end |
| integration | | | TTAT | | of E. coli glmS gene extending for |
| sequences | | | | | ~44 bp |
| Excision | C(A/T)GTA | None | None | NNNTANNN | None |
| footprint | | | | | |
| Cargo | ~12 kb | ~100 kb | N/A | N/A | >50 kb |
| capacity | | | | | |
| Key variants | SB100X, SB11, | 7 pB, hyPBase | 25 > 50× (L1) | TcBuster V₅₉₆A | “Gain of Function” TnsC* mutants |
| | SB10, HSB5 | (7 aa subs) | 20 > 50× (L2) | | allowing random transposition |
| | | w/10× activity | | | using tnsABC* gene products. |

*SB: Sleeping Beauty, a random eukaryotic transposon;
PB: piggyBac, a random eukaryotic transposon;
Tn5: a random prokaryotic transposon, and
Tn7: a site-specific prokaryotic transposon [Portions adapted from Skipper et al (2013) J Biomedical Sci 20(1): 92].

TABLE 3
Comparing Site-Specificity and Efficiency of Gene Editing/Gene Insertion Tools*

| | CRISPR/Cas | CRISPR/Tn (CAST) | Tn7 | Tn7-like elements |
|---|---|---|---|---|
| Key | Cas nuclease and a | CRISPR-associated | tnsABCD genes encoding | Homologues of tnsABCD |
| Components | single-stranded | transposase from | transposases, and Tn7L and | genes, and L and R arms of |
| | guide RNA | cyanobacteria and | Tn7R sequences, and specific | Tn7-like elements, some of |
| | | natural nuclease | target sites | which have target sites that are |
| | | deficient effector | | completely different from |
| | | Cas12k and a gRNA | | homologues of the E. coli |
| | | | | glmS gene |
| Technical | The gRNA can be | Insertion of up to | Large cargo capacity | Tn7 like elements may not be |
| Advantages | designed to target | 2.5 kb cargo | (20-50 kb) in the mini-Tn7 | subject to transposition |
| | many but not all | segment occurs at an | donor element, site-specific | immunity, allowing sequential |
| | sequences, efficient | efficiency of 60% | integration into target | insertions into target sites in a |
| | for producing | | sequence in a stable location | genomic island on a vector or a |
| | nucleotide | | on a vector or host cell | host cell chromosome; Arrays |
| | substitutions or | | chromosome; Arrays of | of synthetic target sites may |
| | deletions | | synthetic target sites may | allow sequential insertions of |
| | | | allow sequential insertions of | many synthetic Tn7-like |
| | | | many synthetic Tn7 elements | elements |
| Limitations | Off target alterations, | Off target mutations | Need to alter regulatory | Components have been |
| | inefficient for | mostly at genes with | sequences and coding | identified by bioinformatics |
| | insertions >1 kb, and | high rates of | sequences for use in many | studies, but not reassembled |
| | insertions require | transcription | non-enteric bacterial or | into complete systems; Need to |
| | homology arms of | | eukaryotic systems | alter sequences to work in other |
| | up to 1 kb on | | | host cell systems. |
| | either side of the | | | |
| | double-stranded | | | |
| | break (DSB) | | | |
| Challenges | Reducing off | Reducing off target | 3-4 gene products are required | Reconstructing Donor, Helper, |
| | target alterations | insertions or | for random or site-specific | Target Vector Systems |
| | caused by | deletions, and | transposition, respectively | |
| | homology directed | increasing cargo | | |
| | repair HDR) or | capacity. | | |
| | non-homologous | | | |
| | end joining | | | |
| | (NHEJ) | | | |

*[This work (2020)].

### Critical Needs in Synthetic Biology

There exists a need to improve existing methods of introducing cassettes comprising one or more genes of interest into one or more locations on large plasmids or shuttle vectors propagated in bacteria. Improvements to the donor plasmid, the helper plasmid, and the target site located on the plasmid or shuttle vector, which reduce the amount of time, or cost of generating a recombinant vector, and methods which facilitate the rapid analysis of mutagenized genes of interest inserted into a vector will dramatically accelerate R&D activities leading to improved products and services in a wide variety of fields of use.

Several fields of biology can immediately benefit by using and extending the technology disclosed in this application. Improved baculovirus vectors can be developed, which will allow more rapid generation of recombinant viruses used to express heterologous proteins in cultured insect cells and insect larvae. Modular DNA segments comprising the gene cassettes encoding novel gene fusions comprising synthetic mini-attTn7 target sequences can also be moved to a variety of mammalian virus shuttle vectors, plasmids having the capability of transforming plant cells, fungal shuttle vectors and a wide variety of non-enteric bacteria, suitable for use in environmental monitoring and bioremediation applications.

### SUMMARY OF THE INVENTION

A major aspect of the invention relates to a nucleotide sequence comprising a target site for a site-specific transposon, wherein said target site comprises a target sequence comprising a transcriptionally or translationally fused marker sequence encoding a selectable marker sequence or a screenable marker sequence operably-linked to a sequence comprising a specific target sequence for recognition and insertion of a site-specific transposon or a site-specific recombinase, wherein said fused marker sequence encodes an inactive or an active polypeptide capable of conferring a selectable or screenable phenotype upon a cell comprising the fused marker sequence, wherein insertion of the site-specific transposon into the target sequence to create a composite target sequence changes the phenotype of a cell comprising the composite screenable or selectable marker sequence compared to a cell comprising just the selectable or screenable marker sequence.

Another major aspect of the invention relates to a method of screening or selecting for transposition of a site-specific transposon into a nucleotide sequence comprising an attachment site for a site-specific transposon operably-linked to a screenable or selectable marker sequence, comprising the steps of (i) introducing into a bacterial cell a target vector comprising a marker sequence that encodes one or more active or inactive polypeptides capable of conferring a screenable or selectable phenotype upon a cell comprising the marker sequence, wherein insertion of the site-specific transposon into the attachment site to create a composite marker sequence changes the phenotype of a cell comprising the screenable or selectable marker sequence; (ii) introducing into said cell comprising said target vector, a donor vector comprising sequences capable of transposing the wild type or a variant form of the site-specific transposon, and optionally a helper vector comprising sequences encoding one or more transposase gene products; (iii) culturing and optionally plating bacteria comprising the target vector, and optionally donor and helper vectors, (iv) screening or selecting for bacterial colonies where transposition of the site-specific transposon into the attachment site on the target vector to create a composite marker sequence changes the phenotype of the bacterial cell harboring the target vector.

A better understanding of the invention will be obtained from the following detailed descriptions and accompanying drawings, which set forth illustrative embodiments that are indicative of the various ways in which the principals of the invention may be employed.

### BRIEF DESCRIPTION OF THE DRAWINGS

### Statement Concerning Drawings Executed in Color

This patent or application file contains at least one drawing executed in color. Copies of this patent or patent application publication with color drawing(s) will be provided by the Patent Office upon request and payment of the necessary fee.

### Statement Concerning Aspects of the Invention Understood by Reference to the Drawings

The foregoing aspects and many of the attendant advantages of this invention will become more readily appreciated as the same become better understood by reference to the following detailed description, when taken in conjunction with the accompanying drawings, wherein:

FIG. 1 sets forth an illustration entitled “Tn7-based site-specific transposition” that shows how Tn7 recognizes target sequences at the 3′ end of the E. coli glmS gene and inserts into an intergenic region between the phoS and glmS genes.

FIG. 2 sets forth an illustration entitled “Sequences at the 5′ and 3′ ends of the left and right arms of Tn7” that shows the sequences of repeat sequences at the ends of Tn7 and the relative locations of binding sites for the TnsB protein.

FIG. 3 sets forth an illustration entitled “Sequences near the attachment site for Tn7 (attTn7) at the 3′ end of the E. coli glmS gene” that shows the sequences of the ends of Tn7 and its target sequence before and after transposition.

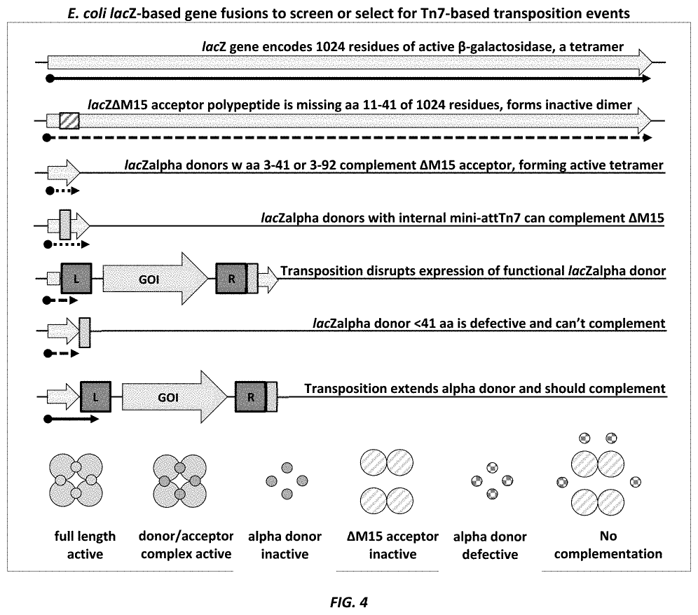

FIG. 4 sets forth an illustration entitled “E. coli lacZ-based gene fusions to screen or select for Tn7-based transposition events” that shows how insertion of a transposon into a synthetic mini-attTn7 sequence in the middle of the lacZalpha gene disrupts expression of the alpha peptide that is needed to complement the activity of the lacZΔM15 acceptor polypeptide, and a second type of gene fusion where insertion of Tn7 extends the sequence of an truncated, inactive alpha peptide to produce an extended alpha peptide that is active, and can complement the acceptor polypeptide.

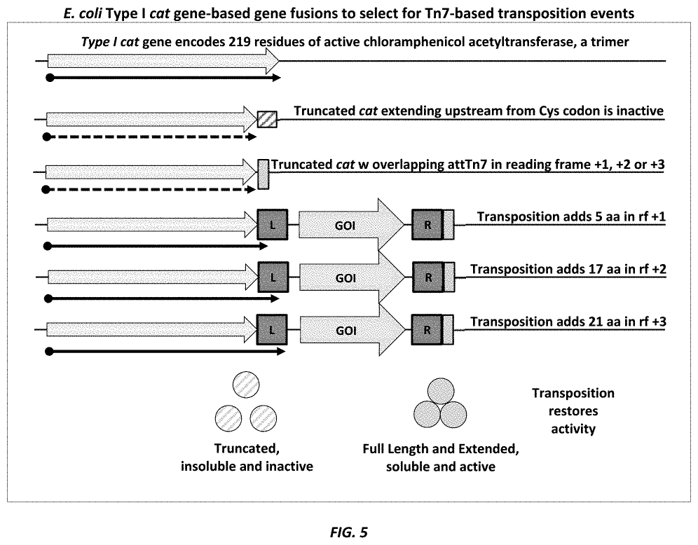

FIG. 5 sets forth an illustration entitled “E. coli Type I cat gene-based gene fusions to select for Tn7-based transposition events” that shows how a gene encoding truncated CAT protein can be extended after transposition to express an active fusion protein that confers resistance to chloramphenicol.

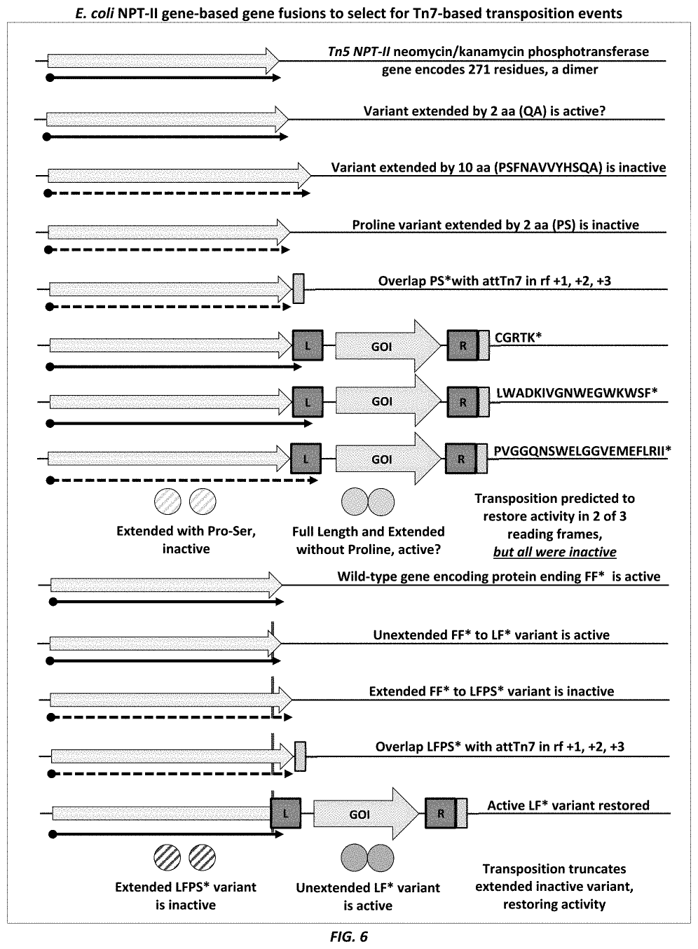

FIG. 6 sets forth an illustration entitled “E. coli NPT-II gene-based gene fusions to select for Tn7-based transposition events” that shows two types of gene fusions, one where an inactive, slightly extended variant of the NPT-II protein is replaced by a sequence encoding extended forms in three reading frames with amino acid sequences derived from the 5′ end of Tn7L. The second type of gene fusion comprises an altered 3′ end of the NPT-II gene comprising a Phe (F) to Leu (L) mutation two amino acids upstream from the natural C-terminal end of the enzyme, plus an extension encoding Phe (F) and Ser (S), which results in an inactive enzyme. Transposition into the second gene fusion with a mini-transposon comprising an altered Tn7L, generates a gene fusion that encodes an unextended, active variant protein.

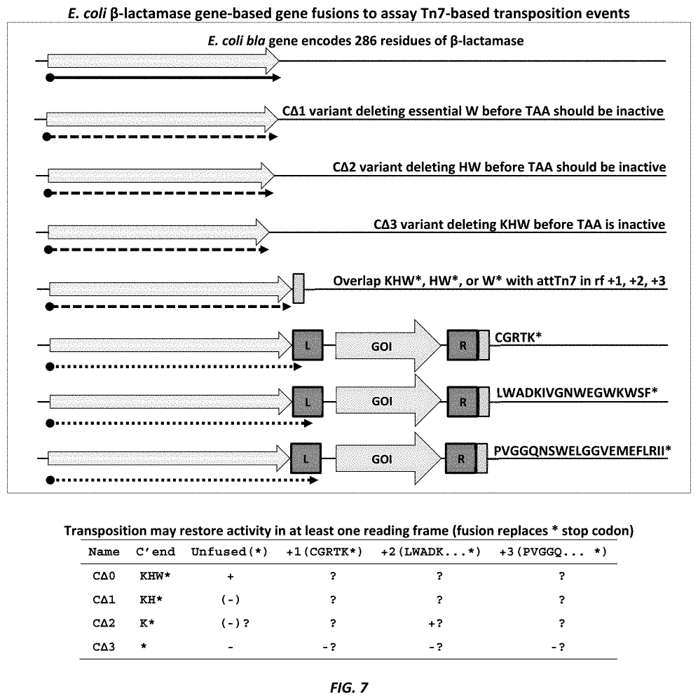

FIG. 7 sets forth an illustration entitled “E. coli β-lactamase gene-based gene fusions to assay Tn7-based transposition events” showing several schemes where extension of truncated versions of the bla gene encode longer fusion proteins that may or may not have activity compared to the wild-type enzyme.

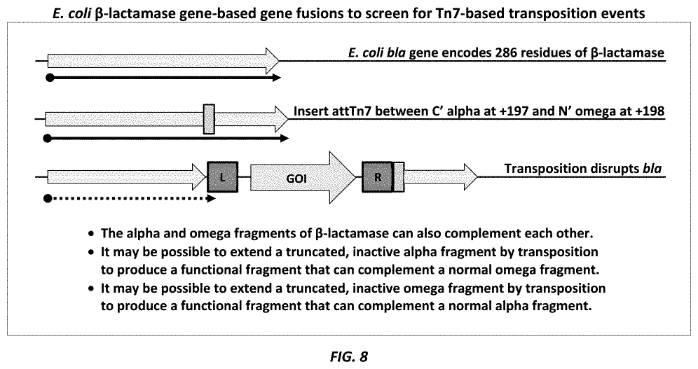

FIG. 8 sets forth an illustration entitled “E. coli β-lactamase gene-based gene fusions to screen for Tn7-based transposition events” showing insertion of a transposon into a target sequence located between the left and right halves of the protein, to encode a product that is inactive.

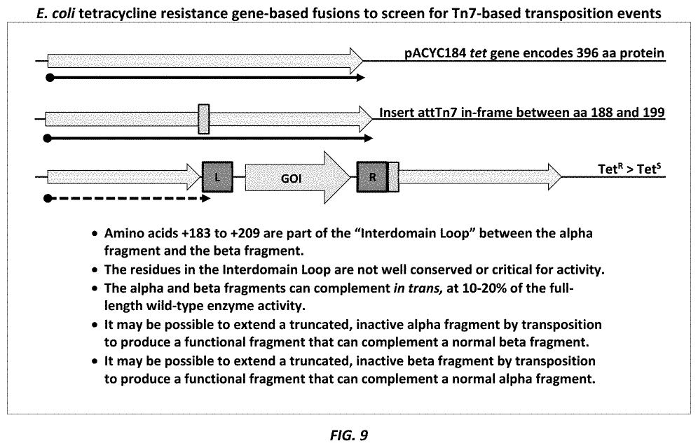

FIG. 9 sets forth an illustration entitled “E. coli tetracycline resistance gene-based fusions to screen for Tn7-based transposition events” showing a scheme of a transposon into a target sequence located in the “interdomain loop region” between the left and right halves of the protein, to encode a product that is inactive.

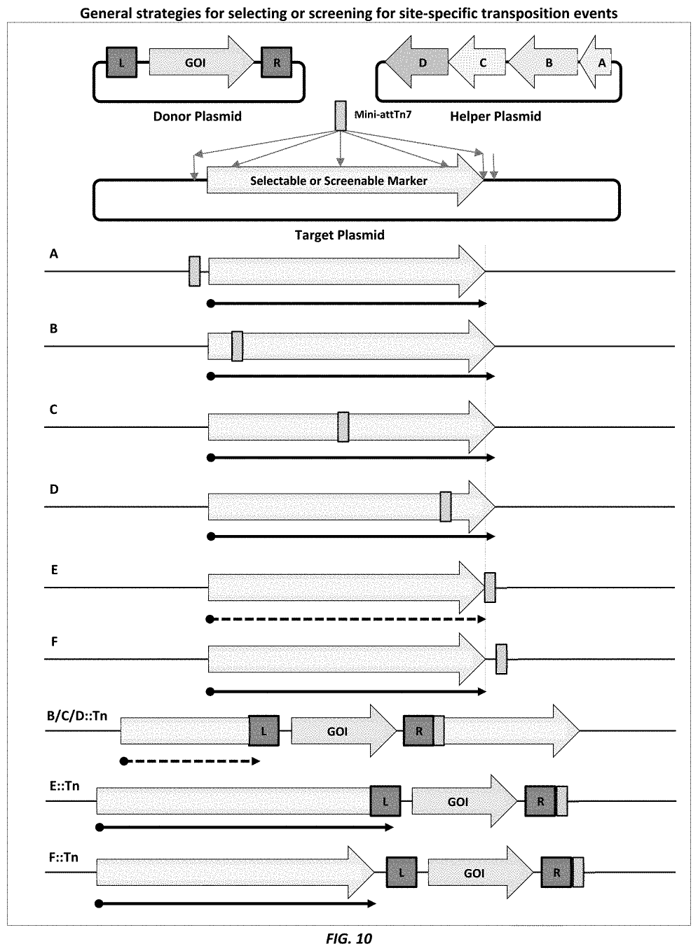

FIG. 10 sets forth an illustration entitled “General strategies for selecting or screening for site-specific transposition events” showing the relative locations of synthetic target sites that can be placed before, within, at the 3′ end, or beyond the 3′ end of the coding sequence of a gene encoding a protein that confers a screenable or selectable phenotype on a cell.

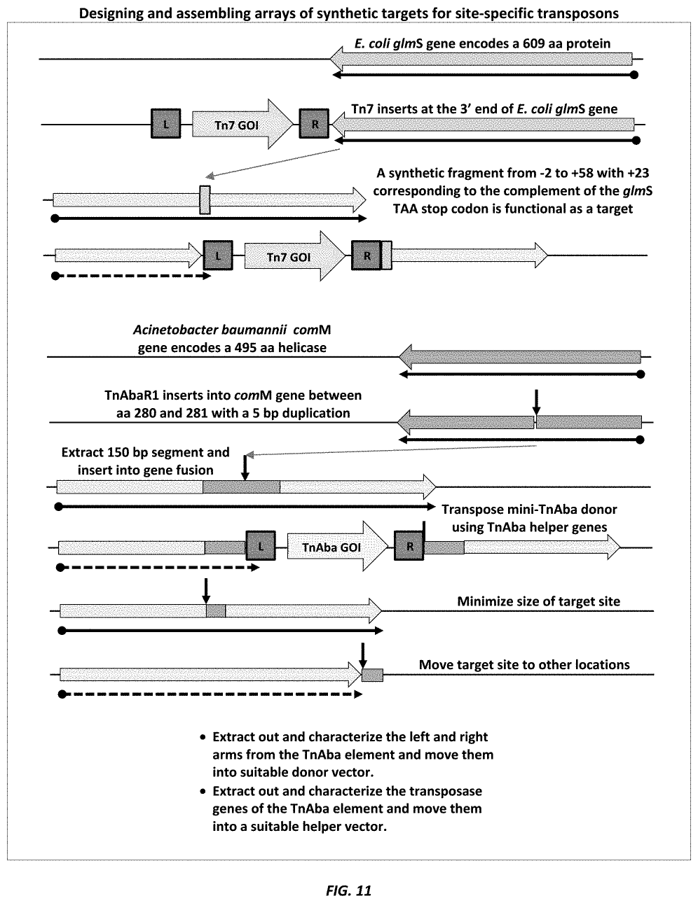

FIG. 11 sets forth an illustration entitled “Designing and assembling arrays of synthetic targets for site-specific transposons” comparing insertion of Tn7 into a synthetic target site derived from the essential E. coli glmS gene, with cloning and targeting a sequence derived from the Acinetobacter baumannii comM gene that can be used to monitor transposition of TnAbaR1 or related Tn7-like elements using a vector comprising a target sequence encoding an active or inactive fusion protein.

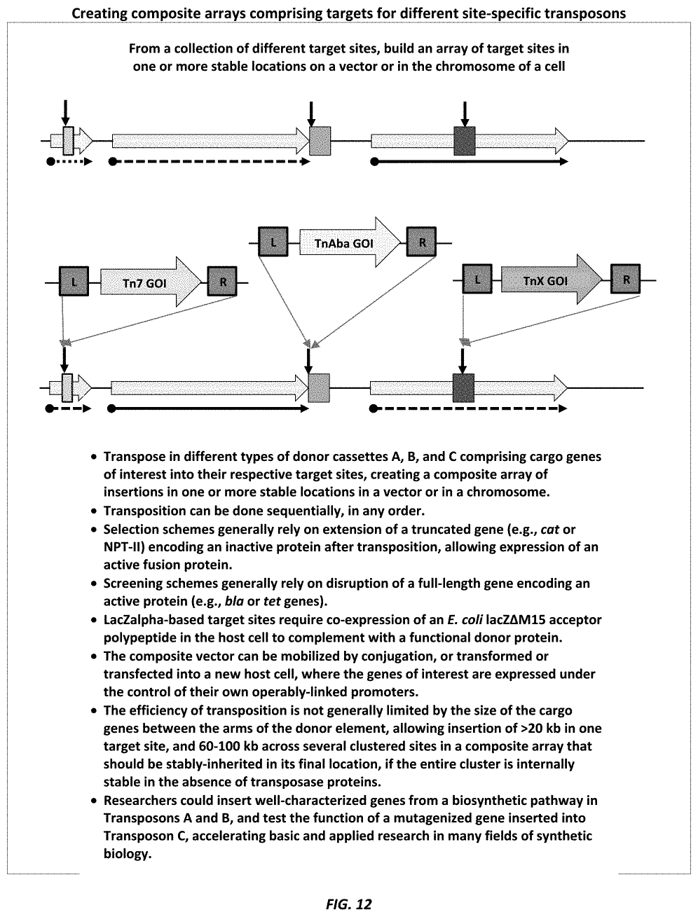

FIG. 12 sets forth an illustration entitled “Creating composite arrays comprising targets for different site-specific transposons” which shows methods for building an array of different kinds of gene fusions that allows for selection or screening of cells comprising composite vectors with sequences derived from several site-specific transposons.

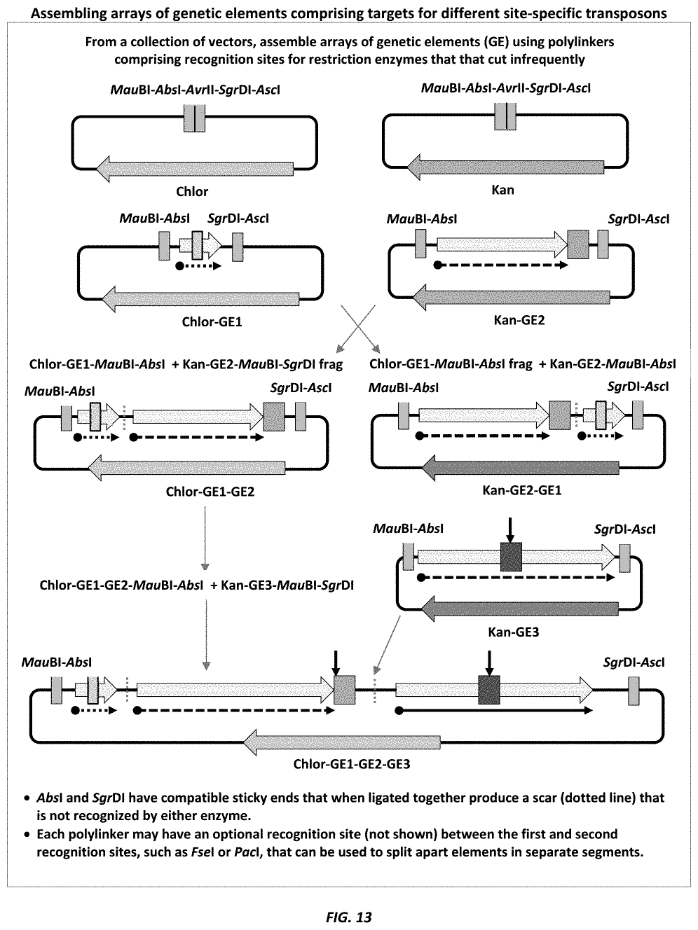

FIG. 13 sets forth an illustration entitled “Assembling arrays of genetic elements comprising targets for different site-specific transposons” shows how target vectors comprising several two to three fusions can be assembled from parent vectors comprising one or two gene fusions by traditional cloning methods.

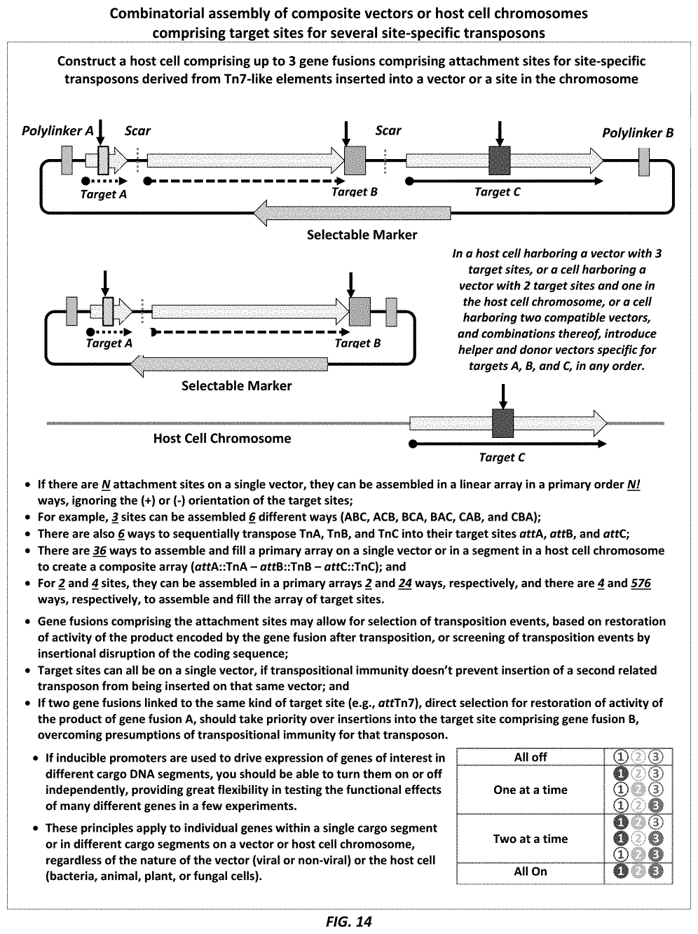

FIG. 14 sets forth an illustration entitled “Combinatorial assembly of composite vectors or host cell chromosomes comprising target sites for several site-specific transposons” shows how a cell harboring a target vector comprising 3 target sites, or a host cell comprising a target vector with 2 target sites, and a target site on the chromosome can be used to analyze the function of complex sets of genes within a cell.

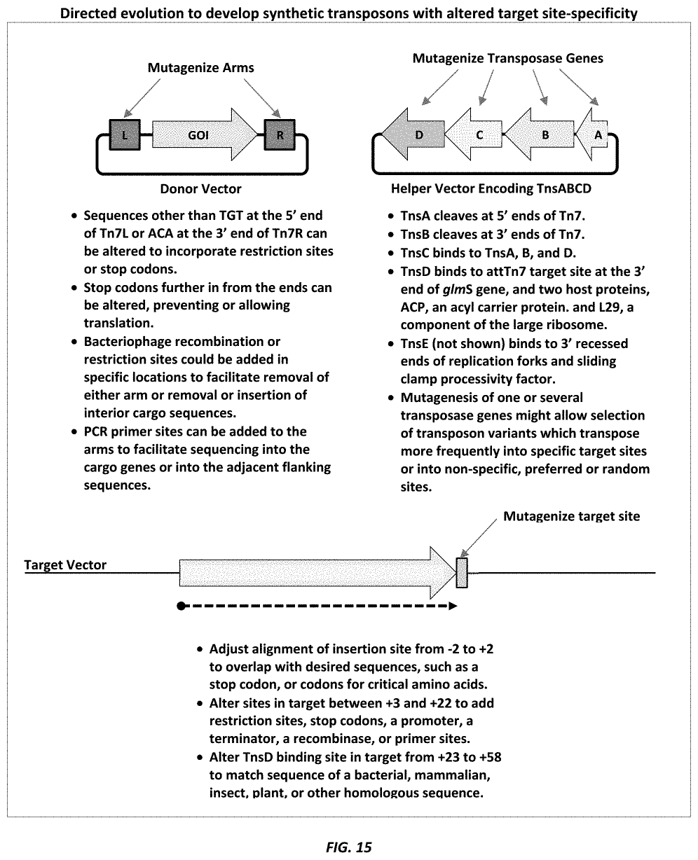

FIG. 15 sets forth an illustration entitled “Directed evolution to develop synthetic transposons with altered target site-specificity” shows basic features of a set of donor/helper/target vectors to facilitate the mutagenesis and selection of transposase genes that have altered specificities or enhanced levels of transposition compared to the wild-type transposase genes, or have altered arms of the transposon to comprise restriction sites or stop codons for specific applications.

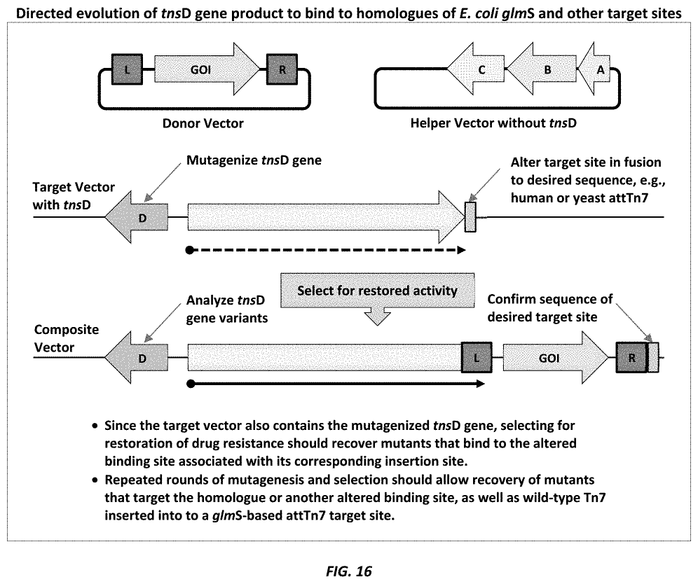

FIG. 16 sets forth an illustration entitled “Directed evolution of tnsD gene product to bind to homologues of E. coli glmS and other target sites” showing a system where the tnsD gene is deleted from the helper vector and mutagenized versions of that gene included in a library of altered target vectors, which allow for selection of cells harboring composite vectors with insertions into target sequences that might not otherwise be recoverable using wild-type transposase genes. Target sequences of interest include homologues found in mammalian cells, such as human, non-human primate, bovine, mouse, and rat sequences, plus fungal homologues found in filamentous and non-filamentous fungi, including yeast.

### ABBREVIATIONS, TERMS AND THEIR DEFINITIONS

The following is a list of abbreviations, plus terms and their definitions, used throughout the text of the specification, the figures, the sequence listing, supplementary data tables (if any), and the claims:

TABLE 4
List of Abbreviations

A = adenosine;
A = absorbance (1 cm);
aa or AA = amino acid;
Ab = antibody(ies);
AcNPV = Autographa californica Nuclear Polyhedrosis Virus, a member
of the Baculoviridae family of insect viruses;
Amp, Ap = ampicillin;
ATP = Adenosine triphosphate;
attTn7 = attachment site for Tn7 (a preferential site for Tn7 insertion into
bacterial chromosomes);
βGal, β-Gal = β-galactosidase;
b = E. coli-derived bacmid;
bc = E. coli-derived composite bacmid;
bch = mixture of E. coli-derived composite bacmid and helper plasmid;
bla = beta lactamase gene conferring resistance to beta-lactam antibiotics,
particularly ampicillin;
Bluo-gal = halogenated indolyl-β-D-galactoside;
BmNPV = Bombyx mori nuclear polyhedrosis virus;
bp, Bp = base pair(s);
BSA = bovine serum albumin;
C = cytidine;
Cam or CM = chloramphenicol;
cAMP = cyclic adenosine 3′,5′-monophosphate;
CAT = chloramphenicol acetyltransferase;
cat = gene encoding CAT;
CBB = Coomassie Brilliant Blue;
ccc = covalently closed circular;
cDNA = DNA complementary to RNA;
CHO = Chinese hamster ovary;
CIAP = calf intestinal alkaline phosphatase;
Cm = chloramphenicol;
CMP = cytidine monophosphate;
cp = chloroplast;
cpm = counts per minute;
CTP = cytidine triphosphate;
Δ = deletion;
d = deoxyribo;
dd = dideoxyribo;
DMF = N,N-dimethylformamide;
DMSO = dimethylsulfoxide;
DNase = deoxyribonuclease;
dNTP = deoxyribonucleoside triphosphate;
ds = double strand(ed);
DTT = dithiothreitol;
EF = elongation factor;
ELISA = enzyme-linked immunosorbent assay;
Er = erythromycin;
EST = expressed sequence tag;
EtBr, EtdBr = ethidium bromide;
FITC = fluorescein isothiocyanate;
g = gram(s);
G = guanosine;
G418 = Geneticin;
Gen or Gent = gentamicin;
GLC-MS = Gas-liquid chromatography-mass spectrometry;
Gm = gentamicin;
HPLC = high performance liquid chromatography;
Hy = hygromycin;
IF = initiation factor;
Ig = immunoglobulin(s);
IL = interleukin;
IPTG = isopropyl β-D-thiogalactopyranoside;
IS = insertion sequence(s);
Kan = kanamycin;
kb or kbp = kilobase(s) = 1000 bp(s);
kDa = kilodalton(s);
Km = kanamycin;
lacZpo = lac promoter-operator;
LB = Luria-Bertani (medium);
LTR = long terminal repeat(s);
MAb, mAb = monoclonal Ab;
Mb = megabase(s);
MCS = multiple cloning site(s);
Me = methyl;
mg = milligram(s);
ml or mL = milliliter(s);
mm = millimeter(s);
mM = millimolar;
moi, MOI = multiplicity of infection;
Mr = relative molecular mass (dimensionless);
N = any nucleoside;
NAD/NADH = nicotinamide-adenine dinucleotide, and
its reduced form;
Nm = neomycin;
nmol = nanomole(s);
NMR = nuclear magnetic resonance;
NPT-II = Neomycin phosphotransferase gene or protein derived from Tn5
conferring resistance to kanamycin and neomycin and related antibiotics;
NPV = Nuclear polyhedrosis virus;
nt = nucleotide(s);
o, O = operator;
oligo = oligodeoxyribonucleotide;
ONPG = o-nitrophenyl β-D-galactopyranoside;
ORF = open reading frame;
ori = origin(s) of DNA replication;
p = plasmid;
p, P = promoter;
PA = polyacrylamide;
PAGE = PA-gel electrophoresis;
PCR = polymerase chain reaction, a gene amplification procedure;
PEG = poly(ethylene glycol);
PEP = phosphoenolpyruvate;
pfu = plaque-forming unit(s);
Pi = inorganic phosphate;
pmol = picomole(s);
PMSF = phenylmethylsulfonyl fluoride;
Pol k = Klenow (large) fragment of E. coli DNA polymerase I;
PPi = inorganic pyrophosphate;
ppm = parts per million;
PPO = 2,5-diphenyloxazole;
R = (superscript) resistance/resistant;
R = purine (or restriction);
r or R or superscripted r or R = resistant or resistance
RBS = ribosome-binding site(s);
rDNA = DNA coding for rRNA;
RFLP = restriction-fragment length polymorphism;
Rif = rifampicin;
RNase = ribonuclease;
RP-HPLC = reverse phase high performance liquid chromatograph;
rRNA = ribosomal RNA;
RT = reverse transcriptase;
RT = room temperature;
RT-PCR = reverse transcriptase polymerase chain reaction;
S or S = (superscript) sensitivity/sensitive;
S = sedimentation constant;
SAM = 5-adenosylmethionine;
SD = Shine-Dalgarno (sequence);
SDS = sodium dodecyl sulfate;
SDS-PAGE = sodium dodecyl sulfate-polyacrylamide gel electrophoresis;
Sf = Spodoptera frugiperda;
Sf9 = Spodoptera frugiperda (Sf9) cells/cell line;
Sf21 = Spodoptera frugiperda (IPLB Sf21) cells/cell line;
SIDNO or SID# = SEQ ID NO;
Sm = streptomycin;
Spc/Str = spectinomycin/streptomycin;
ss = single strand(ed);
SSC = 0.15M NaCl/0.015M Na3 · citrate pH 7.6;
T = thymidine;
t, T = terminator of transcription;
Tc, TC = tetracycline;
tet = gene conferring resistance to tetracycline and related antibiotics;
TK = thymidine kinase;
In = transposon or transposable element;
Tni, T. ni = Trichoplusia ni cells/cell line;
Tni368 = Trichoplusia ni (Tni368) cells/cell line;
tns = transposition genes;
ts = temperature-sensitive;
tsp = transcription start point(s);
U, u = unit(s);
U = uridine;
ug or μg = microgram(s);
ul or μl = microliter(s);
URF = unidentified open reading frame;
UTR = untranslated region(s);
UV = ultraviolet;
v = insect cell-derived baculovirus;
vc = insect cell-derived composite baculovirus;
vch = mixture of insect cell-derived composite baculovirus and helper
plasmid;
wt = wild type;
Xgal, X-gal = 5-bromo-4-chloro-3-indolyl β-D-galactopyranoside;
Xgluc, X-gluc = 5-bromo-3-chloro-indolyl-β-D-glucopyranoside;
Y = pyrimidine;
( ) = denotes prophage (lysogenic) state;
[ [ = denotes plasmid-carrier state;
“::” = novel junction (fusion or insertion, transposon insertion);
′(prime) = denotes a truncated gene at the indicated side;
Nucleotide symbol combinations:
Pairs: K = G/T; M = A/C; R = A/G; S = C/G; W = A/T; Y = C/T;
Triples: B = C/G/T; D = A/G/T; H = A/C/T; V = A/C/G; N = A/C/G/T;

Array: A series of genetic elements, in a linear order along the primary sequence of a DNA molecule, typically referring to a series of target sequences for a site-specific transposase or recombinase.

Bacmid: A baculovirus shuttle vector capable of replication in bacteria and in susceptible insect cells.

Bacteria: Any prokaryotic organism capable of supporting the function of the genetic elements described below. In one aspect, the bacteria should support the replication of a low copy number replicon operationally linked to the baculovirus in the bacmid, most preferably mini-F. The bacteria should support the replication of the donor plasmids, preferably moderate or high copy number plasmids or the host genome, most preferably either the bacteria chromosome, plasmids based on pUC8 or pMAK705. The bacteria should support the replication of helper plasmids, preferably moderate copy plasmids, most preferably based on pBR322. The bacteria should support the site-specific transposition of a transposon, most preferably one derived from Tn7. The bacteria should also support the expression and detection or selection of differentiable or selectable markers. In the preferred mode, the selectable markers are antibiotic resistance markers, most preferably genes conferring resistance to the following drugs: chloramphenicol, gentamicin, kanamycin, tetracycline, and ampicillin. In the preferred mode the differentiable markers should confer the ability of cells possessing them to metabolize chromogenic substrates. Most preferably, the differentiable marker encodes .alpha.-complementing fragment of .beta.-galactosidase.

BaculoBrick™: A synthetic adapter comprising one or more recognition sites for restriction enzymes that are typically 7 or more nucleotides, in length, generally 8 nt, and typically palindromic with double-stranded DNA cleavage sites entirely within the recognition site that leaving 5 or 3′ sticky overhangs, or blunt ends suitable for ligation to DNA fragments having complementary sticky or blunt ends. In this context, the adapter comprises sequences for restriction enzymes that cleave wild-type baculovirus DNAs, such as AcNPV or BmNPV DNA, zero to 5 times, permitting the rapid cloning and assembly of modular genetic elements suitable for insertion as cassettes into modified baculovirus genomes. These adapters can also be used to facilitate assembly of other large plasmids and shuttle vectors, including those intended for use in mammalian, plant, fungal, and other eukaryotic systems, plus enteric and non-enteric bacterial systems.

Baculovirus: A member of the Baculoviridae family of viruses with covalently closed double-stranded DNA genome and which are pathogenic for invertebrates, primarily insects of the order Lepidoptera.

Cis-Acting: cis-acting elements are genes or DNA segments which exert their functions on another DNA segment only when the cis-acting elements are linked to that DNA segment.

Combinatorial assembly of an ordered array: Assembly of a series of functionally- or structurally-similar sets of genetic elements in an array, where the sets may be assembled in any order, typically by traditional or modern cloning or gene assembly methods involving assembly of a large segment of DNA from two or more smaller segments of DNA.

Composite array: A partially or completely filled array of genetic elements comprising one or more segments of DNA inserted at specific target sequences for site-specific transposons or site-specific recombinases.

Composite Bacmid: A bacmid containing a wild-type or altered transposon inserted into a nonessential locus, usually the preferential target site for the transposon.

Donor DNA Molecule: Any replicating double-stranded DNA element such as the bacterial chromosome or a bacterial plasmid which carries a transposon capable of site-specific transposition into a bacmid. Preferably, the transposon contains a heterologous DNA and a genetic marker.

Donor Plasmid: A plasmid containing a wild-type or altered transposon, preferably a mini-Tn7 or Tn7-like transposon, comprising the left and right arms of Tn7 or a Tn7-like element flanking a cassette typically containing a genetic marker, a promoter, and one or more operably-linked genes of interest. The mini-transposon is preferably on a pUC-based or pMAK705-based plasmid.

Fusion proteins or fusion polypeptides: A single continuous linear polymer of amino acids which generally comprise the complete or partial sequences of two or more domains from distinct proteins. They are generally encoded by a linear segment of DNA and transcribed as a unit under the control of an operably-linked promoter, where the two or more coding sequences are contiguous with each other, optionally separated by one or more polypeptide linker sequences. The polypeptide linker sequences may also be present at the amino terminus, the carboxy-terminus, or both ends, contributing to the activity or inactivity of the fusion polypeptide compared to an unaltered parental polypeptide, or may provide other types of functions, such as binding to another molecule to facilitate purification during extraction from lysed cells or from cell culture media containing a variety of secreted molecules. In some aspects, the fusion polypeptide may comprise two or domains from a single parental molecule, in the same relative N-terminal to C-terminal orientation, or permuted, such that a domain from the C-terminal region of the parental polypeptide is located before a domain derived from the N-terminal region of the parental polypeptide. In other aspects, a fusion protein may comprise one or more segments derived from one or more natural proteins, and a synthetic segment that encodes a polypeptide not normally found in natural proteins.

Helper Plasmid or Helper Vector: A plasmid or vector which contains a bacterial replicon, a genetic marker and any genes which encode trans-acting factors which are required for the transposition of a given transposon.

Heterologous DNA: A sequence of DNA, from any source, which is introduced into an organism and which is not naturally contained within that organism.

Heterologous Protein: A protein which is synthesized in an organism, specifically from an introduced heterologous DNA, and which is not naturally synthesized within that organism.

Hyperactive transposase: A variant of a parental transposase gene encoded by a transposon that increases the frequency of transposition of a parental or variant transposon compared to the parental transposase gene.

Locus: A specific site or region of a DNA molecule which may or may not be a gene.

Mini-attTn7: The minimal DNA sequence required for recognition by Tn7 transposition factors and insertion of a Tn7 transposon or preferably mini-Tn7.

Mini-F: A derivative of the 100 kb Fertility (F) plasmid, which contains the RepF1A replicon, comprising seven genes including repE, and two DNA regions, oriS and incC, required for replication, maintenance, and regulation of mini-F replication.

Mini-Tn7: A transposon derived from Tn7 which contains the minimal amount of cis-acting DNA sequence required for transposition, a heterologous DNA and a genetic marker.

Nonessential: A locus is non-essential, if it is not required for replication of an vector, virus, cell, or organism as judged by the survival of that biological object following disruption or deletion of that locus.

NR1: A large (90 kb), stable, low copy number, IncFII drug resistance plasmid that confers resistance to chloramphenicol, fusidic acid, streptomycin, spectinomycin, sulfonamide, and tetracycline, which is compatible with the large (100 kb) stable, low copy number, IncFI Fertility (F) plasmid.

Passage: Infection of a host with a virus (or a mixture of viruses) and subsequent recovery of that virus from the host (usually after one infection cycle).

Plasmid Incompatibility: Plasmids are incompatible if they interact in such a way that they cannot be stably maintained in the same cell in the absence of selection for both plasmids.

Pₚₒₗₕ: A very late baculovirus promoter which is capable of promoting high level mRNA synthesis from any gene, preferably a heterologous DNA, placed under its control.

Preferential Target Site: A defined sequence of DNA specifically recognized and preferentially utilized by a transposon, preferably the attTn7 site for Tn7.

Random transposon: A naturally-occurring, variant, or synthetic transposon that has low to no specificity with respect to the sequences where it is inserted after transposition from one site to another. Common examples of random eukaryotic transposons include the synthetic Sleeping Beauty transposon, derived from consensus sequences in salmon, and the piggyBac transposon, derived from Trichoplusia ni, a caterpillar, and the random bacterial transposon Tn5, derived from a plasmid conferring resistance to kanamycin and other antibiotics. Variant and synthetic versions are often used with vectors comprising genes encoding hyperactive transposases, to enhance the frequency of random transposition a vector or the chromosome of a prokaryotic or eukaryotic cell.

Replicon: A replicating unit from which DNA synthesis initiates.

Screenable marker: A reporter gene introduced into a cell that confers a trait suitable for screening, typically allowing a researcher to distinguish between cells harboring a vector or no vector, or a cells harboring a vector and a variant form of a vector, such as bacteria form white colonies in a background of blue colonies in the presence of a chromogenic substrate, such as E. coli cells comprising vectors that do and do not have insertions disrupting expression of the alpha complementation polypeptide encoded by a lacZalpha gene in a cell comprising a lacZΔM15 gene on its chromosome.

Selectable marker: A reporter gene introduced into a cell that confers a trait suitable for artificial selection, commonly resistance to antibiotics, such as ampicillin, chloramphenicol, tetracycline, kanamycin, among many others, for vectors propagated in E. coli., and a wide variety of other antibiotics that allow selection of vectors that propagate in eukaryotic cells.

Shuttle Vector: A vector (usually a plasmid) that can propagate in two different types of host cell species, generally where one replicon permits propagation in prokaryotic cell, such as bacteria. A eukaryotic shuttle vector comprises at least one replicon permits propagation in a eukaryotic cell. A mammalian eukaryotic shuttle vector comprises at least one replicon which is derived from a mammalian cell, generally allowing the shuttle vector to propagate in a mammalian cell. A non-mammalian eukaryotic shuttle vector comprises at least one replicon which is derived from a non-mammalian cell, generally allowing the shuttle vector to propagate in a non-mammalian cell. A viral shuttle vector comprises at least one replicon which is derived from a virus, generally allowing the shuttle vector to propagate as a virus. A mammalian viral shuttle vector comprises at least one replicon which is derived from a mammalian virus, generally allowing the shuttle vector to propagate in mammalian cells as a virus. An insect viral shuttle vector comprises at least one replicon which is derived from an insect virus, generally allowing the shuttle vector to propagate in insect cells as a virus. A baculovirus shuttle vector comprises at least one replicon which is derived from an insect virus, generally allowing the shuttle vector to propagate in Lepidopteran insect cells as a virus.

Synthemid: A modular viral or non-viral vector comprising one or more target sites for a synthetic-site specific transposon, particularly those comprising gene fusions allowing for the direct selection of transposition events.

The term “amino acid(s)” means all naturally occurring L-amino acids, including norleucine, norvaline, homocysteine, and ornithine.

The term “degenerate” means that two nucleic acid molecules encode for the same amino acid sequences but comprise different nucleotide sequences.

The term “fragment” means a nucleic acid molecule whose sequence is shorter than the target or identified nucleic acid molecule and having the identical, the substantial complement, or the substantial homologue of at least 10 contiguous nucleotides of the target or identified nucleic acid molecule.

The term “fusion protein” means a protein or fragment thereof that comprises one or more additional peptide regions not derived from that protein.

The term “isolated” when used with respect to a polynucleotide (e.g., single- or double-stranded RNA or DNA), an enzyme, or more generally a protein, means a polynucleotide, an enzyme, or a protein that is substantially free from the cellular components that are associated with the polynucleotide, enzyme, or protein as it is found in nature. In this context, “substantially free from cellular components” means that the polynucleotide, enzyme, or protein is purified to a level of greater than 80% (such as greater than 90%, greater than 95%, or greater than 99%).

The term “probe” means an agent that is utilized to determine an attribute or feature (e.g. presence or absence, location, correlation, etc.) of a molecule, cell, tissue, or organism.

The term “promoter” is used in an expansive sense to refer to the regulatory sequence(s) that control mRNA production. Such sequences include RNA polymerase binding sites, enhancers, etc.

The term “protein fragment” means a peptide or polypeptide molecule whose amino acid sequence comprises a subset of the amino acid sequence of that protein.

The term “recombinant” means any agent (e.g., DNA, peptide, etc.), that is, or results from, however indirectly, human manipulation of a nucleic acid molecule.

The term “selectable or screenable marker genes” means genes whose expression can be detected by a probe as a means of identifying or selecting for transformed cells.

The term “specifically bind” means that the binding of an antibody or peptide is not competitively inhibited by the presence of non-related molecules.

The term “specifically hybridizing” means that two nucleic acid molecules are capable of forming an anti-parallel, double-stranded nucleic acid structure.

The term “substantial complement” means that a nucleic acid sequence shares at least 80% sequence identity with the complement.

The term “substantial fragment” means a nucleic acid fragment which comprises at least 100 nucleotides.

The term “substantial homologue” means that a nucleic acid molecule shares at least 80% sequence identity with another.

The term “substantially hybridizing” means that two nucleic acid molecules can form an anti-parallel, double-stranded nucleic acid structure under conditions (e.g., salt and temperature) that permit hybridization of sequences that exhibit 90% sequence identity or greater with each other and exhibit this identity for at least about a contiguous 50 nucleotides of the nucleic acid molecules.

The term “substantially-purified” means that one or more molecules that are or may be present in a naturally-occurring preparation containing the target molecule will have been removed or reduced in concentration.

The term “transposon” refers to mobile genetic elements capable of transposition between the genetic material in a cell (e.g., from one chromosomal location to one or more other locations in the chromosome, from a virus or a plasmid to the chromosome, from the chromosome to a virus or a plasmid, and from a plasmid or virus to a different plasmid or virus). The term also refers mobile DNA element, including those which recognize specific DNA target sequences, which can be made to move to a new site by recombination or insertion and does not require extensive DNA sequence homology between itself and the target sequence for recombination or insertion. A non-limiting list of transposons that may be used with the invention described herein, includes piggyBac, Sleeping Beauty (SB), Tn3, Tn5, Tn7, Tn916, Tcl/mariner, Minos and S elements, Quetzal elements, Txr elements, maT, most, HimarI, Hermes, Toll element, Pokey, P-element, and Tc3. In preferred aspects, the transposon is the site-specific Tn7, which inserts preferentially into a specific target or attachment site called attTn7. In other aspects, site-specific transposons, such as those classified as Tn7-like transposons or Tn7-like mobile genetic elements that insert into comparable attachment sites within the chromosome or on a plasmid harbored within a cell, are considered to be within the scope of the invention.

The terms “cell” and “cells”, which are meant to be inclusive, refer to one or more cells which can be in an isolated or cultured state, as in a cell line comprising a homogeneous or heterogeneous population of cells, or in a tissue sample, or as part of an organism, such as an insect larva or a transgenic mammal.

Trans-Acting: Trans-acting elements are genes or DNA segments which exert their functions on another DNA segment independent of the trans-acting elements genetic linkage to that DNA segment.

The phrase “Transpositional inactivation of a (selectable/screenable) marker/reporter gene” refers to inactivation of a marker or reporter gene by insertion of a site-specific or random transposon, disrupting or preventing expression of a functionally-active product encoded by the marker or reporter gene.

The phrase “Transpositional activation/reactivation of a (selectable/screenable) marker/reporter gene” refers to activation of a marker or reporter gene by insertion of a site-specific or random transposon, allowing expression of a functionally-active product encoded by the marker or reporter gene.

### DETAILED DESCRIPTION OF THE INVENTION

A major aspect of the invention relates to a nucleotide sequence comprising a target site for a site-specific transposon, wherein said target site comprises a target sequence comprising a transcriptionally or translationally fused marker sequence encoding a selectable marker sequence or a screenable marker sequence operably-linked to a sequence comprising a specific target sequence for recognition and insertion of a site-specific transposon or a site-specific recombinase, wherein said fused marker sequence encodes an inactive or an active polypeptide capable of conferring a selectable or screenable phenotype upon a cell comprising the fused marker sequence, wherein insertion of the site-specific transposon into the target sequence to create a composite target sequence changes the phenotype of a cell comprising the composite screenable or selectable marker sequence compared to a cell comprising just the selectable or screenable marker sequence.

Another aspect relates to a nucleotide sequence, wherein said target site comprises a target sequence for a site-specific transposon comprising a translationally-fused selectable marker sequence or a screenable marker sequence operably-linked to a sequence comprising a specific target sequence for recognition and insertion of a site-specific transposon, wherein said fused marker sequence encodes an inactive or an active polypeptide capable of conferring a selectable or screenable phenotype upon a cell comprising the fused marker sequence, wherein insertion of the site-specific transposon into the target sequence to create a composite target sequence changes the phenotype of a cell comprising the composite screenable or selectable marker sequence compared to a cell comprising just the selectable or screenable marker sequence.

Another aspect relates to a nucleotide sequence wherein said sequence comprises a target site for a site-specific transposon comprising a translationally-fused selectable marker sequence operably-linked to a sequence comprising a specific target sequence for recognition and insertion of a site-specific transposon, wherein said fused marker sequence encodes an inactive polypeptide capable of conferring a selectable phenotype upon a cell comprising the fused marker sequence, wherein insertion of the site-specific transposon into the target sequence to create a composite target sequence changes the phenotype of a cell comprising the composite selectable marker sequence compared to a cell comprising just the selectable marker sequence.

Another aspect relates to a sequence wherein said wherein said fused marker sequence encodes a truncated or extended inactive polypeptide which is extended or truncated, respectively, after transposition to form a composite target sequence which encodes an active polypeptide conferring a selectable phenotype upon the cell.

Still another aspect relates to a sequence, wherein said fused marker sequence encodes a truncated, inactive polypeptide which is extended after transposition to form a composite target sequence which encodes an active polypeptide conferring a selectable phenotype upon the cell.

Another aspect relates to a sequence wherein the selectable marker sequence encodes an inactive bacterial chloramphenicol acetyl transferase (CAT) fusion protein.

Another aspect relates to a sequence wherein the sequence encoding the inactive bacterial chloramphenicol acetyl transferase (CAT) fusion protein comprises in a 5′ to 3′ direction (i) a sequence encoding an inactive bacterial chloramphenicol acetyl transferase (CAT) polypeptide; (ii) a sequence comprising one or more stop codons; (iii) a sequence comprising the attachment site for the site-specific transposon and encoding a synthetic polypeptide; and (iv) a sequence comprising one or more in frame stop codons.

Another aspect relates to a nucleotide sequence wherein the composite selectable marker sequence encodes an active bacterial chloramphenicol acetyl transferase (CAT) fusion protein.

Still another aspect relates to a nucleotide sequence wherein the sequence encoding the active bacterial chloramphenicol acetyl transferase (CAT) fusion protein comprises in a 5′ to 3′ direction (i) a sequence encoding an inactive bacterial chloramphenicol acetyl transferase (CAT) polypeptide domain; (ii) a sequence comprising one or more out of reading frame stop codons; and (iii) a sequence comprising one end of the transposon and one or more in frame stop codons; wherein the addition of polypeptides encoded by (ii) (iii) to the inactive CAT polypeptide domain restore CAT activity to the fusion protein.

A major aspect relates to a nucleotide sequence wherein said fused marker sequence encodes an extended, inactive polypeptide which is truncated after transposition to form a composite target sequence which encodes an active, polypeptide conferring a selectable phenotype upon the cell.

Another aspect relates to a nucleotide sequence of claim 10, wherein the selectable marker sequence encodes an inactive NPT-II fusion protein.

Still another aspect relates to a nucleotide sequence wherein the sequence encoding the inactive NPT-II fusion protein comprises in a 5′ to 3′ direction (i) a sequence encoding an inactive NPT-II polypeptide; (ii) a sequence comprising one or more stop codons; (iii) a sequence comprising the attachment site for the site-specific transposon and encoding a synthetic polypeptide; and (iv) a sequence comprising one or more in frame stop codons.

Another aspect relates to a nucleotide sequence wherein the composite selectable marker sequence encodes an active NPT-II fusion protein.

Still another aspect relates to a nucleotide sequence, wherein the sequence encoding the active NPT-II fusion protein comprises in a 5′ to 3′ direction (i) a sequence encoding an inactive NPT-II polypeptide domain; (ii) a sequence comprising one or more out of reading frame stop codons; and (iii) a sequence comprising one end of the transposon and one or more in frame stop codons; wherein the removal of amino acids encoded by (ii) (iii) to the inactive NPT-II polypeptide domain restores NPT-II activity to the fusion protein.

Still another aspect relates to a nucleotide sequence, wherein the sequence encoding the active NPT-II fusion protein comprises in a 5′ to 3′ direction (i) a sequence encoding an inactive NPT-II polypeptide domain; (ii) a sequence comprising one or more out of reading frame stop codons; and (iii) a sequence comprising one end of the transposon and one or more in frame stop codons; wherein the addition of amino acids encoded by (ii) (iii) to the inactive NPT-II polypeptide domain restores NPT-II activity to the fusion protein.

Still another aspect relates to a nucleotide sequence, wherein said sequence comprises a target site for a site-specific transposon comprising a translationally-fused to screenable marker sequence operably-linked to a sequence comprising a specific site for recognition and insertion of a site-specific transposon, wherein said fused marker sequence encodes an active polypeptide capable of conferring a screenable phenotype upon a cell comprising the fused marker sequence, wherein insertion of the site-specific transposon into the target sequence to create a composite target sequence changes the phenotype of a cell comprising the composite screenable marker sequence compared to a cell comprising the just the selectable marker sequence.

Specific aspects of the invention relate to a nucleotide sequence, wherein the screenable marker sequence encodes an active lacZ alpha peptide fusion protein, including aspect where wherein the sequence encoding the active lacZ alpha fusion protein comprises in a 5′ to 3′ direction (i) a sequence encoding the N-terminal sequence of a lacZalpha polypeptide, (ii) a sequence comprising the attachment site for the site-specific transposon and encoding a synthetic polypeptide; (iii) and the C-terminal sequence of a lacZalpha polypeptide; and (iv) a sequence comprising one or more stop codons,

Related aspects include a sequence wherein the composite screenable marker sequence encodes an inactive lacZ alpha peptide fusion protein.

Related aspects include, a nucleotide sequence wherein the sequence encoding the active lacZ alpha fusion protein comprises in a 5′ to 3′ direction (i) a sequence encoding the sequence of a lacZalpha polypeptide, (ii) a sequence comprising the attachment site for the site-specific transposon and encoding a synthetic polypeptide; and (iii) a sequence comprising one or more in frame stop codons.

A related aspect includes a nucleotide sequence wherein the composite screenable marker sequence encodes an inactive lacZ alpha peptide fusion protein.

A related aspect includes a nucleotide sequence wherein the sequence encoding the active lacZ alpha fusion protein comprises in a 5′ to 3′ direction (i) a sequence comprising the attachment site for the site-specific transposon and encoding a synthetic polypeptide; (ii) a sequence encoding the sequence of a lacZalpha polypeptide; and (iii) a sequence comprising one or more in frame stop codons.

A related aspect includes a nucleotide sequence wherein the composite screenable marker sequence encodes an inactive lacZ alpha peptide fusion protein.

Related aspects include a nucleotide sequence wherein the screenable marker sequence encodes an active CAT fusion protein.

A related aspect includes a nucleotide sequence of wherein the sequence encoding the active CAT fusion protein comprises in a 5′ to 3′ direction (i) a sequence encoding the N-terminal sequence of a CAT polypeptide, (ii) a sequence comprising the attachment site for the site-specific transposon and encoding a synthetic polypeptide; (iii) and the C-terminal sequence of a CAT polypeptide; and (iv) a sequence comprising one or more stop codons.

A related aspect includes a nucleotide sequence, wherein the composite screenable marker sequence encodes an inactive CAT fusion protein.

Related aspects include a nucleotide sequence wherein the screenable marker sequence encodes an active NPT-II fusion protein.

A related aspect includes a nucleotide sequence, wherein the sequence encoding the active NPT-II fusion protein comprises in a 5′ to 3′ direction (i) a sequence encoding the N-terminal sequence of a NPT-II polypeptide, (ii) a sequence comprising the attachment site for the site-specific transposon and encoding a synthetic polypeptide; (iii) and the C-terminal sequence of a NPT-II polypeptide; and (iv) a sequence comprising one or more stop codons.

A related aspect includes a nucleotide sequence, wherein the composite screenable marker sequence encodes an inactive NPT-II fusion protein.

Related aspects include a nucleotide sequence, wherein the screenable marker sequence encodes an active β-lactamase fusion protein.

Specific aspects include a nucleotide sequence, wherein the sequence encoding the active β-lactamase fusion protein comprises in a 5′ to 3′ direction (i) a sequence encoding the N-terminal sequence of a β-lactamase polypeptide, (ii) a sequence comprising the attachment site for the site-specific transposon and encoding a synthetic polypeptide; (iii) and the C-terminal sequence of a β-lactamase polypeptide; and (iv) a sequence comprising one or more stop codons.

A related aspect includes a nucleotide sequence, wherein the composite screenable marker sequence encodes an inactive β-lactamase fusion protein.

Related aspects include a nucleotide sequence, wherein the screenable marker sequence encodes an active tetracycline resistance fusion protein.

Specific aspects include a nucleotide sequence, wherein the sequence encoding the active tetracycline resistance fusion protein comprises in a 5′ to 3′ direction (i) a sequence encoding the N-terminal sequence of a tetracycline resistance polypeptide, (ii) a sequence comprising the attachment site for the site-specific transposon and encoding a synthetic polypeptide; (iii) and the C-terminal sequence of a tetracycline resistance polypeptide; and (iv) a sequence comprising one or more stop codons.

Related aspects include a nucleotide sequence, wherein the composite screenable marker sequence encodes an inactive tetracycline resistance fusion protein.

Another aspect of the invention relates to a nucleotide sequence, wherein said sequence comprises a target site for a site-specific transposon comprising a translationally-fused selectable marker sequence operably-linked to a sequence comprising a specific target sequence for recognition and insertion of a site-specific transposon, wherein said fused marker sequence encodes an inactive polypeptide capable of conferring a selectable phenotype upon a cell comprising the fused marker sequence, wherein insertion of the site-specific transposon into the target sequence to create a composite target sequence changes the phenotype of a cell comprising the composite selectable marker sequence compared to a cell comprising just the selectable marker sequence.

Related aspects include a nucleotide sequence, wherein the selectable marker sequence encodes an inactive lacZ alpha fusion protein.

Specific aspects include a nucleotide sequence, wherein the sequence encoding the inactive lacZ alpha fusion protein comprises in a 5′ to 3′ direction (i) a sequence encoding the inactive lacZ alpha fusion protein; (ii) a sequence comprising one or more stop codons; (iii) a sequence comprising the attachment site for the site-specific transposon and encoding a synthetic polypeptide; and (iv) a sequence comprising one or more in frame stop codons.

A related aspect includes a nucleotide sequence, wherein the composite selectable marker sequence encodes an active lacZ alpha fusion protein.

Specific aspects include a nucleotide sequence, wherein the sequence encoding the active lacZ alpha fusion protein comprises in a 5′ to 3′ direction (i) a sequence encoding an inactive lacZ alpha fusion protein domain; (ii) a sequence comprising one or more out of reading frame stop codons; and (iii) a sequence comprising one end of the transposon and one or more in frame stop codons; wherein the addition of polypeptides encoded by (ii) (iii) to the an inactive lacZ alpha fusion domain restores activity to the lacZ alpha fusion protein.

Another aspect relates to a nucleotide sequence, wherein the selectable marker sequence encodes an inactive bacterial chloramphenicol acetyl transferase (CAT) fusion protein.

Specific aspects relate to a nucleotide sequence, wherein the sequence encoding the inactive bacterial chloramphenicol acetyl transferase (CAT) fusion protein comprises in a 5′ to 3′ direction (i) a sequence encoding an inactive bacterial chloramphenicol acetyl transferase (CAT) polypeptide; (ii) a sequence comprising one or more stop codons; (iii) a sequence comprising the attachment site for the site-specific transposon and encoding a synthetic polypeptide; and (iv) a sequence comprising one or more in frame stop codons.

Another aspect relates to a nucleotide sequence, wherein the composite selectable marker sequence encodes an active bacterial chloramphenicol acetyl transferase (CAT) fusion protein.

Specific aspects relate to a nucleotide sequence, wherein the sequence encoding the active bacterial chloramphenicol acetyl transferase (CAT) fusion protein comprises in a 5′ to 3′ direction (i) a sequence encoding an inactive bacterial chloramphenicol acetyl transferase (CAT) polypeptide domain; (ii) a sequence comprising one or more out of reading frame stop codons; and (iii) a sequence comprising one end of the transposon and one or more in frame stop codons; wherein the addition of polypeptides encoded by (ii) (iii) to the inactive CAT polypeptide domain restore CAT activity to the fusion protein.

Another aspect includes a nucleotide sequence, wherein the selectable marker sequence encodes an inactive NPT-II fusion protein.

Specific aspects relate to a nucleotide sequence, wherein the sequence encoding the inactive NPT-II fusion protein comprises in a 5′ to 3′ direction (i) a sequence encoding an inactive NPT-II polypeptide; (ii) a sequence comprising one or more stop codons; (iii) a sequence comprising the attachment site for the site-specific transposon and encoding a synthetic polypeptide; and (iv) a sequence comprising one or more in frame stop codons.

Another aspect relates to a nucleotide sequence, wherein the composite selectable marker sequence encodes an active NPT-II fusion protein.

Specific aspects relate to a nucleotide sequence, wherein the sequence encoding the active NPT-II fusion protein comprises in a 5′ to 3′ direction (i) a sequence encoding an inactive NPT-II polypeptide domain; (ii) sequence comprising one or more out of reading frame stop codons; and (iii) a sequence comprising one end of the transposon and one or more in frame stop codons; wherein the addition of polypeptides encoded by (ii) (iii) to the inactive NPT-II polypeptide domain restores NPT-II activity to the fusion protein.

Another aspect relates to a nucleotide sequence, wherein the selectable marker sequence encodes an inactive β-lactamase fusion protein.

Specific aspects relate to a nucleotide sequence, wherein the sequence encoding the inactive β-lactamase fusion protein comprises in a 5′ to 3′ direction (i) a sequence encoding an inactive β-lactamase polypeptide; (ii) a sequence comprising one or more stop codon; (iii) a sequence comprising the attachment site for the site-specific transposon and encoding a synthetic polypeptide; and (iv) a sequence comprising one or more in frame stop codons.

Another aspect relates to a nucleotide sequence, wherein the composite selectable marker sequence encodes an active β-lactamase fusion protein.

Specific aspects relate to a nucleotide sequence, wherein the sequence encoding the inactive β-lactamase fusion protein comprises in a 5′ to 3′ direction (i) a sequence encoding an active β-lactamase polypeptide domain; (ii) a sequence comprising one or more out of reading frame stop codons; and (iii) a sequence comprising one end of the transposon and one or more in frame stop codons; wherein the addition of polypeptides encoded by (ii) (iii) to the inactive β-lactamase polypeptide domain restores β-lactamase activity to the fusion protein.

Another aspect relates to a nucleotide sequence, wherein the selectable marker sequence encodes an inactive tetracycline resistance fusion protein.

Specific aspects relate to a nucleotide sequence, wherein the sequence encoding the inactive tetracycline resistance fusion protein comprises in a 5′ to 3′ direction (i) a sequence encoding an inactive tetracycline resistance polypeptide; (ii) a sequence comprising one or more stop codon; (iii) a sequence comprising the attachment site for the site-specific transposon and encoding a synthetic polypeptide; and (iv) a sequence comprising one or more in frame stop codons.

Another aspect relates to a nucleotide sequence, wherein the composite selectable marker sequence encodes an active tetracycline resistance fusion protein.

Specific aspects relate to a nucleotide sequence, wherein the sequence encoding the active tetracycline resistance fusion protein comprises in a 5′ to 3′ direction (i) a sequence encoding an inactive tetracycline resistance polypeptide domain; (ii) a sequence comprising one or more out of reading frame stop codons; and (iii) a sequence comprising one end of the transposon and one or more in frame stop codons; wherein the addition of polypeptides encoded by (ii) (iii) to the inactive tetracycline resistance polypeptide domain restores activity to the tetracycline resistance fusion protein.

Major aspects of the invention relate to a vector, designated a synthemid, comprising any of the target sequence or composite target sequences noted above.

Other aspects relate to a vector, wherein said vector propagates in a gram negative bacteria, a vector which propagates in a gram negative enteric bacteria, and a vector which propagates in Escherichia coli.

Other aspects relate to a vector, wherein said vector propagates in a gram positive bacteria.

Other aspects relate to a vector, wherein said vector is a shuttle vector capable of propagating in bacteria and a non-bacterial host cell.

Still another aspect relates to a vector wherein said shuttle vector is a eukaryotic viral shuttle vector capable of propagating in bacteria and in cell line capable of propagating a eukaryotic virus.

Still another aspect relates to a vector wherein said eukaryotic viral shuttle vector is a baculovirus shuttle vector, capable of propagating in bacteria and in Lepidopteran insect cells susceptible to infection by the baculovirus.

Still another aspect relates to a vector, wherein said baculovirus shuttle vector is capable of propagating in Escherichia coli and insect cells selected from the group consisting of Spodoptera frugiperda, Trichoplusia ni cells, and Bombyx mori cells.

Still another aspect relates to a vector wherein said eukaryotic viral shuttle vector is a mammalian virus shuttle vector, capable of propagating in bacteria and in mammalian cells susceptible to infection by the mammalian virus.

Another aspect relates to a vector comprising the target sequence.

Another aspect relates to a vector comprising the composite target sequence.

Related aspects include a nucleotide sequence comprising an array of two or more target sequences, and a vector, designated a synthemid, comprising said array.

Related aspects include a nucleotide sequence comprising a composite array of two or more composite target sequences, and a composite vector, designated a composite synthemid, comprising said composite array.

Major aspects relate to a nucleotide sequence wherein site-specific transposon is Tn7 or a Tn7-like transposon.

A specific aspect relates to a nucleotide sequence wherein said site-specific transposon is Tn7.

A specific aspect relates to a nucleotide sequence wherein said site-specific transposon is a Tn7-like transposon.

Another aspect relates to a nucleotide sequence, wherein said attachment site and site specific transposon are derived from a Tn7-like transposable element. In one aspect, said attachment site is attTn7 and the transposon is Tn7.

A major aspect of the invention also relates to a method of screening or selecting for transposition of a site-specific transposon into a nucleotide sequence comprising an attachment site for a site-specific transposon operably-linked to a screenable or selectable marker sequence, comprising the steps of (i) introducing into a bacterial cell a target vector comprising a marker sequence that encodes one or more active or inactive polypeptides capable of conferring a screenable or selectable phenotype upon a cell comprising the marker sequence, wherein insertion of the site-specific transposon into the attachment site to create a composite marker sequence changes the phenotype of a cell comprising the screenable or selectable marker sequence; (ii) introducing into said cell comprising said target vector, a donor vector comprising sequences capable of transposing the wild type or a variant form of the site-specific transposon, and optionally a helper vector comprising sequences encoding one or more transposase gene products; (iii) culturing and optionally plating bacteria comprising the target vector, and optionally donor and helper vectors, (iv) screening or selecting for bacterial colonies where transposition of the site-specific transposon into the attachment site on the target vector to create a composite marker sequence changes the phenotype of the bacterial cell harboring the target vector.

Specific aspects relate to a method, wherein step (iv) is screening for bacterial colonies where transposition of the site-specific transposon into the attachment site on the target vector changes the phenotype of the bacterial cell harboring the target vector.

More specific aspects relate to a method, wherein the screenable method is by a change from a Lac positive (+) to a Lac minus (−) phenotype, a change from an NPT-II positive (+) to an NPT-II minus (−) phenotype, a change from a β-lactamase positive (+) to a β-lactamase minus (−) phenotype, a change from a tetracycline resistant (+) to a tetracycline sensitive (−) phenotype.

Specific aspects relate to a method wherein step (iv) is selecting for bacterial colonies where transposition of the site-specific transposon into the attachment site on the target vector changes the phenotype of the bacterial cell harboring the target vector.

More specific aspects include a method, wherein the selectable method is by a change from a Cm sensitive (S) to a Cm resistant (R) phenotype, including a change from a Lac positive (+) to a Lac minus (−) phenotype, a change from a Lac minus (−) to a Lac positive (+) phenotype, a change from a NPT-II minus (−) to a NPT-II plus (+) phenotype, a change from a β-lactamase minus (−) to a β-lactamase plus (+) phenotype, and a change from a tetracycline sensitive (−) to a tetracycline resistant (+) phenotype.

### EXAMPLES

The foregoing discussion may be better understood in connection with the following representative examples which are presented for purposes of illustrating the principle methods and compositions of the invention, and not by way of limitation. Various other examples will be apparent to the person skilled in the art after reading the present disclosure without departing from the spirit and scope of the invention. It is intended that all such other examples be included within the scope of the appended claims.

### General Materials and Methods

Simulated cloning and display of linear DNA segments and circular plasmid maps was facilitated through the use of the SnapGene program obtained from GSL Biotech. Analysis of sequences permitting silent mutations in coding sequences was facilitated by “WatCut: An on-line tool for restriction analysis, silent mutation scanning, and SNP-RFLP analysis”, maintained by Michael Palmer, University of Waterloo, Ontario, Canada (watcut.uwaterloo.ca). General features and annotated maps of a wide variety of DNA segments and cloning or expression vectors can be obtained from online databases maintained by NCBI, such as GenBank, Addgene, SnapGene, Thermo Fisher, and New England Biolabs.

Standard general methods of cloning, expressing, and characterizing proteins are found in T. Maniatis, et al, Molecular Cloning, A Laboratory Manual, Cold Spring Harbor Laboratory, 1982, and references cited therein, incorporated herein by reference; and in J. Sambrook, et al, Molecular Cloning, A Laboratory Manual, 2nd edition, Cold Spring Harbor Laboratory, 1989, and references cited therein, incorporated herein by reference. General methods for the cloning and expression of genes in mammalian cells are also found in Colosimo et al, Biotechniques 29:314-331, 2000. Baculovirus- and insect cell culture-related procedures are performed as described (O'Reilly et al, 1992).

Restriction enzymes were purchased from Thermo Fisher (Waltham, Mass.) and New England Biolabs (Beverly, Mass.), unless otherwise indicated. Synthetic vectors and oligonucleotides were purchased from Twist Biosciences or IDT, unless otherwise indicated. Structural analysis of vectors, by DNA sequencing was performed by GeneWiz (South Plainfield, N.J.). All parts are by weight (e.g., % w/w), and temperatures are in degrees Centigrade (° C.), unless otherwise indicated.

Brief descriptions of key materials required for the studies described below are provided in the following tables, noted below in different sections of the Examples, including Table: 5—Key Features of Bacterial Strains, Table: 6—Plasmids Used in These Studies; and Table: 7—Summary Table of Sequences.

Bacterial strains and plasmid vectors are obtained from the sources listed in each table, or constructed for these studies. The nucleotide sequences of plasmid vectors, if known, are indicated by their GenBank Accession Numbers. The sequences of oligonucleotides that are annealed to complementary nucleotides, or used as primers for amplifying segments of dsDNA are also shown below, and assigned specific SEQ ID NOS, as recited in the Sequence Listing, and in one or more tables summarizing key features of nucleotide and amino acid sequences set forth in the Sequence Listing.

### Bacterial Media

Rich media, such as 2XYT broth and LB broth and agar, are purchased or prepared as described by (Miller, 1972). Supplements are incorporated into liquid and solid media typically at the following concentrations (μg/ml): Amp, 100; Gen, 7; Tet, 10; Kan, 50; X-gal or Bluo-gal, 100; IPTG, 40. Ampicillin, kanamycin, tetracycline, and IPTG (isopropyl-beta-D-thiogalactoside) are purchased from Teknova (Hollister, Calif.) and Millipore Sigma (St. Louis, Mo.). Gentamicin, X-gal (5-bromo-3-chloro-indolyl-beta-D-galactoside), and Bluo-gal (halogenated indolyl-beta-D-galactoside) are purchased from GIBCO/BRL. Pre-poured agar plates, antibiotic solutions, and liquid media were also purchased from Teknova (Hollister, Calif.), Thermo Fisher (Carlsbad, Calif.), and Millipore Sigma (St. Louis, Mo.).

### Bacterial Transformation

Plasmids were transformed into frozen competent E. coli DH10B (Grant et al, 1990), obtained from Thermo Fisher, using the procedures recommended by the manufacturer. Briefly, frozen cells were thawed on ice and 33-100 μl of cells are incubated with 0.01-1.0 μg of plasmid DNA for 30-60 minutes. The cells were shocked by heating at 42° C. for 30 seconds, diluted to 1.0 ml with antibiotic-free S.O.C. buffer, and grown at 37° C. for 1-3 hours. A 20 to 100 ul sample of culture was spread on agar plates supplemented with the appropriate antibiotics. Colonies are purified by restreaking on the same selection plates prior to analysis of drug resistance phenotype and isolation of plasmid DNAs. Plasmids are also transformed into competent E. coli DH10B cells prepared by suspending early log phase cells in transformation buffer using a TransformAid kit obtained from Thermo Fisher. Plasmids may be transformed into competent cells prepared by the calcium chloride method described by Sambrook et al, (1989), or by transformation into electrocompetent cells suspended in buffered glycerol using protocols and equipment provided by BioRad.

### DNA Preparation and Plasmid Manipulation

DNA samples are prepared from 1-250 ml cultures grown in LB or 2XYT medium supplemented with appropriate antibiotics. Cultures are harvested and lysed by an alkaline lysis method and the plasmid DNA samples are purified over resin columns provided by Thermo Fisher.

TABLE 5
Key Features of Bacterial Strains

| Designation | Genotype | Description | Reference | Source |
|---|---|---|---|---|
| DH5aF′IQ | F′ proAB⁺ laclqΔZM15 zzf::Tn5 (Kan^{R}) | Original source of the | | GIBCO/BFL |
| | isolated from strain DH5alphaF′IQ | mini-F replicon and the | | |
| | | kanamycin resistance gene | | |
| | | inserted into the bacmid | | |
| | | bMON14272. | | |
| E. coli | F⁻endA1 reck1 galE15 galK16 nupG rpsL | DH10B has been | Grant et al, | Thermo |
| DH10B | ΔlacX74 Φ80lacZΔM15 araD139 | classically reported to be | 1990; | Fisher |
| | Δ(ara, leu)7697 mcrA Δ(mrr-hsdRMS-mcrBC) λ⁻ | galU galK, the genomic | Blattner | |
| | | sequence indicates that | | |
| | | DH10B is actually galE | | |
| | | galK galU+, and is also | | |
| | | deoR⁺. | | |
| E. coli | F⁻ mcrA Δ(mrr-hsdRMS-mcrSC) Φ80lacZΔM15 | DH10B harboring the | Luckow et al | Thermo |
| DH10Bac ™ | ΔlacX74 recA1 endA1 araD139 | baculovirus shuttle vector | (1993) | Fisher |
| | Δ(ara, leu)7697 galU galK λ⁻ rpsl | (bacmid) bMON7124 and the | | |
| | nupG/bMON14272/pMON7124 | helper plasmid pMON7124. | | |

TABLE 6
Plasmids Used in These Studies

| | | Size | | | |
|---|---|---|---|---|---|
| Designation | Markers | (bp) | Description | Reference | Source |
| pACYC177 | Amp^{R}, | 3941 | pACYC177 is an E. coli | Chang, A. and Cohen, | NEB |
| | Kan^{R} | | plasmid cloning vector | S. (1978) J. Bacteriol. | |
| | | | comprising an ampicillin | 134: 1114-1156. | |
| | | | resistance (Amp^{R}) gene | | |
| | | | derived from Tn3, and a | | |
| | | | kanamycin resistance gene | | |
| | | | (Kan^{R}) derived from Tn903. It | | |
| | | | contains a p15A origin of | | |
| | | | replication derived from | | |
| | | | pSC101, allowing it to coexist | | |
| | | | in cells with plasmids of the | | |
| | | | ColE1 compatibility group | | |
| | | | (e.g., pBR322, pUC19), and | | |
| | | | considered to be a low- | | |
| | | | medium number vector, with | | |
| | | | about 15 copies per cell. | | |
| pACYC184 | Tet^{R}, | 4245 | pACYC184 carries a gene | Chang, A. and Cohen, | Boca |
| | Cat^{R} | | conferring resistance to | S. (1978) J. Bacteriol. | Scientific |
| | | | tetracycline (Tet^{R}) and a gene | 134: 1114-1156;; | |
| | | | encoding chloramphenicol | Sequence reported by | |
| | | | acetyltransferase, conferring | Rose, R. E. (1988) | |
| | | | resistance to chloramphenicol | Nucleic Acids. | |
| | | | (Cat^{R}). It has the same | Res.16: 355. | |
| | | | replicon as pACYC177. | | |
| pTwist- | Cat^{R} | 1953 | Synthetic cloning vector | | Twist |
| Chlor-MC | | | conferring resistance to | | Biosciences |
| | | | chloramphenicol and | | |
| | | | comprising a medium copy | | |
| | | | number (MC) p15A bacterial | | |
| | | | replicon used to facilitate | | |
| | | | cloning of synthetic sequences. | | |
| pTwist-Kan- | Kan^{R} | 2105 | Synthetic cloning vector | | Twist |
| MC | | | conferring resistance to | | Biosciences |
| | | | kanamycin and comprising a | | |
| | | | medium copy number (MC) | | |
| | | | p15A bacterial replicon used | | |
| | | | to facilitate cloning of | | |
| | | | synthetic sequences. | | |
| pTwist-Amp- | Amp^{R} | 2221 | Synthetic cloning vector | | Twist |
| HC | | | conferring resistance to | | Biosciences |
| | | | Ampicillin and comprising a | | |
| | | | high copy number (HC) | | |
| | | | pMB1/ColE1/pUC bacterial | | |
| | | | replicon used to facilitate | | |
| | | | cloning of synthetic | | |
| | | | sequences. | | |
| pMAK705 | Cat^{R}, | 5593 | Derived from pH01 and | Hamilton et al, | |
| | lacZ | | pMAK700 containing a | (1989) | |
| | alpha | | pSC101^{ts} replicon, a cat gene | | |
| | | | and partial amp gene from | | |
| | | | pBR325, and lacZalpha | | |
| | | | segment from pUC19. | | |
| pFastBac1 | Amp^{R}, | 4775 | Mini-Tn7 donor plasmid | Ciccarone et al | Thermo |
| | Gent^{R} | | derived from pMON14327, | (1997), based on | Fisher |
| | | | containing the AcNPV | Luckow et al (1993) | |
| | | | polyhedrin promoter, a | | |
| | | | multiple cloning site (MCS) | | |
| | | | and SV40 poly(A) | | |
| | | | transcriptional | | |
| | | | terminator segment between | | |
| | | | the left and right arms of Tn7. | | |
| pMON7124 | Tet^{R} | 13,328 | pBR322 comprising Tn7 | Barry (1988); | Thermo |
| | | | transposase genes tns A, B, | (Sequenced by D. | Fisher |
| | | | C, D, and E, plus the right end | Esposito, pers. com.) | |
| | | | of Tn7 (Tn7R). | | |
| bMON14272 | Kan^{R} | ~142,278 | Baculovirus shuttle vector | Luckow et al (1993); | Thermo |
| | | | comprising contiguous | (Sequenced by D. | Fisher |
| | | | segment encoding a | Esposito, pers. com.) | |
| | | | kanamycin resistance gene | | |
| | | | (Kan^{R}), a lacZalpha-mini- | | |
| | | | attTn7, and a mini-F replicon | | |
| | | | (stable, IncFl, very low copy | | |
| | | | number) inserted into the | | |
| | | | polyhedrin locus of the | | |
| | | | baculovirus Autographa | | |
| | | | californica Nuclear | | |
| | | | Polyhedrosis Virus (AcNPV) | | |
| | | | E2 variant. | | |

Table 7 summarizes features sequences and vectors represented by SEQ ID NOS 1-198.

Tables 24 and 26 summarize features of Twist vectors 1-40 represented by SEQ ID NOS 199-240.

TABLE 7
Summary Table of Sequences

| | | | | SEQ |
|---|---|---|---|---|
| | | | | lD |
| Name | Description | Length | Type | NO |
| Tn7 | Nucleotide sequence | 14067 | DNA | 01 |
| | of wild-type Tn7 (GenBank | | | |
| | Acc. No. BM_NC_002525), | | | |
| | found in a plasmid isolated | | | |
| | from E. coli. | | | |
| attTn7 near 3′ | Sequences extending from −2, −1, | 61 | DNA | 02 |
| end of E. coli glmS | 0, +1 +2, and +3 to +58 of the | | | |
| gene | attachment site for Tn7 near | | | |
| | the E. coli glmS gene, where | | | |
| | positions −2 to +2 are | | | |
| | duplicated as 5 bp sequences | | | |
| | at both ends of a Tn7 element | | | |
| | after transposition into this | | | |
| | sequence. | | | |
| 5-bp duplication | Junction of 5-bp duplication | 13 | DNA | 03 |
| at Tn7L in | nearTn7L inserted between | | | |
| attTn7 | positions −2 to +2 of attTn7 | | | |
| | near 3′ end of E. coli glmS | | | |
| | gene | | | |
| 5-bp duplication | Junction of 5-bp duplication | 69 | DNA | 04 |
| at Tn7R in | near Tn7R inserted between | | | |
| attTn7 | positions −2 to +2 of attTn7 | | | |
| | near 3′ end of E. coli glmS | | | |
| | gene. | | | |
| mini-attTn7 | Synthetic lacZ-alpha-mini- | 549 | DNA | 05 |
| | attTn7 sequence | | | |
| Truncated lacZalpha- | Synthetic truncated lacZalpha- | 366 | DNA | 06 |
| mini-attTn7 | mini-attTn7 | | | |
| 3′ end of Type I cat | Sequences From the TatI/ScaI | 76 | DNA | 07 |
| gene adding | site to the BaeGI/Bme1508I | | | |
| SrfI/XmaI sites | at the 3′ end of the Type I | | | |
| | cat gene, adding SrfI and | | | |
| | XmaI sites | | | |
| | Polypeptide sequence encoded | 10 | PRT | 08 |
| | at carboxy terminal region of | | | |
| | Type I CAT protein, represented | | | |
| | by QYCDEWQGGA* | | | |
| 3′ end of Type I | Sequences From the Tat/ScaI | 76 | DNA | 09 |
| cat gene changing | site to the BaeGI/Bme1508I | | | |
| GAT to TAA stop | at the 3′ end of the Type I | | | |
| codon | cat gene, adding SrfI and | | | |
| | XmaI sites, changing the | | | |
| | GAT to a TAA stop codon. | | | |
| 3′ end of Type I | Sequences From the Tat/ScaI | 76 | DNA | 10 |
| cat gene | site to the BaeGI/Bme1508I | | | |
| changing GAT codon | at the 3′ end of the Type I | | | |
| to TGA stop | cat gene, adding SrfI and | | | |
| codon | XmaI sites, changing the | | | |
| | GAT to a TGA, stop codon. | | | |
| 3′ end of Type I | Sequences From the Tat/ScaI | 76 | DNA | 11 |
| cat gene | site to the BaeGI/Bme1508I | | | |
| changing GAT | at the 3′ end of the Type I | | | |
| codon to a TAG | cat gene, adding SrfI and | | | |
| stop codon | XmaI sites, changing the | | | |
| | GAT to a TAG stop codon. | | | |
| 3′ end of the Type | 3′ end of the Type I cat | 100 | DNA | 12 |
| I cat gene, adding | gene, adding SrfI and XmaI | | | |
| SrfI and XmaI sites, | sites, before changing the | | | |
| Before changing the | GAT to a TAA, TGA, or TAG | | | |
| GAT to a TAA, TGA, | stop codon, and adding an | | | |
| or TAG stop codon, | overlapping mini-attTn7 site | | | |
| and adding | | | | |
| an overlapping mini- | | | | |
| attTn7 site | | | | |
| 3′ end of Type I | Sequences From the Tat/ScaI | 100 | DNA | 13 |
| cat gene with | site to the BaeGI/Bme1508I | | | |
| TAA stop codon | at the 3′ end of the Type I | | | |
| and overlapping | cat gene, adding SrfI and | | | |
| mini-attTn7 | XmaI sites, changing the | | | |
| | GAT to a TAA stop codon, | | | |
| | and adding an overlapping | | | |
| | mini-attTn7 site. | | | |
| 3′ end of Type I cat | Sequences From the Tat/ScaI | 100 | DNA | 14 |
| gene with TGA stop | site to the BaeGI/Bme1508I | | | |
| codon and overlapping | at the 3′ end of the Type I | | | |
| mini-attTn7 | cat gene, adding SrfI and | | | |
| | XmaI sites, changing the GAT | | | |
| | to a TGA, stop codon, and | | | |
| | adding an overlapping | | | |
| | mini-attTn7 site. | | | |
| 3′ end of Type I cat | Sequences From the Tat/ScaI | 100 | DNA | 15 |
| gene with TAG | site to the BaeGI/Bme1508I | | | |
| stop codon and | at the 3′ end of the Type I | | | |
| overlapping | cat gene, adding SrfI and | | | |
| mini-attTn7 | XmaI sites, changing the | | | |
| | GAT to a TAG stop codon, | | | |
| | and adding an overlapping | | | |
| | mini-attTn7 site | | | |
| 3′ end of Type I | Sequences From the TatI/ScaI | 93 | DNA | 16 |
| cat gene adding | site to the BaeGI/Bme1508I | | | |
| SrfI and XmaI sites, | at the 3′ end of Type I cat | | | |
| before changing | gene, adding SrfI and XmaI | | | |
| TGCGAT to double stop | sites, changing the TGC to | | | |
| codons | a TAA, TGA, or TAG stop codon, | | | |
| | and the GAT to a TAA stop | | | |
| | codon, adding mini-attTn7 | | | |
| | overlapping with the first | | | |
| | stop codon | | | |
| 3′ end of Type I | Sequences From the TatI/ScaI | 93 | DNA | 17 |
| CAT gene with | site to the BaeGI/Bme1508I | | | |
| TGCGAT changed | at the 3′ end of Type I cat | | | |
| to TAATAA double | gene, adding SrfI and XmaI | | | |
| stop codons and | sites, changing the TGC to | | | |
| overlapping mini- | a TAA stop codon, and the | | | |
| attTn7 | GAT to a TAA stop codon, | | | |
| | adding mini-attTn7 | | | |
| | overlapping with the | | | |
| | first stop codon | | | |
| 3′ end of Type I | Sequences From the TatI/ScaI | 93 | DNA | 18 |
| cat gene with | site to the BaeGI/Bme1508I | | | |
| TGCGAT changed to | at the 3′ end of Type I cat | | | |
| TGATAA double stop | gene, adding SrfI and XmaI | | | |
| codons and | sites, changing the TGC to | | | |
| overlapping mini- | a TAA stop codon, and the | | | |
| attTn7 | GAT to a TAA stop codon, | | | |
| | adding mini-attTn7 | | | |
| | overlapping with the firs | | | |
| | t stop codon | | | |
| 3′ end of Type I | Sequences From the TatI/ScaI | 93 | DNA | 19 |
| cat gene with | site to the BaeGI/Bme1508I | | | |
| TGCGAT changed to | at the 3′ end of Type I cat | | | |
| TAGTAA double stop | gene, adding SrfI and XmaI | | | |
| codons and | sites, changing the TGC to | | | |
| overlapping mini- | a TGA stop codon, and the | | | |
| attTn7 | GAT to a TAA stop codon, | | | |
| | adding mini-attTn7 | | | |
| | overlapping with the | | | |
| | first stop codon | | | |
| 3′ end of a Type I | Sequences at the 3′ end | 39 | DNA | 20 |
| cat gene after | of a Type I cat gene | | | |
| transposition into | after transposition of a | | | |
| an overlapping | mini-Tn7 into an over | | | |
| mini-atTn7 | overlapping mini- attTn7 | | | |
| | site. | | | |
| | Polypeptide sequences 3′ | 12 | PRT | 21 |
| | end of a Type I cat gene | | | |
| | after transposition of a | | | |
| | mini-Tn7 into an over | | | |
| | overlapping mini- attTn7 | | | |
| | site | | | |
| 3′ end of Tn7R | 3′ end of Tn7R after | 22 | DNA | 22 |
| after transposition | transposition an over | | | |
| an over overlapping | overlapping mini- attTn7 | | | |
| mini-attTn7 | site | | | |
| site | | | | |
| 3′ end of Type I | Sequences at the 3′ end | 67 | DNA | 23 |
| cat gene to | of a Type I cat gene | | | |
| mimic insertion | that mimic Tn7L at the | | | |
| of Tn7L replacing | junction of mini-Tn7 | | | |
| stop codon for | replacing a stop codon | | | |
| Cys codon | for a Cys codon in an | | | |
| | overlapping mini-attTn7 | | | |
| | site | | | |
| | Polypeptide sequence that | 7 | PRT | 24 |
| | mimics insertion of the | | | |
| | Tn7L replacing the stop | | | |
| | codon for a Cys codon, | | | |
| | restoring activity to | | | |
| | the encoded CAT fusion | | | |
| | protein | | | |
| lacZ nt 1-180 | 5′ end of E. coli lacZ | 180 | DNA | 25 |
| | gene nucleotides 1-180 | | | |
| | Polypeptide encoded by 5′ | 60 | PRT | 26 |
| | end of E. coli lacZ gene | | | |
| | nucleotides 1-180 | | | |
| lacZdeltaM15 nt 1-57 | 5′ end of lacZ delta M15 | 57 | DNA | 27 |
| | gene of E. coli encoding | | | |
| | amino acids 1-11 and | | | |
| | 42-49 | | | |
| | Polypeptide 5′ end of lacZ | 19 | PRT | 28 |
| | delta M15 gene of E. coli | | | |
| | encoding amino acids 1-11 | | | |
| | and 42-49 | | | |
| pUC19 lacZalpha gene | LacZ alpha gene with MCS | 360 | DNA | 29 |
| | region pUC19 from | | | |
| | positions 1-360 | | | |
| | Polypeptide encoded by LacZ | 106 | PRT | 30 |
| | alpha gene with MCS region | | | |
| | pUC19 from positions 1-360 | | | |
| lacZ 1 to 260 | Sequences from 1−260 of the | 260 | DNA | 31 |
| | lacZ gene, but polypeptide | | | |
| | sequence diverges around | | | |
| | nucleotide 186 compared | | | |
| | to those in pUC19 | | | |
| | Polypeptide encoded by | 62 | PRT | 32 |
| | sequences from 1−260 of | | | |
| | the lacZ gene, but | | | |
| | polypeptide sequence | | | |
| | diverges around nucleotide | | | |
| | 186 compared to those | | | |
| | in pUC19 | | | |
| PuvII to KasI | PuvII to KasI sites of | 120 | DNA | 33 |
| sites of LacZ alpha | LacZ alpha gene pUC18 or | | | |
| gene pUC18 or pUC19 | pUC19 | | | |
| | Polypeptide encoded by PuvII | 40 | PRT | 34 |
| | to KasI sites of LacZ alpha | | | |
| | gene pUC18 orpUC19 | | | |
| PuvII to KasI | PuvII to KasI sites of LacZ | 120 | DNA | 35 |
| sites of LacZ | alpha gene pUC18 or pUC19 | | | |
| alpha gene pUC18 | with synthetic | | | |
| or pUC19 with | oligonucleotides comprising | | | |
| synthetic | two TAA stop codons near | | | |
| oligonucleotides | codons encoding NS | | | |
| comprising two | | | | |
| TAA stop codons | | | | |
| replacing codons | | | | |
| encoding NS | | | | |
| | Polypeptide encoded by PuvII | 16 | PRT | 36 |
| | to KasI sites of LacZ alpha | | | |
| | gene pUC18 or pUC19 with | | | |
| | synthetic oligonucleotides | | | |
| | comprising two TAA stop | | | |
| | codons near codons encoding | | | |
| | NS | | | |
| PuvII to KasI sites | PuvII to KasI sites of LacZ | 120 | DNA | 37 |
| of LacZ alpha | alpha gene pUC18 or pUC19 | | | |
| gene pUC18 or pUC19 | with synthetic | | | |
| with synthetic | oligonucleotides | | | |
| oligonucleotides | comprising two TAA stop | | | |
| comprising two | codons near codons encoding | | | |
| TAA stop codons | SE | | | |
| near codons encoding | | | | |
| SE | | | | |
| | Polypeptide encoded by PuvII | 16 | PRT | 38 |
| | to KasI sites of LacZ alpha | | | |
| | gene pUC18 or pUC19 with | | | |
| | synthetic oligonucleotides | | | |
| | comprising two TAA stop | | | |
| | codons near codons encoding | | | |
| | SE | | | |
| PuvII to KasI sites | PuvII to KasI sites of LacZ | 120 | DNA | 39 |
| of LacZ alpha | alpha gene pUC18 or pUC19 with | | | |
| gene pUC18 or pUC19 | synthetic oligonucleotides | | | |
| with synthetic | comprising two TAA stop | | | |
| oligonucleotides | codons near codons encoding | | | |
| comprising two TAA | EE | | | |
| stop codons near | | | | |
| codons encoding EE | | | | |
| | Polypeptide encoded by PuvII | 16 | PRT | 40 |
| | to KasI sites of LacZ alpha | | | |
| | gene pUC18 or pUC19 with | | | |
| | synthetic oligonucleotides | | | |
| | comprising two TAA stop | | | |
| | codons near codons encoding | | | |
| | EE | | | |
| PuvII to KasI sites | PuvII to KasI sites of LacZ | 120 | DNA | 41 |
| of LacZ alpha | alpha gene pUC18 or pUC19 | | | |
| gene pUC18 or pUC19 | with synthetic | | | |
| with synthetic | oligonucleotides comprising | | | |
| oligonucleotides | two TAA stop codons nea | | | |
| comprising two | r codons encoding EA | | | |
| TAA stop codons | | | | |
| near codons | | | | |
| encoding EA | | | | |
| | Polypeptide encoded by PuvII | 16 | PRT | 42 |
| | to KasI sites of LacZ alpha | | | |
| | gene pUC18 or pUC19 with | | | |
| | synthetic oligonucleotides | | | |
| | comprising two TAA stop | | | |
| | codons near codons encoding | | | |
| | EA | | | |
| PuvII to KasI sites | PuvII to KasI sites of LacZ | 120 | DNA | 43 |
| of LacZ alpha gene | alpha gene pUC18 or pUC19 | | | |
| pUC18 or pUC19 with | with synthetic | | | |
| synthetic | oligonucleotides comprising | | | |
| oligonucleotides | two TAA stop codons near | | | |
| comprising two TAA | codons encoding AR | | | |
| stop codons near | | | | |
| codons encoding AR | | | | |
| | Polypeptide encoded by PuvII | 16 | PRT | 44 |
| | to KasI sites of LacZ alpha | | | |
| | gene pUC18 or pUC19 with | | | |
| | synthetic oligonucleotides | | | |
| | comprising two TAA stop | | | |
| | codons near codons encoding | | | |
| | AR | | | |
| PuvII to just beyond | PuvII to KasI sites of LacZ | 84 | DNA | 45 |
| the KasI sites | alpha gene pUC18 or pUC19 | | | |
| of LacZ alpha gene | | | | |
| pUC18 or | | | | |
| pUC19 | | | | |
| | Polypeptide encoded by PuvII | 28 | DNA | 46 |
| | to KasI sites of LacZ alpha | | | |
| | gene pUC18 or pUC19 | | | |
| PuvII to KasI sites | PuvII to KasI sites of LacZ | 84 | DNA | 47 |
| of LacZ alpha gene | alpha gene pUC18 or pUC19 | | | |
| pUC18 or pUC19 | with stop codons replacing | | | |
| with stop codons | SE codon | | | |
| replacing NS codons | | | | |
| PuvII to KasI sites | PuvII to KasI sites of LacZ | 84 | DNA | 48 |
| of LacZ alpha gene | alpha gene pUC18 or pUC19 | | | |
| pUC18 or pUC19 with | with stop codons replacing | | | |
| stop codons | NS codons | | | |
| replacing NS codons | | | | |
| PuvII to KasI sites | PuvII to KasI sites of LacZ | 84 | DNA | 49 |
| | alpha gene pUC18 or pUC19 | | | |
| of LacZ alpha gene | with stop codons replacing | | | |
| pUC18 or pUC19 with | EE codons | | | |
| stop codons replacing | | | | |
| EE codons | | | | |
| PuvII to KasI sites | PuvII to KasI sites of LacZ | 84 | DNA | 50 |
| of LacZ alpha gene | alpha gene pUC18 or pUC19 | | | |
| pUC18 or pUC19 with | with stop codons replacing | | | |
| stop codons replacing | EA codons | | | |
| EA codons | | | | |
| PuvII to KasI sites | PuvII to KasI sites of LacZ | 84 | DNA | 51 |
| of LacZ alpha gene | alpha gene pUC18 or pUC19 | | | |
| pUC18 or pUC19 with | with stop codons replacing | | | |
| stop codons replacing | AR codons | | | |
| AR codons | | | | |
| Overlapping mini-Tn7 | Synthetic mini-attTn7 from −2 | 85 | DNA | 52 |
| ending with KasI site | to +2 with unknown nucleotides | | | |
| | at the insertion site, | | | |
| | followed by +3 to +58, then | | | |
| | Synthetic SalI, KasI and | | | |
| | other restriction sites | | | |
| Sequences near double | Sequences near double stop | 43 | DNA | 53 |
| stop codons replacing | codons replacing EA codons | | | |
| EA codons in lacZalpha | in lacZalpha peptide after | | | |
| peptide after | transposition of a mini-Tn7 | | | |
| transposition of a | into an overlapping | | | |
| mini-Tn7 into an | mini-attTn7 site | | | |
| overlapping | | | | |
| mini-attTn7 site | | | | |
| Junction near target | Junction near target site | 14 | DNA | 54 |
| site reading | after transposition into | | | |
| frame +1 | TAA stop codon reading | | | |
| | frame +1 | | | |
| Junction near target | Junction near target site | 15 | DNA | 55 |
| site reading frame +2 | after transposition into | | | |
| | TAA stop codon reading | | | |
| | frame +2 | | | |
| Junction near target | Junction near target site | 16 | DNA | 56 |
| site reading frame +3 | after transposition into | | | |
| | TAA stop codon reading | | | |
| | frame +3 | | | |
| pUC18 with EcoRI-SalI | pUC18 lacZalpha region | 381 | DNA | 57 |
| mini- attTn7 | containing an EcoRI-SalI | | | |
| | fragment from bMON 14272 | | | |
| | comprising a mini-attTn7 | | | |
| | fragment | | | |
| | Chimeric fusion protein | 126 | PRT | 58 |
| | comprising lacZalpha fragment | | | |
| | with insertion of EcoRI-SalI | | | |
| | fragment comprising a synthetic | | | |
| | mini- attTn7 fragment | | | |
| pACYC177 near PstI | Sequences near the unique PstI | 60 | DNA | 59 |
| site | site in the beta lactamase | | | |
| | gene of pACYC177 | | | |
| | Polypeptide encoded by sequences | 20 | PRT | 60 |
| | near the unique PstI site in | | | |
| | the beta lactamase gene of | | | |
| | pACYC177 | | | |
| pACYC177 PstI to EagI | Sequences near unique PstI | 60 | DNA | 61 |
| | site in pACYC177 mutated | | | |
| | to EagI site | | | |
| pACYC177 PstI to PuvII | Sequences near unique PstI | 60 | DNA | 62 |
| | site mutated to unique | | | |
| | PuvII site | | | |
| pACYC177 near 3′ end | pACYC177 with PstI site near | 60 | DNA | 63 |
| of NPT-II gene | the 3′ end of the NPT-II | | | |
| | gene that don′ t change the | | | |
| | amino acids “LQ” encoded by | | | |
| | the wild-type gene | | | |
| | Polypeptide encoded in | 15 | PRT | 64 |
| | pACYC177 with PstI site | | | |
| | near the 3′ end of the | | | |
| | NPT-II gene that don′ t | | | |
| | change the amino acids | | | |
| | “LQ” encoded by the | | | |
| | wild-type gene | | | |
| ACYC177 with PstI site | Sequences near 3′ end of | 60 | DNA | 65 |
| near 3′ end of NPT-II | pACYC177 with a new PstI | | | |
| gene | site that don′ t change | | | |
| | amino acids “LQ” encoded | | | |
| | at that position in the | | | |
| | NPT-II gene | | | |
| | Polypeptide encoded by | 15 | PRT | 66 |
| | sequences near 3′ end of | | | |
| | pACYC177 with a new PstI | | | |
| | site that don′ t change | | | |
| | amino acids “LQ” encoded | | | |
| | at that position in the | | | |
| | NPT-II gene | | | |
| pKM2 3′ end of | pKM2 3′ end of NPT-II | 51 | DNA | 67 |
| NPTII gene | gene | | | |
| | Polypeptide encoded by pKM2 | 6 | PRT | 68 |
| | 3′ end of NPT-II gene | | | |
| pKM243 3′ end of | pKM243 3′ end of NPT-II | 27 | DNA | 69 |
| NPT-II gene | gene | | | |
| | Polypeptide encoded by | 8 | PRT | 70 |
| | pKM243 3′ end of NPT-II | | | |
| | gene | | | |
| pKM243/1 3′ end of | pKM243/1 3′ end of NPT-II | 18 | DNA | 71 |
| NPT-II gene | gene | | | |
| | Polypeptide encoded by | 6 | PRT | 72 |
| | pKM243/1 3′ end of NPT-II | | | |
| | gene | | | |
| pKM243-1 3′ end of | pKM143-1 3′ end of NPT-II | 51 | DNA | 73 |
| NPT-II gene | gene | | | |
| | Polypeptide encoded by | 16 | PRT | 74 |
| | pKM143-l 3′ end of NPT-II | | | |
| | gene | | | |
| pACYC177 3′ end of | pACYC177 3′ end of | 43 | DNA | 75 |
| NPT-II gene | NPT-II gene | | | |
| | Polypeptide encoded by | 6 | PRT | 76 |
| | pACYC177 3′ end of | | | |
| | NPT-II gene | | | |
| pACYC177-QA 3′ end | pACYC177-QA 3′ end of | 43 | DNA | 77 |
| of NPT-II gene | NPT-II gene | | | |
| | Polypeptide encoded by | 6 | PRT | 78 |
| | pACYC177-QA 3′ end of | | | |
| | NPT-II gene | | | |
| PACYC177-PS | pACYC177-PS 3′ end of NPT-II | 43 | DNA | 79 |
| | gene | | | |
| | Polypeptide encoded by | 8 | PRT | 80 |
| | pACYC177-PS 3′ end of NPT-II | | | |
| | gene | | | |
| pACYC177-PSFNAVVYHS | pACYC177-PSFNAWYHS 3′ end of | 51 | DNA | 81 |
| | NPT-II gene | | | |
| | Polypeptide encoded by | 16 | PRT | 82 |
| | pACYC177-PSFNAWYHS 3′ end of | | | |
| | NPT-II gene | | | |
| pACYC177-Q** | pACYC177-Q** with two TAA stop | 43 | DNA | 83 |
| | codons after Q codon | | | |
| | Polypeptide encoded by | 7 | PRT | 84 |
| | pACYC177-Q** with two TAA stop | | | |
| | codons after Q codon | | | |
| pACYC177 P** | pACYC177-P** with two TAA stop | 43 | DNA | 85 |
| | codons after a P codon | | | |
| | Polypeptide encoded by pACYC177-P** | 7 | PRT | 86 |
| | with two TAA stop codons after a | | | |
| | P codon | | | |
| pACYC177 3′ end of | pACYC177 3′ end of | 50 | DNA | 87 |
| beta-lactamase gene | beta-lactamase | | | |
| | gene | | | |
| | Polypeptide encoded by pACYC177 3′ | 8 | PRT | 88 |
| | end of beta-lactamase gene | | | |
| pACYC177-K*** | pACYC177-K*** with two TAA stop | 50 | DNA | 89 |
| | codons before the normal TAA stop | | | |
| | codon | | | |
| | Polypeptide encoded by pACYC177- | 6 | PRT | 90 |
| | K*** with two TAA stop codons | | | |
| | before the normal TAA stop codon | | | |
| pACYC177~KH** | pACYC177-KH** with two stop | 50 | DNA | 91 |
| | codons after KH, one replacing | | | |
| | “essential Tryptophan (W) codon | | | |
| | Polypeptide encoded | 7 | PRT | 92 |
| | by pACYC177-KH** | | | |
| | with two stop codons after KH, | | | |
| | one replacing “essential | | | |
| | Tryptophan (W) codon | | | |
| pACYC177-KH** with | pACYC177-KHW** with | 50 | DNA | 93 |
| two stop codons | two stop codons | | | |
| after KH, one | at site of normal | | | |
| replacing “essential | TAA stop codon | | | |
| Tryptophan (W) codon | | | | |
| | Polypeptide encoded by | 8 | PRT | 94 |
| | pACYC177-KHW** | | | |
| | with two stop | | | |
| | codons at site of | | | |
| | normal TAA stop codon | | | |
| pAYC177-AAG | pACYC177-AAG | 11 | DNA | 95 |
| pACYC177-AAGT | pACYC177-AAGT | 12 | DNA | 96 |
| pACYC177-AAGTA | pACYC177-AAGTA | 13 | DNA | 97 |
| pACYC177-AAGCAT | pACYC177-AAGCAT | 14 | DNA | 98 |
| pACYC177-AAGCATT | pACYC177-AAGCATTT | 15 | DNA | 99 |
| pACYC177-AAGCATTA | pACYC177-AAGCATTA | 16 | DNA | 100 |
| PACYC177-AAGCATTGG | pACYC177-AAGCATTGG | 17 | DNA | 101 |
| pACYC177-AAGCATTGGT | pACYC177-AAGCATTGGT | 18 | DNA | 102 |
| pACYC177-AAGCATTGGTA | pACYC177-AAGCATTGGTA | 19 | DNA | 103 |
| pACYC177-PstI-BglI | pACUC177-PstI-BglI spanning | 141 | DNA | 104 |
| | junction between alpha and | | | |
| | omega fragments of beta- | | | |
| | lactamase | | | |
| | Polypeptide encoded by | 47 | PRT | 105 |
| | pACUC177-PstI-BglI spanning | | | |
| | junction between alpha and | | | |
| | omega fragments of beta- | | | |
| | lactamase | | | |
| pACYC177-PstI-Asel | pACYC177-PstI-Asel with | 105 | DNA | 106 |
| with linker | synthetic linker at junction | | | |
| | of alpha and omega fragments | | | |
| | of beta lactamase | | | |
| | Polypeptide encoded by | 35 | PRT | 107 |
| | pACYC177-PstI-Asel with | | | |
| | synthetic linker at junction | | | |
| | of alpha and omega fragments | | | |
| | of beta lactamase | | | |
| pACYC177-bla- | pACYC177-bla-alpha-omega-mini- | 180 | DNA | 108 |
| alpha-omega- | attTn7 with mini-attTn7 at the | | | |
| mini-attTn7 | junction of the alpha and omega | | | |
| | peptides of beta-lactamase | | | |
| | Polypeptide encoded by pACYC177- | 60 | PRT | 109 |
| | bla-alpha-omega-mini- attTn7 | | | |
| | with mini-attTn7 at the junction | | | |
| | of the alpha and omega peptides | | | |
| | of beta-lactamase | | | |
| Tn10 Tetracycline | lnterdomain loop in Tn10 | 401 | PRT | 110 |
| resistance protein | tetracycline resistance | | | |
| | protein | | | |
| | ETKNTRDNTDTEVGVETQSNSVYlTLF | | | |
| pACYC184 Tetracycline | lnterdomain loop in pACYC184 | 396 | DNA | 111 |
| resistance protein | tetracycline gene indirectly | | | |
| | derived from pSClOl | | | |
| | isolated from Shigella | | | |
| | flexneri | | | |
| | ESHKGERRPMPLRAFNPVSSFRWARGM | | | |
| pACYC184 reverse | Sequence from the reverse | 210 | DNA | 112 |
| complement | complement of pACYC184 | | | |
| spanning Tet | flanking the interdomain | | | |
| Interdomain | loop of the tetracyclin | | | |
| Loop | e resistance protein | | | |
| | Polypeptide encoded by | 70 | PRT | 113 |
| | sequence from the reverse | | | |
| | complement of pACYC184 | | | |
| | flanking the interdomain | | | |
| | loop of the tetracycline | | | |
| | resistance protein | | | |
| pACYC184 reverse | pACYC184 reverse complement | 297 | DNA | 114 |
| complement | Tet-mini-attTn7, with | | | |
| Tet-mini-attTn7 | synthetic mini-attTn7 | | | |
| | inserted near SalI site | | | |
| | in the sequences encoding | | | |
| | the interdomain linker of | | | |
| | the tetracycline resistance | | | |
| | protein | | | |
| | Polypeptide encoded by pACYC184 | 99 | PRT | 115 |
| | reverse complement Tet- | | | |
| | mini-attTn7, with synthetic | | | |
| | mini-attTn7 inserted near | | | |
| | SalI site in the sequences | | | |
| | encoding the interdomain | | | |
| | linker of the tetracycline | | | |
| | resistance protein | | | |
| EcoRI-SalI fragment | An EcoRI-SalI fragment | 95 | DNA | 116 |
| comprising | comprising a synthetic | | | |
| a synthetic mini-attTn7 | mini-attTn7 | | | |
| NotI-PspOMI linker | Synthetic NotI-PspOMI | 22 | DNA | 117 |
| | linker | | | |
| NotI-scar-PspOMI linker | Synthetic Linker with | 37 | DNA | 118 |
| | NotI-scar-PspOMI sites | | | |
| PspOMI-NotI linker | PspOMI-NotI linker | 22 | DNA | 119 |
| PspOMI-scar-NotI linker | Synthetic PspOMI-scar- | 37 | DNA | 120 |
| | NotI linker | | | |
| AbsI-SgrDI linker | Synthetic AbsI-SgrDI | 24 | DNA | 121 |
| | linker | | | |
| AbsI-scar-SgrDI linker | Synthetic AbsI-scar- | 40 | DNA | 122 |
| | SgrDI linker | | | |
| SgrDI-AbsI linker | Synthetic SgrDI-AbsI | 24 | DNA | 123 |
| | linker | | | |
| SgrDI-scar-AbsI linker | Synthetic SgrDI-scar- | 40 | DNA | 124 |
| | AbsI linker | | | |
| MauBI-AscI linker | Synthetic MauBI-AscI | 24 | DNA | 125 |
| | linker | | | |
| MauBI-scar-AscI linker | Synthetic MauBI-scar- | 40 | DNA | 126 |
| | AscI linker | | | |
| AscI-MauBI linker | Synthetic AscI-MauBI | 24 | DNA | 127 |
| | linker | | | |
| AscI-scar-MauBI linker | Synthetic AscI-scar- | 40 | DNA | 128 |
| | MauBI linker | | | |
| MauBI-AbsI linker | MauBI-AbsI | 24 | DNA | 129 |
| MauBI-SgrDI linker | MauBI-SgrDI | 24 | DNA | 130 |
| AscI-Abs linker | AscI-AbsI | 24 | DNA | 131 |
| AscI-SgrDI linker | AscI-SgrDI | 24 | DNA | 132 |
| AbsI-MauBI linker | AbsI-MauBI | 24 | DNA | 133 |
| Abs-AscI linker | AbsI-Asd | 24 | DNA | 134 |
| SgrDI-MauBI linker | SgrDI-MauBI | 24 | DNA | 135 |
| SgrDI-AscI linker | SgrDI-AscI | 24 | DNA | 136 |
| MauBI-PacI-AbsI | MauBI-PacI-AbsI | 24 | DNA | 137 |
| MauBI-PacI-SgrDI | MauBI-PacI-SgrDI | 24 | DNA | 138 |
| AscI-PacI-AbsI linker | AscI-PacI-AbsI | 24 | DNA | 139 |
| AscI-PacI-SgrDI linker | AscI-PacI-SgrDI | 24 | DNA | 140 |
| AbsI-PacI-MauBI linker | AbsI-PacI-MauBI | 24 | DNA | 141 |
| AbsI-PacI-AscI linker | AbsI-PacI-AscI | 24 | DNA | 142 |
| SgrDI-PacI-MauBI linker | SgrDI-PacI-MauBI | 24 | DNA | 143 |
| SgrDI-PacI-AscI linker | SgrDI-PacI-AscI | 24 | DNA | 144 |
| SgrDI-PacI-AbsI-AvrII- | MauBI-PacI-AbsI- | 54 | DNA | 145 |
| SgrDI-PacI-AscI linker | AvrII-SgrDI-PacI- | | | |
| | AscI | | | |
| MauBI-PacI-SgrDI-AvrII- | MauBI-PacI-SgrDI- | 54 | DNA | 146 |
| AbsI-PacI- AscI linker | AvrII-AbsI-PacI- | | | |
| | AscI | | | |
| AscI-PacI- AbsI-AvrII- | AscI-PacI-AbsI- | 54 | DNA | 147 |
| SgrDI-PacI- MauBI linker | AvrII-SgrDI-PacI- | | | |
| | MauBI | | | |
| AscI-PacI- SgrDI-AvrII- | AscI-PacI-SgrDI- | 54 | DNA | 148 |
| AbsI-PacI- MauBI linker | AvrII-AbsI-PacI- | | | |
| | MauBI | | | |
| AbsI-PacI-MauBI- AvrII- | AbsI-PacI-MauBI- | 54 | DNA | 149 |
| AscI-PacI- SgrDI linker | AvrII-AscI-PacI- | | | |
| | SgrDI | | | |
| AbsI-PacI-AscI-AvrII-MauBI- | AbsI-PacI-AscI- | 54 | DNA | 150 |
| PacI- SgrDI linker | AvrII-MauBI-PacI- | | | |
| | SgrDI | | | |
| SgrDI-PacI-MauBI-AvrII- | SgrDI-PacI-MauBI- | 54 | DNA | 151 |
| AscI-PacI- AbsI linker | AvrII-AscI-PacI- | | | |
| | AbsI | | | |
| SgrDI-PacI-AscI-AvrII- | SgrDI-PacI-AscI- | 54 | DNA | 152 |
| MauBI-PacI- AbsI linker | AvrII-MauBI-PacI- | | | |
| | AbsI | | | |
| MauBI-PacI-AscI linker | MauBI-PacI-AscI | 24 | DNA | 153 |
| AscI-PacI-MauBI linker | AscI-PacI-MauBI | 24 | DNA | 154 |
| AscI-PacI-SgrDI linker | AbsI-PacI-SgrDI | 24 | DNA | 155 |
| SgrDI-PacI-AbsI linker | SgrDI-PacI-AbsI | 24 | DNA | 156 |
| pTwist+Kan+MC | Twist Biosciences | 2007 | DNA | 157 |
| | cloning vector for | | | |
| | insertion of synthetic | | | |
| | DNA sequences, | | | |
| | comprising a medium | | | |
| | copy p15A bacterial | | | |
| | replicon and conferring | | | |
| | resistance to kanamycin | | | |
| pTKM-MaAbAvSgAs | pTwist-Kan-MC vector | 2159 | DNA | 158 |
| | with MauBI-PacI-AbsI- | | | |
| | AvrII-SgrDI-PacI- | | | |
| | AscI polylinker | | | |
| pTKM-CATd8 | cat gene from pACYC184 | 876 | DNA | 159 |
| | polypeptide | 219 | PRT | 160 |
| pTKM-CAT-TAA | cat gene from pACYC184 | 876 | DNA | 161 |
| | with one TAA stop codon | | | |
| | polypeptide | 212 | PRT | 162 |
| pTKM-CAT-TAATAA | cat gene from pACYC184 | 876 | DNA | 163 |
| | with two TAA stop codons | | | |
| | polypeptide | 211 | PRT | 164 |
| pTKM-CAT-TAATAA- | cat gene from pACYC184 | 889 | DNA | 165 |
| mini-attTn7 | and two TAA stop codons | | | |
| | followed by mini-attTn7 | | | |
| | target site | | | |
| | polypeptide | 211 | PRT | 166 |
| pTKMC-CAT-Tn7Lrf1 | gene fusion comprising | 896 | DNA | 167 |
| | cat gene from pACYC194 | | | |
| | fused to reading frame 1 | | | |
| | from end of Tn7L | | | |
| | polypeptide | 216 | PRT | 168 |
| pTKMC-CAT-Tn7Lrf2 | gene fusion comprising cat | 897 | DNA | 169 |
| | gene from pACYC194 fused | | | |
| | to reading frame 2 from | | | |
| | end of Tn7L | | | |
| | polypeptide | 228 | PRT | 170 |
| pTKMC-CAT-Tn7Lrf3 | gene fusion comprising cat | 898 | DNA | 171 |
| | gene from pACYC194 fused to | | | |
| | reading frame 3 from end of | | | |
| | Tn7L | | | |
| | polypeptide | 220 | PRT | 172 |
| pTwist-Chlor-MC cloning | pTwist-Chlor-MC cloning vector | 1953 | DNA | 173 |
| vector | | | | |
| pTwist+Chlor+MC | pTwist+Chlor+MC vector with | 2007 | DNA | 174 |
| vector with MauBI-PacI- | MauBI-PacI-AbsI-AvrII-SgrDI- | | | |
| AbsI-AvrII-SgrDI- | PacI-AscI polylinker | | | |
| PacI-AscI | | | | |
| polylinker | | | | |
| pTCM-Kan-CGRT | gene fusion comprising kanamycin | 1028 | DNA | 175 |
| | gene from pACYC177 extended to | | | |
| | also encode CGRTK and one stop | | | |
| | codon | | | |
| | polypeptide | 276 | PRT | 176 |
| pTCM-Kan-PSFNAVVYHS | gene fusion comprising kanamycin | 1040 | DNA | 177 |
| | gene from pACYC177 extended to | | | |
| | also encode PSFNAVVYHS and one | | | |
| | stop codon | | | |
| | polypeptide | 281 | PRT | 178 |
| pTCM-Kan-PS | gene fusion comprising kanamycin | 1016 | DNA | 179 |
| | gene from pACYC177 extended to | | | |
| | also encode PS and one stop codon | | | |
| | polypeptide | 273 | PRT | 180 |
| pTCM-Kan-Tn7Lrf1 | gene fusion comprising kanamycin | 1074 | DNA | 181 |
| | gene from pACYC177 extended to | | | |
| | also encode CGRTK and one stop | | | |
| | codon followed by partial Tn7L | | | |
| | polypeptide | 276 | PRT | 182 |
| pTCM-Kan-Tn7Lrf2 | gene fusion comprising kanamycin | 1075 | DNA | 183 |
| | gene from pACYC177 extended to | | | |
| | also encode LWADKlVGNWEGWKWSF | | | |
| | and one stop codon followed by | | | |
| | partial Tn7L in reading frame 2 | | | |
| | polypeptide | 288 | PRT | 184 |
| pTCM-Kan-Tn7Lrf3 | gene fusion comprising kanamycin | 1076 | DNA | 185 |
| | gene from pACYC177 extended to | | | |
| | also encode PVGSQNSWELGGVEMEFLRII | | | |
| | and one stop codon in reading | | | |
| | frame 3 | | | |
| | polypeptide | 290 | PRT | 186 |
| pTCM-Kan-PS-mini-attTn7 | gene fusion comprising kanamycin | 1069 | DNA | 187 |
| | gene from pACYC177 extended to | | | |
| | also encode PS and one stop | | | |
| | codon and overlapping | | | |
| | mini-attTn7 site | | | |
| | polypeptide | 273 | PRT | 188 |
| pTCM-Kan-PS | gene fusion comprising kanamycin | 1016 | DNA | 189 |
| | gene from pACYC177 extended | | | |
| | to also encode PS and one | | | |
| | stop codon | | | |
| | polypeptide | 193 | PRT | 190 |
| pTCM-Kan | Unaltered kanamycin gene | 1016 | DNA | 191 |
| | from pACYC177 and one TAA | | | |
| | stop codon | | | |
| | polypeptide | 271 | PRT | 192 |
| pTKM-lacZalpha- | lacZalpha gene comprising | 837 | DNA | 193 |
| mini-attTn7 | mini-attTn7 target site | | | |
| | polypeptide | 180 | PRT | 194 |
| pTKM-lacZalpha- | lacZalpha gene comprising | 687 | DNA | 195 |
| micro-attTn7 | micro-attTn7 target site | | | |
| | polypeptide | 130 | PRT | 196 |
| pTwist-Amp-HC | pTwist-Amp-HC cloning vector | 2221 | DNA | 197 |
| pTAH-MaAbAvSgAs | pTwist+Amp+HC with MauBI-AbsI- | 2275 | DNA | 198 |
| AvrII-SgrDI-AscI | | | | |
| polylinker | | | | |

Tables 24 and 26 also summarize features of Twist vectors 1-40 represented by SEQ ID NOS 199-240.

### Example 1—Design of Modular Sequences Encoding an Active LacZalpha-Mini-attTn7 Fusion Polypeptide

The development of cloning vectors comprising a multiple cloning site (MCS) within or between several segments of genes allowing rapid and easy screening for vectors comprising inserts greatly facilitated the cloning and analysis of a wide variety of prokaryotic and eukaryotic genes. High copy number vectors, such as pUC8 and pUC9, typically have an MCS inserted into a short segment at the 5′ end of the lacZ gene encoding an inactive fragment of β-galactosidase called the alpha peptide. The alpha peptide (“α-donor”) can bind to and complement an inactive α-acceptor, lacking a segment at the N-terminal region of the full length β-galactosidase, to restore activity of the enzyme [Juers et al (2012) Protein Science 21:1792-1807].

Two variants of β-galactosidase were observed in early studies, one deleting residues 23-31 and the other residues 11-41, caused the tetrameric enzyme to dissociate into inactive dimers. Peptides that included some of all of the missing residues, such as 3-41 or 3-92, restored the activity of the enzyme. Crystallographic studies have since shown that the donor binds to the site previously occupied by the deleted N-terminal residues, stabilizing and helping to restore the tetrameric structure. Residues from about 13 to 20 in adjacent subunits contact each other, and residues 29-33 occupy a tunnel in Domain 1 and the remainder of the acceptor polypeptide. Because critical catalytic residues are located in several domains, dissociation of the tetramer into the dimer disrupts all four active sites, abolishing the activity of the enzyme. The length of the complementing peptide is not important, as long as about 41 amino acid residues are present.

In many common E. coli strains used for cloning, the acceptor polypeptide is encoded by the lacZΔM15 gene which lacks residues 11-41 of the full length enzyme, having 1,024 residues. (In many older papers, the polypeptide numbering schemes apparently omit the amino-terminal methionine residue which is processed off in bacteria, so the second encoded amino acid is designated as +1). Many of these cells also contain the lacI gene encoding a repressor protein that binds to the lac operator in the vector, suppressing transcription of the lacZalpha gene in the cloning vector. When transformed host cells are spread on agar plates containing an appropriate antibiotic (typically ampicillin for many vectors), plus IPTG (isopropyl-β-D-thiogalactoside), and a chromogenic substrate, such as X-gal (5-bromo-4-chloro-3-intolyl-β-D-galactopyranoside), the IPTG induces transcription of the lac promoter and expression of the expression of the lacZalpha complementing peptide. Cells harboring vectors where the lacZalpha gene is intact, form blue colonies due to conversion of the X-gal and H₂O to galactose and 5-bromo-4-chloro-3-hydroxy-indole, which is converted in the presence of oxygen to the insoluble dimeric blue product, 5-5′-dibromo-4-4′-dichloro-indigo. Cells containing vectors where a segment of DNA is inserted into the multiple cloning site, disrupting the expression of the lacZalpha complementing peptide are white. White colonies are typically purified by restreaking a second time on the same type of plate, to ensure that they are not derived from a mixture of cells with a large white colony covering a small blue colony on a crowded plate. Plasmid DNA samples purified from white colonies are then characterized by analysis with restriction enzymes, gene amplification, DNA sequencing, or many other techniques.

While blue/white or similar colony color screening methods based on complementation between fragments of beta-galactosidase were developed in the early 1980s [Viera Messing (1982) Gene 19(3): 259-268], the first apparent use of this system to screen for insertions into or near a site comprising an attachment site for a transposon, was reported by the developers of the baculovirus shuttle vector (bacmid) system [Luckow et al, (1993)]. In their studies, a synthetic mini-attTn7 segment comprising the 3′ end of the glmS gene and extending into the intergenic region towards the phoS gene was inserted into the multiple cloning site of a lacZalpha gene derived from a cloning vector, but in the opposite orientation of its natural transcriptional direction, and in-frame with sequences upstream from the MCS and downstream from the MCS to encode a functional trimeric fusion protein that could complement the acceptor polypeptide encoded by the lacZΔM15 gene on the chromosome. DH10B cells harboring plasmids comprising this segment formed blue colonies on agar plates in the presence of an antibiotic, the inducer IPTG, and the chromogenic substrate, X-gal. DH10B cells harboring the bacmid, bMON14272, conferring resistance to Kanamycin, and the compatible helper plasmid pMON7124, conferring resistance to Tetracycline, also form blue colonies on plates containing these antibiotics, plus IPTG and X-gal, or similar types of chromogenic substrates (e.g., Bluo-gal, which produces a darker blue product than X-gal, which is turquoise).

When a donor plasmid, such as pMON14327 comprising the β-glucuronidase gene under the control of the polyhedrin promoter, or vectors derived from the pFastBac series of vectors noted above, is introduced into E. coli DH10B harboring the bacmid and the helper plasmid, the mini-Tn7 cassette from the donor plasmid in many cases will transpose into the synthetic mini-attTn7 target site located on the low copy number bacmid, or into the attTn7 located near the 3′ end of the glmS gene on the chromosome. Insertion into the synthetic site on the bacmid produces colonies that are white, in the presence of Kanamycin, Tetracycline, IPTG, and X-gal, in a background of blue colonies, that have the mini-Tn7 inserted into the unique site on the chromosome. Sectored colonies, part blue and part white, were sometimes observed on plates spread with bacteria, and when the white portions were restreaked on similar plates, white colonies always gave rise to white colonies.

Despite the remarkable success of this system to facilitate the expression of a wide variety of proteins in cultured insect cells for use in basic and applied research, particularly therapeutic polypeptides, vaccines, and components of cell and gene therapy vector systems over the past 26 years, there is a continuing need to develop new and improved vectors that facilitate the cloning and insertion of gene expression cassettes into large plasmids and viral shuttle vectors. Improvements to shuttle vectors comprising the target site, the donor plasmid, and the helper plasmid, may permit the development of more rapid methods for the assembly and characterization of complex vectors comprising one or more genes of interest, suitable for use in a wide variety of applications, compared to vectors and methods that are currently available from academic and corporate institutions.

The synthetic lacZ-alpha-mini-attTn7 target site used in the bacmid system described above, was derived from pMON7134, which contains a 523 HincII fragment of pEAL1 containing attTn7 into the HincII site of pEMBL9 [Barry (1988)]. A 112 bp fragment was amplified by polymerase chain reaction (PCR) using two primers to generate a fragment containing a 87 bp functional attTn7 corresponding to positions −23 to +61 with respect to the insertion site at position 0) with EcoRI and SalI 5′ sticky ends. The 112 bp amplified fragment was cloned into the lacZalpha region of the cloning vector pBCSKP to generate the vector pMON14192. E. coli DH10B harboring pMON14192 formed blue colonies on plates containing X-gal or Bluo-gal. This plasmid was linearized with ScaI and amplified with primers containing BbsI sites to generate a 708 bp product with EcoRI and SalI compatible sticky ends, and ligated to pMON14181 (containing a Kanamycin resistance gene linked to a mini-F replicon) to form pMON14231 (mini-F-Kan-lacZalpha-mini-attTn7), which formed light blue colonies containing X-gal or Bluo-gal due to its much lower copy number. This plasmid was partially digested with BamHI to generate full-length linear molecules and ligated to the baculovirus transfer vector pMON14118 (˜8,538 bp) digested with BglII to produce two transfer vectors pMON14271 and pMON14272 (each ˜18,053 bp), which were used to generate the baculovirus shuttle vectors bMON14271 and bMON14272, that conferred resistance to Kanamycin, and formed blue colonies on plates containing X-gal or Bluo-gal, that were infectious when introduced into Spodoptera frugiperda Sf9 cells.

Key features of a 2033 bp fragment extracted from the sequence of bMON14272 extending from an SbfI site located 124 bp upstream from the 5′ end of the CAP binding site near the lac promoter and operator to a sequence including a SexAI site in the 5′ end of the ytc gene in the cloned mini-F replicon include the following genetic elements:
the lac promoter and operator upstream from the coding sequence for the first 5 amino acids of the lacZalpha polypeptide;
the left part of a multiple cloning site (MCS) derived from pBCSKP;
the synthetic sequence comprising the attTn7 target;
the right second part of the MCS derived from pBCSKP, a sequence encoding amino acids 7-59 of the lacZalpha polypeptide; and
a 123 bp segment encoding 40 additional amino acid extending beyond the BbsI site to the SexAI site near a TAA stop codon in the 5′ end of the ytc gene of the mini-F replicon sequences.

It seems remarkable, now more than 26 years after these genetic elements were first designed and assembled, that the system for screening insertions of a transposon into a synthetic attachment site worked as well as it did, and very few attempts, if any, were made by others to improve this aspect of the baculovirus shuttle vector system. It is desirable, though, to remove unnecessary sequences, particularly those within the residual parts of the multiple cloning site, and to systematically shorten and test sequences comprising the synthetic mini-attTn7 target site.

The sequences from the ATG start codon of the lacZalpha peptide through the end of the SexAI recognition site near the TAA stop codon are shown below. The underlined portions are derived from the multiple cloning sites or extend from the 3′ end of the original pBCSKP cloning vector into adjacent sites in the 5′ end of a non-essential gene found in the F plasmid.

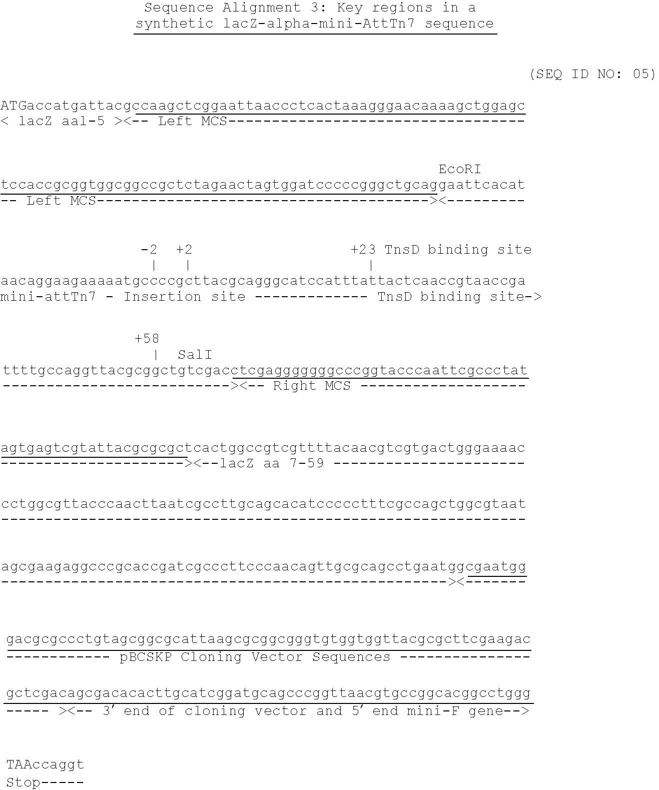

All of the underlined sequences are not essential to the synthetic target site, and could be deleted to produce a much shorter synthetic attTn7 target, while preserving key features of the screenable method of detecting transpositions of mini-Tn7 elements into this sequence. While the short sequences at the end of the mini-attTn7 comprising recognition sites for EcoRI or SalI are not critical to targeting or insertion of mini-Tn7 elements, and not underlined, they are still useful for extracting and moving this segment from one cloning vector to another, or as a source of material used in a variety of gene amplification techniques.

One of many possible truncated versions of this sequence is shown below.

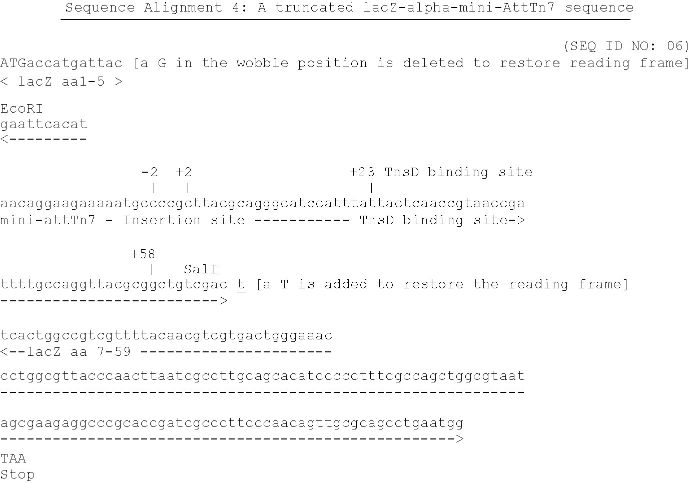

Sequences shown above and similar sequences are most easily prepared by direct DNA synthesis which are also flanked by sequences comprising one or more recognition sites for restriction enzymes, to facilitate insertion into vectors comprising compatible restriction sites under the control of inducible promoters, such as the lac promoter and operator, and variants thereof. This segment may also be directly linked to a suitable promoter in coupled gene amplification reactions where segments of an upstream promoter and/or a downstream transcriptional terminator are included in the reaction mixture, where there are suitable overlaps between the promoter sequence and the 5′ end of the synthetic lacZalpha-mini-attTn7 target sequence noted above, and the 3′ portion of this sequence overlapping with the 5′ portion of a segment comprising a transcriptional terminator sequence.

Variants of the synthetic target site are also prepared by systematically deleting nucleotide sequences between the ATG start codon of the lacZalpha polypeptide and sequences just upstream and downstream from the 5-bp Tn7 insertion site that is located 5′ to the TnsD protein binding sites in the 3′ end of the retained portion of the glmS gene. Systematic sets of deletions, designed to retain the reading frame of the chimeric fusion protein, will help define the boundaries and essential residues needed for targeting of mini-Tn7 elements, and synthetic derivatives, where the left and right arms of Tn7 are altered by mutagenesis, or genes encoding any of the relevant transposition proteins are mutagenized, and characterized by their ability to transpose into mini-attTn7 targets sites, or altered variants of the target site, in this system.

Modular versions of the genetic cassette comprising the lacZ-attTn7 target site, operably linked to a suitable prokaryotic or eukaryotic promoter may be moved to other plasmids or shuttle vectors by traditional cloning methods, or by more modern methods assembling segments of genes into multifunctional vectors.

A wide variety of vectors comprising the synthetic lacZ-attTn7 target site and longer or shorter variants, may also be used with this system to screen for insertions of mini-Tn7 sequences into a single target maintained on an autonomous replicon or the chromosome of a host cell. These include small and large plasmids that propagate in enteric and non-enteric bacteria, viral shuttle vectors, such as insect and mammalian dsDNA viruses, particularly baculovirus- and herpesvirus-derived shuttle vectors, TI plasmid and chloroplast-derived vectors used to facilitate the insertion of genes into transformed plant cells, tissues, allowing the generation of transgenic plants, and in fungal systems used to facilitate the expression of gene products for research and in industrial biotechnology applications.

The following table illustrates phenotypes of colonies of E. coli DH10B harboring different plasmids used in the transposition system colonies on agar media containing a chromogenic substrate specific for β-galactosidase, such as X-gal or Bluo-gal, in the presence of one or more kinds of antibiotics.

TABLE 8
Phenotypes of DH108 Harboring Plasmids in lacZalpha-mini-attTn7 Transposition Studies

| Designation | | | | | |
|---|---|---|---|---|---|
| DH10B/ | | Inc | Phenotype on | | |
| plasmid(s) | Markers | Group | X-gal plates | Stable | Description |
| bMON14272 | Kan^{R} | IncFl | Lac plus (blue) | Yes | E. coli DH10B harboring |
| (bacmid) | | | | | just the bacmid |
| | | | | | bMON 14272 comprising |
| | | | | | a contiguous segment |
| | | | | | encoding resistance to |
| | | | | | Kanamycin, the lacZ-mini- |
| | | | | | attTn7 target sequence, |
| | | | | | and the mini-F replicon |
| pMON1724 | Tet^{R} | IncColE1 | Lac minus (white) | Yes | pMON7124 encodes |
| (helper) | | | | | tnsA, B, C, D, and E, near |
| | | | | | Tn7R on a pBR322-based |
| | | | | | replicon. |
| pFastBac1 | Amp^{R}, | IncColE1 | Lac minus (white) | Yes | The donor plasmid |
| (donor) | Gent^{R} | | | | encodes Ampicillin |
| | | | | | resistance gene on the |
| | | | | | backbone and |
| | | | | | Gentamycin Resistance |
| | | | | | Gene, plus baculovirus |
| | | | | | polyhedrin promoter, |
| | | | | | MCS and SV40 poly(A) |
| | | | | | between Tn7L and Tn7R. |
| bMON14272 + | Kan^{R}, | IncFl + | Lac plus | Yes | Bacmid plus helper |
| pMON7124 | Tet^{R} | IncColE1 | (blue) | | plasmids |
| bMON14272 + | [Kan^{R}, | IncFl + | Lac plus (blue) >> | No, until | Bacmid plus compatible |
| pMON7124 + | Tet^{R}, | [IncColE1 + | Lac minus (white) | transposition | helper and incompatible |
| pFastBac1 | Amp^{R}, | IncColE1] | (by insertion into | from donor | donor plasmids |
| | Gent^{R}] >> | >> IncFl + | bacmid to create | to bacmid or | |
| | Kan^{R}, | IncColE1 | composite bacmid) | chromosome, | |
| | Tet^{R}, | | or Lac plus (blue) | losing vector | |
| | Amp^{S}, | | (by insertion into | backbone of | |
| | Gent^{R} | | chromosome) | donor | |
| | | | | plasmid | |

FIG. 4 sets forth an illustration entitled “E. coli lacZ-based gene fusions to screen or select for Tn7-based transposition events”.

### Example 2—Design and Assembly of Vectors Allowing for Direct Selection of Site Specific Transposons Inserted into their Attachment Site and Methods Thereof Based on Cassettes Comprising CAT-attTn7 Gene Fusions

Indirect screenable methods for detecting insertions of site-specific transposons into synthetic target sequences such as those disclosed in the Background of the Invention and Example 1, noted above, work remarkably well. Variant sequences, which eliminate small segments upstream or downstream from the minimal set of attTn7 sequences may also improve the contrast between events that result in insertions and background levels of expression of the chimeric protein comprising segments that can complement a chromosomally-encoded acceptor protein on different types of agar plates or other types of media that result in color changes in the presence of a chromogenic substrate.

There is a need, however, for methods that allow for the direct selection of bacteria harboring vectors comprising synthetic attTn7 target sites. Direct selection will allow for directed evolution of mutagenized mini-Tn7 transposons, target sites, and sequences encoding transposition proteins, leading to the development of synthetic gene insertion systems, which may have altered efficiencies of transposition into a specific target site or altered abilities to transpose into variants of the wild-type target site compared to systems generally based on unaltered parental transposon and target sequences.

Chloramphenicol (Cam or CM, Formula: C₁₁H₁₂Cl₂N₂O₅, IUPAC name: 2,2-dichloro-N-[(1R,2R)-1,3-dihydroxy-1-(4-nitrophenyl)propan-2-yl]acetamide) is an old antibiotic, now typically used to treat ocular infections caused by Staphylococcus aureus, Streptococcus pneumoniae, and Escherichia coli. Chloramphenicol is a bacteriostatic drug, binding to two residues in the 23S rRNA of the 50S subunit of the ribosome, preventing the elongation of protein chains. Chloramphenicol is also a potent inhibitor of cytochrome P450 isoforms CYP2C19 and CYP3A4 in the liver, which decrease the metabolism and increasing the circulating levels of a wide variety of other drug products.

Resistance to chloramphenicol (CMR) can diminish its effectiveness in clinical settings. Reduced permeability of bacterial membranes is a common mechanism, that confers a low level of resistance to the drug. Mutations in the 50S subunit of the ribosome also confer resistance, but are rare. High level resistance is conferred by a gene encoding chloramphenicol acetyl transferase (CAT; EC 2.3.1.28), which inactivates the molecule by adding one or two acetyl groups derived from acetyl-S-coenzyme A to hydroxyl groups on the molecule, which prevents the drug from binding to the ribosome.

A wide variety of genes encoding chloramphenicol acetyl transferase have been isolated and compared Commonly studied are the Type I and the Type III enzymes, which have been shown to be trimers of identical subunits (MW 25,000) with a histidine residue at position 195 identified as having a key role in the catalytic reactions involved in acetylation of chloramphenicol bound to a deep pocket in the trimer complex. The crystal structure of the Type III enzyme, isolated from E. coli, bound to chloramphenicol has been determined.

Gene cassettes encoding CAT are widely used in bacteriology and molecular genetics to facilitate the selection of plasmids carrying DNA segments with a promoter operably-linked to the cat gene. One common application is to clone an intact cat gene downstream from a promoter of interest, as a gene fusion in a reporter system, to measure the relative activity of different promoters, or the same promoter in different types of tissues. It is also commonly used to facilitate cloning of DNA segments into plasmid vectors, within the cat gene, destroying its activity, or within cloning sites located elsewhere on a plasmid that confers resistance to CM.

Genes encoding Type I CAT are located in a wide variety of cloning vectors. The plasmid pACYC184, for example, has a cat gene derived from Tn9, that encodes a Type I CAT protein, containing a p15A origin of replication [Chang, A. C. Y. and Cohen, S. N. (1978) J. Bacteriol. 134: 1141-1156.]. This plasmid, which is 4,245 bp, also confers resistance to tetracycline (TET). Plasmids containing DNA segments inserted into the unique EcoRI site of this plasmid are resistant to TET, but not CM. Plasmids containing DNA segments inserted into the unique EcoRV, BamHI, SalI, or many other sites of this plasmid are resistant to CM, but not TET.

NR1/R100, R1, and many other large plasmids that confer resistance to several types of antibiotics (drug resistance or R plasmids), also carry genes related to Tn9, which encode the type I CAT polypeptide. R plasmids may also carry genes which confer tolerance to heavy metal ions, including mercury, silver, and cadmium, arsenic [Foster, T. J. (1983) “Plasmid-determined Resistance to Antimicrobial Drugs and Toxic Metal Ions in Bacteria. Microbiology Rev 47(3):361-409]. Plasmid-specified resistance to compounds comprising bismuth, lead, boron, chromium, cobalt, nickel, tellurium, and zinc have also been described [Summers and Silver (1979) Microbial transformation of metals. Ann Rev Microbiol. 32: 637-372].

What is not well known, however, is that the CAT protein tolerates small deletions or insertions (to produce larger fusions) at its amino and carboxy termini. A series of HIV-1 Vpr-CAT N- and C-terminal fusion proteins were constructed and evaluated, which had the activity of both Vpr and CAT domains [Yao et al (1999), Gene Therapy]. Small deletions at the carboxy terminus, are also possible, provided that they do not extend upstream from a conserved cysteine residue near the carboxy terminus of the CAT protein [Robben et al, (1995)] [Van der Schueren et al, 1998]. This residue is located at position 8 residues from the end of the 219 residue Type I CAT protein, and at 6 residues from the end of 213 aa Type III CAT protein. Note the following key observations:
Insertion of a TAA stop codon immediately at or upstream from the Cysteine codon in the gene for the Type I CAT protein results in a polypeptide that is inactive.
Insertion of the TAA stop codon after the Cysteine codon and before the normal stop codon should allow expression of a truncated polypeptide that is functional.
Deletion of the conserved Cysteine residue is believed to prevent assembly of CAT into its active trimer complex.

DNA cassettes encoding the Type I or Type III CAT proteins, where a stop codon, such as TAA, TGA, or TAG, are located after a codon encoding Cysteine, and one or more codons for non-conserved amino acid residues upstream from the conserved Cysteine codon are designed as noted below. If a site for a restriction enzyme is located after the Cysteine codon is used as part of a cloning site that destroys the stop codon, then the reading frame of the mRNA encoding the upstream portion of the CAT protein may be altered, allowing readthrough into the mRNA segment transcribed from the downstream DNA segment. Sequences of novel gene fusions where site-specific insertions of a segment from a transposon alters the reading frame at the stop codon, allowing expression of a fusion polypeptide is active are noted in more detail below.

One way to directly select for insertions of site specific transposons into their target site, is to design and assemble an array of genetic elements to include a promoter and optional operator, operably-linked to a sequence encoding a drug resistance marker, and a synthetic sequence encoding the target site for the transposon. The design and assembly of genetic cassettes encoding a fusion between the gene encoding Chloramphenicol Acetyl Transferase (CAT) and the mini-attTn7, or a variant that includes a portion of the coding sequence for the lacZ alpha protein, as a CAT-attTn7-lacZ fusion protein, are described below.

The junction of the fusion is after a codon for a conserved Cysteine residue near the 3′ end of the gene, adding a TAA stop codon, and then most of the mini-AttTn7 segment. By carefully selecting the relative position of the tnsB binding site so that the duplicated target site (−2 to +2) is within the TAA stop codon after the Cys codon, so that when the Tn7 is inserted, it disrupts the stop codon allowing readthrough into the 5′ end of the left arm of Tn7 (Tn7L, which begins TGT, and then 5 more bases, before the start of several conserved tnsD binding sites).

CAT fusions can be created at both ends of the gene, but those that extend upstream from the conserved Cys codon are inactive. By restoring a few amino acids beyond the Cys codon, the protein is active again. In one type of fusion, the target site is in a segment that normally does not confer resistance to CM, but if a transposition event occurs, CAT resistance is restored. This arrangement allows one to directly select for CM resistance, and all of the expected structures should be gene fusions with the CAT reading into Tn7L. Direct selection should allow for the detection of rare transposition events (1×10⁻⁵).

Different promoters can be used to drive expression of CAT-attTn7 fusion polypeptide, such as its native promoter, or the inducible lac promoter. These strategies should apply to equally well to gene fusions assembled from the Type I cat gene, as well as those derived from the Type III cat gene. The Type I cat gene is more widely available on a variety of medium copy number cloning vectors (such as pACYC184) and low copy number drug resistance plasmids (NR1/R100).

The plasmid pACYC184 (4,345 bp) has two genes encoding resistance to Tetracycline (TC) and to Chloramphenicol (CM). It also has replicon derived from the plasmid p15A, allowing it to co-exist in cells comprising ColE1-derived replicons, such as pBR322 and the pUC series of plasmids. It is a medium copy number vector, maintained at about 15 copies per cell, which can be amplified by treatment with spectinomycin under specific growth conditions. The Type I cat gene in pACYC184 encodes a protein having 219 aa. Several unique restriction sites are located just within the 3′ end of the gene, and just downstream from its TAA stop codon.

Several plasmids are constructed to demonstrate feasibility of a new system designed to allow direct selection for insertions of mini-Tn7 segments into synthetic CAT-attTn7 target sites, as noted below. They can be derived directly from pACYC184 by traditional cloning methods using cleavage and ligation of restriction fragments into cloning vectors, or by synthesizing gene fusions of interest that are directly inserted into a common base vector (such as those provided by Twist Biosciences) and characterized by DNA sequencing, gene amplification, restriction fragment analysis, or similar methods to characterize the structure of a vector molecule. Twist Biosciences provides a variety of vectors comprising medium (p15A) or high (pUC) copy number replicons, and a selectable marker conferring resistance to chloramphenicol, kanamycin, or ampicillin that comprise a common site where the DNA sequence of interest is inserted. Given the low cost and ease of ordering synthetic DNA molecules, ordering complete vectors from a vendor are now usually preferred, compared to traditional methods of cloning gene fusions of interest that are described In the following examples.

Initially, pACYC184 DNA is digested with the enzyme TatI (A′GTAC,T) which produces a 5′ sticky ends, or with ScaI (AGT′ACT) which produces blunt ends, and with the enzyme BaeGI or Bme1508I (both of which G,KGCM′C). The start of the TatI site is located at position +410 in the vector, and the end of the BaeGI/Bme1508I site is at position +467. There are 30 bases from the beginning of the TatI site to the start of the TAA stop codon, encoding a the C-terminal peptide sequence QYCDEWQGGA*.

Synthetic oligonucleotides are prepared and annealed to replace the segment of DNA extending from the TatI or ScaI site to the BaeGI/Bme1508I site. Additional unique restriction sites are located at longer distances downstream from the BaeGI/Bme1508I site, including Tth111I, DrdI, BtsaI, and Bsu36I, if the BaeGI/Bme1508I site is unsuitable for some reason. The synthetic oligonucleotides also contain a recognition site for a rare cutting restriction enzymes (such as those having an 8-bp recognition sequence, preferably a SrfI (GCCC|GGGC) site and an internal XmaI (C′CCGG,G) site, to facilitate extraction of the gene cassette comprising the synthetic CAT-attTn7 sequences when used in conjunction with other unique sequences located within the N-terminal sequence of the cat gene or sequences 5′ from that start of the gene also includes a promoter sequence.

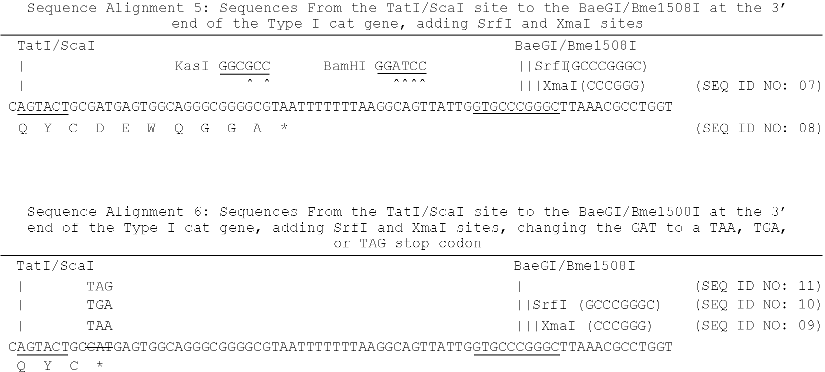

The wild-type TatI to BaeGI fragment can be replaced by several altered versions, one comprising a BamHI site in the untranslated region downstream from the natural TAA stop codon, and variants where one or two stop codons are inserted at the positions where the critical Cysteine (C) residue, and the Aspartic Acid (D) residue are located upstream from the natural TAA stop codon. Inserting one stop codon at the position of the Asp codon should truncate the protein, to encode a truncated variant that is active. Inserting two stop codons, replacing the adjacent Cys and Asp codons, should also truncate the protein, to encode a truncated variant that is inactive.

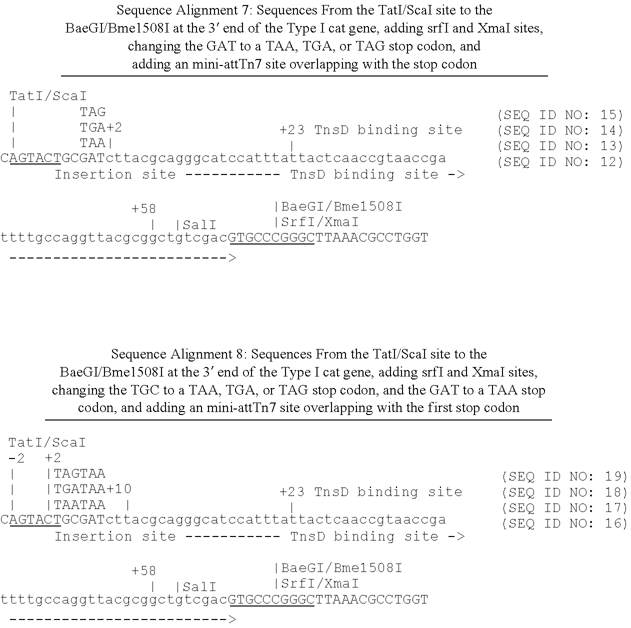

Transposing a mini-Tn7 element into the attTn7 site will alter the reading frame of the encoded polypeptide, adding extra amino acids to the CAT-attTn7 fusion protein restoring its activity, allowing for the direct selection bacteria harboring composite vectors comprising transposition events.

A sequence containing the mini-attTn7 site that has its insertion site positioned to be just before the first TAA should allow transposition in replacing the stop codon by the TGT of the left arm of Tn7, restoring activity.

The segments shown below illustrate the junction between a Type I cat gene and a mini-Tn7 element inserted into an a target site where the TAA stop codon overlaps with positions 0 to +2 of a 5-bp insertion site (from −2 to +2) of a mini-attTn7 target site, restoring expression of a longer, active CAT fusion protein. The relative position of the transposition site can be adjusted by a single base across the desired insertion site.

Note that the extended CAT fusion protein extends for varying lengths depending on the reading frame of the gene (+1, +2, or +3), where the TGT represents the first 3 nucleotides of the left arm of Tn7.

The segment shown below illustrates the junction between a Type I cat gene and a Tn7 element inserted into an overlapping mini-attTn7 target site, restoring expression of a longer, active CAT fusion protein.

Sequence Alignment 9: Sequences at the 3' end of a Type I cat gene after
transposition of a mini-Tn7 into an over overlapping mini-attTn7 site

(SEQ ID NO: 20) Omitted (SEQ ID NO: 22)

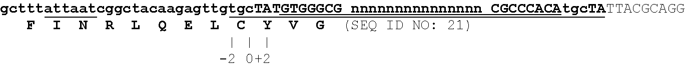

The relative position of the 5-bp insertion site can be moved slightly to the left or right of the sequences encompassing the critical Cysteine codon or sequences in adjacent codons to produce different types of truncated proteins, or longer fusion proteins that result by changing the reading frame of downstream intervening segments and sequences in the left arm of Tn7, where a variety of stop codons are located at different distances from the end of Tn7L.

Sequence Alignment 10: Sequences at the 3' end of a Type I cat
gene that mimic Tn7L at the junction of mini-Tn7 replacing a
stop codon for a Cys codon in an overlapping mini-attTn7 site

The following sequence mimics insertion of the Tn7L replacing the stop codon for a
Cys codon, restoring activity to the encoded CAT fusion protein.
−2 +2
| | BamHI BaeGI/SrfI/XmaI

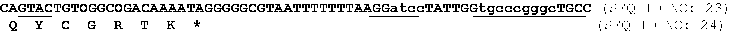

Bacteria harboring synthetic gene fusions comprising truncated, wild-type, or extended forms of the cat gene should have different phenotypes when plated on different concentrations of chloramphenicol, as shown below.

TABLE 9
Colony Phenotypes of pACYC184 derivatives encoding CAT-attTn7 fusion proteins

| | | Markers | | Reference or | |
|---|---|---|---|---|---|
| | | Cat^{R} = + | | SEQ ID NO of | |
| Designation | Markers | Cat^{S} = − | Description | Inserted Sequence | Source |
| pACYC184 | Tet^{R}, | + | pACYC184 carries genes conferring | Chang, A. and | Boca |
| | Cat^{R} | | resistance to tetracycline and | Cohen, S. (1978); | Scientific |
| | | | chloramphenicol (Type I cat gene encoding | Sequence reported | |
| | | | 219 aa residues). It has the same replicon | by Rose, R. E. | |
| | | | as pACYC177. | (1988). | |
| pACYC184-SrfI | Tet^{R}, | + | pACYC184 digested with TatI or ScaI and | (SEQ ID NO: 7) | This |
| | Cat^{R} | | BaeGI or Bme1508I and ligated to or | | study |
| | | | amplified to include an oligonucleotide | | |
| | | | encoding a SrfI/XmaI site. | | |
| GAT > TAA | Tet^{R}, | − | pACYC184 containing an oligonucleotide | (SEQ ID NO: 9) | This |
| | Cat^{S} | | changing the codon following the Cysteine | | study |
| | | | Codon from GAT to TAA. | | |
| GAT > TGA | Tet^{R}, | − | pACYC184 containing an oligonucleotide | (SEQ ID NO: 10) | This |
| | Cat^{S} | | changing the codon following the Cysteine | | study |
| | | | Codon from GAT to TGA. | | |
| GAT > TAG | Tet^{R}, | − | pACYC184 containing an oligonucleotide | (SEQ ID NO: 11) | This |
| | Cat^{S} | | changing the codon following the Cysteine | | study |
| | | | Codon from GAT to TAG. | | |
| GAT > TAA | Tet^{R}, | − | pACYC184 containing an oligonucleotide | (SEQ ID NO: 12) | This |
| overlapping | Cat^{S} | | changing the codon following the Cysteine | | study |
| mini-AttTn7 | | | Codon from GAT to TAA with an attTn7 | | |
| | | | sequence overlapping with the Cysteine | | |
| | | | Codon. | | |
| GAT > TGA | Tet^{R}, | − | pACYC184 containing an oligonucleotide | (SEQ ID NO: 13) | This |
| overlapping | Cat^{S} | | changing the codon following the Cysteine | | study |
| mini-AttTn7 | | | Codon from GAT to TGA with an attTn7 | | |
| | | | sequence overlapping with the Cysteine | | |
| | | | Codon. | | |
| GAT > TAG | Tet^{R}, | − | pACYC184 containing an oligonucleotide | (SEQ ID NO: 14) | This |
| overlapping | Cat^{S} | | changing the codon following the Cysteine | | study |
| mini-AttTn7 | | | Codon from GAT to TAG with an attTn7 | | |
| | | | sequence overlapping with the Cysteine | | |
| | | | Codon. | | |
| TAA > TAT::Tn7 | Tet^{R}, | + | Insertion of Tn7 at the TAA Stop codon | SEQ ID NO: 23 | This |
| | Cat^{R} | | restores CAT activity. | | study |
| TGA > TGT::Tn7 | Tet^{R}, | + | Insertion of Tn7 at the TGA Stop codon | | This |
| | Cat^{R} | | restores CAT activity. | | study |
| TAG > TAT::Tn7 | Tet^{R}, | + | Insertion of Tn7 at the TAG Stop codon | | This |
| | Cat^{R} | | restores CAT activity. | | study |

Variants of plasmids based on pACYC184 can also be created using any of a variety of other replicons. Vectors provided by Twist Biosciences, for example, can also be used. In the series noted below, key segments derived from the chloramphenicol resistance gene of pACYC184 are synthesized and inserted into pTwist-Kan-MC (also abbreviated as pTKM), which confers resistance to chloramphenicol and has a medium copy number replicon derived from the plasmid p15A. Polylinker sequences flank the entire kanamycin resistance gene, including its promoter, that containing for two or more 8-bp recognition sites for rare cutting restriction enzymes, such as MauBI, AbsI, SgrDI, and AscI.

TABLE 10
Expected Phenotypes of DH10B Harboring pTwist-Kan-MC plasmids comprising CAT-mini-attTn7
fusion proteins with staggered sets of TAA stop codons

| | Base | | | | |
|---|---|---|---|---|---|
| | Vector | Insert | Expected | | SID |
| Short Name | Markers | Marker | Phenotype | Insert Segments | NOS |
| pTwist + Kan + MC | KAN | None | KanR | None | 157 |
| pTKM- | KAN | None | KanR | MauBI-AbsI-AvrII-SgrDI-AscI polylinker | 158 |
| MaAbAySgAs | | | | | |
| pTKM-CATd8 | KAN | None | KanR, | CAT gene from pACYC184 not extended or truncated | 159/ |
| | | | CamR | and deleted 8 bases from the right polylinker | 160 |
| pTKM-CAT | KAN | CAT | KanR, | CAT gene from pACYC184 not extended or truncated | |
| | | | CamR | | |
| pTKM-CAT-TAA | KAN | CAT | KanR, | TAA replaced Asp Codon | 161/ |
| | | | CamR | | 162 |
| pTKM-CAT- | KAN | CAT | KanR, | TAATAA replaced CysAsp Codons | 163/ |
| TAATAA | | | CamS | | |
| pTKM-CAT- | KAN | CAT | KanR, | TAATAA replaced CysAsp Codons-overlapping mini- | 165/ |
| TAATAA-mini- | | | CamS | AttTn7 | 166 |
| attTn7 | | | | | |
| pTKMC-CAT- | KAN | CAT | KanR, | CAT extended with CGRTK with partial Tn7L rf1 | 167/ |
| Tn7Lrf1 | | | CamR | | 168 |
| pTKMC-CAT- | KAN | CAT | KanR, | CAT extended with LWADKIVGNWEGWKWSF with | 169/ |
| Tn7Lrf2 | | | Cam??? | partial Tn7L rf2 | 170 |
| pTKMC-CAT- | KAN | CAT | KanR, | CAT extended with PVGGQNSWELGGVEMEFLRII with | 171/ |
| Tn7Lrf3 | | | Cam??? | partial Tn7L rf3 | 172 |

If the phenotypes are as expected, then the plasmid containing the mini-attTn7 sequence can be used as the basis for additional experiments where a helper plasmid is introduced into the cells, and a donor plasmid transformed in, and plating out in the presence of tetracycline and chloramphenicol. (The marker on the helper plasmid may need to be changed so it is different from that used by the target plasmid). All target plasmids that confer resistance to Tc and CM should have a mini-Tn7 inserted at the 3′ end of the truncated/extended cat gene.

E. coli DH10B harboring the pACYC184 series of vectors and a variant of the helper plasmid, pMON7124, that encodes a drug resistance marker, such as Kanamycin instead of Tetracycline, can be transformed with a donor plasmid, such as pFastBac1 or a variant thereof (each conferring resistance to Ampicillin and Gentamycin), to test transposition of the mini-Tn7 element from the donor into the target site on different pACYC184 variants containing synthetic attTn7 sites. E coli DH10B cells comprising the unmodified patent plasmid or each of the variant plasmids are then spread on agar plates comprising tetracycline if pMON7124 is used as a helper vector, plus different concentrations of chloramphenicol to determine the relative sensitivity to chloramphenicol. The phenotypes should match what is predicted in tables noted below.

Transposition events in cells containing the overlapping attTn7 sequence should restore CAT activity, compared to those having the longer attTn7 sequence linked downstream from the truncated cat genes. The Gentamycin resistance marker, which is located on the mini-Tn7 element on the donor plasmid, with the 3′ end of its gene oriented to terminate near Tn7R, should be irrelevant in transposition schemes where the direct selection of transposition events occur by insertion into a gene fusion comprising a truncated cat gene, and where CAT activity is restored after transposition of the mini-Tn7 element into the target site on the pACYC184 derived vector containing an overlapping mini-attTn7 sequence.

Screening for resistance or sensitivity to Gentamycin, from colonies that confer resistance to Chloramphenicol after transposition should facilitate confirmation of transposition events into the target site on a plasmid, compared to the chromosome. Eliminating the need for a drug resistance marker within the mini-Tn7 element, allows the donor plasmid to be much smaller, before and after transposition, greatly facilitating the design and cloning of cassettes to be inserted into one or more related attachment sites on a target vector, and avoiding the need to remove the gentamycin or other resistance markers after transposition for specific applications.

Segments from any of these plasmids may then be moved to other plasmids with different replicons by digesting them with restriction enzymes that cut outside the critical genetic elements, by amplifying the key sequences using PCR-like techniques, or by synthesizing and assembling one or more segments and ligating them into appropriate vectors.

The plasmid pACYC177, which has the same replicon as pACYC184 and encodes genes conferring resistance to Ampicillin and Kanamycin, can be used to clone segments derived from the pACYC184 derivatives noted above and below, that contain variable lengths of a sequence comprising a mini-attTn7 target site, to facilitate testing of transposition in cells where the target confers resistance to Kanamycin, the donor confers resistance to Amp and Gentamycin, and the helper confers resistance to Tetracycline.

Vectors having much lower copy numbers, such as the mini-F replicon used in the baculovirus shuttle vectors and in many Bacterial Artificial Chromosomes (BAC) vectors, available from a variety of academic, non-profit, or commercial sources, can also be used to facilitate analysis of transposition events using selectable and screenable marker schemes.

The following table illustrates phenotypes of colonies of E. coli DH10B harboring different plasmids used in the transposition system colonies on agar media in the presence of one or more kinds of antibiotics. Agar plates containing rosanilin dyes such as crystal violet can be used in agar plates to score chloramphenicol resistance types by colony color, such as CM-sensitive sectors in CM-resistant colonies [Proctor and Rownd, 1982]. This procedure, typically used to facilitate screening during cloning by insertional inactivation of cat gene encoding an active enzyme, may not work for cells harboring a nearly full length, but inactive enzyme, if the dye binds to one or more domains outside regions comprising key residues of its catalytic site.

TABLE 11
Colony Phenotypes of DH10B Harboring Plasmids in CAT-mini-attTn7
Transposition Studies

| | | | Phenotype | | |
|---|---|---|---|---|---|
| | | | on | | |
| Designation | | | crystal | | |
| DH10B/ | | Inc | violet | | |
| plasmid(s) | Markers | Group | plates | Stable | Description |
| pACYC17 | Amp^{R}, | p15A | CAT | Yes | pACYC177 carries |
| (control) | Kan^{R} | | minus (−) | | genes conferring |
| | | | (light) | | resistance to ampicillin |
| | | | | | and kanamycin |
| | | | | | resistance gene. |
| pACYC184 | Tet^{R}, | p15A | CAT | Yes | pACYC184 carries |
| (control) | Cat^{R} | | plus (+) | | genes conferring |
| | | | (dark) | | resistance to |
| | | | | | tetracycline and |
| | | | | | chloramphenicol. |
| pMON1724 | Tet^{R} | ColE1 | CAT | Yes | pMON7124 encodes |
| (helper) | | | minus (−) | | tnsA, B, C, D, and E, |
| | | | (light) | | nearTn7R on a |
| | | | | | pBR322-based |
| | | | | | replicon. |
| pFastBac1 | Amp^{R}, | ColE1 | CAT | Yes | The donor plasmid |
| (donor) | Gent^{R} | | minus (−) | | encodes Ampicillin |
| | | | (light) | | resistance gene on the |
| | | | | | backbone and |
| | | | | | Gentamycin Resistance |
| | | | | | Gene, plus baculovirus |
| | | | | | polyhedrin promoter, |
| | | | | | MCS and SV40 |
| | | | | | poly(A) between Tn7L |
| | | | | | and Tn7R. |
| pACYC184 | Kan^{R}, | Fl and | CAT | Yes | pACYC184 and |
| (control) + | Tet^{R} | ColE1 | plus (+) | | pMON7124 are in |
| pMON7124 | | | (dark) | | different compatibility |
| (helper) | | | | | groups and should |
| | | | | | stably co-exist in the |
| | | | | | same cell, selecting for |
| | | | | | kanamycin or |
| | | | | | chloramphenicol |
| | | | | | resistance and |
| | | | | | tetracycline resistance, |
| | | | | | respectively. |

FIG. 5 sets forth an illustration entitled “E. coli Type I cat gene-based gene fusions to select for Tn7-based transposition events”.

### Example 3—Design of Modular Sequences Encoding an Inactive LacZalpha-Mini-attTn7 Fusion Polypeptide

Strategies similar to those described above for the design and construction of CAT-attTn7 gene fusions can also be applied to generate lacZalpha-mini-attTn7 fusions, where a stop codon is inserted at or near the codon for amino acid 41 (counting from the second codon, after the ATG codon encoding the N-terminal methionine residue, which is processed off in E. coli) of the lacZalpha polypeptide. LacZalpha polypeptides that are shorter than 41 amino acids long cannot efficiently bind to and complement the LacZ acceptor polypeptide encoded by the lacZΔM15 gene [Juers et al (2012)].

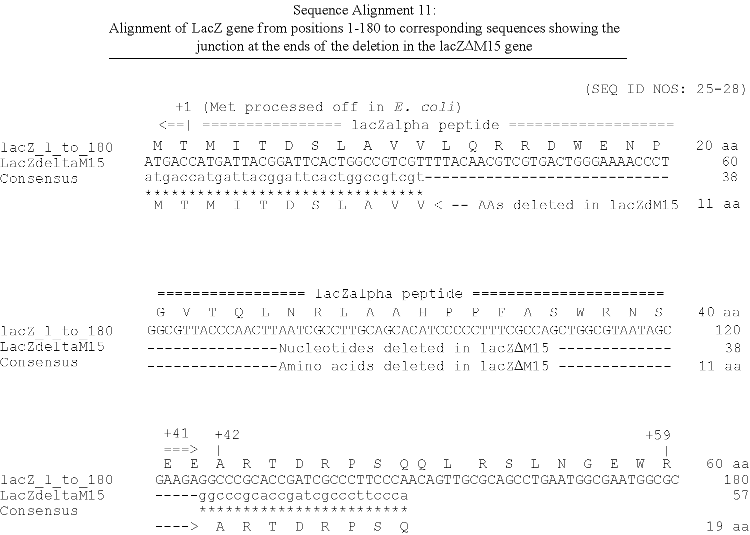

In this design, gene cassettes encoding a truncated lacZalpha protein and an overlapping mini-attTn7 are assembled and tested. Cassettes containing a lacZalpha that encode a polypeptide that is 42 or more amino acids long should complement and be lac plus on selection plates, or indicator plates comprising a chromogenic substrate. Those that are 41 amino acids or shorter should not efficiently complement and be lac minus on selection or indicator plates.

Transposition of a mini-Tn7 sequence into a truncated lacZ-alpha gene with an overlapping mini-attTn7 should restore the reading frame of the lacZalpha gene enabling expression of a longer alpha polypeptide that can complement, changing the phenotype from lac minus before transposition to lac plus after transposition.

In this design, blue colonies in a background of white colonies are picked and analyzed for the presence of the mini-Tn7 cassette inserted into the synthetic target sequence. Methods allowing outgrowth of lac plus cells in liquid minimal media comprising an appropriate carbon source before spreading on agar plates may facilitate the amplification and direct selection of colonies containing transposition events.

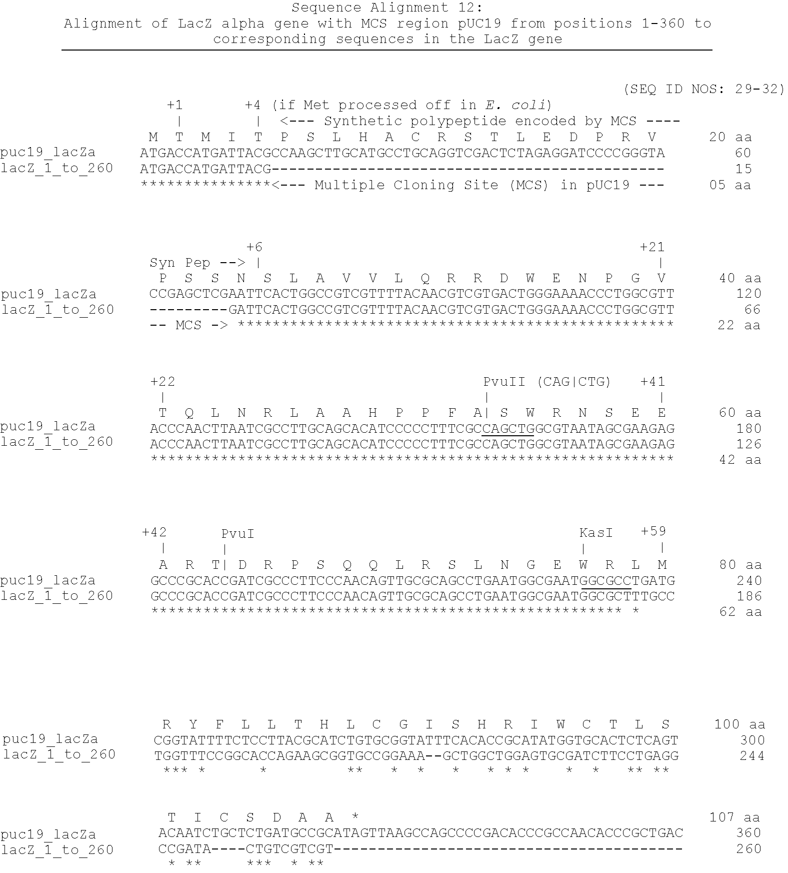

Plasmid pUC18 or pUC19 DNA ([Yanish-Peron (1985)], obtained from Thermo Fisher or New England Biolabs) is partially-digested with PvuII, to create a linearized full length version of the plasmid, and treated with alkaline phosphatase, or a functionally similar phosphatase, to remove terminal phosphate residues. A synthetic linker is then added containing one or more unique restriction sites which do not cut in the parent plasmid sequence, and ligated to the linearized plasmid DNA, and transformed into competent E. coli cells. Two types of plasmids with linkers are recovered, one where the PvuII site in an intergenic region upstream from lac promoter contains the unique linker containing at least the one or more unique restriction sites and is not digestible by PvuII, and a second type where the linker is located in the lacZalpha gene.

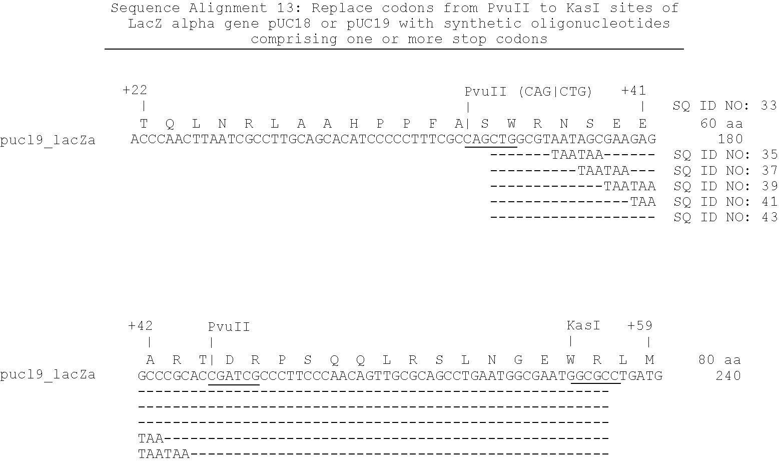

The nucleotide sequences are represented by even SEQ ID NOS and the encoded polypeptides by odd Seq ID NOS.

The plasmid variant that retains the natural PvuII site within the lacZalpha gene is selected for additional studies. DNA from that plasmid variant is digested with PvuII and KasI and a series of synthetic oligonucleotides comprising a series of one or more stop codons in frame with the lacZalpha polypeptide reading frame that have a blunt end and a compatible sticky end are inserted into the vector backbone, ligated, and transformed into competent bacteria comprising the lacZΔM15 gene. A series of ampicillin resistant vectors are recovered and their phenotypes characterized on chromogenic indicator plates.

In one series of vectors, noted above, the synthetic oligonucleotides contain two sequential TAA stop codons. At least one variant plasmid where double TAA stop codons are inserted is recovered, where expression of an alpha peptide of a functionally competent fragment is prevented, that can complement the acceptor fragment encoded by the lacZΔM15 gene on the chromosome.

If the transition encompasses the codons for consecutive E and A residues, as noted below, then a synthetic oligonucleotide is prepared comprising downstream sequences comprising an overlapping mini-attTn7 target sequence and ligated into the vector between the PvuII and KasI sites.

Sequence Alignment 14: Staggered sets of synthetic nucleotides
encoding double TAA stop codons from PvuII to KasI sites of LacZ alpha
gene pUC18 or pUC19 lined up with a synthetic mini-attTn7 sequence

```
                                                                                                                        (SEQ ID NOS: 45/46, 47-51)
    PvuII (CAG|CTG) +41 +42 PvuI KasI +59
    | | | | | |
  A| S W E N S E E A R T| D R P S Q Q L R S L N G E W R L M

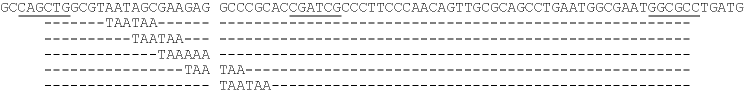

                                    −2 +2 +23 tnsD binding site
                                      | TAA TAA |
                      --------nnnnn ttacgcagggcatccatttattactcaaccgtaaccga (SEQ ID NO: 52)
                    Insertion site ------------------ tnsD binding site->
                                                                                    |BaeGI/Bme1508I
                                                    +58 |SafI/XmaI
                                                        | |SaiI | |KasI
                      ttttgccaggttacgcggctgtcgacGTGCCCGGGCGGCGCC
                      ------------------------->
```

The plasmid variant comprising the stop codon upstream from the overlapping mini-attTn7 target sequence is then tested in a transposition system comprising a compatible helper plasmid and an incompatible mini-Tn7 donor plasmid. The sequences near the end of the insertion site showing the 5 bp duplication at the left and right arms of Tn7 are shown below. In this example, three sets of insertions are shown, shifted by one nucleotide, where the conserved TGT from the left end of Tn7 replace 3, 2, or 1 nucleotides of the first of two TAA stop codons bordering the junction between the codons for amino acids 41 and 42 of the lacZ polypeptide. Sequences upstream from the insertion point encode amino acids S and E, before being joined to 3 types of polypeptides encoded by the transition sequences extending into the left arm of Tn7 where they terminate at varying distances by TAA, TGA, or TAG stop codons farther into Tn7L (not shown).

Sequence Alignment 15: Sequences near double stop codons
replacing EA codons in lacZalpha peptide after transposition
of a mini-Tn7 into an overlapping mini-attTn7 site

```
                −2 +2 +23 tnsD binding site
                  | TAA TAA |
  --------AAGAG ttacgcagggcatccatttattactcaaccgtaaccga (SEQ ID NO: 53)
Insertion site ------------------ tnsD binding site->

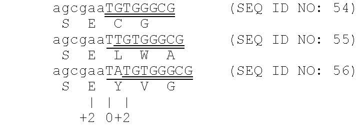

```

It is desirable to prepare a control plasmid derived from a plasmid encoding the lacZ alpha peptide, such as pUC18 or pUC 19 vector, to insert the mini-attTn7 target site into the middle of the multiple cloning site such that the reading frame of the sequence encoding the target site is in frame with the sequences encoding the first few amino acids of the lacZalpha polypeptide, and sequences downstream from the multiple cloning site are also in frame through the stop codon 3′ to the sequences encoding amino acids 42 and beyond of the lacZ polypeptide.

In one of many possible examples, pUC18 can be used to clone the EcoRI-SalI mini-attTn7 fragment from the bacmid bMON14272, which has the EcoRI-SalI sites in the same reading frame as that in pUC18. The background may be high, since both the parent and resulting plasmid are both Ampicillin resistant and Lac plus on selection or indicator plates.

Plasmid pUC18 DNA is also digested with an enzyme that cuts in the middle of the MCS, the ends filled in with DNA polymerase or nibbled back, and re-ligated and transformed into bacteria and a Lac minus derivative is recovered and characterized. That plasmid is digested with EcoRI and SalI and ligated with EcoRI-SalI fragment from bMON14272 DNA to create a pUC18 derivative with the mini-attTn7 target site that confers resistance to Ampicillin and is lac plus on indicator plates. The sequence of one derivative is shown below.

Sequence Alignment 16: Clone mini-attTn7 of bMON14272 into EcoRl-
SalI sited of LacZ alpha gene of pUC18 restoring reading frame

```
      +1 +4EcoRI
        | lacZ || < Synthetic polypeptide encoded by mini-AttTn7
  M T M I T| N S H N R K K N A P L T Q G I (SEQ ID NO: 58)
ATGACCATGATTACGaattcacataacaggaagaaaaatgccccgcttacgcagggcatc (SEQ ID NO: 57)
                                                                                  | |
                                                                                −2 +2
                            <-------------------- Insertion Site ---------
                                                                                        SalI
--------------------------------------------|---------------
  H L L L N R N R F C Q V T R L| S T C R H

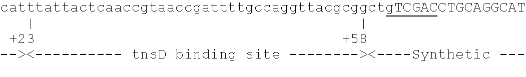

      +6 +21
-> |------------------ LacZalpha ---------------------|
  A S L A L A V V L Q R R D W E N P G V T
GCAAGCTTGGCACTGGCCGTCGTTTTACAACGTCGTGACTGGGAAAACCCTGGCGTTACC
-->
                                                                                                          +41+42
----------------------- LacZalpha ---------------------| |
  Q L N R L A A H P P F A S W R N S E E A
CAACTTAATCGCCTTGCAGCACATCCCCCTTTCGCCAGCTGGCGTAATAGCGAAGAGGCC
----------------------- LacZalpha --------------------------
  R T D R P S Q Q L R S L N G E W R L M R
CGCACCGATCGCCCTTCCCAACAGTTGCGCAGCCTGAATGGCGAATGGCGCCTGATGCGG
----------------------- LacZalpha --------------------------
  Y F L L T H L C G I S H R I W C T L S T
TATTTTCTCCTTACGCATCTGTGCGGTATTTCACACCGCATATGGTGCACTCTCAGTACA
--- LacZalpha ---
  I C S D A A *
ATCTGCTCTGATGCCGCATAG
```

Restriction fragments containing this segment can be moved to other modular plasmids or shuttle vectors by using enzymes that cut 5′ to and 3′ to this segment, or various derivatives, or by amplifying the DNA segment using PCR primers that have desirable sites for one or more restriction enzymes that are compatible with those used in the vector to clone the digested or amplified DNA segment. Transposition events using vectors comprising this segment are detected by screening on plates containing a chromogenic substrate, such as X-gal, where white colonies will contain insertions that disrupt the expression of the lacZalpha polypeptide, preventing complementation with the acceptor polypeptide encoded by the lacZΔM15 gene.

Similar strategies can also be used to obtain and clone or insert DNA fragments encoding active and truncated forms of the lacZalpha polypeptide fused to a synthetic mini-attTn7 sequence, allowing the direct selection of transposition events, in the presence of substrates for β-galactosidase, and by screening in the presence of a chromogenic substrate, where lac plus colonies, that are blue, will contain inserts, extending the sequence of the lacZalpha polypeptide, compared to a truncated version that cannot bind to and complement the acceptor polypeptide encoded by the lacZΔM15 gene.

MacConkey agar is a selective and differential medium that be used to distinguish colonies that can ferment lactose (Lac plus) from those that cannot (Lac minus). MacConkey medium contains peptones and lactose as nutrients, plus bile salts and crystal violet to inhibit most Gram-positive bacteria, and the dye neutral red. Bacteria that metabolize lactose produce acid, lowering the pH of the agar below pH 6.8, turning the dye red, and creating pink (Lac plus) colonies in a background of pale yellow (Lac minus) colonies.

Some strains of enteric bacteria that carry a mutation in the galE gene that encodes galactose epimerase, are highly sensitive to galactose, due to accumulation of a toxic intermediate, UDP-galactose, that promotes cell lysis [Fukasawa, T. and H. Nikaido. (1961)]. Mutant galE strains that are also Lac plus, are sensitive to lactose or its analogue phenyl-β-D-galactoside, since β-galactosidase converts lactose to glucose and galactose, leading to the accumulation of the toxic metabolite UDP-galactose. A variety of common laboratory E. coli strains harboring different types of cloning vectors encoding the lacZalpha polypeptide, that also comprise the lacZΔM15 gene encoding the acceptor polypeptide were evaluated on rich and minimal media supplemented with 0.1% D-galactose or 0.1% lactose [Reddy (2004)]. Some strains harboring plasmids that express the lacZalpha polypeptide and complement the acceptor polypeptide encoded by the chromosomal lacZΔM15 gene, performed better than others on test plates, which may be related to the copy number of the plasmid, or activity of the reconstituted enzyme. The author noted that agar plates containing nutrient poor media generally worked better than rich media, and that outgrowth in minimal liquid media supplemented with lactose before plating may enrich the population of Lac minus cells comprising recombinant plasmids with insertions in their lacZalpha genes. Comparable results were obtained when an E. coli C strain, that is lacZ minus and galE minus harboring a plasmid pUR288 which encodes all of lacZ were plated on rich (LB) and poor (LB/M9 in a 1/9 vol/vol ratio, containing 0.05% phenylgalatcoside), suggesting that these methods, while promising, require careful evaluation of a variety of minimal media components [Gossen et al (1992)].

### Example 4—Design of Modular Sequences Encoding Inactive and Active Forms of NPT-II (KAN)-Mini-attTn7 Fusion Proteins

Transposon Tn5 encodes a variety of genes including one, neomycin phosphotransferase II (NPT-II) confers resistance to neomycin and kanamycin in bacteria. NPT-II also confers resistance to G418 (Geneticin, G418 sulfate) in mammalian cells. These and other closely related antibiotics bind to components of the ribosome, inhibiting protein translation. NPT-II phosphorylates the antibiotics, interfering with their active transport into the cell. A wide variety of cloning vectors contain the gene encoding NPT-II to facilitate selection of bacteria in the presence of kanamycin on agar plates and in liquid cultures. This gene and variants encoding several types of fusion proteins are also widely used to facilitate selection of vectors commonly used in transformed plant cells and tissues.

Reiss et al (1984) observed that a series of genes comprising alterations at the 3′ end of the NPT-II gene encoding truncated proteins or extended fusion proteins were generated, which vary in activity compared to the native enzyme. A plasmid designated pKM2, comprising the wild-type gene conferred resistance to Kanamycin on at levels exceeding >1000 ug/ml. The gene used in these studies encodes a polypeptide ending with the sequence “LLDEFF” before ending with a TGA stop codon.

Two plasmids encoding extended variant forms, ending with “LLDEFFQA” and “LLDEFFPSFNAVVYHS” before terminating with TAG stop codons also conferred resistance comparable to the wild-type enzyme of >1000 ug/ml kanamycin. One extended variant encoding an additional 263 aa segment derived from a tetracycline resistance gene was inactive, while a second extended variant encoding an additional 303 aa segment was partially active, conferring resistance on plates containing 200 ug/ml kanamycin, and a third variant encoding an additional 300 aa segment, much less active, conferring resistance on plates containing 20 ug/ml kanamycin.

The extensions in each of these variants differed though, the first two encoding Gln-Ala (QA) immediately after the Phe-Phe (FF) residues in the wild-type enzyme, and the third variant comprising Pro-Asp (PN) after the Phe-Phe (FF) residues and extending beyond that for another 298 residues.

Most remarkable, however, are the properties of a fourth variant, which encodes Pro-Ser and 8 other residues (PSFNAVVYHS) immediately after the Phe-Phe (FF) residues before terminating at a TAA stop codon. Bacteria harboring the plasmid encoding the fourth variant could not grow on agar plates containing any amount of kanamycin, providing strong evidence that the encoded fusion protein was completely inactive.

The authors concluded that length alone, is insufficient to alter the activity of the NPT-II fusion protein and that biochemical characteristics of additional amino acids immediately near the carboxy terminal residues of the wild-type protein can also dramatically influence the activity of the fusion protein.

These and other observations concerning the identification of critical residues near the carboxy terminus of specific enzymes can be considered in the design of a variety of fusion proteins comprising synthetic mini-attTn7 target sites. In the CAT-attTn7 gene fusions noted earlier, the critical amino acid residue is a Cysteine, located several positions before the last amino acid of the CAT protein, and insertions by transposition into a stop codon at or near the Cys codon, will extend the protein, restoring its activity. In the experiments described below, alterations near the normal stop codon for NPT-II, including those encoding Gln (Q) and Pro (P) are made, and tested for their influence on the activity of slightly extended NPT-II fusion proteins. Bacteria harboring plasmids comprising genes encoding inactive variants are then used as targets in transposition experiments to determine if insertion of a mini-Tn7 element into a synthetic mini-attTn7 site restores activity, allowing direct selection for bacteria in the presence of kanamycin that should harbor plasmids comprising site specific insertions.

Plasmid pACYC177, which confers resistance to Ampicillin and Kanamycin, is digested with PflMI (CCAN,NNN′NTGG) and BsmFI (GGGAC(N)₉₋₁₀′NNNN,), and compatible sets of synthetic oligonucleotides are inserted between those sites to generate a series of plasmid variants encoding the sequences noted below.

The start of the recognition site for PflMI through is 125 nucleotides upstream from (5′ to) the start of the TAA stop codon at the end of the NPT-II gene, and the end of the cleavage site for BsmFI site 70 nucleotides downstream from (3′ to) the end of TAA stop codon, so it is desirable to prepare an altered form of pACYC177, where at least one new, unique restriction site is located near the end of the gene, which does not alter the sequence of any encoded polypeptide. This would facilitate insertion of sets of oligonucleotides that are much shorter than those required for insertion between the unique PflMI and BsmFI sites in pACYC177 (˜200 nt) needed for these studies.

There is a site comprising the sequence “TTGCAG” encoding “LQ” near the 3′ end of the NPT-II gene in pACY177 that can be mutated to “C,TGCA′G” comprising a recognition site for PstI, while encoding “LQ” since TTG and CTG are both codons for Leucine (L).

There is also an existing PstI (C,TGCA′G) site in the beta-lactamase gene of pACYC177 from position +299 to +304 overlapping 3 codons encoding “PAA”. The T and A residues can be both be mutated since they are in wobble positions for these codons, allowing changes from PstI CTGCAG to EagI C′GGCC,G or PstI to PvuII (CAG|CTG) creating unique sites, since they do not cut in parental pACYC177. A unique SacII (CC,GC′GG) is located near one end of the sequences comprising the p15A origin of replication.

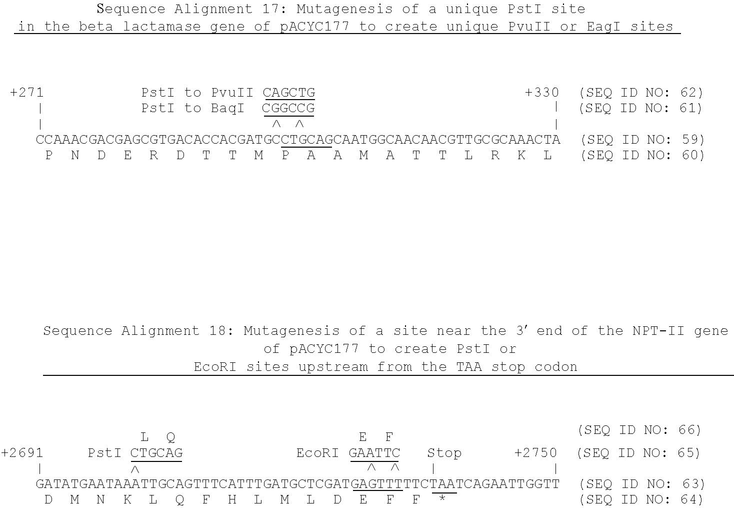

Two derivatives of pACYC177 are made by site directed mutagenesis, pACY177-PvuII, and pACYC177-EagI which remove the PstI site starting at position +299.

Both of these derivatives are then used as templates in a second experiment, changing the T at position +2703 to C, creating a unique PstI site at that position, in plasmids called pACYC177-PvuII-3′-PstI and pACYC177-EagI-3′-PstI. Another derivative can also be made, creating an EcoRI site near the 3′ end of the gene, that does not alter the two consecutive amino acids encoded at those positions.

Plasmid DNAs are purified and subjected to restriction enzyme analysis confirming the presence or absence of the expected restriction enzyme sites, and sequenced across the boundaries of the mutagenized sequences.

Bacteria comprising the parental pACYC177 plasmid and the variants are tested on a series of agar plates, and the variants are expected to confer resistance to Ampicillin and Kanamycin at the same level as the parental plasmid.

Sequence Alignment 19: Junction sequences at the 3' end of genes
encoding C-terminal NPT-II (KAN)-mini-attTn7 fusion proteins

```
pKM2
cttcttgacgagttcttc TGAgcgggactctggggttcgaaatgaccacca (SEQ ID NO: 67/68)
  L L D E F F *
pKM243

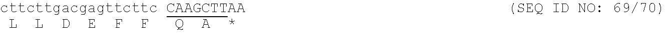

pKM243/1
cttcttgacgagttcttc (SEQ ID NO: 71/72)
  L L D E F F
pKM243-1
cttcttgacgagttcttc CCAAGCTTTAATGCGGTAGTTTATCACAGTTAA (SEQ ID NO: 73/74)
  L L D E F F P S F N A V V Y H S *
pACYC177
ATGCTCGATGAGTTTTTC TAATCAGAATTGGTTAATTGGTTGT (SEQ ID NO: 75/76)
  M L D E F F *
pACYC177-QA

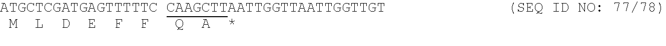

pACYC177-PS

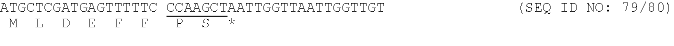

pACYC177-PSFNAVVYHS
ATGCTCGATGAGTTTTTC CCAAGCTTTAATGCGGTAGTTTATCACAGTTAA (SEQ ID NO: 81/82)
  M L D E F F P S F N A V V Y H S *
```

Plasmid DNAs comprising the synthetic oligonucleotides noted above are recovered, and sequenced to confirm their expected structure, and bacteria harboring the unaltered pACYC177 and the variant plasmids are spread on a series of agar plates containing increasing concentrations of kanamycin to determine their phenotype.

TABLE 12
Expected Phenotypes of DH10B Harboring Plasmids Comprising KAN-mini-attTn7 Fusion Proteins

| Designation | | | Expected | | | |
|---|---|---|---|---|---|---|
| DH10B/plasmid(s) | Markers | Inc Group | Phenotype | Stable | SEQ ID NOS | Source |
| pKM2 | Cam^{R}, Kan^{R} | | Kan plus (+) | Yes | 67/68 | [Reiss et al (1984)] |
| pKM243 | Cam^{R}, Kan^{R} | | Kan plus (+) | Yes | 69/70 | [Reiss et al (1984)] |
| pKM243/1 | Cam^{R}, Kan^{R} | | Kan plus (+) | Yes | 71/72 | [Reiss et al (1984)] |
| pKM243-1 | Cam^{R}, Kan^{S} | | Kan minus (−) | Yes | 73/74 | [Reiss et al (1984)] |
| pACYC177 | Amp^{R}, Kan^{R} | P15A | Kan plus (+) | Yes | 75/76 | This study |
| pACYC177-QA | Amp^{R}, Kan^{R} | P15A | Kan plus (+) | Yes | 77/78 | This study |
| pACYC177-PS | Amp^{R}, Kan^{S} | P15A | Kan minus (−) | Yes | 79/80 | This study |
| pACYC177-PSFNAVVYHS | Amp^{R}, Kan^{R} | P15A | Kan minus (−) | Yes | 81/82 | This study |

A series of additional plasmids are prepared, which contain a synthetic mini-attTn7 that overlaps with the normal stop TAA codon, or codons just upstream from it that encode other amino acids, particularly those, such as Proline (P) that may encode an inactive form of a slightly extended NPT-II fusion protein. Transposition into a sequence comprising an inactive NPT-II-overlapping mini-attTn7 fusion protein should restore activity, allowing direct selection and recovery of bacteria harboring plasmids with transposition events.

Sequence Alignment 20: Staggered sets of synthetic nucleotides
encoding double TAA stop codons from near the 3' end of the NPT-II
gene of pACYC177 lined up with a synthetic mini-attTn7 sequence

```
      EcoRI GAATTC SpeI ACTAGT
                      \hat{ } \hat{ } \hat{ } \hat{ }
ATGCTCGATGAGTTTTTC TAA TCAGAATTGGTTAATTGGTTGT (SEQ ID NO: 75/76)
  M L D E F F *

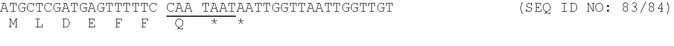

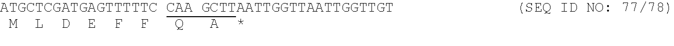

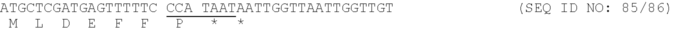

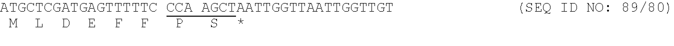




pACYC177-PSFNAVVYHS
ATGCTCGATGAGTTTTTC CCAAGCTTTAATGCGGTAGTTTATCACAGTTAA (SEQ ID NO: 81/82)
  M L D E F F P S F N A V V Y H S *
                −2 +2 +23 TnsD binding site
                  | TAA TAA |
                  --------nnnnn ttacgcagggcatccatttattactcaaccgtaaccga (SEQ ID NO: 52)
                Insertion site ------------------ tnsD binding site->
                                                                              |BaeGI/Bme1508I
                              +58 |SrfI/XmaI
                                  | |SaiI | |KasI
                ttttgccaggttacgcggctgtcgacGTGCCCGGGCGGCGCC
                ------------------------->
```

TABLE 13
Expected Phenotypes of DH10B Harboring pACYC177-based
plasmids comprising KAN-mini-attTn7 fusion proteins with
staggered sets of TAA stop codons

| Designation | | Inc | | | |
|---|---|---|---|---|---|
| DH10B/plasmid | Markers | Group | Phenotype | Stable | Source |
| pACYC177-MLDEFF* | Amp^{R}, | P15A | Kan plus | Yes | This |
| | Kan^{R} | | (+) | | study |
| pACYC177-MLD** | Amp^{R}, | P15A | Kan minus | Yes | This |
| | Kan^{?} | | (−) | | study |
| pACYC177-MLDE** | Amp^{R}, | P15A | Kan minus | Yes | This |
| | Kan^{?} | | (−) | | study |
| pACYC177-MLDEF** | Amp^{R}, | P15A | Kan minus | Yes | This |
| | Kan^{?} | | (−) | | study |
| pACYC177-MLDEF*** | Amp^{R}, | P15A | Kan minus | Yes | This |
| | Kan^{?} | | (−) | | study |
| pACYC177-MLDEFQ** | Amp^{R}, | P15A | Kan plus | Yes | This |
| | Kan^{R} | | (+) | | study |
| pACYC177-MLDEFQA* | Amp^{R}, | P15A | Kan plus | Yes | This |
| | Kan^{R} | | (+) | | study |
| pACYC177-MLDEFP** | Amp^{R}, | P15A | Kan minus | Yes | This |
| | Kan^{?} | | (−) | | study |
| pACYC177-MLDEFPS* | Amp^{R}, | P15A | Kan minus | Yes | This |
| | Kan^{?} | | (−) | | study |

E coli DH10B cells comprising the unmodified patent plasmid or each of the variant plasmids are then spread on agar plates comprising Ampicillin, plus different concentrations of Kanamycin to determine the relative sensitivity to Kanamycin. The phenotypes should match what is predicted in tables noted above.

If the phenotypes are as expected, then the plasmid containing the mini-attTn7 sequence can be used as the basis for additional experiments where a helper plasmid is introduced into the cells, and a donor plasmid transformed in, and plating out in the presence of ampicillin and kanamycin. (The marker on the donor plasmid may need to be changed so it is different from that used by the target plasmid). All target plasmids that confer resistance to Amp and Kan should have a mini-Tn7 inserted at the 3′ end of the truncated/extended NPT-II (Kan) gene.

Variants of plasmids based on pACYC177 can also be created using any of a variety of other replicons. Vectors provided by Twist Biosciences, for example, can also be used. In the series noted below, key segments derived from the kanamycin resistance gene of pACYC177 are synthesized and inserted into pTwist-Chlor-MC (also abbreviated as pTCM), which confers resistance to chloramphenicol and has a medium copy number replicon derived from the plasmid p15A. Polylinker sequences flank the entire kanamycin resistance gene, including its promoter, that containing for two or more 8-bp recognition sites for rare cutting restriction enzymes, such as MauBI, AbsI, SgrDI, and AscI.

TABLE 14
Expected Phenotypes of DH10B Harboring pTwist-Chlor-MC plasmids comprising KAN-mini-attTn7
fusion proteins with staggered sets of TAA stop codons

| | Base Vector | Insert | Expected | | SEQ ID |
|---|---|---|---|---|---|
| Short Name | Markers | Markers | Phenotype | Insert Segments | NOS |
| pTwist + | CAT | None | CamR | None | 173 |
| Chlor + MC | | | | | |
| pTCM- | CAT | None | CamR | MauBI-AbsI-AvrII-SgrDI-AscI polylinker | 174 |
| MaAbAySgAs | | | | | |
| pTCM-Kan- | CAT | Kan | CamR, KanR | Kan extended with CGRTK to mimic Tn7Lrf1 | 175/ |
| CGRT | 176 | | | | |
| pTCM-Kan- | CAT | Kan | CamR, KanS | Kan extended with PSFNAVVYHS to mimic prior art | 177/ |
| PSFNAVVYHS | | | | reference | 178 |
| pTCM-Kan-PS | CAT | Kan | CamR, KanS | Kan extended with PS to mimic prior art reference | 179/ |
| | | | | with silent EcoRI and SpeI sites | 180 |
| pTCM-Kan- | CAT | Kan | CamR, KanR | Kan extended with CGRTK with partial Tn7L rf1 | 181/ |
| Tn7Lrf1 | 182 | | | | |
| pTCM-Kan- | CAT | Kan | CamR, | Kan extended with LWADKIVGNWEGWKWSF with | 183/ |
| Tn7Lrf2 | | | Kan??? | partial Tn7L rf2 | 184 |
| pTCM-Kan- | CAT | Kan | CamR, | Kan extended with PVGGQNSWELGGVEMEFLRII | 185/ |
| Tn7Lrf3 | | | Kan??? | with partial Tn7L rf3 | 186 |
| pTCM-Kan-PS- | CAT | Kan | CamR, KanS | Kan extended with PS and overlapping mini-attTn7 | 187/ |
| mini-attTn7 | 188 | | | | |
| pTCM-Kan-PS | CAT | Kan | CamR, KanS | Kan extended with PS to mimic prior art reference | 189/ |
| | | | | without silent EcoRI or Spel sites | 190 |
| pTCM-Kan | CAT | Kan | CamR, KanR | Kan gene from pACYC177 not extended or | 191/ |
| | | | | truncated without silent EcoRI or SpeI sites | 192 |

FIG. 6 sets forth an illustration entitled “E. coli NPT-II gene-based gene fusions to select for Tn7-based transposition events”.

### Example 5—Design of Modular Sequences Encoding an Inactive β-Lactamase (BLA)-Mini-attTn7 Fusion Polypeptide

A large class of enzymes, called β-lactamases (BLAs), catalyze the hydrolysis of β-lactam antibiotics, such as penicillins and cephalosporins, allowing bacteria harboring genes encoding these enzymes to confer resistance to these compounds. Four general classes (A-D) of β-lactamases are recognized, based sequence similarity and functionality by their hydrolysis rates against a predefined panel of drug products. The physiological targets of β-lactam antibiotics are membrane DD-peptidases, which are responsible for the biosynthesis of peptidoglycan, a major component involved in the maintaining the shape and rigidity of the bacterial cell wall in Gram-positive and Gram-negative bacteria. β-lactam antibiotics acylate the active site serine residue of DD-peptidases, forming stable covalent non-catalytic acyl-enzymes, resulting in the formation of defective peptidoglycan and cell death. While the widespread emergence of drug resistant strains of pathogenic bacteria has tempered the development of new β-lactam antibiotics, analysis of substrate specificities of β-lactamases encoded by genes isolated from pathogenic strains, and from systematic mutagenesis by various combinations of substitution, insertion, or deletion, of amino acids across the entire length of related enzymes, has greatly facilitated 3-dimensional structure/function studies, and the roles of highly conserved amino acid residues involved in binding of a substrate, thermostability, or folding of the molecule [Matagne et al (1998)] [Axe (2000)] [Hecky and Muller (2005)]. These and many other studies have facilitated the development of other applications involving the use of genes encoding β-lactamases to facilitate the selection of vectors comprising cloned genes. Many of the commonly used cloning vectors comprise a bla_{TEM-1} gene encoding the broad spectrum TEM-1β-lactamase (class A) that is present on transposons Tn2 and Tn3 found in many Gram-negative bacteria.

An alignment of 20 Class A β-lactamases facilitated the numbering of specific amino acid residues within this complex family of related enzymes [Ambler et al (1991) A standard numbering scheme for Class A β-lactamases. Biochem J. 276: 269-272]. The plasmid encoded enzyme designated as R-TEM in this paper, starts with the amino acids “MSIQH” and terminating with “LIKHW” corresponds to positions +3 to +290 on the aligned consensus sequence. The alignment of TEM-1 against the consensus sequence, also shows postulated deletions “.”, at positions 239 and 253, for R-TEM, accounting for its size from the N-terminal methionine, to carboxy terminal tryptophan, of 286 amino acids. Class A β-lactamases from other bacteria in this alignment, range in size from 283 to 295 amino acids.

The bla gene In the cloning vector pBR322 encodes an enzyme that is 286 amino acids long, which includes a 23 amino acid signal peptide linked to a 263 amino acid secreted product. The same polypeptide is encoded by the bla gene on the popular cloning vectors pACYC177, pUC18, and pUC19.

One notable study carried out randomized three contiguous codons to create a library of all possible amino acid residues for the region randomized within the gene encoding TEM-1 β-lactamase, finding that 43 of 263 amino acids do not tolerate substitutions, and are critical for the structure and activity of the enzyme [Huang et al (1996) J. Mol. Biol. 258: 688-703.]. A remarkable observation was that Trp165 of four tryptophan residues in TEM-1 (at standard positions +165, +210, +229, and +290) could tolerate substitutions. The carboxy-terminal tryptophan at standard position +290, was identified as being a member of Class 4, where 30 residues were invariant in TEM-1, but not other Class A enzymes, compared to those in Class 1, which has 210 residues that vary in class A and TEM-1, Class 2, which has 23 residues that are invariant in Class A and TEM-1, and Class 3, where 10 residues are invariant in Class A, but not TEM-1.

Analysis of a series of N-terminal and C-terminal deletion variants of TEM-1 β-lactamase demonstrated impaired resistance to ampicillin on agar plates, and impaired ability of the purified enzymes to hydrolyze the chromogenic β-lactam compound nitrocefin as a substrate [Hecky and Muller (2005)]. Four variants were studied, two designated NΔ3 and NΔ5 deleting the first 3 and first 5 amino acids, respectively, from the amino terminus of the mature protein, and CΔ1 and CΔ3 deleting last 1 and last 3 amino acids, respectively, from the carboxy terminus of the mature protein. No colonies were observed for the NΔ5 and the CΔ3 clones on agar plates containing up to 50 ug/ml of ampicillin, suggesting important role for the terminal residues. Reduced numbers of colonies were also observed for the NΔ3 and the CΔ1 clones, compared to control clones comprising a non-truncated version of the gene. These and other experiments clearly demonstrated that deletion of 5 amino acids from the N-terminus decreased its thermostability in vivo and in vitro, but noting a difference in opinion regarding the “essential” nature of the single C-terminal tryptophan residue observed by Huang et al (1996). Many of the experiments by Hecky and Muller, though, focused on mutagenesis and directed evolution of ampicillin-resistant variants derived from the inactive NΔ5 clone, than on additional analysis of the CΔ1 and CΔ3 truncated variants.

The demonstrations by Huang et al (1996) and Hecky and Muller (2005) of critical residues near the carboxy terminal end of the TEM-1 β-lactamase provide the opportunity to design and assemble synthetic genes encoding most of the bla gene in common cloning vectors fused to sequences derived from the attachment site for Tn7, (attTn7), and comparable site-specific target sties from other Tn7-like, and site-specific mobile genetic elements.

Strategies similar to those described above for the design and construction of CAT-attTn7 gene fusions can also be applied to generate bla_{TEM-1}mini-attTn7 fusions (which may also be referred to as BLA- or AMP-mini-attTn7 fusions), where a TAA, TGA, or TAG stop codon is inserted at or near the codons for encoding for the amino acid Lysine (K), Histidine (H), or Tryptophan (W) that are located at the 3′ end of the gene just before the normal TAA stop codon. These studies can be performed using many common cloning vectors comprising a TEM-1 bla gene, including pBR322, pACYC177, pUC-based plasmids, as noted below, or carried out using bla genes derived from other Class A, B, C, or D β-lactamases encoded on conjugative plasmids or the chromosomes of other bacteria.

Sequence Alignment 21: 3' end of 6-lactamase gene from pACYC177 showing
TGG codon for essential tryptophan residue before the TAA stop codon

```
  BanI (G'GYRC,C)
  |
AGGTGCCTCACTGATTAAGCATTGG TAACTGTCAGACCAAGTTTACTCAT (SEQ ID NO: 87/88)
    G A S L I K H W *
                                              |
                                  “Essential” Trp
-------------------TAATAA ------------------------- (SEQ ID NO: 89/90)
---------------------TAA TAA----------------------- (SEQ ID NO: 91/92)
------------------------ TAATAA-------------------- (SEQ ID NO: 93/94)

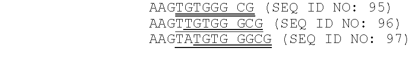

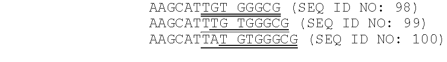

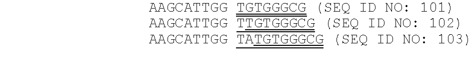



```

The predicted amino acid sequences from these fusions are not shown, but would terminate at different points in the left arm of the mini-Tn7 sequences transposed into the insertion site on the mini-attTn7 (not shown, but similar to those noted earlier) used that overlaps with codons near the 5′ end of the beta-lactamase gene in pACYC177.

FIG. 7 sets forth an illustration entitled “E. coli β-lactamase gene-based gene fusions to assay Tn7-based transposition events”.

### Example 6—Design of Modular Sequences Encoding an Active β-Lactamase (BLA)-Mini-attTn7 Fusion Polypeptide Conferring Resistance to Ampicillin (AMP)

Plasmids encoding inactive alpha and omega fragments of β-lactamase that can complement to form a functional enzyme in both bacteria and in mammalian cells were first reported over 25 years ago [Wehrman et al (2002)]. In these studies, the junction between the alpha fragment (α197) and the omega fragment (ω198) is between at glutamic acid (E) residue at position +197 using the standard numbering scheme, and a leucine (L) residue starting at position +198. In the TEM-1β-lactamases encoded by pBR322, pACYC177, and the pUC series of plasmids, this junction is between the E and L amino acid residues at positions +195 and +196, respectively, where the Methionine (M) residue at the start of the gene is considered +1. These two fragments complemented to produce detectible activity in bacteria to when fused to flexible (Gly₄Ser₃)₃ linkers and two helices (the carboxy terminus of the Jun helix and the amino terminus of the Fos helix) that formed a leucine zipper. Extension of the carboxy terminus of the alpha197 peptide by 3 amino acids to include the amino acids Asn-Gly-Arg (NGR) before the flexible linker and the Jun helix, dramatically increased the ability of the extended alpha fragment to bind to the omega fragment by 4 orders of magnitude. Comparable experiments were also performed in mammalian cells, where a gene encoding an alpha fragment comprising FRB was co-expressed with an omega fragment comprising FKB12, with both fusion proteins lacking the bacterial signal peptide. In the presence of rapamycin, a small cell permeable molecule that can bind to both FRB and FKB12, the α197FRB and FKB12ω198 fragments could bind and complement, indicating reconstitution of β-lactamase activity. Use of this system as a biosensor was proposed, to probe novel protein-protein interactions, comparable to several other types of mammalian two hybrid assay systems.

The clear identification of the junction between two contiguous fragments of β-lactamase, allows for the design of novel fusion proteins where a different type of synthetic polypeptide is inserted between the junction of the alpha and omega fragments. In these studies, the synthetic polypeptide is similar to polypeptide encoded by the sequence inserted into the lacZalpha gene on the bacmid bMON142, noted above, where the attTn7 target site is inserted in frame between the start of the lacZalpha polypeptide (amino acids 1-5), and sequences encoding amino acids 7-41 and beyond, with additional amino acids encoded by different parts of the synthetic multiple cloning site in the vector used to assemble the chimeric gene.

Sequence Alignment 22: Sequences from the PstI site to BglI site in
pACYC177 spanning a junction encoding the carboxy terminal end of an alpha
fragment and the N-terminal end of an omega fragments of beta-lactamase

+295
|PstI(C,TGCA'G) FspI(TGC1GCA) AseI(AT'TA,TT)

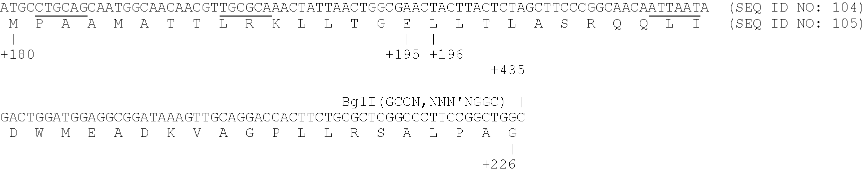

pACYC177 is digested with PstI and BglI and a synthetic oligonucleotide with compatible sticky ends is ligated to it that has an EcoRI site located after the junction of the sequences encoding the alpha fragment of β-lactamase and a SalI site located before the start of the sequences encoding the start of the omega fragment. The PstI and BglI sites are unique in pACYC177. The reading frame is adjusted so that the start of the EcoRI site and the SalI sites are both in the +3 relative reading frame (the wobble position for a codon). In the example noted above, additional nucleotides are added before and after the EcoRI and SalI sites to adjust the reading frame appropriately. In the illustrated example, a site for NotI is added to separate the EcoRI and SalI sites, though the exact sequences before, after, or in between these sites, are not critical to the design of this vector. Other sites, such as those encoding TAA, TAG, or TGA stop codons, or ATG start codons may also be used, depending on the nature of subsequent experiments.

Sequence Alignment 23: Sequences in a variant pACYC177 comprising a synthetic
linker spanning a junction encoding the carboxy terminal end of an alpha
fragment and the N-terminal end of an omega fragments of beta-lactamase

+295 (SEQ ID NOS: 106/107)
|PstI(C,TGCA'G) FspI(TGCIGCA) EcoRI NotI SalI AatII AseI(AT'TA,TT)
| | | | | | |

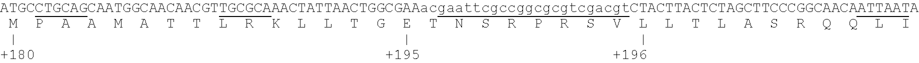

The resulting plasmid is then digested with EcoRI and SalI to insert the synthetic min-attTn7 derived from the bacmid bMON14272, to produce a vector designated pACYC177-bla-mini-attTn7. In this case, the new plasmid should confer resistance to Ampicillin and Kanamycin, since the synthetic oligonucleotide encodes a flexible linker between the alpha and omega fragments of the bla gene. The new plasmid can then be used in a series of experiments demonstrating that transposition into the attTn7 target site disrupts expression of the fusion protein encoded by synthetic bla gene. A plasmid comprising a Tn7 element inserted into the middle of the synthetic target site should confer resistance to Kanamycin, but not Ampicillin.

Sequence Alignment24: Sequences in a pACYC177 variant comprising a synthetic
mini-attTn7at the junction the alpha omega fragments of beta-lactamase

```
+295
|PstI(C,TGCA'G) FspI(TGCIGCA)
| |
ATGCCTGCAGCAATGGCAACAACGTTGCGCAAACTATTAACTGGCGAA (SEQ ID NO: 108)
  M P A A M A T T L R K L L T G E (SEQ ID NO: 109)
  | |
+180 +195
    EcoRI
    |< Synthetic polypeptide encoded by mini-AttTn7
acgaattcacataacaggaagaaaaatgccccgcttacgcagggcatc
  T N S H N R K K N A| P |L T Q G I
                                                          −2 +2
      <-------------------- Insertion Site ---------
                                                                                      SalI
------------------------------------------ |-----

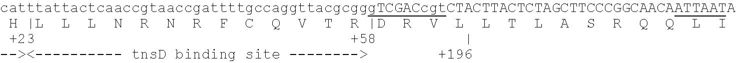

```

Nitrocefin is a chromogenic substrate for beta lactamase. Colonies on agar plates that confer resistance to Ampicillin or related β-lactam antibiotics are red, compared to pale yellow for colonies that do not confer resistance to the antibiotic. Nitrocefin and its product are much more soluble than the indigo dye produced when beta-galactosidase react with a chromogenic substrate such as X-gal or Bluo-gal.

Strategies similar to those noted above for the CAT-mini-attTn7 and Kan-mini-attTn7 fusions can also be used to design comparable bla-alpha-mini-attTn7 fusions, where one or more stop codons are inserted before the codon at the carboxy terminus of the alpha peptide. In a system where both alpha and omega polypeptides are needed to complement and restore activity of the β-lactamase, transposition by a mini-Tn7 into a sequence encoding a truncated alpha fragment with an overlapping mini-attTn7 sequence will restore expression of the alpha polypeptide or an extended form of it, that can complement with an omega fragment expressed under the control of a different promoter. These strategies should work for both prokaryotic and eukaryotic systems, if the sequences encoding the alpha and omega polypeptide fragments are operably linked to promoters that are functional in the host cells, and if the two fragments can bind to each other by non-covalent bonds, optionally mediated by a third molecule. In prokaryotic systems, signal peptides may be needed to facilitate delivery of each fragment to an appropriate location in the cell, compared to eukaryotic cells, where they may be omitted, as noted above, in the experiments reported by Wehrman et al (2002).

FIG. 8 sets forth an illustration entitled “E. coli β-lactamase gene-based gene fusions to screen for Tn7-based transposition events”.

### Example 7—Design of Modular Sequences Encoding Active and Inactive Tetracycline Resistance (Tet)-Mini-attTn7 Fusion Polypeptide

At least 30 major classes of genes (A-Z and beyond) have been identified that confer resistance to tetracycline in Gram-negative bacteria, all showing significant homology at the nucleotide amino acid levels [Levy et al (1999)]. The encoded products are cytoplasmic membrane-bound antiporter proteins, which mediate energy dependent export of tetracycline from the cell in exchange for a proton. Class A and C proteins, Tet(A) and Tet(C), respectively, are 78% identical, but only 48% identical to the class B protein, Tet(B) [Rubin and Levy (1991)]. The Class B proteins have 12 transmembrane (TM1-TM12) regions comprising α-helices arranged in two bundles of 6 helices, 1-6 and 7-12, apparently from a gene duplication, that was the result of a duplication of a 3 helix motif [Waters et al (1983)]. Genes encoding proteins from many of these classes have been studied extensively using random and systematic methods of mutagenesis, creating protein variants having one or more substitutions, insertions, or deletions at or spanning across nearly every position of their primary sequence, contributing greatly to identification of key residues involved the transport of molecules across a bacterial membrane. The N- and C-terminal ends of the protein (˜8 and ˜15 aa long) are located in the cytoplasm. The interdomain loop, separating the α and β domains (N- and C-terminal halves, comprising helices 1-6 and 7-12, respectively) of the Class B and C proteins, is much larger (˜27 aa) than other loop segments exposed to the cytoplasmic (9-10 aa) or periplasmic (3-11 aa) sides of the membrane, and less conserved in across families of related proteins, and generally more tolerant of alterations than membrane-bound segments of the transporter protein [Saraceni-Richards and Levy (2000) 275(9): 6101-6106]. Other studies have suggested that the interdomain loop may be larger, encompassing as many as 40 amino acids, because the predicted sequence of the Class B protein diverges strongly (˜10% identity) from the Class A and C proteins throughout this region [Waters et al (1983)].

Analysis of a variety of deletion mutants in a Tn10 derived gene have noted that deletions corresponding to Δ204-207, Δ195-199, Δ182-197, Δ195-200, Δ202-207, Δ193-199, Δ201-207, Δ180-1987, Δ182-189, and Δ200-207, all conferred resistance to at least 50 uM tetracycline (minimal inhibitory concentration, MIC). on agar plates [Wright and Tate (2015)]. A larger deletion of 9 contiguous amino acids as Δ198-207, and double deletion mutants Δ195-199; 204-207, Δ182-187; 204-207, Δ182-187; 195-199, Δ182-187; 200-208, Δ182-187; 196-207, conferred resistance to 10-20 uM tetracycline, suggesting that larger deletions, or double deletions extending from Δ182-187, plus the central to carboxy terminal portion of this region 195 to 199, 196-207, 200-208, or 204-207, impair the activity of the protein, more than sets of single contiguous deletions of 4-8 residues starting at positions 180, 182, 193, 195, 200, 202, and 204. None of the variants analyzed deleted 4 contiguous amino acids “TDTE” from positions 189-192, which correspond to “PMPL” spanning positions 191-194 for the pACYC184 derived protein. These results suggest that while nucleotides and amino acids in this region are not highly conserved, deletions of 9-19 additional residues affect the activity of the protein.

A series of 2 codon insertions into the SalI or AccI sites of pBR322, corresponding to sequences encoding RRP from 189-191 did not appear to impair activity of the protein (allowing growth on 100 ug/ml oxytetracycline), while two codon insertions at a HpaII and HhaI sites partially encoding “FR” from 203-204 and “AR” from 206-207 near the C-terminal part of the interdomain loop grew on plates containing 15 or 30 or less ug/ml oxytetracycline, respectively [Barany, F (1975) PNAS 82: 4202-4206]. These results demonstrated that high tolerance for insertions of sequences encoding two amino acids at the SalI, and perhaps other nearby sites, consistent with experiments noted above, that deletions of 8 or less contiguous amino acids of are also tolerated in this segment encoding the interdomain loop.

A series of elegant experiments by Levy and coworkers also demonstrated that two inactive proteins, each containing a mutation in the opposite domain, are capable of complementation to produce an active enzyme [R. A. Rubin and S. B. Levy, (1990)]. Inactive interdomain hybrid proteins between class B and C Tet proteins [Tet(B)α/Tet(C)β and Tet(C)α/Tet(B),β] together produce can complement in trans to produce an active enzyme. Cells comprising genes encoding interdomain hybrids, where a frameshift mutation and a terminator were inserted at the fusion junction resulted in expression of the four domains on separate polypeptides, showed trans complementation without production of full length proteins [Rubin and Levy (1991)]. The activity of the reconstituted enzyme was slightly lower, but still substantial (˜20% of the wild-type level), strongly suggesting that the Tet (B) α and β domains were expressed as separate functional proteins. These and other extensive mutagenesis experiments support the idea that the α and β domains can complement in trans at least as effectively as full length hybrid proteins, which is typically 10-20% of the full length wild type enzyme.

Transposon Tn10 comprises a Class B gene, designated tetA(B), which encodes a tetracycline-inducible protein, which is sufficient to confer resistance to the antibiotic. The transposon also has a gene tetR(B), which encodes a repressor, and several other genes, including tetC(B) and tetD(B), jenA, jenB, and jenC, flanked by long (1209 nt) inverted IS10 insertion sequences encoding a transposase.

Tn10 was derived from a drug resistance plasmid found in the enteric bacterium Shigella flexneri, and referred to as NR1, R22, or R100 by several different laboratories. This plasmid, which has a very low copy number (1-2 copies/cell), and is classified in the IncFII incompatibility group, confers resistance to chloramphenicol, fusidic acid, streptomycin/spectinomycin, mercuric salts, and tetracycline. NR1 is compatible with the fertility plasmid, F, first characterized in E. coli.

Genes conferring resistance to tetracycline are found in many common cloning vectors. The plasmid pSC101 is a natural plasmid isolated from Salmonella panama that confers resistance only to tetracycline. Plasmid pACYC184, which confers resistance to chloramphenicol and tetracycline, was derived from pSC101. The synthetic vector pBR322, is derived from 3 plasmids, the Class C tetracycline resistance gene of pSC101, the ampicillin resistance gene of RSF2124, and a replicon derived from pMB1, a close relative of the ColE1 plasmid. Plasmid pBR322, which has a variety of unique restriction sites located in the genes conferring resistance to ampicillin and tetracycline was widely used for many years to facilitate cloning of genes, by inserting plasmid or amplified DNA fragments digested with appropriate enzymes allowing ligation and recovery of plasmids that confer resistance to amplicillin but not tetracycline, or tetracycline, but not ampicillin. Cloning by Insertional of the bla or tet genes is facilitated by a unique EcoRI site, which is located between both genes, along with unique EcoRV, NheI, BamHI, and SalI sites among others in the tet gene, and unique ScaI, PvuI, and PstI sites, among others in the bla gene. The unique SalI site is located in a segment near the middle of the tet gene in pSC101, pBR322, and pACYC184, that encodes the interdomain loop region.

Several studies have reported methods for the direct selection of bacteria that are sensitive to tetracycline. One group reported development of a medium containing the lipophilic chelating agents fusaric acid or quinaldic acid, which was effective for the selection of revertants of Salmonella typhimurium which were resistant to due to insertion of Tn10 into their chromosomes [Bochner, B. R. et al (1980)] An improved media comprising fusaric acid and chlortetracycline and zinc chloride, with lower levels of nutrient supplements, like tryptone, and no glucose improved differentiation between tetracycline-sensitive and tetracycline-resistant strains [Maloy S R, and Nunn W D. (1981)] Two other studies noted that over expression of the membrane bound protein renders cells more sensitive to toxic metal salts, such as nickel chloride or cadmium [Podolsky T, Fong S T, Lee B T. (1996)] [Griffith J K, et al (1982)].

These and other studies provide the basis for the design and assembly of novel gene fusions comprising one or more segments of a gene encoding a protein conferring resistance to tetracycline, and a segment comprising an attachment site for a site-specific transposon. In the sections noted below, segments of the tetracycline resistance gene of pACYC184 are altered, allowing insertion of a segment comprising a mini-attTn7, particularly within the non-conserved interdomain loop region, which should tolerate insertions of DNA encoding a variety of amino acids. Transposition of Tn7 or a mini-Tn7 segment into the mini-attTn7 should disrupt expression of the fusion protein, which can be monitored by screening on ampicillin resistant colonies on plates containing or lacking tetracycline, or by selecting for colonies that confer resistance to ampicillin that are tetracycline sensitive in the presence of fusaric acid, quinaldic acid, nickel salts, or cadmium salts, as noted above.

The alignment shown below, illustrates conserved residues in the tet proteins derived from Tn10 and pACYC184/pSC101/pBR322 and the location of the interdomain loop near the middle of both proteins. The interdomain loop in pACYC184 corresponds to residues +183 to +209, while this region in Tn10 corresponds to residues +181 to +207.

Sequence Alignment 25: Alignment of tetracycline resistance
proteins from Tn10 and pACYC184 showing conserved residues within
cytoplasmic, membrane-boound, and periplasmic polypeptide domains

```
CLUSTAL O(1.2.4)multiple sequence alignment (SEQ ID NOS:110/111)
Tn10 MN--SSTKIALVITLLDAMGIGLIMPVLPTLLREFIASEDIANHFGVLLALYALMQVIFA 58
pACYC184 MKSNNALIVILGTVTLDAVGIGLVMPVLPGLLRDIVHSDSIASHYGVLLALYALMQFLCA 60
                                      *: .: : * . ***:****:***** ***::: *:.**.*:***********.: *
Tn10 PWLZKMSDRFGRRPVLLLSLIGASLDYLLLAFSSALWMLYLGRLLSGITGATGAVAASVI 118
pACYC184 PVLGALSDRFGRRPVLLASLLGATIDYAIMATTPVLWILYAGRIVAGITGATGAVAGAYI 120
                                      * ** :*********** **:**::** ::* : .**:** **:::**********.: *
Tn10 ADTTSASQRVKWFGWLGASFGLGLIAGPIIGGFAGEISPHSPFFIAALLNIVTFLVVMFW 178
pACYC184 ADITDGEDRARHFGLMSACFGVGMVAGPVAGGLLGAISLHAPFLAAAVLNGLNLLLGCFL 180
                                      ** *...:*.: ** :.*.**:*::***: **: * ** *:**: **:** :.:*: *
                                          <---- Interdomain loop --->

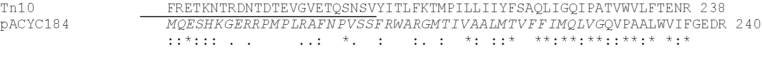

Tn10 FGWNSMMVGFSLAGLOLLHSVFQAFVAGRIATKWGEKTAVLLGFIADSSAFAFLAFISEG 298
pACYC184 FRWSATMIGLSLAVFGILHALAQAFVTGPATKRFGEKQAIIAGMAADALGYVLLAFATRG 300
                                      * *.: *:*:*** :*:**:: ****:* :.::*** *:: *: **: .:.:*** :.*
Tn10 WLVFPVLILLAGGGIALPALQGVMSIQTKSHQQGALQGLLVSLTNATGVIGPLLFAVIYN 358
pACYC184 WMAFPIMILLASGGIGMPALQAMLSRQVDDDHQGQLQGSLAALTSLTSIIGPLIVTAIYA 360
                                      *:.**::****.***.:****.::* *....:** *** *.:**. *.:****:.:.**
Tn10 HSLPIWDGWIWIIGLAFYCIIILLSMTFMLTPQAQGSKQETSA* 401
pACYC184 ASASTWNGLAWIVGAALYLVCLPALRRGA-------WSRATST* 396
                                        * *:* **:* *:* : : .: **:*
```

Sequence Alignment 26: Sequence from the reverse complement of pACYC184 flanking the Interdomain Loop of
the tetracycline resistance protein

```
                          +2052 SphI(G,CATG′C)
                                  | |
```

| | |
|---|---|
| pACYC184 | TCCTTGCATGCACCATTCCTTGCGGCGGCGGTGCTCAACGGCCTCAACCTACTACTGGGC SEQ ID NO: 112 |
| reverse | S L H A P F L A A A V L N G L N L L L G SEQ ID NO: 113 |
| complement | \| |
| | +183 |
| | 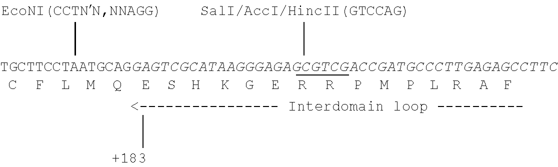 |
| | PshAI(GACNN\|NNGTC) BbsI(GAAGACNN′NNNN,) |
| | \| \| |
| | AACCCAGTCAGCTCCTTCCGGTGGGCGCGGGGCATGACTATCGTCGCCGCACTTATGACT |
| | N P V S S F R W A R G M T I V A A L M T |
| | -----------------------------------> |
| | \| |
| | +209 |
| | +2261 |
| | \| |
| | GTCTTCTTTATCATGCAACTCGTAGGACAG |
| | V F F I M Q L V G Q |

The SphI, EcoNI and SalI recognition and cleavage sites illustrated in the sequence noted above, are unique in pACYC184. The AccI, HincII, and PshAI, each have two sites, and BbsI has three sites in this plasmid. Variant plasmids comprising unique AccI, HincII, PshAI and/or BbsI sites are made by altering the corresponding sites outside the region shown above by site directed mutagenesis, substituting one or more nucleotides in their recognition sequences for other residues, or adding or deleting one or more nucleotide residues, destroying one or more of the unwanted recognition sites.

The easiest variant to make is one where the second PshAI site is removed by insertion of a linker containing a site for another restriction enzyme, since the second site is located in a large intergenic region between the 3′ end of the cat gene encoding resistance to chloramphenicol, and the 3′ end of the tet gene. Synthetic oligonucleotides are prepared replacing one or more segments between the EcoNI and SalI sites, the SalI and PshAI sites, or the EcoNI and PshAI sites, substituting, inserting, or deleting nucleotide residues, typically in units of 3, to replace, add, or delete codons encoding one or more amino acids in the interdomain loop region. Other strategies for performing site-directed mutagenesis may also be used, to generate variants of pACYC184 vectors, or derivatives thereof, comprising the altered sequences noted below.

One of the simplest variants to make is to replace the EcoNI-SalI fragment in pACYC184 with a synthetic fragment comprising part of this segment and a synthetic mini-attTn7 target sequence similar to those used in the construction of synthetic lacZalpha-mini-attTn7 sequences noted above, with the relative location of the restriction enzyme recognition sites altered to maintain the reading frame of the interdomain loop and the synthetic polypeptide encoded by the mini-attTn7 target sequences. Many other locations for insertion of a segment encoding a mini-attTn7 target sequences are possible, taking into account the relative activities of the variant proteins compared to the full length unaltered Tet protein noted in earlier mutagenesis studies. The size of the synthetic mini-attTn7 can also be altered, primarily at the 5′ to and after the Tn7 insertion site (−2 to +2), maintaining key sequences extending into those corresponding to the binding site of the protein encoded by the tnsD gene (+23 to +58).

Sequence Alignment 27: Insertion of a synthetic mini-attTn7 into a SalI site near
sequences encoding the Interdomain Loop of the tetracycline resistance protein

```
                  +2052 SphI(G,CATG'C)
                          | |
pACYC184 TCCTTGCATGCACCATTCCTTGCGGCGGCGGTGCTCAACGGCCTCAACCTACTACTGGGC SEQ ID NO: 114
  reverse S L H A P F L A A A V L N G L N L L L G SEQ ID NO: 115
complement |
                        +158
EcoNI(CCTN'N,NNAGG) EcoRI
                | |<------------ Synthetic mini-AttTn7 ---------
TGCTTCCTAATGCAGGAGTCGCATAAGGGAGAgaattcacataacaggaagaaaaatgccccgcttacgcagggcatc
  C F L M Q E S H K G E N S H N R K K N A| P |L T Q G I
                                | | −2 +2
                          +183 +188
                              <Interdomain loop><-------------------- Insertion site --------
                                                                                                SalI/AccI/HincII(GTCCAG)
----------------------------------------------> |

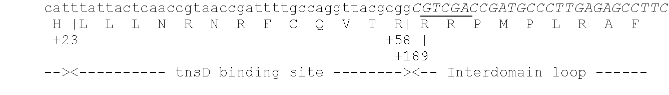

                                                                                          PshAI(GACNN|NNGTC) BbsI(GAAGACNN'NNNN,)
                                                                                                              | |
                                AACCCAGTCAGCTCCTTCCGGTGGGCGCGGGGCATGACTATCGTCGCCGCACTTATGACT
                                  N P V S S F R W A R G M T I V A A L M T
                                ------- Interdomain loop ---------->
                                                                                                    |
                                                                                              +209
                                                                                  +2261
                                                                                          |
                                GTCTTCTTTATCATGCAACTCGTAGGACAG
                                  V F F I M Q L V G Q
```

Sequence Alignment 28: An EcoRI-Sall fragment comrpising a synthetic mini-attTn7
Small versions of the synthetic mini-attTn7 site can be placed in frame with other segments
of the tetracycline resistance protein.

EcoRI
|<------------ Synthetic mini-AttTn7 ---------
Gaattcacataacaggaagaaaaatgccccgcttacgcagggcatccat (SEQ ID NO: 116)

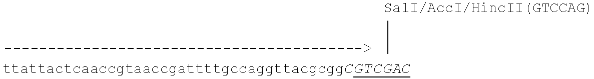

Insertion by transposition of Tn7 or a mini-attTn7 derivative into the synthetic target site in a gene encoding a tet-mini-attTn7 fusion protein, should result in expression of an altered α-fragment, extended by amino acid residues encoded by the left arm of Tn7 (in different amounts depending on the reading frame), and disrupt the expression of a β-fragment, preventing assembly of a functional tetracycline resistance protein.

In a test system where host bacterial cells harbor a target vector comprising a synthetic tet-mini-attTn7 gene encodes a functional protein, and a compatible helper plasmid, encoding essential transposition proteins, are transformed with a mini-Tn7 donor plasmid that is incompatible with the helper plasmid, transposition of the mini-Tn7 into the mini-attTn7 on the target vector, will disrupt expression of the tet gene. The phenotypic change from tetracycline resistant to sensitive can be monitored by spreading bacteria on plates containing chloramphenicol to select for the pACYC184 vector, plus the antibiotic encoded by a resistance marker on the helper plasmid, and purifying and testing colonies on similar plates with varying amounts of tetracycline. Plasmid DNAs isolated from colonies that are sensitive to tetracycline is purified and analyzed to determine their structures compared to parental vectors used in the experiment.

Bacteria comprising the target vector, helper plasmid, and donor plasmid can also be spread on agar plates containing the appropriate antibiotics, plus different concentrations of nickel salts, fusaric acid, or quinaldic acid, to select for bacteria that are sensitive to tetracycline. In this scheme, cells harboring plasmids having transposition events should survive, and those harboring the parental target plasmid, or the pACYC184 control plasmid, should not.

FIG. 9 sets forth an illustration entitled “E. coli tetracycline resistance gene-based fusions to screen for Tn7-based transposition events”.

### Example 8—Summary of Direct Selection for or Screening of Transposition Events into Synthetic Min-attTn7 Target Sites

FIG. 10 sets forth an illustration entitled “General strategies for selecting or screening for site-specific transposition events”.

The following table summarizes key features of the methods described in each of the Examples, for direct selection or screening of insertions by transposition of a Tn7-based sequence into a target site comprising a synthetic attachment operably-linked to a regulatory and coding sequence for a selectable or screenable marker gene.

TABLE 15
Key Examples of Direct Selection for or Screening of
Transposition Events Into Synthetic min-attTn7 Target Site*

| | | | | Selection/ | |
|---|---|---|---|---|---|
| Ex | Scheme | Target before transposition | After transposition | Screening | Key Reagent |
| 1a | lacZalpha- | lacZalpha gene with synthetic mini- | Expression of trimeric | Screening | Blue/White |
| 1b | mini-attTn7 | attTn7 inserted between codons 6-7; | lacZalpha polypeptide | | colonies; |
| | | Extra sequences from legacy MCS | disrupted preventing | | Lac Plus (+) |
| | | regions flanking mini-attTn7 are | complementation with | | to Minus (−) |
| | | removed allowing reuse of restriction | acceptor polypeptide | | |
| | | sites in the MCS regions in construction | | | |
| | | of modular genetic elements | | | |
| 2 | ΔCAT-mini- | 3′ end of cat gene near codon for Cys | Frameshift after | Selection | Cm S to |
| | attTn7 | overlapping with mini-attTn7 | transposition, CAT | | Cm R |
| | | | protein extended, | | |
| | | | restoring function | | |
| 3 | ΔlacZalpha- | ΔlacZalpha with stop codons | Frameshift after | Selection | Blue/White |
| | mini-attTn7 | overlapping with synthetic mini-attTn7 | transposition, | | colonies; |
| | | near codons 40-41-mini-attTn7 | LacZalpha extended, | | Lac minus (−) |
| | | | restoring ability to | | to Plus (+) |
| | | | complement with | | |
| | | | acceptor polypeptide | | |
| 4a | ΔNPT-II- | NPT-II gene with proline residue | Frameshift after | Selection | Kan S to |
| | mini-attTn7 | replacing TAA stop codon-min-attTn7 | transposition, NPT-II | | Kan R |
| | | | protein extended, | | |
| | | | restoring function | | |
| 4b | ΔNPT-II- | NPT-II gene with proline residue | Frameshift after | Selection | Kan S to |
| | mini-attTn7 | replacing TAA stop codon-min-attTn7 | transposition, NPT-II | | Kan R |
| | | | protein truncated, | | |
| | | | restoring function | | |
| 5 | Δβ- | bla gene with essential Trp codon near | Frameshift after | Selection | Nitrocefin: |
| | lactamase- | normal TAA stop codon with synthetic | transposition, BLA | | Amp S to |
| | mini-attTn7 | mini-attTn7 | protein extended, | | Amp R |
| | | | restoring function | | |
| 6 | β-lactamase- | bla gene with mini-attTn7 inserted | BLA protein disrupted, | Screening | Amp R to |
| | mini-attTn7 | between junction for alpha and omega | destroying function | | Amp S |
| | | fragments | | | |
| 7a | Tet-mini- | Tet gene with mini-attTn7 inserted into | TET protein disrupted, | Screening/ | Select TC |
| | attTn7 | “interdomain loop” between left and | destroying function | Selection | sensitive on |
| | | right half for domain fragments | | | special plates; |
| | | | | | TcR toTc S |
| 7b | ΔTet-mini- | Tet gene with TAA stop codon at end | Truncated left or right | Selection | TcS to |
| | attTn7 | of left or right domain fragment with | domain fragment | | Tc R |
| | | overlapping mini-attTn7 | extended restoring | | |
| | | | function and, allowing | | |
| | | | complementation | | |

*The original synthetic mini-attTn7 in Example 1a was on an EcoRI-SalI fragment comprising sequences that are 5′ to the Tn7 insertion site at relative positions −2 to +2, and the binding site for the product of the tnsD gene at relative positions +23 to +58. The composition of sequences at the insertion site are irrelevant to the binding of the TnsD recombinase protein. The relative position of the insertion site can be adjusted to the left or the right of the nucleotide sequences in the overlapping target gene by single nucleotide residues, allowing insertion of the transposon in an orientation-specific manner beginning at the left arm of Tn7 at the insertion site. The sequences from −2 to +2 are duplicated to the left of Tn7L and the right of Tn7R. Inverted repeats are at the ends of Tn7 with TGT nucleotides at the 5′ end of Tn7L, and ACA nucleotides at the 3′ end of Tn7R.

These and similar approaches (CAT-mini-attTn7 and Kan-mini-attTn7), which allow the direct selection of transposition events, dramatically increase the power of systems designed to insert one or more large segments of DNA into one or more specific sites on a plasmid, a shuttle vector, or the chromosome.

Promoters driving expression of the fusion proteins encoded synthetic target sites may be altered, changing them to tightly inducible promoters, allowing control of expression only in the presence of specific inducing agents.

These methods have the potential to dramatically alter strategies for gene insertion in a wide variety of fields, including the development of synthetic transposition systems, where the ends of the transposon, genes encoding transposases, and the target site can be altered by random or site specific mutagenesis, and rare variants recovered by methods involving direct selection of transposition events.

### Example 9—Design of Modular Baculovirus Shuttle Vectors Comprising Different Synthetic Mini-Tn7 Target Sequences

The development of baculovirus vectors capable of expressing heterologous proteins in cultured insect cells and larvae have transformed many fields of biology, particularly applications in the field of healthcare research leading to the development of therapeutic drug products, vaccines, components of diagnostic kits, cell and gene therapy vector systems, and general research tools [Luckow and Summers (1988b)] [O'Reilly, D. R., Miller, L. K., and Luckow, V. A. (1992)]. Proteins expressed at high levels greatly facilitate research studies that reveal the structure and function of polypeptide domains capable of carrying out catalytic reactions, the binding of co-factors, and other residues involved in the binding of a protein to other molecules within or outside a cell.

A wide variety of strategies have been developed to generate recombinant viruses suitable for the rapid production of heterologous proteins in insect cells susceptible to infection by a virus, which generally rely on homologous recombination between a wild-type or engineered virus and a transfer vector, or by site-specific transposition of a DNA cassette comprising a promoter and a gene of interest into a desired location within an engineered virus. General features of these approaches have been reviewed and compared in several reports, particularly for viral vector backbones and transfer vectors or donor plasmids that are available from a variety of commercial sources [Roy and Noad (2012)] [Lun et al (2011)] [Possee et al (2019)].

There is a persistent need, however, to develop improved methods for the generation of recombinant baculoviruses, that are easier and more rapid than existing methods, or lead to higher levels of expression of one or more heterologous proteins expressed in cultured cells or insect larvae. Many strategies have been developed to improve the structural organization of DNA segments comprising one or more baculovirus promoters operably-linked to one or more genes of interest (GOIs), that are present in transfer vectors or donor plasmids, or to express the products of these genes as fusion proteins comprising amino- or carboxy-terminal tags to facilitate targeting, secretion. or purification of the heterologous protein from samples comprising host cell proteins and other viral proteins.

Nearly every laboratory involved in this type of research, is capable of generating modified transfer vectors or donor plasmids, because they are small, and easy to manipulate by traditional cloning methods, and by strategies designed to mutate one or more nucleotide residues by substitution, insertion, or deletion, permitting the systematic functional analysis of one or more genes of interest. Strategies generally designed to manipulate the backbone of the viral vector, are much less common, due in part to the large size of the virus. The sequence of wild-type C6 and E2 variants of the Autographa californica Nuclear Polyhedrosis Virus (AcNPV) are known, each are over 128 kb in length. Development of the baculovirus shuttle vector (bacmid) system permitted the systematic analysis of the >150 genes in these and other related viruses by allowing mutagenesis of a gene in the bacmid propagated in bacteria, before transfecting insect cells with the modified vector to determine if the gene is essential or non-essential for propagation of the budded or occluded forms of the virus. The budded form which is required for transmission from cell to cell in the insect, or in cultured insect cells, is formed about 24 hpi, compared to the stable occluded form, which is produced 48-72 hpi, that can survive in the environment. The occluded form of the virus dissolves in the alkaline environment in the gut of caterpillars that fed on contaminated plant materials, leading to a new cycle of cell-cell infection and eventual release of occluded viral particles.

Excellent sources of information various aspects of the molecular biology of baculoviruses are the online chapters in a book published by Rohrmann [2019], particularly sections annotating the functions of all known genes in AcNPV and Bombyx mori NPV (BmNPV), among others. The following table provides a list of those genes and whether they are considered core genes, found in many other related viruses, and essential or non-essential based on functional studies in transfected insect cell or injected into larvae, but also noting they are appear to be clustered in groups of two or more contiguous genes. Genes that are not essential, whether they appear alone, or in clusters, may be good targets for mutagenesis, allowing the insertion of gene cassettes located on transfer vectors or donor plasmids, or insertion of bacterial replicons and drug resistance markers used in baculovirus shuttle vector systems.

TABLE 16
Characteristics of AcNPV genes

| | | | | Non- | Clustered | Clustered Non- | Clustered |
|---|---|---|---|---|---|---|---|
| Gene | Gene (Protein) | Core | Essential | Essential? | Essential | Essential | Core |
| Ac1 | Ac001 (Protein tyrosine | | | Non- | E | Clustered Non- | E |
| | phosphatase (ptp)) | | | Essential | | Essential | |
| Ac2 | Ac002 (BRO (Baculovirus | | | Non- | E | Clustered Non- | E |
| | repeated orf)) | | | Essential | | Essential | |
| Ac3 | Ac003 (Conotoxin like (Ctl)) | | | Non- | E | Clustered Non- | E |
| | | | | Essential | | Essential | |
| Ac4 | | | | Non- | E | Clustered Non- | E |
| | | | | Essential | | Essential | |
| Ac5 | | | | Non- | E | N | E |
| | | | | Essential | | | |
| *Ac6 | Ac006* (Lef2) | * | Essential | | N | E | N |
| Ac7 | | | | Non- | E | Clustered Non- | E |
| | | | | Essential | | Essential | |
| Ac8 | Ac008 (Polyhedrin ) | | | Non- | E | N | E |
| | | | | Essential | | | |
| Ac9 | Ac009 (Pp78/83; orf1629) | | Essential | | Clustered | E | E |
| | | | | | Essential | | |
| Ac10 | Ac010 (PK1 | | Essential | | N | E | E |
| | (Protein kinase 1)) | | | | | | |
| Ac11 | | | | Non- | E | Clustered Non- | E |
| | | | | Essential | | Essential | |
| Ac12 | | | | Non- | E | Clustered Non- | E |
| | | | | Essential | | Essential | |
| Ac13 | | | | Non- | E | N | E |
| | | | | Essential | | | |
| *Ac14 | Ac014* (Lef1) | * | Essential | | N | E | N |
| Ac15 | Ac015 (EGT) | | | Non- | E | Clustered Non- | E |
| | | | | Essential | | Essential | |
| Ac16 | Ac016 (BV/ODV-E26) | | | Non- | E | N | E |
| | | | | Essential | | | |
| Ac17 | Ac016 (DA26) | | Essential | | N | E | E |
| Ac18 | | | | Non- | E | Clustered Non- | E |
| | | | | Essential | | Essential | |
| Ac19 | | | | Non- | E | N | E |
| | | | | Essential | | | |
| Ac20 | Ac020/021 (ARIF1 (Actin | | Essential | | N | E | E |
| | rearranging factor1)) | | | | | | |
| *Ac22 | Ac022* (Pif-2) | * | | Non- | E | Clustered Non- | Clustered |
| | | | | Essential | | Essential | Core |
| Ac23 | Ac023 (F (fusion protein | | | Non- | E | N | E |
| | homolog)) | | | Essential | | | |
| Ac24 | Ac024 (PKIP (Protein kinase | | Essential | | Clustered | E | E |
| | interacting factor)) | | | | Essential | | |
| Ac25 | Ac025 (DBP (DNA binding | | Essential | | N | E | E |
| | protein)) | | | | | | |
| Ac26 | | | | Non- | E | Clustered Non- | E |
| | | | | Essential | | Essential | |
| Ac27 | Ac027 (lap-1) | | | Non- | E | N | E |
| | | | | Essential | | | |
| Ac28 | Ac028 (Lef6) | | Essential | | N | E | E |
| Ac29 | | | | Non- | E | Clustered Non- | E |
| | | | | Essential | | Essential | |
| Ac30 | | | | Non- | E | Clustered Non- | E |
| | | | | Essential | | Essential | |
| Ac31 | Ac031 (SOD superoxide | | | Non- | E | Clustered Non- | E |
| | dismutase) | | | Essential | | Essential | |
| Ac32 | Ac032 (FGF (fibroblast | | | Non- | E | Clustered Non- | E |
| | growth factor)) | | | Essential | | Essential | |
| Ac33 | Ac033 (Histodinol | | | Non- | E | N | E |
| | phosphatase) | | | Essential | | | |
| Ac34 | Ac033 (PNK polynucleotide | | Essential | | N | E | E |
| | kinase) | | | | | | |
| Ac35 | Ac035 (Ubiquitin) | | | Non- | E | N | E |
| | | | | Essential | | | |
| Ac36 | Ac036 (39K, pp31) | | Essential | | Clustered | E | E |
| | | | | | Essential | | |
| Ac 37 | Ac036 (Pp31; 39K) | | Essential | | Clustered | E | E |
| | | | | | Essential | | |
| Ac38 | Ac037* (Lef11) | | Essential | | N | E | E |
| Ac39 | Ac038 (Nudix) | | | Non- | E | N | E |
| | | | | Essential | | | |
| *Ac40 | Ac039 (P43) | * | Essential | | Clustered | E | N |
| | | | | | Essential | | |
| Ac41 | Ac041* (Lef12) | | Essential | | N | E | E |
| Ac42 | Ac042 (Gta (global | | | Non- | E | N | E |
| | transactivator)) | | | Essential | | | |
| Ac43 | | | Essential | | N | E | E |
| Ac44 | Ac046 (Chondroitinase, odv- | | | Non- | E | Clustered Non- | E |
| | e66) | | | Essential | | Essential | |
| Ac45 | Ac046 (ODV-E66) | | | Non- | E | Clustered Non- | E |
| | | | | Essential | | Essential | |
| Ac46 | Ac047 (ETS) | | | Non- | E | Clustered Non- | E |
| | | | | Essential | | Essential | |
| Ac47 | Ac047 (TRAX-like) | | | Non- | E | Clustered Non- | E |
| | | | | Essential | | Essential | |
| Ac48 | Ac048 (ETM) | | | Non- | E | Clustered Non- | E |
| | | | | Essential | | Essential | |
| Ac49 | Ac049 (ETL (PCNA)) | | | Non- | E | N | E |
| | | | | Essential | | | |
| *Ac50 | Ac049 (PCNA) | * | Essential | | Clustered | E | Clustered |
| | | | | | Essential | | Core |
| Ac51 | Ac050* (Lef8) | | Essential | | Clustered | E | E |
| | | | | | Essential | | |
| Ac52 | Ac051 (DnaJ domain | | Essential | | Clustered | E | E |
| | protein) | | | | Essential | | |
| *Ac53 | Ac051 (J domain) | * | Essential | | Clustered | E | Clustered |
| | | | | | Essential | | Core |
| Ac53a | | | Essential | | Clustered | E | E |
| | | | | | Essential | | |
| *Ac54 | Ac054* (Vp1054 ) | * | Essential | | N | E | N |
| Ac55 | | | | Non- | E | Clustered Non- | E |
| | | | | Essential | | Essential | |
| Ac56 | | | | Non- | E | Clustered Non- | E |
| | | | | Essential | | Essential | |
| Ac57 | | | | Non- | E | Clustered Non- | E |
| | | | | Essential | | Essential | |
| Ac58, | Ac059 (ChaB homolog) | | | Non- | E | Clustered Non- | E |
| Ac58/59 | | | | Essential | | Essential | |
| Ac60 | Ac060 (ChaB homolog) | | | Non- | E | Clustered Non- | E |
| | | | | Essential | | Essential | |
| Ac61 | Ac061 (FP (few polyhedra), | | | Non- | E | N | E |
| | fp-25k) | | | Essential | | | |
| *Ac62 | Ac062* (Lef9) | * | Essential | | N | E | N |
| Ac63 | Ac064 (Fusolin (gp37)) | | | Non- | E | Clustered Non- | E |
| | | | | Essential | | Essential | |
| Ac64 | Ac064 (GP37) | | | Non- | E | N | E |
| | | | | Essential | | | |
| *Ac65 | Ac065* (DNA polymerase) | * | Essential | | Clustered | E | N |
| | | | | | Essential | | |
| *Ac66 | Ac066* (Desmoplakin-like) | * | Essential | | N | E | N |
| Ac67 | Ac067 (Lef3) | | | Non- | E | Clustered Non- | E |
| | | | | Essential | | Essential | |
| *Ac68 | Ac068* (Pif-6) | * | | Non- | E | N | N |
| | | | | Essential | | | |
| Ac69 | Ac069 (MTase (methyl | | Essential | | N | E | E |
| | transferase)) | | | | | | |
| Ac70 | Ac070 (Hcf-1 (host cell | | | Non- | E | Clustered Non- | E |
| | factor 1)) | | | Essential | | Essential | |
| Ac71 | Ac071 (lap-2) | | | Non- | E | Clustered Non- | E |
| | | | | Essential | | Essential | |
| Ac72 | | | | Non- | E | Clustered Non- | E |
| | | | | Essential | | Essential | |
| Ac73 | | | | Non- | E | N | E |
| | | | | Essential | | | |
| Ac74 | | | Essential | | Clustered | E | E |
| | | | | | Essential | | |
| Ac75 | | | Essential | | Clustered | E | E |
| | | | | | Essential | | |
| Ac76 | | | Essential | | Clustered | E | E |
| | | | | | Essential | | |
| *Ac77 | Ac077* (VLF-1 very late | * | Essential | | Clustered | E | Clustered |
| | factor 1) | | | | Essential | | Core |
| *Ac78 | | * | Essential | | Clustered | E | Clustered |
| | | | | | Essential | | Core |
| Ac79 | | | Essential | | Clustered | E | E |
| | | | | | Essential | | |
| *Ac80 | Ac080 (GP41) | * | Essential | | Clustered | E | N |
| | | | | | Essential | | |
| *Ac81 | Ac082 (TLP telokin-like) | * | Essential | | N | E | N |
| Ac82 | Ac083* (P95, p91) | | | Non- | E | N | E |
| | | | | Essential | | | |
| *Ac83, VP91, | Ac083* (Pif-8, vp91, vp94) | * | Essential | | N | E | N |
| PIF-8 | | | | | | | |
| Ac84 | Ac083* (Vp91, p95) | | | Non- | E | Clustered Non- | E |
| | | | | Essential | | Essential | |
| Ac85 | Ac086 (PNK/PNL | | | Non- | E | Clustered Non- | E |
| | PO lynucleotide | | | Essential | | Essential | |
| | kinase/ligase) | | | | | | |
| Ac86 | Ac087 (P15) | | | Non- | E | Clustered Non- | E |
| | | | | Essential | | Essential | |
| Ac87 | Ac088 (Cg30) | | | Non- | E | N | E |
| | | | | Essential | | | |
| Ac88 | Ac089* (Vp39, capsid) | | Essential | | Clustered | E | E |
| | | | | | Essential | | |
| *Ac89 | Ac090* (Lef4) | * | Essential | | Clustered | E | N |
| | | | | | Essential | | |
| *Ac90 | Ac092* (P33 sulfhydryl | * | Essential | | N | E | N |
| | oxidase) | | | | | | |
| Ac91 | Ac092* (Sulfhydryl oxidase, | | | Non- | E | N | E |
| | sox) | | | Essential | | | |
| *Ac92 | Ac093 (P18) | * | Essential | | Clustered | E | Clustered |
| | | | | | Essential | | Core |
| *Ac93 | Ac094* (ODV-E25, p25, 25k) | | Essential | | Clustered | E | Clustered |
| | | | | | Essential | | Core |
| *Ac94 | Ac095* (Helicase, p143) | * | Essential | | Clustered | E | N |
| | | | | | Essential | | |
| *Ac95 | Ac095* (P143 (helicase)) | * | Essential | | N | E | N |
| *Ac96 | Ac096* (19K (pif-4)) | * | | Non- | E | Clustered Non- | Clustered |
| | | | | Essential | | Essential | Core |
| Ac97 | Ac096* (Pif-4 (19K)) | * | | Non- | E | N | E |
| | | | | Essential | | | |
| *Ac98 | Ac098* (38K) | * | Essential | | Clustered | E | Clustered |
| | | | | | Essential | | Core |
| *Ac99 | Ac099* (Lef5) | * | Essential | | Clustered | E | Clustered |
| | | | | | Essential | | Core |
| *Ac100 | Ac100* (P6.9) | * | Essential | | Clustered | E | Clustered |
| | | | | | Essential | | Core |
| *Ac101 | Ac101* (BV/ODV-C42) | * | Essential | | Clustered | E | Clustered |
| | | | | | Essential | | Core |
| Ac102 | Ac102 (C42) | | Essential | | Clustered | E | E |
| | | | | | Essential | | |
| *Ac103 | Ac102 (P12) | | Essential | | Clustered | E | N |
| | | | | | Essential | | |
| Ac104 | Ac102* (P40) | | Essential | | N | E | E |
| Ac105 | Ac103* (P45, p48) | | | Non- | E | N | E |
| | | | | Essential | | | |
| Ac106/107 | Ac104 (Vp80, vp87) | | Essential | | N | E | E |
| Ac108 | Ac105 (He65 ) | | | Non- | E | N | E |
| | | | | Essential | | | |
| *Ac109 | | * | Essential | | N | E | N |
| *Ac110 | Ac110* (Pif-7) | * | | Non- | E | Clustered Non- | Clustered |
| | | | | Essential | | Essential | Core |
| Ac111 | | | | Non- | E | Clustered Non- | E |
| | | | | Essential | | Essential | |
| Ac112/113 | Ac112/113 (Apsup) | | | Non- | E | Clustered Non- | E |
| | | | | Essential | | Essential | |
| Ac114 | | | | Non- | E | Clustered Non- | E |
| | | | | Essential | | Essential | |
| *Ac115 | Ac115* (Pif-3) | * | | Non- | E | Clustered Non- | Clustered |
| | | | | Essential | | Essential | Core |
| Ac116 | | | | Non- | E | Clustered Non- | E |
| | | | | Essential | | Essential | |
| Ac117 | | | | Non- | E | Clustered Non- | E |
| | | | | Essential | | Essential | |
| Ac118 | | | | Non- | E | Clustered Non- | E |
| | | | | Essential | | Essential | |
| *Ac119 | Ac119* (Pif-1) | * | | Non- | E | Clustered Non- | Clustered |
| | | | | Essential | | Essential | Core |
| Ac120 | Ac123 (PK2 | | | Non- | E | Clustered Non- | E |
| | (Protein kinase 2)) | | | Essential | | Essential | |
| Ac121 | Ac125 (Lef7) | | | Non- | E | Clustered Non- | E |
| | | | | Essential | | Essential | |
| Ac122 | Ac126 (Chitinase) | | | Non- | E | Clustered Non- | E |
| | | | | Essential | | Essential | |
| Ac123 | Ac127 (Cathepsin) | | | Non- | E | Clustered Non- | E |
| | | | | Essential | | Essential | |
| Ac124 | Ac128 (GP64) | | | Non- | E | N | E |
| | | | | Essential | | | |
| Ac125 | Ac129 (P24) | | Essential | | N | E | E |
| Ac126 | Ac130 (GP16) | | | Non- | E | Clustered Non- | E |
| | | | | Essential | | Essential | |
| Ac127 | Ac131 (Calyx, polyhedron | | | Non- | E | N | E |
| | envelope) | | | Essential | | | |
| Ac128 | Ac131 (PEP polyhedron | | Essential | | N | E | E |
| | envelope protein) | | | | | | |
| Ac129 | Ac131 (Pp34, polyhedron | | | Non- | E | Clustered Non- | E |
| | envelope) | | | Essential | | Essential | |
| Ac130 | | | | Non- | E | N | E |
| | | | | Essential | | | |
| Ac132 | | | Essential | | Clustered | E | E |
| | | | | | Essential | | |
| *Ac133 | Ac133* (Alkaline nuclease) | * | Essential | | N | E | N |
| Ac134 | Ac134 (P94 ) | | | Non- | E | N | E |
| | | | | Essential | | | |
| Ac135 | Ac135 (P35) | | Essential | | N | E | E |
| Ac136 | Ac136 (P26) | | | Non- | E | Clustered Non- | E |
| | | | | Essential | | Essential | |
| Ac137 | Ac137 (P10) | | | Non- | E | Clustered Non- | E |
| | | | | Essential | | Essential | |
| *Ac138 | Ac138 (P74, Pif-O) | * | | Non- | E | N | N |
| | | | | Essential | | | |
| Ac 139 | Ac138* (Pif-0, p74) | | Essential | | N | E | E |
| Ac140 | Ac139 (Me53) | | | Non- | E | N | E |
| | | | | Essential | | | |
| Ac141 | Ac141 (Exon-O) | | Essential | | Clustered | E | E |
| | | | | | Essential | | |
| *Ac142 | Ac142* (49K) | * | Essential | | Clustered | E | Clustered |
| | | | | | Essential | | Core |
| *Ac143 | Ac142* (P49) | * | Essential | | Clustered | E | N |
| | | | | | Essential | | |
| *Ac144 | Ac143* (ODV-E18) | * | Essential | | N | E | N |
| Ac145 | Ac144 (ODV-EC27) | | | Non- | E | N | E |
| | | | | Essential | | | |
| Ac146 | Ac145 (P11) | | Essential | | Clustered | E | E |
| | | | | | Essential | | |
| Ac147 | Ac147 (le1 ) | | Essential | Non- | N | E | E |
| Ac147-0 | Ac147-0 (le0) | | | Essential | E | Clustered Non- | E |
| | | | | | | Essential | |
| *Ac148 | Ac148* (ODV-E56, Pif-5) | * | | Non- | E | Clustered Non- | Clustered |
| | | | | Essential | | Essential | Core |
| Ac149 | Ac148* (Pif-5, ody-e56) | | | Non- | E | Clustered Non- | E |
| | | | | Essential | | Essential | |
| Ac150 | | | | Non- | E | N | E |
| | | | | Essential | | | |
| Ac151 | Ac151 (le2) | | Essential | | N | E | E |
| Ac152 | Ac153 (Pe38) | | | Non- | E | N | E |
| | | | | Essential | | | |
| Ac153 | Ac53a (Lef10) | | Essential | | N | E | E |
| Ac154 | | | | Non- | E | Clustered Non- | E |
| | | | | Essential | | Essential | |

Over 347 nucleotide sequences have been deposited in Gen Bank providing the complete genomes of a wide variety of insect viruses, including baculoviruses and granulosis viruses, among others. Similar tables can be prepared for each virus, by comparing the homology for each gene against annotated sets of genes for other related viruses. Viruses of most interest to researchers involved in the development of novel expression vector systems, are AcNPV and BmNPV.

TABLE 17
Relevant AcNPV and BmNPV sequences

| Name | Size | Acc No | Acc. No. |
|---|---|---|---|
| Autographa californica | 133,926 bp | KM609482.1 | GI: 851968049 |
| multiple | | | |
| nucleopolyhedrovirus | | | |
| isolate WP10, complete | | | |
| genome | | | |
| Autographa californica | 133,894 bp | L22858.1 | GI: 510708 |
| nucleopolyhedrovirus | | | |
| clone | | | |
| C6, complete genome | | | |
| Autographa californica | 133,966 bp | KM667940.1 | GI: 700275637 |
| nucleopolyhedrovirus | | | |
| strain | | | |
| E2, complete genome | | | |
| Autographa californica | 133,894 bp | NC_001623.1 | GI: 9627742 |
| nucleopolyhedrovirus, | | | |
| complete genome | | | |
| Bombyx mori NPV strain | 127,465 bp | JQ991009.1 | GI: 393659939 |
| Cubic, complete genome | | | |
| Bombyx mori NPV strain | 126,843 bp | JQ991011.1 | GI: 393717332 |
| Guangxi, complete | | | |
| genome | | | |
| Bombyx mori NPV strain | 126,879 bp | JQ991010.1 | GI: 393717193 |
| India, complete genome | | | |
| Bombyx mori NPV strain | 126,125 bp | JQ991008.1 | GI: 393717051 |
| Zhejiang, complete | | | |
| genome | | | |
| Bombyx mori NPV, | 128,413 bp | NC_001962.1 | GI: 9630816 |
| complete genome | | | |
| Bombyx mori nuclear | 128,413 bp | L33180.1 | GI: 3745835 |
| polyhedrosis virus isolate | | | |
| T3, complete genome | | | |
| Bombyx mori | 127,459 bp | LC150780.1 | GI: 1227954165 |
| nucleopolyhedrovirus | | | |
| DNA, complete genome, | | | |
| isolate: H4 | | | |
| Bombyx mori | 127,901 bp | KF306215.1 | GI: 548577843 |
| nucleopolyhedrovirus | | | |
| isolate C1, complete | | | |
| genome | | | |
| Bombyx mori | 126,406 bp | KF306216.1 | GI: 548578068 |
| nucleopolyhedrovirus | | | |
| isolate C2, complete | | | |
| genome | | | |
| Bombyx mori | 125,437 bp | KF306217.1 | GI: 548578211 |
| nucleopolyhedrovirus | | | |
| isolate C6, complete | | | |
| genome | | | |
| Bombyx mori | 126,861 bp | KJ186100.1 | GI: 695132325 |
| nucleopolyhedrovirus | | | |
| strain Brazilian, complete | | | |
| genome | | | |
| Mutant Autographa | 118,582 bp | KU697902.1 | GI: 1040495973 |
| californica | | | |
| nucleopolyhedrovirus | | | |
| isolate vAcRev-1, | | | |
| complete genome | | | |
| Mutant Autographa | 138,991 bp | KU697903.1 | GI: 1040496108 |
| californica | | | |
| nucleopolyhedrovirus | | | |
| isolate vAcRev-2, | | | |
| complete genome | | | |

Analysis of the nucleotide sequences of the C6 and E2 variants of AcNPV, and the bacmid bMON14272, derived from AcNPV-E2 revealed the frequency of cuts by restriction enzymes available from commercial sources. The following table summarizes these results.

TABLE 18
Frequency of cuts by non-redundant restriction enzymes in AcNPV-E2
and bMON14272

| Cuts | AcNPV-E2 | bMON14272 |
|---|---|---|
| 0 | Bsu36I, SrfI, Sse83987I, I-CeuI, | Bsu36I, I-CeuI, PI-SceI, I-PpoI, |
| | PI-SceI, I-PpoI, I-SceI, MauBI, | I-SceI, MauBI, PI-PspI |
| | PI-PspI | |
| 1 | AvrII, AbsI, FseI | AvrII, SrfI, FseI |
| 2 | SfiI, AscI | AbsI, Sse8387I, SfiI, AscI |
| 3 | SexAI, EcoNI, SgrDI, SgfI, KflI | SgrDI, KflI |
| 4 | SmaI/XmaI, PasI, MreI, NotI | SexAI, MreI, SgfI |
| 5 | AarI, AflII | AarI, PasI, EcoNI |
| 13 | PacI | PacI |

It is desirable to create variants of AcNPV-E2 and BmNPV, and shuttle vectors derived from them, where one or more of the restriction sites that cut 1-3 times, plus the NotI sites, which cuts 4 times in AcNPV are removed by site directed mutagenesis. These sites include AvrII, AbsI, FseI, SrfI, SdaI, SfiI, AscI, SgrDI, KflI, SexAI, SgfI, and NotI, with the AvrII, SrfI, FseI, AbsI, and AscI sites removed initially. Some of these enzymes produce compatible cohesive ends that can be used to assemble other DNA cassettes, and when the ends of two fragments are ligated together are not cleaved by either enzyme, similar to the BioBricks and related gene assembly schemes noted in the Background of the Invention.

Synthetic linkers comprising one or more recognition sequences for Bsu36I, SrfI, Sse83987I, and MauBI, that don't cut AcNPV plus AvrII, AbsI, FseI, SrfI, SfiI, AscI, SgrDI, KflI, SexAI, SgfI, and NotI, that cut 1-4 times, or fewer times in a variant lacking one or more of these sites can be prepared, that facilitate the design modular genetic elements that can be assembled into functional baculovirus shuttle vectors. Pad, which has an AT-rich recognition sequence cuts 13 times each in AcNPV and bMON14272, in the backbone of the virus, but not within the contiguous mini-F-Kan-mini-attTn7 sequences of the bMON14272 shuttle vector.

TABLE 19
Recognition sites of restriction enzymes useful in the design of modular vectors

| Site | Name | Compatible Enzymes |
|---|---|---|
| CC↓TNA↑GG | Bsu36I | Compatible with BlpI (GC′TNA, GC) which is |
| | (Overhang: 5′ | symmetric and Bpu10I (CC′TNA, GC) which is |
| | TNA)- | asymmetric) and DdeI (C′TNA,G) |
| TAACTATAACGGTC↑CTAA↓GGTAGCGAA | I-CeuI | Not compatible with anything else |
| | (Overhang: 3′ | |
| | CTAA) | |
| TAGGG↑ATAA↓CAGGGTAAT | I-SceI | Not compatible with anything else |
| | (Overhang: 3′ | |
| | ATAA ) | |
| TGGCAAACAGCTA↑TTA↓TGGGTATTATGGGT | PI-PspI | Not compatible with anything else |
| | (Overhang: 3′ | |
| | TTAT ) | |
| CG↓CGCG↑CG | MauBI | Compatible with AscI (GG′CGCG, CC), BssHII |
| | (Overhang: 5′ | (G′CGCG, C), MluI (A, CGCG, G) |
| | CGCG) | |
| TAACTATGACTCTC↑TTAA↓GGTAGCCAAAT | I-PpoI | Not compatible with anything else |
| | (Overhang: 3′ | |
| | TTAA) | |
| ATCTATGTCGG↑GTGC↓GGAGAAAGAGGTAATGAAATGG | PI-SceI | Not compatible with anything else |
| | (Overhang: 3′ | |
| | GTGC) | |
| CC↑TGCA↓GG | SbfI (Overhang: | Compatible with NsiI (A, TGCA′T), PstI |
| | 3′ TGCA) | (C, TGCA′G) |
| GCCCT↑↓GGGC | SrfI (Overhang: | BLUNT ENDS |
| | Blunt) | |
| CC↑TGCA↓GG | Sse8387I | |
| | (Overhang: 3′ | |
| | TGCA)- | |
| C↓CTAG↑G | AvrII | Compatible with NheI (G′CTAG, C), SpeI |
| | (Overhang: 5′ | (A′CTAG, T), and XbaI (T′CTAG, A) |
| | CTAG) | |
| CC↓TCGA↑GG | AbsI | Compatible with AbsI (CC′TCGA, GG), PaeR7I |
| | (Overhang: 5′ | (C′TCCGA, G), PspXI (VC,TCGA, GB), SalI |
| | TCGA) | (G′TCGA, C), SgrDI (CG′TCGA, CG), XhoI |
| | | (C′TCGA, G) |
| GG↑CCGG↓CC | FseI (Overhang: | Not compatible with anything else |
| | 3′ CCGG) | |
| GG↓CGCG↑CC | AscI | Compatible with BssHII (G′CGCG,C), MauBI |
| | (Overhang: 5′ | (CG,CGCG,CG), MluI (A′CGCG,T) |
| | CGCG)- | |
| GGCCN↑NNN↓NGGCC | SfiI (Overhang: | Compatible with many enzymes, including |
| | 3′ NNN)- | BglI |
| CG↓TCGA↑CG | SgrDI | Compatible with AbsI (CC′TCGA, GG), PaeR7I |
| | (Overhang: 5′ | (C′TCGA,G), PspXI (VC, TCGA, GB), SalI |
| | TCGA)- | (G′TCGA,C), SgrDI (CG′TCGA, CG), XhoI |
| | | (C′TCGA, G) |
| GCG↑AT↓CGC | SgfI (Overhang: | Compatible with AsiSI (GCG, ST′CGC), PacI |
| | 3′ AT)- | (TTA, AT′TAA), PvuI (CG, AT′CG) |
| GC↓GGCC↑GC | NotI | Compatible with EagI (C′GGCC, G |
| | (Overhang: 5′ | |
| | GGCC) | |
| TTA↑AT↓TAA | PacI | Compatible with AsiSI (GCG, AT′CGAA), PvuI |
| | | (CG, AT′CG) |

Pairs of linkers containing recognition sites for rare cutting restriction enzymes, typically with sequences that are 8 or more nucleotides in length, can be used to flank genetic elements in cassettes, such that digestion and annealing of two sets of genetic elements flanked by similar pairs are assembled into one contiguous fragment, similar to the BioBrick system noted earlier. In this scheme, pairs such as NotI/EagI, AbsI/SgrDI, MauBI/AscI can be used to assemble larger DNA cassettes, since they are unlikely to have recognition sequences in the middle of the genetic elements being assembled for insertion into cloning or expression vectors designed. for particular applications.

Linkers comprising recognition sites suitable for assembly of modular baculovirus vectors are called “BaculoBricks”, as noted in the Terms and Definitions section of this application. These and similar linkers comprising recognition sites for rare-cutting restriction enzymes can also be used in creating modular mammalian shuttle vectors, plant shuttle vectors, fungal shuttle vectors, and many plasmids from other large enteric or non-enteric bacterial plasmid systems, which may have applications in many fields of synthetic biology.

Modular baculovirus shuttle vectors need to contain a bacterial replicon, preferably one that is stable, and propagates at a low copy number, like the mini-F replicon used in bMON14272. They also need a drug resistance marker to facilitate selection of bacteria harboring the shuttle vector. In bMON14272, this was a gene conferring resistance to Kanamycin, but other selectable markers, such as those conferring resistance to ampicillin, tetracycline, chloramphenicol, gentamycin, among many others, or metabolic markers, such as one carrying a gene that can complement in trans, a gene that is mutated in the host cell. Shuttle vectors may optionally comprise one or more target sites for site specific transposons, such as a mini-Tn7 element liked to a lacZalpha gene, or other selectable or screenable markers noted in other examples of the application.

The key genetic elements added to a shuttle vector are independent, and need not be contiguous to each other, as they are in bMON14272. The replicon, drug resistance marker, and the optional target site can be in distinct locations within the viral genome, and in opposite orientations with respect to each other, as long as the resulting virus is stably propagated in bacteria, and in cultured eukaryotic host cells.

It may be desirable to randomly mutagenize a viral backbone, to identify locations that allow insertions of different DNA cassettes, such as a synthetic mini-attTn7, into many locations, which may be equal to or more stable than other locations. Tn5-based mutagenesis systems are now available from Lucigen, that facilitate the random transposition of DNA segments flanked by synthetic left and right arms of Tn5 into target DNA samples in vitro, in the presence of purified transposition proteins, or in vivo in a cell harboring a vector comprising the target sequence and a helper plasmid providing transposition proteins in trans. A viral shuttle vector comprising a replicon and a drug resistance marker, can be subjected mutagenesis with a mini-Tn5 element comprising one or more mini-attTn7 target sites. This approach allows the identification of locations within the viral backbone that may be more suited for stable, long term use, than those traditionally used for construction of recombinant viruses, or those identified by methods directed to sites within one or several clustered non-essential genes, as noted above.

These general approaches can also be applied to a wide variety of shuttle vectors that propagate only in bacteria, or in bacteria and in other types of eukaryotic cells. Viral and non-viral mammalian vectors, plant cell-based vectors, fungal vectors, for example, can all be redesigned, and used as modular targets for the insertion of DNA cassette carried on site specific transposons that are similar to those described in this application. The powerful new ability to directly select for insertions into a target site, coupled with other novel screening methods, dramatically increases the utility of systems designed to study the structure and function of a wide variety of genes, and facilitates the development of vectors that are capable of expression of heterologous proteins at high levels suitable for use in a variety of commercial applications.

### Example 10—Design of Synthetic Linkers Comprising Recognition Sequences for Restriction Enzymes that Cut Infrequently to Facilitate Cloning of One or More Segments of Genetic Elements into Large Plasmids and Shuttle Vectors for Use in Prokaryotic or Eukaryotic Cells

As noted above, pairs of synthetic linkers containing recognition sites for restriction enzymes that cut infrequently in large plasmids that generally propagate only in bacteria or in shuttle vectors that can propagate in at least two types of host cells, typically with sequences that are 8 or more nucleotides in length, can be used to flank genetic elements in cassettes, such that digestion and annealing of two sets of genetic elements flanked by similar pairs are assembled into one contiguous fragment, similar to the BioBrick system noted earlier.

In the many of the BioBrick standard assembly schemes, the linkers comprise recognition sites for restriction enzymes that are only 6 nucleotides in length, with one set using a prefix linker comprising sites for EcoRI and XbaI separated by site for NotI, and a suffix linker comprising sites for SpeI and PstI, also separated by a NotI site. For example, a vector comprising a first sequence of interest is digested with EcoRI and SpeI, and a second vector comprising a second sequence of interest and a replicon and selectable marker is digested with EcoRI and XbaI. Samples from both digests are mixed and ligated together, to form a larger vector comprising two sequences of interest with a “scar” site formed by the ligation of the compatible XbaI and SpeI sticky ends that is not recognized by either enzyme. The two contiguous sequences of interest in the larger product vector can be released from digestion with EcoRI and SpeI, or retained in a vector digested with EcoRI and XbaI that are used in subsequent reactions to assemble vectors comprising three or more contiguous sequences of interest, separated by scar sequences. Another standard uses linkers comprising recognition sites for EcoRI, BglII, BamHI, XhoI, where BglII and BamHI generate compatible sticky ends, while another standard uses linkers that contain recognition sites for AgeI and NgoMIV.

The biggest limitation of many of these assembly schemes is that the DNA segment to be flanked by these types linkers must not contain a recognition site used in the prefix or suffix linkers. If it does, it needs to be removed by mutagenesis, perhaps involving careful design to introduce mutations that do not affect the reading frame of a nucleotide sequence encoding a polypeptide, or by altering nucleotide residues in codons within the recognition site that do not alter the sequence of the encoded polypeptide, or by replacing codons with those encoding amino acids that are similar to those in the parental sequence, or are generally conserved, when a variety of related residues are compared in a multiple sequence alignment.

For applications that require assembly of larger segments of DNA, such as those derived from large plasmids, or shuttle vectors comprising stable low copy number replicons, such as mini-F, or large operons comprising linked sets of genes operably-linked to one or more promoters, it is desirable to use synthetic linkers that comprise sequences for restriction enzymes that do not cut, or very rarely cut in the sequences of interest that will be flanked at their 5′ and 3′ ends by prefix and suffix linkers, respectively.

The frequency by which a Class II restriction enzyme will cut is a function of the length of the sequence it is sensitive to. An enzyme with a 4-bp recognition sequence and 4 possible bases at each position, will theoretically cut 1 in 4⁴ (256) 4-bp long recognition sites. An enzyme with a 6-bp recognition sequence and 4 possible bases at each position, will theoretically cut 1 in 6⁴ (4,096) 6-bp long recognition sites. An enzyme with an 8-bp recognition sequence and 4 possible bases at each position, will theoretically cut 1 in 8⁴ (65,536) 8-bp long recognition sites. GC content affects these frequencies, increasing the probability that enzymes that have GC-rich recognition sites will cut more often in large segments of DNA that are more GC-rich than average, compared to the probability that enzymes that have AT-rich recognition sequences will cut in the same large segment of DNA.

While a variety of Class II restriction enzymes have been characterized that have recognition sites that are 8 or more bp in length, they are much less commonly available from commercial sources than enzymes that have recognition sites that are 4, 5, 6, or 7 bp in length. Of these, many fewer can be assigned to sets where one or more enzymes generate sticky 5′ or 3′ ends suitable for use in ligation experiments where a scar is formed by the annealing and ligation of two compatible sticky ends.

To facilitate the modular assembly of large plasmids that propagate only in prokaryotes, or shuttle vectors that can propagate in two types of host cells, one typically in bacteria, such as laboratory strains of E. coli, an enteric bacterium, and the other in non-enteric bacteria or eukaryotic cells, such as insect, mammalian, and fungal cells, it is appropriate to determine the relative frequency of cleavage sites for a variety of Class II restriction enzymes. The relative frequency (from 0 to 5) of cuts by non-redundant restriction enzymes in the AcNPV-E2 E2 strain of baculovirus, and the shuttle vector designated bMON14272 are provided in a table noted above. The recognition sites of a variety of restriction enzymes that are potentially useful in the design of modular vectors, are also provided in a table noted above. After eliminating enzymes that produce blunt ends, those that produce sticky ends that are not compatible with any other enzyme, and those that produce sticky ends with one or more ambiguous nucleotides (e.g., Bsu36I), very few enzymes remain that can be considered for use in linkers where one or more of the recognition sites in the prefix or suffix linker that rarely cut within the plasmid or shuttle vector of interest, such as AvrII (C′CTAG,G), which cuts AcNPV and bMON14272 only once, or those that have recognition sites that are 8 or more bp in length.

Linkers comprising recognition sites for specific pairs of enzymes such as NotI/EagI, AbsI/SgrDI, MauBI/AscI can be used to design and assemble larger DNA cassettes, since they are unlikely to have recognition sequences in the middle of the genetic elements being assembled for insertion into cloning or expression vectors designed. for particular applications. While these may be the most appropriate pairs of enzymes suitable for use in the assembly of modular baculovirus vectors, they are not necessarily limited to these types of vectors, but may also be used to facilitate the design and assembly of large modular mammalian, plant, and fungal shuttle vectors, as well as other large plasmids and shuttle vectors that propagate in one or more types of prokaryotic cells.

### Sequence Alignment 29: Synthetic Pairs of Linkers Comprising Recognition Sites for NotI, EagI, and PspOMI

NotI (GC′GGCC,GC) has a 5′ overhang of GGCC, which is compatible with PspOMI (G′GGCC,C) and EagI (C′GGCC,G). The recognition site for EagI is an internal subset of NotI. NotI cuts AcNPV four (4) times, and bMON14272 six (6) times. PspOMI cuts AcNPV seven (7) times, and bMON14272 nine (9) times. EagI cuts AcNPV forty (40) times, and bMON14272 forty-two (42) times.

Synthetic DNA sequences comprising recognition sites for NotI and PspOMI are shown below, separated by a series of unspecified nucleotides, specified here as a series of 8 “n” residues, which may comprise recognition sites for other restriction enzymes. The number of unspecified or ambiguous residues can vary, to be larger or smaller than 8 residues, depending on the desired application. In the first example below, ligation of a linker digested to expose a PspOMI site at its 3′ end with a linker digested to expose a NotI site at its 5′ end produces a fragment with an internal scar that is not digestible by either enzyme. In the second example below, ligation of a linker digested to expose a NotI site at its 3′ end with a linker digested to expose a PspOMI site at its 5′ end produces a fragment with an internal scar that is not digestible by either enzyme.

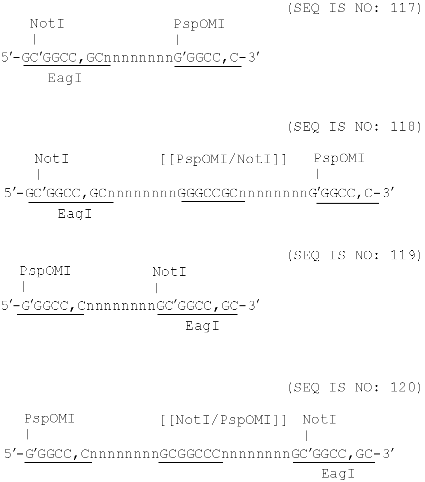

TABLE 20
Frequency of cuts by restriction enzymes in used in synthetic linkers in AcNPV-E2 and bMON14272

| | | AcNPV- | | |
|---|---|---|---|---|
| Enzyme | Site | E2 | bMON14272 | Comments |
| NotI | GC′GGCC, GC | 4 | 6 | All NotI sites contain internal EagI sites |
| EagI | C′GGCC, G | 40 | 42 | EagI PspOMI produces sticky ends that are compatible with NotI |
| | | | | and PspOMI sites |
| PspOMI | G′GGCC, C | 7 | 9 | PspOMI produces sticky ends that are compatible with NotI and |
| | | | | EagI sites |
| AbsI | CC′TCGA, GG | 1 | 2 | One AbsI/PaeR7I/XhoI site in AcNPV is near the 5′ end of the |
| | | | | Ac-sod gene at position 25,926, and the AbsI site in the bacmid |
| | | | | is right after the SalI site in the mini-attTn7 segment |
| SgrDI | CG′TCGA, CG | 3 | 3 | SgrDI/SalI sites are in the Ac-ORF1629 gene at position 6,698, |
| | | | | the non-essential AcORF-18 gene at 14,944, and Ac-Orf54 gene at |
| | | | | 45,700. |
| XhoI | C′TCGA, G | 14 | 17 | XhoI sites are compatible with AbsI, SgrDI, and SalI sites |
| PspXI | VC′TCGA, GB | 8 | 11 | Some PspXI sites are AbsI sites and both contain internal XhoI |
| | | | | sites |
| SalI | G′TCGA, C | 54 | 55 | One SalI site is at the 3′ end of the mini-attTn7 segment in |
| | | | | the middle of the lacZalpha gene in the bacmid |
| MauBI | CG′CGCG, CG | 0 | 0 | Does not cut AcNPV or the bacmid. MauBI sites contain internal |
| | | | | BssHII sites |
| AscI | GG′CGCG, CC | 2 | 2 | Cuts twice in AcNPV, once in Ac-arif-1 gene at position 16,573, |
| | | | | plus Ac-pkip-1 gene at 20,948 |
| BssHII | G′CGCG, C | 34 | 38 | All AscI and MauBI sites contain internal BssHII sites. |
| MluI | A′CGCG, G | 80 | 80 | Does not cut in Kan-lacZalpha-mini-attTn7-mini-F replicon |
| | | | | region in the bacmid, but cuts in the flanking Ac-ORF603 and |
| | | | | Ac-ORF-12 genes in the AcNPV and the bacmid |
| FseI | GG, CCGG′CC | 1 | 1 | Cuts once near 5′ end of Ac-gta gene at position 34,285 in |
| | | | | AcNPV |
| PacI | TTA↑AT↓TAA | 13 | 13 | PacI cuts 13 times each in the viral backbone of AcNPV and |
| | | | | bMON14272, but not within the contiguous mini-F-Kan-mini-attTn7 |
| | | | | sequences of bMON14272. |

Sequence Alignment 30: Synthetic pairs of linkers comprising recognition sites for AbsI and SgrDI AbsI (CC′TCGA,GG) has a 5′ overhang of TCGA, which is compatible with SgrDI (CG′TCGA,CG), and the 6-base cutters, PaeR7I (C′TCCGA,G), PspXI (VC′TCGA,GB [where V=A or C or G, and B=C or G or T]), SalI (G′TCGA,C), and XhoI (C′TCGA,G). AbsI cuts AcNPV one (1) time, and bMON14272 two (2) times. SgrDI cuts AcNPV three (3) times, and bMON14272 three (3) times.

Synthetic DNA sequences comprising recognition sites for AbsI and SgrDI are shown below, separated by a series of unspecified nucleotides, specified here as a series of 8 “n” residues, which may comprise recognition sites for other restriction enzymes. The number of unspecified or ambiguous residues can vary, to be larger or smaller than 8 residues, depending on the desired application. In the first example below, ligation of a linker digested to expose a AbsI site at its 3′ end with a linker digested to expose a SgrDI site at its 5′ end produces a fragment with an internal scar that is not digestible by either enzyme. In the second example below, ligation of a linker digested to expose a SgrDI site at its 3′ end with a linker digested to expose a AbsI site at its 5′ end produces a fragment with an internal scar that is not digestible by either enzyme.

The restriction enzyme XhoI (C′TCGA,G) recognizes the center 6 bp of the AbsI site (CC′TCGA,GG) and SalI (G′TCGA,C) recognizes the center 6 bp of the SgrDI (CG′TCGA,CG) site. The hybrid scar site is also not recognized or digestible by XhoI or SalI.

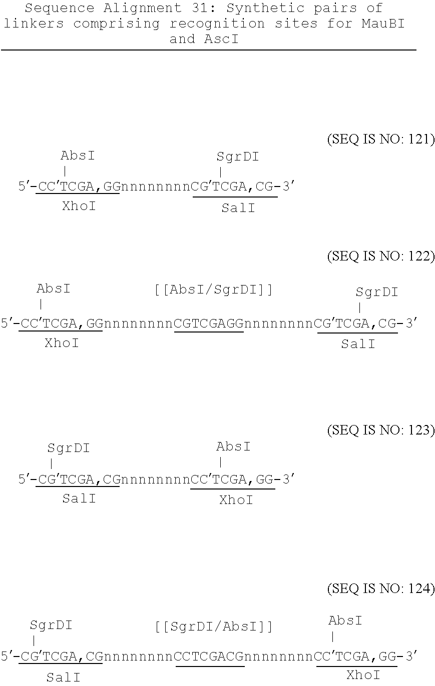

MauBI (CG′CGCG,CG) has a 5′ overhang of CGCG, which is compatible with AscI (GG′CGCG,CC), and the 6-base cutters BssHII (G′CGCG,C) and M/ul (A′CGCG,G). MauBI cuts AcNPV zero (0) times, and bMON14272 zero (0) times. AscI cuts AcNPV two (2) times, and bMON14272 two (2) times.

Synthetic DNA sequences comprising recognition sites for MauBI and AscI are shown below, separated by a series of unspecified nucleotides, specified here as a series of 8 “n” residues, which may comprise recognition sites for other restriction enzymes. The number of unspecified or ambiguous residues can vary, to be larger or smaller than 8 residues, depending on the desired application. In the first example below, ligation of a linker digested to expose a AscI site at its 3′ end with a linker digested to expose a MauBI site at its 5′ end produces a fragment with an internal scar that is not digestible by either enzyme. In the second example below, ligation of a linker digested to expose a MauBI site at its 3′ end with a linker digested to expose a AscI site at its 5′ end produces a fragment with an internal scar that is not digestible by either enzyme.

The restriction enzyme BssHII (G′CGCG,C) which recognizes the center 6 bp of both MauBI and AscI can cut at either site, plus the hybrid scar site that is not recognized or digestible by MauBI or AscI.

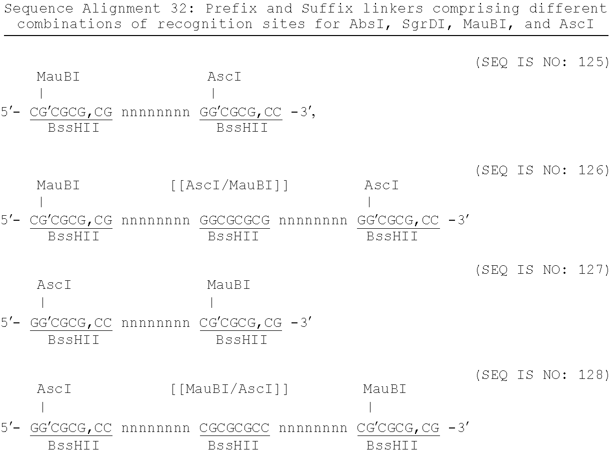

In view of the hybrid scar sites produced by ligating the sticky ends on DNA fragments digested with restriction enzymes that have recognition sites that are typically 8 bp in length illustrated in Sequence Alignments 28-30, a variety of prefix and suffix linkers can be considered for general use in the design and assembly of genetic elements for use in modular vector systems. The following table outlines 8 combinations of recognition sites for compatible restriction enzymes that can used in pairs on synthetic prefix and suffix linkers that flank a DNA fragment of interest. In each pair, the recognition site for the second enzyme listed in the prefix is compatible with the first enzyme listed in the suffix.

The recognition site for each enzyme in a prefix or suffix illustrated below is separated by a series of unspecified nucleotides, specified here as a series of 8 “n” residues, which may comprise recognition sites for other restriction enzymes. The number of unspecified or ambiguous residues can vary, to be larger or smaller than 8 residues, depending on the desired application.

TABLE 21
Pairs of recognition sites for restriction enzymes
useful in the design of synthetic linkers suitable
for use in the assembly of modular vectors

| Prefix | SEQ ID NO | Suffix | SEQ ID NO |
|---|---|---|---|
| MauBI-AbsI | 129 | SgrDI-AscI | 136 |
| MauBI-SgrDI | 130 | AbsI-AscI | 134 |
| AscI-AbsI | 131 | SgrDI-MauBI | 135 |
| AscI-SgrDI | 132 | AbsI-MauBI | 133 |
| AbsI-MauBI | 133 | AscI-SgrDI | 132 |
| AbsI-AscI | 134 | MauBI-SgrDI | 130 |
| SgrDI-MauBI | 135 | AscI-AbsI | 131 |
| SgrDI-AscI | 136 | MauBI-AbsI | 129 |

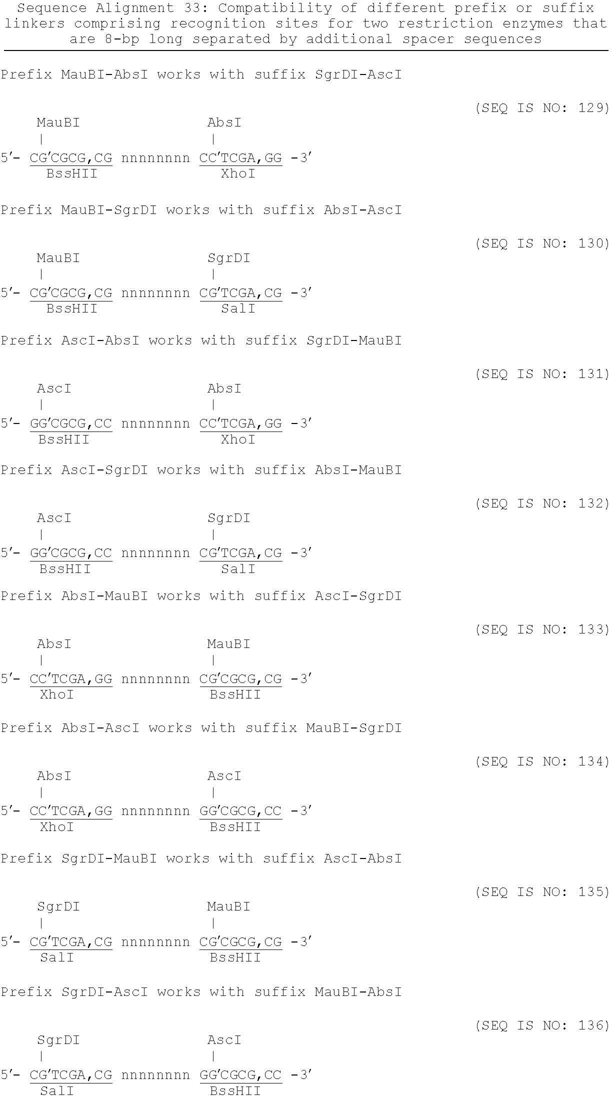

Sequence Alignment 34: Compatibility of different prefix or suffix linkers comprising recognition sites for two restriction enzymes that are 8-bp long separated by additional spacer sequences

In this example, the spacer sequences in the MauBI and AbsI sites in the prefix linker and the SgrDI and AscI suffix linker are both replaced by the recognition site for the Pad (TTA,AT′TAA). Pad cuts 13 times in AcNPV and 13 times in bMON14272 (but not within the min-F-Kan-mini-attTn7 segment), and is compatible with AsiSI (GCG,AT′CGAA), PvuI (CG,AT′CG).

Digestion of the DNA fragment flanked by the prefix and suffix sequences noted below with Pad will allow release of the insert that also contains the 3′ portion of the prefix linker and the 5′ portion of the suffix linker, allowing ligation of the insert fragment into a vector comprising an Pad site in either orientation, or ligation of the vector that retains the 5′ portion of the prefix linker and the 3′ portion of the suffix linker to regenerate a single Pad site.

In one of many possible variations, the spacer sequences in the MauBI and AbsI sites in the prefix linker and the SgrDI and AscI suffix linker are both replaced by the recognition site for the FseI (GG,CCGG′CC). FseI cuts once in AcNPV and once in bMON14272, and is not compatible with any other restriction enzyme since the sticky end that is generated is a 4-bp 3′ CCGG overhang.

Digestion of the DNA fragment flanked by the prefix and suffix sequences noted below with FseI will allow release of the insert that also contains the 3′ portion of the prefix linker and the 5′ portion of the suffix linker, allowing ligation of the insert fragment into a vector comprising an FseI site in either orientation, or ligation of the vector that retains the 5′ portion of the prefix linker and the 3′ portion of the suffix linker to regenerate a single FseI site. An EagI site, which is compatible with NotI, overlaps the FseI and AscI sites (data not shown).

One advantage of using Pad instead of FseI as the spacer sequence is that the Pad recognition sequence is very AT-rich, compared to the recognition sequence for FseI, which is very GC-rich. A long stretch of GC-rich residues across the entire prefix-spacer-prefix and suffix-spacer-suffix sequences may prevent or impair the ability of DNA segments to be synthesized where the prefix and suffix sequences flank a desired set of genetic elements, compared to prefix and suffix sequences where the spacer sequence is more AT-rich. Note also that Pad cuts 13 times in AcNPV and in bMON14272, while FseI cuts once each in AcNPV and bMON14272, which may alter strategies for assembling modular baculovirus vectors using Pad in a spacer sequence, compared to FseI.

TABLE 22
Summary of pairs of synthetic prefix and suffix linkers comprising
two 8-bp recognition sites separated by the recogntion site for
Pact each pair separate by an intervening sequence (IV) comprising
an AvrII site

| | SEQ | | SEQ | | SEQ | Digestion/ | SEQ |
|---|---|---|---|---|---|---|---|
| | ID | | ID | Prefix-AvrII-Suffix | ID | Ligation | ID |
| Prefix | NO | Suffix | NO | Double Polylinker | NO | Product | NO |
| MauBI- | 137 | SgrDI- | 144 | MauBI-PacI-AbsI-AvrII- | 145 | MauBI-PacI- | 153 |
| PacI-AbsI | | PacI-AscI | | SgrDI-PacI-AscI | | AscI | |
| MauBI- | 138 | AbsI-PacI- | 142 | MauBI-PacI-SgrDI-AvrII- | 146 | MauBI-PacI- | 153 |
| PacI-SgrDI | | AscI | | AbsI-PacI-AscI | | AscI | |
| AscI-PacI- | 139 | SgrDI- | 143 | AscI-PacI-AbsI-AvrII- | 147 | AscI-PacI- | 154 |
| AbsI | | PacI-MauBI | | SgrDI-PacI-MauBI | | MauBI | |
| AscI-PacI- | 140 | AbsI-PacI- | 141 | AscI-PacI-SgrDI-AvrII- | 148 | AscI-PacI- | 154 |
| SgrDI | | MauBI | | AbsI-PacI-MauBI | | MauBI | |
| AbsI-PacI- | 141 | AscI-PacI- | 140 | AbsI-PacI-MauBI-AvrII- | 149 | AbsI-PacI- | 155 |
| MauBI | | SgrDI | | AscI-PacI-SgrDI | | SgrDI | |
| AbsI-PacI- | 142 | MauBI- | 138 | AbsI-PacI-AscI-AvrII- | 150 | AbsI-PacI- | 155 |
| AscI | | PacI-SgrDI | | MauBI-PacI-SgrDI | | SgrDI | |
| SgrDI- | 143 | AscI-PacI- | 139 | SgrDI-PacI-MauBI-AvrII- | 151 | SgrDI- PacI- | 156 |
| PacI-MauBI | | AbsI | | AscI-PacI-AbsI | | AbsI | |
| SgrDI- | 144 | MauBI- | 137 | SgrDI-PacI-AscI-AvrII- | 152 | SgrDI-PacI- | 156 |
| PacI-AscI | | PacI-AbsI | | MauBI-PacI-AbsI | | AbsI | |

TABLE 23
Pairs of synthetic prefix and suffix linkers comprising two 8-bp
recognition sites separated by the recogntion site for Pacl, each pair
separated by an intervening sequence (IV) comprising an Avrll site

| | SEQ | IV | | SEQ |
|---|---|---|---|---|
| Prefix or | ID | or | | ID |
| Ligated Digestion Product (LP) | NO | LP | Suffix | NO |
| MauBI PacI AbsI | 137 | // | SgrDI PacI AscI | 144 |
| \| \| \| | | | \| \| \| | |
| CG′CGCG,CG tta,at′taa CC′TCGA,GG | | | CG′TCGA,CG tta,at′taa GG′CGCG,CC | |
| BssHII Xhol | | | SalI BssHII | |
| CG′CGCG,CG tta,at′taa CC′TCGA,GG cctagg CG′TCGA,CG tta,at′taa GG′CGCG,CC | | | | 145 |
| CG′CGCG,CG tta,at′′taa GG′CGCG,CC | | | | 153 |
| MauBI PacI SgrDI | 138 | // | AbsI PacI AscI | 142 |
| \| \| \| | | | \| \| \| | |
| CG′CGCG,CG tta,at′taa CG′TCGA,CG | | | CC′TCGA,GG tta,at′taa GG′CGCG,CC | |
| BssHII SalI | | | XhoI BssHII | |
| CG′CGCG,CG tta,at′taa CG′TCGA,CG cctagg CC′TCGA,GG tta,at′taa GG′CGCG,CC | | | | 146 |
| CG′CGCG,CG tta,at′taa GG′CGCG,CC | | | | 153 |
| AscI PacI AbsI | 139 | // | SgrDI PacI MauBI | 143 |
| \| \| \| | | | \| \| \| | |
| GG′CGCG,CC tta,at′taa CC′TCGA,GG | | | CG′TCGA,CG tta,at′taa CG′CGCG,CG | |
| BssHII XhoI | | | SalI BssHII | |
| GG′CGCG,CC tta,at′taa CC′TCGA,GG cctagg CG′TCGA,CG tta,at′taa CG′CGCG,CG | | | | 147 |
| GG′CGCG,CC tta,at′taa CG′CGCG,CG | | | | 154 |
| AscI PacI SgrDI | 140 | // | AbsI PacI MauBI | 141 |
| \| \| \| | | | \| \| \| | |
| GG′CGCG,CC tta,at′taa CG′TCGA,CG | | | CC′TCGA,GG tta,at′taa CG′CGCG,CG | |
| BssHII SalI | | | XhoI BssHII | |
| GG′CGCG,CC tta,at′taa CG′TCGA,CG cctagg CC′TCGA,GG tta,at′taa CG′CGCG,CG | | | | 148 |
| GG′CGCG,CC tta,at′taa CG′CGCG,CG | | | | 154 |
| AbsI PacI MauBI | 141 | // | AscI PacI SgrDI | 140 |
| \| \| \| | | | \| \| \| | |
| CC′TCGA,GG tta,at′taa CG′CGCG,CG | | | GG′CGCG,CC tta,at′taa CG′TCGA,CG | |
| XhoI BssHII | | | BssHII SalI | |
| CC′TCGA,GG tta,at′taa CG′CGCG,CG cctagg GG′CGCG,CC tta,at′taa CG′TCGA,CG | | | | 149 |
| CC′TCGA,GG tta,at′taa CG′TCGA,CG | | | | 155 |
| AbsI PacI AscI | 142 | // | MauBI PacI SgrDI | 138 |
| \| \| \| | | | \| \| \| | |
| CC′TCGA,GG tta,at′taa GG′CGCG,CC | | | CG′CGCG,CG tta,at′taa CG′TCGA,CG | |
| XhoI BssHII | | | BssHII SalI | |
| CC′TCGA,GG tta,at′taa GG′CGCG,CC cctagg CG′CGCG,CG tta,at′taa CG′TCGA,CG | | | | 150 |
| CC′TCGA,GG tta,at′taa CG′TCGA,CG | | | | 155 |
| SgrDI PacI MauBI | 143 | // | AscI PacI AbsI | 139 |
| \| \| \| | | | \| \| \| | |
| CG′TCGA,CG tta,at′taa CG′CGCG,CG | | | GG′CGCG,CC tta,at′taa CC′TCGA,GG | |
| SalI BssHII | | | BssHII XhoI | |
| CG′TCGA,CG tta,at′taa CG′CGCG,CG cctagg GG′CGCG,CC tta,at′taa CC′TCGA,GG | | | | 151 |
| CG′TCGA,CG tta,at′taa CC′TCGA,GG | | | | 156 |
| SgrDI PacI AscI | 144 | // | MauBI PacI AbsI | 137 |
| \| \| \| | | | \| \| \| | |
| CG′TCGA,CG tta,at′taa GG′CGCG,CC | | | CG′CGCG,CG tta,at′taa CC′TCGA,GG | |
| Sall BssHII | | | BssHII XhoI | |

| | |
|---|---|
| CG′TCGA,CG tta,at′taa GG′CGCG,CC cctagg CG′CGCG,CG tta,at′taa CC′TCGA,GG | 152 |
| CG′TCGA,CG tta,at′taa CC′TCGA,GG | 156 |

### Proof of Concept Experiments

Twenty vectors were designed and synthesized Twist Biosciences (T), which included test, target, and donor vectors. Twist vectors with the prefix pTAH, confer resistance to ampicillin and have a high copy number (H). Vectors with the prefix pTCM, confer resistance to chloramphenicol and have a medium copy number (M). Vectors with the prefix pTKM, confer resistance to kanamycin and have a medium copy number. Test vectors have the suffix -CX or -KX, target vectors have the suffix -CT or -KT, and donor vectors have the suffix -AD.

Test vectors comprise sequences that mimic transposition of Tn7 in a synthetic attachment site in different reading frames to express extended or truncated fusion protein that may or may not confer resistance to an antibiotic such as chloramphenicol or kanamycin. Target vectors are similar, but also contain the synthetic attachment site positioned an appropriate distance away from where the insertion is desired. Donor vectors typically contain the left and right arms of Tn7 flanking a cargo DNA sequence that may contain one or more synthetic polylinkers that contain recognition sites for several restriction enzymes (also referred to as a multiple cloning site or MCS), and other genes, such as the lacZalpha gene derived from pUC18, pUC19, or similar cloning vectors, wild-type and variant forms of the aacC1 gene derived from pFastBac1 conferring resistance to gentamycin, the rpsL gene conferring resistance to streptomycin, and genes encoding products that confer a screenable phenotype upon a cell, such as chromogenic or fluorescent proteins, or the uidA gene encoding E. coli beta glucuronidase.

Dry DNA samples were resuspended in water or Tris-EDTA buffer, and transformed into competent E. coli DH10B cells using a protocol provided by Thermo Fisher, and purified by restreaking on agar plates containing the antibiotic of the drug resistance gene on the backbone of the vector. Liquid LB media supplemented with antibiotics were used to prepare overnight cultures. Glycerol stocks were prepared from overnight cultures and stored at −20 degrees Celsius. The phenotypes of DH10B cells harboring different vectors were determined by restreaking overnight cultures on LB agar plates containing different concentrations of antibiotics, typically, Amp 100, IPTG 40, X-Gal 40, Cam 50, Kan 50, or a series of concentrations on solid agar or liquid LB medium, that included Cam 0, 6.25, 12.5, and 25, or Kan 0, 12.5, 25, and 50.

TABLE 24
Summary of Twist Vectors 1-20

| | | | | | Size | SEQ ID |
|---|---|---|---|---|---|---|
| | | | Expected | Observed | of | NO of |
| ID Code | Short Name | Description | Phenotype | Phenotype | Insert | Insert |
| 01-AD | pTAH-new-mini-Tn7 | New-miniTn7 with smaller flanking | AmpR, Iac | AmpR, Iac | 546 | 199 |
| | | sequences and internal MauBI-PacI- | minus | minus | | |
| | | AbsI-AvrII-SbfI(PstI)-SacII-SgrDI- | | | | |
| | | PacI-AscI polylinker | | | | |
| 02-AD | pTAH-new-mini-Tn7- | New mini-Tn7 with internal | AmpR, Iac | AmpR, Iac | 986/79 | 200/201 |
| | lacZalphapUC18 | lacZalpha region derived from | plus | | | |
| | | pUC18 | | | | |
| 03-CX | pTCM-Kan-CGRT | Kan extended with CGRTK to mimic | CamR, KanR | CamR, KanS | 1028 | 202 |
| | | Tn7LrfI | | | | |
| 04-CX | pTCM-Kan-PS | Kan extended with PS to mimic | CamR, KanS | CamR, KanS | 1028 | 203 |
| | | prior art reference with silent | | | | |
| | | EcoRI and SpeI sites | | | | |
| 05-CX | pTCM-Kan- | Kan extended with PSFNAVVYHS to | CamR, KanS | CamR, KanS | 1040 | 204 |
| | PSFNAVVYHS | mimic prior art reference | | | | |
| 06-CT | pTCM-Kan-PS-mini- | Kan extended with PS and | CamR, KanS | CamR, KanS | 1069 | 205 |
| | attTn7 | overlapping mini-attTn7 | | | | |
| 07-CX | pTCM-Kan-Tn7Lrf1 | Kan extended with CGRTK with | CamR, KanR | CamR, KanS | 1074 | 206 |
| | | partial Tn7L rf1 | | | | |
| 08-CX | pTCM-Kan-Tn7Lrf2 | Kan extended with | CamR, KanR | CamR, KanS | 1075 | 207 |
| | | LWADKIVGNWEGWKWSF with | | | | |
| | | partial Tn7L rf2 | | | | |
| 09-CX | pTCM-Kan-Tn7Lrf3 | Kan extended with | CamR, KanR | CamR, KanS | 1076 | 208 |
| | | PVGGQNSWELGGVEMEFLRII with | | | | |
| | | partial Tn7L rf3 | | | | |
| 10-CX | pTCM-Mau-Abs- | Kan extended with PS to mimic | CamR, KanS | CamR, KanS | 1016 | 209 |
| | Kan177-PS-Sgr-Asc | prior art reference without | | | | |
| | | silent EcoRI or SpeI sites | | | | |
| 11-CX | pTCM-Mau-Abs- | Kan gene from pACYC177 not | CamR, KanR | CamR, KanR | 1016 | 210 |
| | Kan177-Sgr-Asc | extended or truncated without | | | | |
| | | silent EcoRI or SpeI sites | | | | |
| 12-KX | pTKM-CATd8 | CAT gene from pACYC184 not | KanR, CamR | KanR, CamR | 876 | 211 |
| | | extended or truncated and deleted | | | | |
| | | 8 bases from the right polylinker | | | | |
| 13-KX | pTKM-CAT-TAA | TAA replaced Asp Codon | KanR, CamR | KanR, CamR | 876 | 212 |
| 14-KX | pTKM-CAT-TAATAA | TAATAA replaced CysAsp Codons | KanR, CamS | KanR, Cam(S) | 876 | 213 |
| | | | | with micro | | |
| | | | | colonies on | | |
| | | | | Kan 50/Cam | | |
| | | | | 50 | | |
| 15-KT | pTKM-CAT-TAATAA- | TAATAA replaced CysAsp Codons- | KanR, CamS | KanR, Cam(S) | 889 | 214 |
| | mini-attTn7 | overlapping mini-AttTn7 | | with micro | | |
| | | | | colonies Kan | | |
| | | | | 50/Cam 12.5 | | |
| | | | | and Kan | | |
| | | | | 50/Cam 50 | | |
| 16-KX | pTKMC-CAT-Tn7Lrf1 | CAT extended with CGRTK with | KanR, CamR | KanR, CamR | 896 | 215 |
| | | partial Tn7L rf1 | | | | |
| 17-KX | pTKMC-CAT-Tn7Lrf2 | CAT extended with | KanR, CamR | KanR, CamR | 897 | 216 |
| | | LWADKIVGNWEGWKWSF with | | | | |
| | | partial Tn7L rf2 | | | | |
| 18-KX | pTKMC-CAT-Tn7Lrf3 | CAT extended with | KanR, CamR | KanR, CamR | 898 | 217 |
| | | PVGGQNSWELGGVEMEFLRII with | | | | |
| | | partial Tn7L rf3 | | | | |
| 19-KT | pTKM-lacZalpha- | lacZalpha-micro-attTn7 which is | Kan R, Iac | Kan R, Iac | 687 | 218 |
| | micro-attTn7 | 150 nt smaller than pTKM-19-KT | plus | plus | | |
| 20-KT | pTKM-lacZalpha- | lacZalpha-mini-attTn7 similar to | Kan R, Iac | Kan R, Iac | 837 | 219 |
| | mini-attTn7 | the sequence in the bacmid | plus | plus | | |
| | | bMON14272 | | | | |

A first series of gene fusions has the cat gene altered, so that insertions take place near an essential cysteine codon, upstream from the normal stop codon as disclosed in Example 2. Extensions after transposition were expected to restore resistance to chloramphenicol.

Colonies harboring the test vectors, where the extension included sequences derived from the left end of Tn7 in three different reading frames, all grew on agar plates containing kanamycin and chloramphenicol, strongly suggesting that transposition into the gene fusion sequence in the target vector should restore activity to the encoded gene fusion.

Cells harboring the pTKM-14-KX and pTKM-15-KT vectors grew very slowly, forming microcolonies on agar plates after 1 day, containing kanamycin and chloramphenicol, as noted above.

A second series of gene fusions has the NPT-II gene, which confers resistance to kanamycin, altered so that insertions take place near the normal stop codon just upstream from an extension that encodes proline and serine, that were expected to produce a fusion protein that is inactive, as disclosed in Example 4. Colonies harboring the test vectors, where the extension included sequences derived from the left end of Tn7 in three different reading frames, did not confer resistance to chloramphenicol and kanamycin, which was unexpected, compared to the results observed for the cat-attTn7 gene fusions.

A third series of gene fusions has the lacZalpha gene with the mini-attTn7 site inserted into it, to mimic the target site in the bacmid bMON14272, and a smaller version that deletes 150 bp flanking the MCS region in the mini-attTn7 sequence in this gene. Both of these target vectors conferred resistance to kanamycin and were lac plus on agar plates containing IPTG and X-gal.

The donor vector pTAH-01-AD conferred resistance to ampicillin and the donor vector pTAH-02-AD conferred resistance to ampicillin and was lac plus on agar plates containing IPTG and X-gal.

Transposition experiments were carried out by first transforming the helper vector pMON7124 into DH10B cells harboring the target vectors pTKM-CAT-TAATAA-mini-attTn7, pTKM-lacZalpha-micro-attTn7, or pTKM-lacZalpha-mini-attTn7, and isolating pure colonies on agar plates containing chloramphenicol and tetracycline, or kanamycin and tetracycline, depending on the drug resistance marker on the backbone of the target vector. Overnight cultures containing the target and helper vectors were prepared and transformed with a donor vector pTAH-new-mini-Tn7-lacZalphapUC18 or pFastBac1.

Two independent cultures of cells harboring pTKM-CAT-TAATAA-mini-attTn7 and pMON7124 that were transformed with pTAH-new-mini-Tn7-lacZalphapUC18 and spread on LB agar plates containing Kan 50, Cam 25, Tet 20, IPTG and X-gal, contained a mixture of blue and white colonies. Blue colonies from the two independent cultures were restreaked on the same agar plates, and pure overnight cultures prepared and stored as glycerol stocks.

Samples of each glycerol stock were provided to GeneWiz, which prepared DNA samples comprising a mixture of both the composite and the helper vectors that were used as templates for sequencing across the junction of the left end of Tn7 and the expected insertion site in the gene fusion of the target vector. Structural analysis of the both composite vectors confirmed the mini-Tn7-lacZalpha gene from the donor vector was inserted into the pTKM-CAT-TAATAA-mini-attTn7 vector to produce a composite vector, where the gene fusion was extended into the left end of Tn7 to restore resistance to chloramphenicol. This is apparently the first demonstration of transposition into a gene fusion based on selection for restoration of activity of the encoded enzyme.

Sequence Alignment 35: Sequence of 240 bp segment across the insertion site in a
15KCT-2A7-Blue-1 composite target vector derived from pTKM-CAT-TAATAA-mini-attTn7
and a mini-Tn7-lacZalpha donor segment
SEQ ID NO 240
CAATCCCTGGGTGAGTTTCACCAGTTTTGATTTAAACGTGGCCAATATGGACAACTTCTTCGCCCCCGTTTTCACCATGG
<-- Partial coding sequence of 3′ end of the cat gene -------------------------->
GCAAATATTATACGCAAGGCGACAAGGTGCTGATGCCGCTGGCGATTCAGGTTCATCATGCCGTCTGTGATGGCTTCCAT
<------------------------------------------------------------------------------>
GTCGGCAGAATGCTTAATGAATTACAACAGTNC NGTNGNNNGNCAAAATAGTTGGGAACTGGGAGGGGTGGAAATGGAGT
<-------------------------------> <-- Tn7L * Stop Codon -----------------

With unsure nucleotides at positions 192, 194, 197, 199-201, and 203.

Independent cultures of cells harboring pTKM-lacZalpha-mini-attTn7 or pTKM-lacZalpha-micro-attTn7 plus the helper vector pMON7124 were also transformed with pFastBac1, and spread on LB agar plates containing Kan 50, Tet 20, Gent 7, IPTG, and Bluo-gal, which contained a mixture of blue and white colonies after one day. White colonies from the two independent cultures were restreaked on the same agar plates, and pure overnight cultures prepared and stored as glycerol stocks.

Samples of each glycerol stock were provided to GeneWiz, which prepared DNA samples comprising a mixture of both the composite and the helper vectors that were used as templates for sequencing across the junction of the left end of Tn7 and the expected insertion site in the gene fusion of the target vector. Structural analysis of the both types of composite target vectors confirmed that the mini-Tn7-5V40-MCS-PpolH-Gent segment from the pFastBac1 donor vector was inserted into both types of target vectors comprising a lacZalpha-mini-attTn7 gene to produce composite target vectors, where the gene fusion is disrupted by the insertion of the mini-transposon, preventing complementation between the alpha peptide and the acceptor polypeptide, resulting in a lac minus phenotype on agar plates containing IPTG and the chromogenic substrate X-gal or Bluo-gal (Nucleotide sequence data across the junctions in the composite vectors is not shown).

Taken together, all three sets of transposition experiments demonstrated that DH10B cells harboring novel medium copy target vectors and compatible helper vectors could be used to test transposition from a variety of new modular donor vectors, reconstituting in a sense, the donor/helper/target vector system used in the original baculovirus shuttle vector system, but substituting much smaller target vectors that could be used in a systematic analysis of gene fusions that could be used to directly select or screen for transposition events in bacteria.

A second series of vectors were designed and ordered from Twist Biosciences (Vectors 21-41) to test the significance or optimize the effectiveness of different DNA segments in the target or donor vectors.

Cells harboring the first series of cat-attTn7 fusions grew very slowly, and replacing the cat promoter with an inducible lac promoter, and encoding a protein ending with ELQQY instead of ELQQYC may allow them to grow better under uninduced and induced conditions. The sulfhydryl group in the extra Cysteine residue at the end of the protein may react with other molecules within the cell if is expressed at high levels.

Two alterations to the kan gene (adding a silent EcoRI site, without altering the codons upstream from the stop codon, or a SpeI site, downstream from the stop codon) just upstream and downstream from the natural stop codon could have affected the outcome. Extensions added by reading into Tn7L in different reading frames could also prevent restoration of activity to the fusion protein.

New vectors where designed to separate these issues, to remove the altered EcoRI site, and to redesign the kan fusions so that transposition into a vector that has a Pro-Ser extension will truncate it back to the normal stop codon. To do this though, the TGT (encoding Cys) at the left end of Tn7L has to be in the right reading frame, to encode a normal sized enzyme. The last amino acid is Phe (F), and the second to last is also Phe, but the second to last is not always conserved in lineups of related kanamycin phosphotransferases. The second to last codon was altered to encode Leucine (L), which should allow expression of a product that has the same size after transposition, from the gene encoding extended, inactive PS fusion protein.

Several new donor vectors were designed work with the kan gene comprising the F270L mutation to contain stop codons in several different reading frames. While many are possible, three were designed and synthesized, two containing Pad sites (TTAATTAA) in slightly different positions just beyond the TGT, and one containing an XbaI site that has a TAG stop codon within it. Transposition of any of the three new donors should restore kanamycin activity in the target vectors comprising the redesigned kan-attTn7 sequence. Altered sequences near the 5′ end of Tn7L don't need to be palindromic. Other sequences can be used as long as the truncation or extension restores activity to the encoded protein. If TGT is an essential requirement at the 5′ end of Tn7 in a donor vector, it can be inserted into 3 different reading frames as noted below.

TABLE 25
Encoding amino acids by Tn7L after transposition into a target site

| Three Reading | | | TGT | Nnn | |
|---|---|---|---|---|---|
| Frames | Encoded polypeptide | | nTG | Tnn | |
| rf1, rf2, and rf3 | segment | nnn | nnT | GTN | nnn |
| nnn TGT nnn nnn | X-C-X-X | $ | C | $ | $ |
| | | | Excludes | | |
| | | | 19 aa plus * | | |
| nnn nTG Tnn nnn | X-(L/M/V)- | $ | LMV | FLSY*CW | $ |
| | (F/L/S/Y/*/C/W)-X | | Excludes | Excludes | |
| | | | 17 aa plus * | PHQRIMTNKVADE | |
| nnn nnT GTn nnn | X-(FSYCILTVPNAHRDG)-(V)-X | $ | FSYCILTVPNAHRDG | V | $ |
| | | | Excludes | Excludes | |
| | | | WQ*MKE | 19 aa plus * | |

*The symbol “$” represents any amino acid and any of the three stop codons is represented by “*”. “QKE” are common to the list of excluded amino acids, preceded by “#”, for reading frames 2 and 3. The net effect is that polypeptides containing adjacent Q, K, or E residues will be difficult to encode for restoration or disruption of activity by a Tn7-like transposon.

Other site-specific transposons may have sequences at their ends that are different than TGT, which maybe longer or shorter, complicating the algorithm noted above, but fusions created after transposition should be predictable based on genetic code tables for different organisms.

Target and donor vectors comprising the rpsL gene (conferring sensitivity to streptomycin) and a chromogenic staghorn coral protein were also designed. The target vector containing rpsL-attTn7 gene should allow direct selection of transposition events in the presence of streptomycin. The coral-attTn7 gene should allow detection of white colonies in a background of cyan blue colonies (without the need to use IPTG and expensive X-gal or Bluo-Gal chromogenic substrates.

Several donor vectors were synthesized to contain two genes, lacZalpha, rpsL, or CyanFP, plus the gentamycin resistance gene derived from pFastBac1, which can be used to test and monitor transposition events with or without selection of drug resistance conferred by a marker within the cargo segment of the donor vector.

The new “double donors” can easily be reduced in size, removing the first or second gene by digesting with a single restriction enzyme that has a site that flanks either gene, and ligating to circularize the molecule.

Two codons near the 5′ end of the gentamycin resistance gene were altered to have silent changes to encode Serine, since the Twist Sequence Analysis flagged part of the unaltered sequences to be part of a direct repeat just upstream from the ATG start codon. Vectors without these changes could not be synthesized due to the direct repeats flagged by their system.

TABLE 26
Summary of New Vectors 21-40

| | | | | | | SEQ ID |
|---|---|---|---|---|---|---|
| | | | Expected | Observed | Size of | NO |
| ID Code | Short_Name | Description | phenotype | Phenotype | Insert | of Insert |
| 21-CX | pTCM-21C-Kan- | Kan MLDEFF not extended or | CamR, KanR | CamR, KanR | 1016 | 220 |
| | EcoRI | truncated with silent EcoRI site | | | | |
| 22-CX | pTCM-22C-Kan- | Kan MLDEFFCGRTK extended to | CamR, KanS | CamR, KanS | 1025 | 221 |
| | MLDEFFCGRTK | mimic Tn7Lrf1 without silent | if CGRTK | | | |
| | | EcoRI and Spel sites | extension | | | |
| | | | doesn't | | | |
| | | | restore | | | |
| | | | activity | | | |
| 23-CX | pTCM-23C-Kan- | Kan MLDELF-F270L (TTT-Phe to | CamR, KanR, | CamR, KanR | 1016 | 222 |
| | F270L | CTG-Leu) | if F270L is | | | |
| | | | conservative | | | |
| 24-CX | pTCM-24C-Kan- | Kan MLDELFPS-F270L (TTT-Phe to | CamR, KanS, if | CamR, KanS | 1016 | 223 |
| | MLDELFPS-F270L | CTG-Leu) extended PS | F270L and PS | | | |
| | | | fusion is | | | |
| | | | inactive | | | |
| 25-CX | pTCM-25C-Kan- | Kan MLDELFN-TG-TTT-AAT-TAA- | CamR, Kan? | CamR, KanS | 1021 | 224 |
| | MLDELFPSN-F270L | Pacl-1 extended N | | | | |
| 26-CX | pTCM-26C-Kan- | Kan MLDELF-TG-TTT-TAA-TTT-A- | CamR, KanR | CamR, KanR | 1022 | 225 |
| | MLDELF-F270L | Pac1-2, Phe to Leu, plus Phe | | | | |
| | | before TAA stop should be | | | | |
| | | resistant | | | | |
| 27-CX | pTCM-27C-Kan- | Kan MLDELF-TG-TTC-TAG-A-Xbal, | CamR, KanR | CamR, KanR | 1022 | 226 |
| | MLDELF-F270L | Phe to Leu, plus Phe before TAG | | | | |
| | | stop should be resistant | | | | |
| 28-CT | pTCM-28C-Kan- | Kan MLDELFPS-F270L (TTT-Phe to | CamR, KanS | CamR, KanS | 1064 | 227 |
| | MLDELFPS-F270L- | CTG-Leu)-FPS-Stop-mini-attTn7 | | | | |
| | attT | version 1, should be sensitive | | | | |
| 29-CT | pTCM- | LacP-Kan MLDELFQA-F270L (TTT- | CamR, KanR | CamR, KanS | 1188 | 228 |
| | 29CLacPKanMLDEL | Phe to CTG-Leu)-FQA-Stop-mini- | | | | |
| | FQA-F270Latt | attTn7 should be resistant if QA | | | | |
| | | doesn't affect activity | | | | |
| 30-CT | pTCM- | LacP-Kan MLDELFPS-F270L (TTT- | CamR, KanS | CamR, KanS | 1188 | 229 |
| | 30CLacPKanMLDEL | Phe to CTG-Leu)-FPS-Stop-mini- | | | | |
| | FPS-F270Latt | attTn7 version 1, replacing the | | | | |
| | | kan promoter, with lacPO | | | | |
| | | inducible promoter driving kan- | | | | |
| | | mini-attTn7 | | | | |
| 31-KT | pTKM- | Lac promoter-cat gene-TAATAA | KanR, CamS | KanR, CamR | 965 | 230 |
| | 31KTLacPCatTAATA | replaced CysAsp Codons- | | when | | |
| | ACysAspatt | overlapping mini-AttTn7 ending | | spotted, not | | |
| | | ELQQY, replacing the cat | | streaked | | |
| | | promoter with lacPO driving CAT- | | | | |
| | | mini-attTn7 encoding truncated | | | | |
| | | cat protein | | | | |
| 32-KT | pTKM-32KT- | Lac promoter-cat gene-TAA | KanR, CamS | KanR, CamR, | 965 | 231 |
| | LacPCat- | replaced Asp Codon-overlapping | | when | | |
| | TAArepAspatt | mini-AttTn7 ending ELQQYC, | | spotted, not | | |
| | | replacing the cat promoter with | | streaked | | |
| | | lacPO driving CAT-mini-attTn7 | | | | |
| | | encoding truncated cat protein | | | | |
| 33-KT | pTKM-33KT-rpsL- | rpsL-mini-attTn7 with insertion in | KanR, StrepS | KanR, StrepS, | 965 | 232 |
| | mini-attTn7 | codon 122 of 125 encoding | | but very slow | | |
| | | GVKRPKA before insertion, and | | or no growth | | |
| | | replacing PKA after insertion so | | | | |
| | | target with dominant StrepS gene | | | | |
| | | linked to mini-attTn7 is disrupted | | | | |
| | | by transposition and confers | | | | |
| | | StrepR | | | | |
| 34-KT | pTKM-34KT-LacP- | Lac promoter-Cyan chromogenic | KanR, cyan | KanR, white | 1016 | 233 |
| | CyanFP-attTn7 | protein-mini-attTn7 encoding | | | | |
| | | NPLKVQ before insertion near | | | | |
| | | codon 228 of 231 replacing KVQ | | | | |
| | | so transposition disrupts protein | | | | |
| | | (colored to white). | | | | |
| 35-AD | pTAH-35AD- | Mini-Tn7-MauBl-Absl-LacZalpha- | AmpR, GentR, | AmpR, GentS, | 1822 | 234 |
| | miniTn7-lacZalpha- | SgrDI-Absl-Gent-SgrDI-Ascl, with | lac plus | lac plus | | |
| | Gent | wild-type Tn7 ends | | | | |
| 36-AD | pTAH-36AD- | Mini-Tn7L-Pacl-2a-lacZalpha- | AmpR, GentR, | AmpR, GentS, | 1822 | 235 |
| | Tn7LPac1-2a-lacZ- | Gent where Tn7L in rf2 would | lac plus | lac plus | | |
| | Gent | encode Kan-MLDELF*, with | | | | |
| | | altered Tn7L and Padl site | | | | |
| 37-AD | pTAH-37AD-Tn7L- | Mini-Tn7L-Pacl-la-lacZalpha- | AmpR, GentR, | AmpR, GentS, | 1822 | 236 |
| | Pacl-la-lacZaGent | Gent where Tn7L in rf2 would | lac plus | lac plus | | |
| | | encode Kan-MLDELFN* with | | | | |
| | | altered Tn7L and Padl site | | | | |
| 38-AD | pTAH-38AD- | Mini-Tn7L-Xbal-lacZalpha-Gent | AmpR, GentR, | AmpR, GentS, | 1822 | 237 |
| | Tn7LXbal-1a-lacZa- | where Tn7L in rf2 would encode | lac plus | lac plus | | |
| | Gent | Kan-MLDELF* with altered Tn7L | | | | |
| | | and Xbal site | | | | |
| 39-AD | pTAH-39AD-mini- | Mini-Tn7-MauBl-Absl-rpsL-SgrDI- | AmpR, GentR | AmpR, GentS | 1868 | 238 |
| | Tn7-rpsL-Gent | Absl-Gent-SgrDI-Ascl, with rpsL | | | | |
| | | dominant StrepS gene, plus | | | | |
| | | Gentamycin gene | | | | |
| 40-AD | pTAH-40AD-mini- | Mini-Tn7-MauBl-Absl-lacP- | AmpR, GentR | AmpR, GentS | 2278 | 239 |
| | Tn7-CyanFP--Gent | AmilCyanFP-SgrDI-Absl-Gent- | | | | |
| | | SgrDI-Ascl with Cyan | | | | |
| | | chromogenic coral fluorescent | | | | |

Analysis of the phenotypes of colonies harboring different test vectors confirmed that introducing a silent EcoRI site at the 3′ end of the kan gene did not affect activity of the encoded protein, but adding extensions that mimicked reading frames extending into a wild-type Tn7L resulted in fusion proteins that did not confer resistance to kanamycin. Gene fusions comprising a conserved F270L mutation at the 3′ end of the kan gene, did not affect activity of the encoded enzyme, while those encoding extensions adding PS or QA did affect activity of the enzyme. These results strongly suggest that gene fusions comprising an altered form of the kan gene fused to mini-attTn7 can be used to detect transposition events where the insertion truncates an extended, inactive fusion protein back to a sequence that has the same length as the wild-type enzyme that also contains the conserved F270L substitution near the C-terminal end of the enzyme.

Analysis of the phenotypes of colonies harboring target vectors comprising altered cat-mini-attTn7 sequences gave different results when cultures were streaked, compared to spotted onto agar plates containing kanamycin plus chloramphenicol. Colonies comprising these vectors grew well on agar plates containing kanamycin, but not at all or poorly on agar plates containing kanamycin and chloramphenicol. When 20 ul of cells from an overnight culture were spotted onto agar plates containing kan, cam, or kan and cam, both grew well on plates containing kanamycin after 1 day, but grew well on all test plates after 2 days. Chloramphenicol is bacteriostatic, so inactivation of the antibiotic by any mechanism should allow growth if the concentration falls below a minimal inhibitory concentration, compared to kanamycin which is bacteriostatic, and kills cells that cannot inactivate the antibiotic.

Both strategies, restoring activity to cells harboring vectors comprising gene fusions encoding a catalytically-inactive enzyme, one by extension and one by truncation, can be used to with other types of genes encoding enzymes conferring resistance to antibiotics, including ampicillin, tetracycline, gentamycin, hygromycin, among many others, and pairs of toxin/anti-toxin genes, to facilitate the direct selection of transposition events in E. coli, and related bacteria.

Analysis of the phenotypes of colonies harboring new dual donor vectors revealed that the gentamycin gene that was inserted into these vectors was defective, and could not confer resistance to the antibiotic at 7 ug/ml, although they all conferred resistance to ampicillin at 100 ug/ml, and were lac plus on agar plates if they contained also the lacZalpha gene. The gene encoding a chromogenic protein derived from staghorn coral did not produce colonies that were noticeably different in color from lac minus colonies on agar plates containing IPTG and X-gal.

Analysis of the phenotypes of colonies harboring target and donor vectors comprising the rpsL gene did not grow or grew very slowly as microcolonies on different kinds of selection plates, suggesting that the product of this gene is toxic when it is carried on a high copy number vector, even in the absence of induction with IPTG.

Cells harboring each of the new target vectors and the helper vector were prepared by transforming target vector DNA samples into D10B cells harboring pMON7124, and their colony phenotypes compared on agar plates containing tetracycline plus different concentrations of kanamycin and/or chloramphenicol.

Cells harboring the pTCM-28C-Kan-MLDELFPS-F270L-attTn7, pTCM-29CLacPKanMLDELFQA-F270LattTn7, and pTCM-30CLacPKanMLDELFPS-F270LattTn7 target vectors plus pMON7124, all grew when 20 ul of overnight cultures were spotted onto agar plates containing chloramphenicol, but not on plates containing kanamycin, confirming that the PS, QA extensions did not encode an active enzyme.

Cells harboring the pTKM-31KTLacPCatTAATAACysAspattTn7 and pTKM-32KT-LacPCat-TAArepAspattTn7 target vectors plus pMON7124, all grew when 20 ul of overnight cultures were spotted onto agar plates containing chloramphenicol, kanamycin, or both chloramphenicol and kanamycin, which was unexpected, but consistent with observations noted above, where growth of cells on plates containing chloramphenicol, a bacteriostatic agent, might be observed on densely spotted plates, compared to plates where cultures are streaked out to form separate colonies.

Similar results were also obtained, when transposition experiments were carried out when two independent cultures of DH10B harboring the target vector pTKM-31KTLacPCatTAATAACysAspattTn7 or pTKM-32KT-LacPCat-TAArepAspattTn7 and the pMON7124 helper vector were transformed with four different donor vectors, pTAH-new-mini-Tn7-lacZalphapUC18, pTAH-37AD-Tn7L-PacI-1a-lacZaGent, pTAH-38AD-Tn7LXbaI-1a-lacZa-Gent, and pTAH-40AD-mini-Tn7-CyanFP-Gent, to and selecting for colonies that grew on agar plates containing Cam 25 Kan 50 Tet 10 IPTG Xgal Gent 7, Cam Kan Tet IPTG Xgal, Cam Kan Tet Gent, and Cam Kan Tet. Microcolonies were observed for all four combinations of donor vectors transformed into cells harboring pTKM-32KT-LacPCat-TAArepAspattTn7 and the pMON7124 on plates containing Cam Kan Tet IPTG Xgal, but not for cells harboring the pTKM-31KTLacPCatTAATAACysAspattTn7n7 vector, strongly suggesting that the gene fusion in the pTKM-32KT vector is suitable for selecting for transposition events that restore activity by extension of truncated cat gene that ends with the sequence ELQQYC, compared to the sequence encoded by the pTKM-32KT that ends with the sequence ELQQY, which did grew on plates cells containing kanamycin, but not on plates containing chloramphenicol. DNA sequence analysis across the target sites in parental and composite target vectors will be performed to confirm these observations.

Analysis of the sequence of the defective gentamycin resistance genes suggested that the “silent changes” made to two adjacent serine codons at the 5′ end of its coding sequence altered nucleotides at the 3′ end of second of three 15-bp direct repeats, one in the promoter region, and two which were are identical within the coding sequence. The functional nature of these direct repeats are not known, but are reported in the annotated version of the GenBank sequence of the transposon comprising the aacC1 gene.

The defective gentamycin resistance genes in four dual donor vectors pTAH-35AD-miniTn7-lacZalpha-Gent, pTAH-36AD-Tn7LPacI-2a-lacZ-Gent, pTAH-37AD-Tn7L-PacI-1a-lacZaGent, pTAH-38AD-Tn7LXbaI-1a-lacZa-Gent, and pTAH-40AD-mini-Tn7-CyanFP-Gent were repaired by digesting mixing pFastBac1 plus each of the new donor vectors with the restriction enzyme BtgI, which cuts twice in each of the new donors, just upstream from the promoter and downstream from the 3′ end of the gentamycin resistance gene, and three times in in pFastBac1, heat inactivating the restriction enzyme, and ligating with T4 DNA ligase, before transforming the mixture into competent DH10B cells. Two colonies from each ligation mixture that grew on agar plates containing ampicillin, gentamycin, IPTG and X-gal were purified by restreaking and DNA samples and DNA samples prepared were for sequencing. Colonies harboring the repaired pTAH-35AD-miniTn7-lacZalpha-Gent, pTAH-36AD-Tn7LPacI-2a-lacZ-Gent, pTAH-37AD-Tn7L-PacI-1a-lacZaGent, and pTAH-38AD-Tn7LXbaI-1a-lacZa-Gent dual donor vectors were blue on plates containing X-gal, while those harboring the pTAH-40AD-mini-Tn7-CyanFP-Gent vector were white. Miniprep DNA samples were prepared for sequence analysis to confirm that the defective gene was repaired in each of the dual donor vectors.

The new dual donor vectors will greatly facilitate the analysis of transposition events using target vectors comprising modified cat-mini-attTn7 or kan-mini-attTn7 fusions, among others, by allowing for the selection of composite vectors based on the restoration of activity in the gene fusion, and monitoring the expression of the lacZalpha gene, with and without selection for gentamycin resistance carried within the cargo sequence of the mini-transposon, and comparing their efficiencies of transposition under different selection or screening schemes.

### Example 11—Design of Modular Donor Vectors

Many types of donor vectors comprising mini-Tn7 elements have been constructed, where the left and right arms of Tn7 (Tn7L and Tn7R) flank a central cargo DNA segment comprising one or more genes of interest that can all be transposed to a specific attachment site on a target vector or the chromosome by the products of the tnsA-D genes carried on a helper vector, or randomly transposed to a segment on a conjugal plasmid by the products of the tnsA-C and E genes. Random transposition has also been observed in several cases when products of the tnsA and tnsB genes are used with a gain-of-function mutant product encoded by a variant tnsC gene.

The pFastBac series of vectors commonly used to facilitate expression of heterologous proteins by recombinant baculoviruses in cultured insect cells are derived from pMON14327, that contains the left and right arms of Tn7 (Tn7L and Tn7R) flanking an internal region comprising a gene encoding resistance to gentamycin, along with the strong polyhedrin promoter (Ppolh) driving expression of a gene conceding β-glucuronidase, and a sequence comprising an SV40 poly(A) transcriptional terminator [Luckow et al, (1993)]. The order of genetic elements is Tn7L, SV40 poly(A), β-gluc, Ppolh, GentR, and Tn7R, with the promoter and coding sequences for the gentamycin resistance gene oriented towards Tn7R, and the SV40 poly(A)-β-gluc-Ppolh segment oriented in the opposite strand, towards Tn7L. This plasmid also contains an origin of replication from the cloning vector pUC8, and a gene encoding resistance to ampicillin (AmpR), which is incompatible with the replicon in the helper plasmid pMON7124, since they were both derived from replicons commonly used in the ColE1/pMB1/pBR322/pUC series of related cloning vectors.

The pFastBac1 vector (now available from ThermoFisher), which has a size of 4776 bp, contains a variety of genetic elements that are not typically required for many transposition experiments. The mini-Tn7 transposon is 2084 bp long, where Tn7L is 166 bp long, and Tn7R is 225 bp long, with its central cargo DNA segment is 1693 bp long, comprising the SV40 poly(A) transcriptional terminator, a multiple cloning site, the polyhedrin promoter, and the gene conferring resistance to gentamycin. A 159 bp sequence that flanks Tn7L is apparently derived from sequences in the intergenic region between the E. coli phoS gene (also called pstS) and the 5-bp duplication (corresponding to −2 to +2) site beyond the 3′ end of the glmS gene. A 62 bp sequence that flanks Tn7R is apparently derived from the 3′ end of the glmS gene, extending from positions −2 to +2 (the 5-bp duplication), +3 to +22 (including the second but not the first TAA stop codon), +23 to +58 (which is the TnsD binding site, and encodes the last 11 aa of the glmS gene product (*EVTVSKALNRP) and the first stop codon), followed by 6 bp to half of a natural HincII site within the glmS gene. The vector backbone also comprises a 456 bp sequence comprising a bacteriophage f1 origin of replication that is not involved in transposition.

Smaller versions of the pMON14327 and related pFastBac series vectors can constructed by using a smaller backbone without the bacteriophage f1 origin of replication and shorter sequences that flank Tn7L and Tn7R, shorter arms in some case, and a shorter internal cargo segment comprising a multiple cloning site permitting the modular assembly by cloning or direct insertion of synthetic DNA segments to generate synthetic mini-Tn7 transposons, capable of being transposed to a wide variety of random or specific locations on target vectors or the chromosome of a host cell.

In one new version of a donor vector, designated pTAH-new-mini-Tn7, the mini-Tn7 is 495 bp long, with left and right arms that are 166 and 225 bp in length, respectively, flanking a 104 bp central cargo DNA segment comprising a polylinker comprising several 8-bp recognition sites for several rare cutting restriction enzymes (including MauBI, AbsI, AvrII, SgrDI, and AscI) as noted above in Example 9.

A variant form of this vector, designated pTAH-new-mini-Tn7-lacZalphapUC18, was also constructed, that has a 460 bp lacZalpha segment including the lac promoter of the cloning vector pUC18 inserted between the AbsI and SgrDI sites of the polylinker.

Other variant forms, comprising longer or shorter left and right arms of the Tn7 or Tn7-like element, or with altered sequences, adding or removing recognition sites for different restriction enzymes, or adding or removing stop codons within the arms of transposon, and forms comprising one or more marker genes or cargo genes of interest between the arms of the transposon, wherein each marker or cargo gene of interest is operably-linked to at least one promoter that is functional in bacteria or another type of host cell, may also be constructed and used with comparable donor/helper/target vector systems.

Transposition of the mini-Tn7-lacZalpha segment to the chromosome of E. coli DH10B cells should change the phenotype of the host cell from Lac minus (−) to Lac plus (+), or to a target vector comprising the truncated cat or NPT-II genes, restoring resistance to chloramphenicol or kanamycin, respectively, and screening to confirm that their phenotype was changed from Lac minus (−) to Lac plus (+) as well, without the need to select for resistance to gentamycin, that was commonly carried out in the pMON14327 and pFastBac series of vectors.

### Example 12—Design of Modular Helper Vectors Encoding Wild-Type and Variant Transposition Genes

A helper vector, designated pMON7124 comprising the right half of Tn7 cloned onto a derivative of pBR322, contains the Tn7R and the tnsABCDE genes encoding all five proteins needed for site-specific or random transposition of Tn7 into the chromosome or other plasmids within the cell [Barry (1988)]. When E. coli strain DH10B, harbors both the bacmid bMON14272, which confers resistance to Kanamycin, and the helper plasmid pMON7124, which confers resistance to Tetracycline, both plasmids co-exist because their replicons are in different incompatibility groups [Luckow et al (1993)]. When a pUC-based donor plasmid is introduced into a cell harboring the bacmid and pMON7124 (which a replicon that is incompatible with the donor plasmid), the mini-Tn7 segment on the donor plasmid is transposed by a cut/paste mechanism into its attachment site on the bacmid or into the chromosome, if the chromosomal site is not blocked by an existing Tn7 element.

This vector is fairly large, having a predicted length of 13,274 bp (D. Esposito, personal communication) comprising an 3,613 bp EcoRI-PstI fragment derived from pBR322 encompassing all of the tetracycline resistance gene, several genes involved in replication, including the rop, born, the incompatibility RNA, and the origin of replication (oriV), plus the 3′ end of the bla gene. The product of the rop gene is involved in copy number control, and the born (basis of mobility) sequence is described as the origin of transfer for conjugative mobilization using a conjugative broad host range plasmid, such as RP4. The remaining sequences from the PstI site to the EcoRI site apparently comprise a Tn7 element derived from Proteus mirabilis, including a 177 bp segment from the PstI site to an end of Insertion Sequence 1 (IS1), a 344 bp segment identical to the P. mirabilis glmS gene, Tn7R, the tnsA, B, C, D, and E genes, and two other complete genes (ybgA and rbfB) and one partial gene (ybfA) derived from Tn7.

While pMON1724 is adequate for many transposition experiments involving screening of transposition events involving bMON14272 and donor plasmids derived from pMON14327 or any of the pFastBac series of vectors, it is unnecessarily large, and several segments can be deleted without affecting the ability of the plasmid to provide transposition proteins in trans in a cell harboring a bacmid and a donor plasmid. One smaller variant deletes the 3′ two-thirds of the tnsE gene, both ybgA and rbfB genes, and the partial ybfA gene extending from a Pad site to the EcoRI site to produce a plasmid designated R982-X01 that is 10,822 bp, that retains the tetracycline resistance and replication genes from pBR322, and all of the tnsA, B, C, and D genes [Mehalko, J. L., Esposito, D. (2016) J. Biotechnol. 238: 1-8]

Smaller functional variants of pMON7124 and R982-X01 can also be made by deleting all of the tnsE gene (saving ˜393 bp), and sequences extending from one end of the origin of replication near two closely-spaced PpiI sites, across the 3′ end of a disrupted bla gene, a partial IS1 sequence, and most of the glmS-related sequences derived from Proteus mirabilis (saving ˜988 bp), as noted above. Other sequences between the 3′ end of the tetracycline resistance gene and one end of the origin of replication, that include the rop gene and the born sequence might also be deleted.

A very small tetracycline resistant helper plasmid can be constructed from small high copy number cloning vectors provided by Twist Biosciences in several steps, including those that confer resistance to chloramphenicol, ampicillin, or kanamycin resistance, by inserting a gene encoding a product conferring resistance to tetracycline, and deleting other sequences conferring resistance to other antibiotics, and then inserting sequences comprising a promoter operably linked to the tnsA, B, C, and D genes.

Smaller variants can also be prepared, comprising sequences encoding fewer transposition genes, such as the tnsA, B, and C genes, with the tnsD gene located on a target vector to facilitate studies designed to identify variants of the tnsD gene product that have an altered ability to bind to specific glmS-like sequences, such as those derived from homologues glmS found in human, yeast or other prokaryotic or eukaryotic chromosomes. A vector comprising a novel gene fusion comprising a sequence for a selectable marker fused to an attTn7-like target, and a tnsD gene comprising one or more mutagenized segments can be used in directed evolution experiments, in the presence of a helper vector encoding the tnsA, B, and C genes, and a donor plasmid comprising a mini-Tn7 element and one or more genes of interest. If the tnsD gene on the target vector is altered by mutagenesis, then composite variant target vectors that resulted from transposition into the target site, restoring the ability of the target vector to confer resistance to chloramphenicol or kanamycin as noted above, can be recovered by isolating plasmid DNA samples, retransforming composite vector into plasmid-free strain selecting for the target but not the helper or donor vectors, and analyzing its sequence to determine the nature of the mutation(s) in the tnsD gene. Several rounds of mutagenesis and direct selection may be needed to alter the specificity of the tnsD gene product to efficiently bind to specific target sequences that are similar but not identical to the E. coli glmS gene.

Modified target vectors comprising variant tnsC genes can also be constructed, to identify mutants that are similar to the “Gain of Function” mutations identified in earlier studies [Stellwagen, A. E and Craig, N. L. (1997) Genetics 145(3): 573-85]. The tnsD and tnsE genes were not required, and wild-type tnsA and B genes in the presence of an altered tnsC gene (tnsC*) facilitated random transposition of a mini-Tn7 element into other vectors or the chromosome of the host cell. Methods to identify variants of tnsC will differ from those used to identify variants of tnsD, by screening for phenotypic changes that occur as a result of the random transposition into a gene carried on the target vector, perhaps a large gene allowing counterselection or screening of transposition events if an insertion disrupts expression of its gene product. Examples include disruption of the lacZ, cat, NPT-II, bla, or tet genes, as noted in earlier sections of this application.

Variant synthetic forms of Tn7 that can randomly transpose at very high levels may be preferred for particular applications involved in modifying prokaryotic or eukaryotic cells that result in insertions without a plasmid or viral vector backbone, such as cell and gene therapy applications requiring insertion of one or more cargo DNA segments comprising one or several genes of interest.

### Example 13—General Principles Concerning Design of Modular Vectors Comprising One or More Transposon Traps

When key components of a bacterial plasmid or a viral or non-viral shuttle vector will be reused in other variant vectors, it is often useful to design the vectors so segments DNA comprising functionally-distinct genetic elements are modular, allowing easy methods for their extraction and insertion into other vectors, or easy methods for the insertion of other DNA segments into one or more sites on a vector that is adjacent to the 5′ end or the 3′ end of a segment of interest, in a preferred orientation, or in either orientation.

Traditionally simpler methods rely on use of one or more restriction enzymes to digest vectors comprising a DNA segment of interest, to create a mixture of DNA fragments, which may be separated on agarose or acrylamide gels and purified, that are then ligated into a vector digested with one or more enzymes that produce compatible 5′, 3′, or blunt ends, followed by ligation, and recovery of the new variant vector comprising the desired insert.

Other methods can also be used, including amplification of the desired segment using primers that flank the desired segment in the presence of a thermostable DNA polymerase (e.g., polymerase chain reaction, PCR) and comparable methods, to produce linear DNA segments that may be ligated directly into cloning vectors, or treated with other enzymes to add additional nucleotides at either end to facilitate ligation to a compatible vector, or digested with restriction enzymes that have recognition sites in the primer sequences flanking the original ends of the insert.

It may be desirable to build larger modular vectors from a series of smaller modular vectors in a sequential fashion, using functional genetic elements flanked by synthetic linkers comprising recognition sites for restriction enzymes that cut infrequently or not at all within an unmodified parental vector, or a virus that will be engineered to include a replicon, such as a shuttle vector, that allow it to be propagated in two types of host cells. Compatible sets of synthetic linkers, such as those described above in Example 9, may be used, to flank DNA segments comprising functionally distinct genetic elements, in smaller cloning vectors, which may be used as the source of an insert or a vector in a series of steps to assemble a final, product vector.

The baculovirus shuttle vector (bacmid) bMON14272, comprises a large ˜8 kb DNA segment containing several smaller functionally-distinct genetic elements, including a segment encoding a gene which confers resistance to kanamycin in E. coli, a lacZalpha gene comprising a synthetic mini-attTn7 sequence, and mini-F, a stable low copy number replicon derived from the prototype fertility plasmid, F. This large segment is inserted into the non-essential polyhedrin gene, in the baculovirus Autographa californica Nuclear Polyhedrosis Virus (AcNPV). Another bacmid, bMON14271, has this large segment inserted into the opposite orientation at the same location in AcNPV. Functionally-equivalent bacmids could have the DNA segment with the kanamycin resistance marker, the mini-attTn7 target sequence, or the bacterial replicon located elsewhere in the viral genome, in the same or opposite orientation, or all together as one large segment, but in a different order or the same or opposite orientations to each other compared to the order and orientations in bMON14272 and bMON14271.

If these functionally distinct genetic elements are abbreviated as K, L, and F, they could be assembled six congruous segments in the order KLF, KFL, LFK, LKF, FKL, and FLK. The relative orientation each segment may also be flipped, such that the K element could be in one orientation in the order K(+)LF or the opposite orientation as K(−)LF, and so on. In other cases, the K element could be on a segment that is inserted into the AcNPV genome away from a site where the L and F elements are located, or L separated from K and F, or F separated from K and L, or K, L, and F, located at 3 distinct locations in the shuttle vector.

The locations for insertion of functionally distinct genetic elements should be stable, and not prone to loss when the bacterial plasmid, or shuttle vector, are propagated in host cells over time. Inserted segments may be unstable, and prone to deletion by recombining with homologous segments in flanking regions, or somehow toxic to host cells comprising the engineered vector compared to a parental vector.

Rational designs for inserting drug resistance markers, synthetic target sites, and replicons in shuttle vectors rely heavily on existing knowledge concerning whether other genes in the vector are essential or non-essential for growth under specific growth conditions. For AcNPV, a wide variety of genes have been identified as non-essential, by creating shuttle vectors that propagated in bacteria, that were subjected to mutagenesis and then transformed into cultured insect cells for testing. If testing needs to be carried out in an infected caterpillar, then structural proteins needed to produce the occluded form would also be considered essential, even though they are not essential for production of the budded virus that infects cells within a caterpillar, and in cultured cells. A non-essential gene, or clusters of several contiguous non-essential genes may be good locations for inserting a drug resistance marker, synthetic target site, or a replicon in a redesigned shuttle vector.

Semi-rational or random methods for inserting drug resistance markers, synthetic target sites, and other replicons can also be used to introduce genetic elements into a prokaryotic and eukaryotic viral or non-viral shuttle vectors. Simpler methods may rely on linearization of a circular vector and ligation of DNA segment comprising the genetic element of interest, and transformation of the ligated product into bacteria or eukaryotic host cells for propagation and analysis. It may be desirable, in some cases though, to use a transposon that can randomly insert its cargo in another vector or a bacterial chromosome, such as variant forms of Tn5, in vitro using purified proteins, or in cells harboring vectors that encode a modified transposase [Reznikoff, W. S. (2008) Ann. Rev. Genetics 42(1): 269-286].

### Example 14—Design and Assembly of Synthetic Tn7-Like Donor/Helper/Target Vector Systems Based on Transposable Elements Observed in Genomic Islands

A wide variety of site-specific bacterial transposons have been observed in epidemiological studies and bioinformatics studies, where Tn7-like elements that confer resistance to many antibiotics, or carry genes involved in reduction of heavy metals (including gold, silver, mercury, cobalt, and bismuth) are clustered in specific locations, called genomic islands, within a host cell [Peters (2017)]. Many of these elements often comprise genes that are highly similar to the Tn7 tnsABC genes, and a homologue of tnsD called tniQ, that facilitates targeting into specific target sites, that are not similar to the sequence at the 3′ end of the essential and highly conserved E. coli glmS gene. Some of the targets for Tn7-like elements are within non-essential genes. TnAbaR1, for example, inserts in the middle of the comM-like genes in many kinds of bacteria. Representative examples from several other kinds of Tn7-like elements and their target sites are summarized in the Table below.

TABLE 27
Targets for Tn7 and Tn7-like Genetic Elements Associated with Specific Sites or Genomic Islands

| | | | | | Donor/ | |
|---|---|---|---|---|---|---|
| | | Target | | | Helper/Target | |
| Transposon | Host Cell | Gene | Essential? | Gene Function | Vector System? | Reference |
| Tn7 | Escherichia | glmS | Yes | Glutamine-fructose-6- | Yes | Craig (1996); |
| | coli | | | phosphate aminotransferase | | Peters (2014) |
| | | | | (isomerizing), with identical or | | |
| | | | | highly similar homologues in a | | |
| | | | | wide variety of prokaryotic | | |
| | | | | and eukaryotic cells | | |
| TnAbaR1 | Acinetobacter | comM | No | Hexameric helicase capable of | No | Nero (2017) |
| | baumannii | | | binding ssDNA and dsDNA in | | |
| | | | | the presence of ATP, which | | |
| | | | | appears to be a Mg chelatase- | | |
| | | | | like protein comprising an | | |
| | | | | ATPase domain | | |
| Tn6022 | Escherichia | yifB | No? | Mg chelatase subunit D/I | No | Peters (2017) |
| | coli | | | family having ATP-dependent | | |
| | | | | peptidase activity and a | | |
| | | | | member of the comM | | |
| | | | | subfamily | | |
| Tn6230 | | yhiN | No | Putative FAD/NAD(P) binding | No | Peters (2017) |
| | | | | oxidoreductase | | |
| #2 | | yciA | ? | Acyl-CoA thioester hydrolase | No | Peters (2017) |
| #141 | | IMPDH | ? | Inosine-5′-monophosphate | No | Peters (2017) |
| | | | | dehydrogenase | | |
| #298 | | SRP-RNA | ? | Signal recognition particle | No | Peters (2017) |
| | | | | RNA | | |

Several genes that are commonly associated with genomic islands targeted by Tn7-like elements have not been extensively characterized (comM, yifB, yhiN, yciA, IMPDH, and SRP-RNA). Sequences flanking and including sites for insertion in these genes, the left and right arms of these elements, and their transposase genes, can be characterized and developed into comparable donor/helper/target vector systems comprising synthetic transposons for use in a wide variety of applications requiring efficient and reproducible methods for site-specific or random insertions of one or more DNA segments into genetic material within a host cell.

A mini-TnAbaR1 donor vector is constructed by analyzing the sequences of the entire element, and inserting synthetic DNA sequences into a cloning vector such as pTwist-Amp-HC, that comprise the left and right arms of the Tn7-like element plus short sequences flanking it, with a central core cargo region comprising a DNA segment containing one or more genes of interest and/or optionally one or more multiple cloning sites (MCSs) to facilitate insertion of genetic elements derived from other vectors.

A helper mini-TnAbaR1 donor vector is constructed by cloning transposase genes into a vector having a similar replicon as the donor vector, that encodes a gene conferring resistance to a different antibiotic, such as tetracycline, comparable to the pBR322-based pMON7124 vector used in the baculovirus shuttle vector system.

A target vector comprising an attachment site for TnAbaR1 is constructed by synthesizing and cloning segments of the comM gene into a vector such as pTwist-Chlor-MC or pTwist-Kan-MC comprising a gene fusion allowing screening or selection of transposition events, such as those noted above, in Examples 1-7 of the application. One commonly observed insertion site for TnAbaR1 is near the center of the comM gene, such that the ends of the transposon are duplicated as 5-bp sequences after transposition. A 150 bp sequence spanning the insertion site is synthesized and cloned in frame with sequences near the 5′ end of the lacZalpha gene, in a fashion that is similar to the sequences used in the bMON14272 vector disclosed in Example 1, or in smaller versions disclosed in Example 3 of this application.

Transposition experiments can be carried out using donor/helper/target vectors comprising sequences derived from TnAbaR1, and analyzed by comparing the phenotype of bacteria harboring the vectors before and after transposition on agar plates containing antibiotics or chromogenic substrates, and analyzing the structure of target vectors before transposition and a composite vector after transposition.

The length of the sequence spanning the insertion site can be minimized in smaller variant forms of the target vector, and this segment can also be moved into gene fusions derived from truncated cat or NPT-II genes, to generate vectors that can be used in experiments where direct selection of transposition events by synthetic TnAbaR1 elements is allowed.

Comparable donor/helper/target vectors can be designed and assembled from other Tn7-like elements, including those noted in the table above, such as Tn6022, Tn6230, #2, #141, and #298 that target the yifB, yhiN, yciA, IMPDH, and SRP-RNA genes, respectively.

### Example 15—Design and Combinatorial Assembly of Ordered Arrays of Two or More Synthetic Attachment Sites for Site-Specific Transposons Allowing Creation of Ordered Composite Arrays Comprising Transposons Inserted into Stable Locations on Modular Prokaryotic and Eukaryotic Vectors

A target vector comprising a nucleotide sequence comprising an attachment site for a site-specific transposon can be combined with sequences derived from a second target vector to facilitate the construction of a target vector comprising an array of two or more attachment sites by any of a variety of gene assembly methods, including those characterized as being encompassed by traditional sequential methods of cloning, BioBrick assembly, Three Antibiotic (3A) Assembly, Gibson Assembly, In-Fusion™ PCR Cloning, Golden Gate Assembly, Iterative Capped Assembly, TOPO-TA Cloning, and Overlap Extension PCR methods, which are all described above, in the section entitled “Background of the Invention”.

A bacterial cell harboring a target vector comprising two distinct attachment sites may be used in transposition experiments facilitated a helper vector and a donor vector by to allow for the selection or screening of transposition events depending on the nature of the nucleotide sequences comprising gene fusions where one portion encodes a polypeptide that confers a selectable or screenable phenotype to a cell and another portion comprises a sequence derived from the attachment site for the transposon and optionally encodes polypeptide sequences fused within or to one or two portions of the polypeptide that confers the selectable or screenable phenotype to the cell.

For example, a target vector may comprise a nucleotide sequence encoding a lacZalpha polypeptide that also comprises sequences derived from the E. coli glmS gene fused in frame in the same or opposite orientation as the 3′ end of the natural glmS gene, provided that there are no stop codons in the same reading frame as the lacZalpha polypeptide, such as one of the sequences disclosed in Example 1 of the application, noted above, where an synthetic EcoRI-SalI sequence comprising the attachment site is inserted in frame between codons 5 and 7 of the lacZalpha polypeptide. A second target sequence may be derived from a gene fusion encoding an inactive cat gene fused to a mini-attTn7 sequence, such as one of the sequences disclosed in Example 2, that can be included in a contiguous array of two or more target sites, or in a separate, distinct location on the target vector between or among other key genetic elements, such as a drug resistance marker and a replicon sequence.

Transposition experiments can then be carried out, to select or screen for a first insertion into the first target site, or into the second target site, and a second experiment to select or screen for a second insertion into the remaining open target site, and confirming by phenotype and by structural analysis of that the “composite” array comprises two transposons inserted into two sites in an orientation specific manner, and that the entire array is stable, at least, in a recombination-deficient host cell strain, such as a recA minus E. coli strain. Direct repeats of sequences derived from the transposon, or from the target sequences may contribute to instability of the array in host cell strains that promote or allow homologous recombination to occur, particularly if the growth rate of cells harboring deletion variants of the composite target vector is greater than the growth rate for cells harboring a full length version of the composite target vector.

Tn7 and several but not all Tn7-like genetic elements have a property called “transpositional target immunity” where only one Tn7 element is inserted at a target site, and subsequent insertions by the same element at the target site do not occur [Stellwagen, A. E and Craig, N. L. (1997) Genetics 145(3): 573-85]. Two proteins, TnsB and TnsC, bind to the ends of Tn7 on a donor segment and target sequences comprising the ends of Tn7, preventing Tn7 elements from inserting adjacent to itself in the chromosome or in vectors comprising its attachment site.

FIG. 11 sets forth an illustration entitled “Designing and assembling arrays of synthetic targets for site-specific transposons” comparing insertion of Tn7 into a synthetic target site derived from the essential E. coli glmS gene, with cloning and targeting a sequence derived from the Acinetobacter baumannii comM gene that can be used to monitor transposition of TnAbaR1 or related Tn7-like elements using a vector comprising a target sequence encoding an active or inactive fusion protein.

FIG. 12 sets forth an illustration entitled “Creating composite arrays comprising targets for different site-specific transposons” which shows methods for building an array of different kinds of gene fusions that allows for selection or screening of cells comprising composite vectors with sequences derived from several site-specific transposons.

FIG. 13 sets forth an illustration entitled “Assembling arrays of genetic elements comprising targets for different site-specific transposons” shows how target vectors comprising several two to three fusions can be assembled from parent vectors comprising one or two gene fusions by traditional cloning methods.

FIG. 14 sets forth an illustration entitled “Combinatorial assembly of composite vectors or host cell chromosomes comprising target sites for several site-specific transposons” shows how a cell harboring a target vector comprising 3 target sites, or a host cell comprising a target vector with 2 target sites, and a target site on the chromosome can be used to analyze the function of complex sets of genes within a cell.

### Example 16—Directed Evolution of Site-Specific Transposons to Create Synthetic Transposons Having Enhanced Transposition Frequency or Altered Site Specificity

Methods for the directed evolution of a gene typically rely on three steps: (1) subjecting a gene to iterative rounds of mutagenesis creating a library of variants; (2) selection and isolation of cells harboring vectors comprising genes expressing variant products having the desired function or phenotype, and (3) amplifying vectors comprising sequences encoding the best variants for use in subsequent rounds of mutagenesis and selection. These steps can be performed in vivo, or in vitro, to recover variants that may be structurally and functionally different than those obtained by rationally designing and testing the phenotypes of cells harboring one or more modified genes.

The ability to directly select for transposition events, regardless of the nature or size of the cargo sequences carried on a mini-transposon, allows the use of methods for the directed evolution of components of a donor/helper/target vector-based transposition system, to alter the efficiency of transposition (increasing observed level of transposition in the presence of one or more variant products of the transposase genes, compared to results obtained with gene products encoded by unaltered, wild-type or parental genes), or alter the specificity of transposition (allowing the donor segment to insert at one or more specific or even random sites, compared to an assay system where all of the key components are identical or functionally similar to their wild-type counterparts.

A variety of components in a Tn7-based transposition system are suitable as targets for mutagenesis that can be carried out in the course of a series of directed evolution experiments to alter the efficiency or specificity of transposition events, are noted in the following table.

Table 28
Strategies to Alter the Site-Specificity or Efficiency of Transposition of Synthetic Tn7-Like Elements*

| | TnsA | TnsB | TnsC | TnsD | TnsE | Tn7L and Tn7R |
|---|---|---|---|---|---|---|
| Size (aa or bp) | 273 aa | 702 aa | 555 aa | 508 aa | 538 aa | ~150 and ~90 bp |
| Functions | Binds to | Binds to and | Interacts with the | Binds to attTn7 at | Binding to 3′ | Tn7L has an 8-bp DR |
| | and cuts | cuts at the 3′ | product the tnsD | the 3′ end of the | recessed ends | with a 5′ TGT, and |
| | 5-bp from | ends of Tn7L | gene bound to | E. coli glmS gene | of a replicating | Tn7R has an 8-bp DR |
| | the 5′ | and Tn7R, | structural features of | and insertion | DNA structure | with a 3′ ACA; Tn7L |
| | ends of | allowing | target DNA | occurs 24 bp | and a sliding | typically ~150 bp and 3 |
| | Tn7L and | them to be | sequences, and the | beyond the 3′ end | clamp | TnsB binding sites, and |
| | Tn7R, and | paired in a | DNA-bound complex | producing | processivity | Tn7R typically 90 bp |
| | binds to | process | of tnsA and tnsB gene | structure with 5-bp | factor (β-clamp | with 4 overlapping |
| | the | mediated by | products, with a | duplications at | protein), | tnsB binding sites; |
| | product of | the product | central domain | Tn7L and Tn7R. | encoded by the | Both ends are bound |
| | the tnsB | of the tnsA | involved with binding | | host dnaN | or cleaved by the |
| | gene. | gene. | and hydrolysis of ATP | | gene. | products of the tnsA |
| | | | and target immunity, | | | and B genes; Promoter |
| | | | preventing | | | driving expression of |
| | | | transposition into | | | all of the tnsABCDE |
| | | | segments of DNA | | | genes is near the 3′ |
| | | | comprising Tn7. | | | end of Tn7R. |
| Key Role in | | | Random | 3′ end of the E. coli | Random | |
| Targeting | | | | glmS gene and | sequences near | |
| | | | | highly conserved | the replication | |
| | | | | homologues in | fork in conjugal | |
| | | | | other bacteria and | plasmids | |
| | | | | many eukaryotic | | |
| | | | | cells | | |
| Key Variants | | | “Gain of Function” | | | Lengths of Tn7L and |
| | | | TnsC* mutants | | | Tn7R can be |
| | | | identified by | | | minimized, and some |
| | | | Stellwagen and Craig | | | nt residues can be |
| | | | (1997) transpose | | | altered without |
| | | | randomly in the | | | affecting ability of the |
| | | | presence of TnsA, | | | donor segment to |
| | | | TnsB, and TnsC*. | | | transpose. |
| Opportunities | | | New TnsC “Gain of | Variants of TnsD | | These and other types |
| to exploit | | | Function” variants | selected through | | of alterations may |
| through | | | may have higher | directed evolution | | allow transposition of |
| directed | | | efficiencies of | methods should | | Tn7-like elements with |
| evolution to | | | random transposition | allow transposition | | altered sequences |
| produce | | | of Tn7 variants in | to altered target | | within or adjacent to |
| synthetic | | | prokaryotic and | sites, including | | their 5′ and 3′ ends for |
| transposons | | | eukaryotic cells. | wild-type and | | specific applications |
| | | | | variant | | |
| | | | | homologues of the | | |
| | | | | E. coli glmS gene in | | |
| | | | | other prokaryotic | | |
| | | | | and eukaryotic | | |
| | | | | cells. | | |

*[Portions adapted from general reviews on Tn7 by Craig (1997), Peters (2014), and this work (2020)].

The ability to directly select for transposition events based on the use of novel gene fusions, such as the cat-attTn7 or NPT-II-attTn7 sequences disclosed in Examples 2 and 4, plus others noted above, allow for the selection and recovery of vectors comprising sequences encoding variants of tnsD, that should have an altered specificity compared to the wild-type attTn7 target sequence near the 3′ end of the E. coli glmS gene.

In a traditional Tn7-based donor/helper/target vector system, all of the genes encoding transposases, tnsABCD, are located on a helper vector, such as pMON7124, that is on a high copy number bacterial replicon that confers resistance to tetracycline and incompatible with the donor vector, such as pFastBac1, that is on a high copy number replicon that confers resistance to ampicillin from a gene located on the backbone of the vector, and resistance to gentamycin that is located in a gene within the mini-Tn7 element along with other sequences allowing insertion of a gene of interest downstream from an operably-linked polyhedrin promoter that is functional in the baculovirus-infected host cells. Transposition occurs when the donor plasmid is introduced into an E. coli cell harboring the target vector, bMON14272, and the helper vector, and screening for white colonies in a background of blue colonies, on indicator plates comprising the chromogenic substrate, X-gal.

In Examples 2 and 4, the target vector comprises a gene fusion, where the 5′ portion of the chimeric gene encodes an inactivated drug resistance gene, linked to a mini-attTn7 sequence that partially overlaps with codons near the 3′ end of the gene, such as those encoding a Cysteine residue for the cat gene, or a Proline residue for the NPT-II gene. Transposition of a mini-Tn7 element from the donor vector, in the presence of a helper vector should occur, and all of the vectors that are recovered when the chloramphenicol or kanamycin are used in the selection plates, in addition to antibiotics conferring resistance to the gene on the backbone of the vector, should be composite vectors, each having an insertion of the mini-Tn7 element into the target site in the novel gene fusion sequence.

In one of many possible schemes for performing directed evolution of transposase genes, the gene encoding tnsD, is moved from the helper vector, to the target vector, and placed under the control of an inducible promoter. The target vector comprising selectable gene fusion (such as those disclosed in Examples 2 and 4) is altered to comprise a desired sequence, such as a human or yeast homologue of the E. coli glmS attachment site, and the tnsD gene is then mutagenized by a random or a site-specific method, so that all or parts of its coding sequences are altered, primarily by single or multiple nucleotide base substitutions, and then transformed into a host cell comprising the helper vector comprising the tnsABC genes and a donor vector. Cells harboring the modified target vector can also be co-transformed with a helper vector comprising the tnsABC genes and a donor vector. The transformed cells are plated on the antibiotic that is restored after transposition of the mini-transposon into the gene fusion, and cells comprising composite vectors are characterized by their cellular phenotype, and the vectors characterized by structural analysis, such as DNA sequencing across the ends of the transposon, the sizes of fragments amplified fragments, or by the sizes of fragments cleaved by one or more restriction enzymes.

Since the target vector also contains the mutagenized tnsD gene, selecting for restoration of drug resistance should recover bacteria harboring vectors that encode transposase variant gene products that bind to the altered binding site associated with its corresponding insertion site. If the target sequence in the gene fusion is different than the wild-type E. coli glmS gene, it should be possible to recover target vectors with the one or more altered tnsD genes. The variants can be used in subsequent rounds of directed evolution experiments, to recover variants that allow the mini-Tn7 element to be inserted into human, yeast, or other target sites that are substantially different from the wild-type E. coli glmS gene.

It should also be possible to recover variants where the altered target sequence does not naturally occur in any prokaryotic or eukaryotic host cell system, which would permit its transfer and use in a wide variety of vector and host cell systems, dramatically transforming many fields of synthetic biology, including those directed to the discovery and development of novel food and drug products, and components of cell and gene therapy vector systems.

Similar approaches can also be used to mutagenize and recover vectors comprising other altered transposase genes, which transpose more frequently or efficiently into their natural specific target sites (hyper-transposase mutants)), much different perhaps, than tnsC* variants that have 100× the activity of the wild-type gene, efficiently promoting random transposition of a mini-Tn7 donor element into a vector or into chromosome of E. coli [Stellwagen, A. E and Craig, N. L. (1997) Genetics 145(3): 573-85].

Both approaches can also be combined to build a set of donor/helper/target vectors that increase the level of site-specific transposition events, where the helper vector comprises one or more variant tnsA, B, C, and D genes, that encode products that act on the ends of Tn7 in the donor vector, to facilitate its efficient insertion into a specific sequence on a target vector or target sequence integrated into the chromosome of a host cell.

FIG. 15 sets forth an illustration entitled “Directed evolution to develop synthetic transposons with altered target site-specificity” that shows basic features of a set of donor/helper/target vectors to facilitate the mutagenesis and selection of transposase genes that have altered specificities or enhanced levels of transposition compared to the wild-type transposase genes, or have altered arms of the transposon to comprise restriction sites or stop codons for specific applications.

FIG. 16 sets forth an illustration entitled “Directed evolution of tnsD gene product to bind to homologues of E. coli glmS and other target sites” showing a system where the tnsD gene is deleted from the helper vector and mutagenized versions of that gene included in a library of altered target vectors, which allow for selection of cells harboring composite vectors with insertions into target sequences that might not otherwise be recoverable using wild-type transposase genes. Target sequences of interest include homologues found in mammalian cells, such as human, non-human primate, bovine, mouse, and rat sequences, plus fungal homologues found in filamentous and non-filamentous fungi, including yeast.

### Example 17—Design and Assembly of Synthetic Site-Specific Bacterial Transposons that Work Efficiently in Eukaryotic Cells

Major features of the design and assembly of novel vectors and methods for the selection or screening of transposition events carried out with vectors propagated in prokaryotic cells, can be carried over into the development of site-specific transposition systems that work well in eukaryotic cells, where the target sequence is propagated in a shuttle vector, or is integrated into a host cell chromosome that would provide great flexibility for use in many types of cell engineering applications.

Compatible sets of vectors are designed and assembled to take into account factors relating to expression of heterologous genes of interest in different types of host cell systems, including (a) construction of new helper vectors comprising 3-4 codon-optimized genes encoding transposases operably-linked to eukaryotic promoters and termination signals that function in the desired host cell; (b) isolation and characterization of mutant transposases genes that increase overall levels of transposition or alter the specificity towards particular target sites; and (c) demonstration that donor, helper, and target vectors lead to the introduction of a single donor transposon at a specific target site at a stable location on a vector or the host chromosome, or in other circumstances, multiple random insertions into the chromosome, without the potential for or evidence of remobilization.

Helper vectors that encode transposase genes optimized for expression in mammalian cells are constructed by cloning codon-optimized variants of the tnsABCD genes including any tnsD variants that target the E. coli glmS sequence or the human homologue of this sequence, and placed under the control of a strong, perhaps inducible promoter that functions in mammalian cells. Human CMV and HSV Thymidine kinase promoters are commonly used now for a wide variety of applications. A mammalian cell comprising the target vector, or an engineered cell comprising the target sequences integrated into its genome is transformed with the variant helper vector and a donor vector, selecting for resistance to the gene that is reactivated by transposition in the synthetic attTn7 gene fusion.

Synthetic site specific transposons that work well in plant cells can be based on many of the vectors derived from the TI plasmid, and shuttle vectors comprising major parts of the chloroplast genome. Helper vectors comprising transposase genes operably-linked to bacterial or plant host cell promoters are designed and assembled, using the approaches noted above, and used with donor and target shuttle vectors modified appropriately to reflect codon preferences and regulatory signals that are known to function in the host cell. Transposition experiments are carried out with appropriately modified donor and helper vectors, followed by analysis of the phenotype of bacteria harboring the composite vectors and the structures of the composite vectors. The composite vectors are then transferred to plant cells or tissues, and expression of the products encoded in the donor cassette is evaluated. Comparable systems that work well for vectors propagated in Agrobacterium, Xanthomonas, or other phytobacteria can also be developed.

Similar approaches can be used to develop site-specific transposons based on Tn7-like elements that work well in non-enteric bacteria, or fungi (unicellular yeast, or filamentous fungi) can also be developed. Target sequences that work well in other host cell systems can be moved into shuttle vectors propagated in these types of host cells, or directly into the chromosome of a host cell. Helper vectors comprising codon-optimized transposase genes that facilitate insertion of a mini-Tn7-like transposon into the target site are used, including those that encode variants that may target a wild-type of variant form of an attachment sequence within the host cell. A variant form of a helper vector developed through directed evolution techniques, can be used to target the yeast homologue of the E. coli glmS gene, allowing perhaps, targeted insertions of DNA segments into a single, safe location within a yeast cell.

Eukaryotic gene delivery systems based on synthetic site-specific prokaryotic transposons can be a powerful tool to transform many fields of synthetic biology, leading to the discovery and development of many novel food and drug products, and efficient, cost-effective methods for the production of many other products in cultured cells and transgenic organisms.

### Example 18—Design of Modular Target Sites to Assay the Efficiency and Fidelity of Gene Editing Events, Including One or More Combinations of Nucleotide Substitution, Insertion, and Deletion Events

There are two types of DNA substitutions. Transitions involve substitutions of purines comprising two aromatic rings (A↔G), or substitutions of pyrimidines comprising one aromatic ring (C↔T). Transitions involve substitutions of structures comprising one ring with one comprising two rings, and substitutions of structures comprising two rings with one comprising one ring (C↔A, C↔G, T↔A, T↔G). There are four types of transition events: A to G, G to A, C to T, and T to C. There are eight types of transversion events: C to A, A to C, C to G, G to C, T to A, A to T, T to G, and G to T.

Small or large Insertions or deletions can alter the reading frame of a sequence encoding a protein or alter the structure of a sequence in a critical domain of an encoded polypeptide or complementary RNA molecule, generally leading to the expression of functionally impaired or inactive molecules.

Novel methods to assay the efficiency and selectivity of gene editing systems can be designed that are based on methods that alter the level or functional activity of a product encoded by gene. Bacterial plasmids and shuttle vectors comprising at least one of the novel gene fusions noted in earlier examples of this application can be used to facilitate the design of assays to test not only the insertion of transposons at a specific target site, but also the efficiency and specificity of endonuclease based complexes (e.g., CRISPR-Cas, homing enzymes, and chimeric molecules comprising recognition and editing functions) designed to edit nucleotide sequences carried on replicons or integrated into a host chromosome.

In Example 2, novel gene fusions are disclosed, where one or more TAA, TGA, or TAG stop codons are inserted upstream from the 3′ end of the cat gene encoding chloramphenicol acetyltransferase (CAT protein). Transposition of a mini-attTn7 sequence from a donor plasmid into a synthetic mini-attTn7 that is designed to have its insertion site (−2 to +2) overlap with the stop codon, will alter the reading frame of the truncated gene after transposition to generate a sequence encoding a CAT fusion protein that is extended, and active, compared to the inactive truncated CAT protein. The same vector can be used as a target for CRISPR- and other nuclease-based complexes to test their effectiveness in making alterations at the one or more stop codons, allowing expression of a functional CAT protein, restoring the ability of a cell harboring the vector to confer resistance to chloramphenicol.

A variety of nucleotide substitutions and insertions or deletions can be detected with this system, where one or more TAA, TGA, and TAG stop codons are introduced in the middle of or near the 3′ end of a gene encoding a selectable marker or a reporter molecule.

| | |
|---|---|
| TAA, to (A/C/G, not T)AA, to | 1 Transition, 6 Transversions |
| T(C/T, not A/G)A, TA (C/T, not A/G) | |
| TGA, to (A/C/G, not T)GA, to | 2 Transitions, 6 Transversions |
| T(C/T, not A/G)A, TG (C/T/G, not A) | |
| TAG, to (A/C/G, not T)AG, to | 2 Transitions, 6 Transversions |
| T(C/T, not A/G)A, TA (A/C/T, not G) | |

These methods apply not only to truncated, disrupted, or extended versions of cat genes, but also many other types of genes, including NPT-II (conferring resistance to kanamycin), bla (conferring resistance to amplicillin, tet (conferring resistance to tetracycline, and the lacZalpha gene encoding an alpha polypeptide that can bind to and complement an acceptor polypeptide to generate a functional β-galactosidase molecule, which are all disclosed in Examples 1, and 3-7 of this application.

The effectiveness of gene editing systems can be assayed by detecting the efficiency of converting stop codons in synthetic gene fusions comprising truncated versions of genes encoding a protein conferring resistance to an antibiotic or a reporter molecule. Vectors comprising gene fusions noted above, can be used in assays designed to monitor the efficiency of converting a stop codon in a gene encoding a truncated, inactive enzyme to a codon that allows translation of a normal or extended version of an active enzyme. Vectors based on pACYC184, for example, that comprise a TAA, TGA, or TAG stop codon near the 3′ end of the cat gene encoding an inactive truncated chloramphenicol acetyl transferase (CAT protein), can be used as targets for editing by complexes comprising a nuclease and a targeting protein or guide RNA, such as a CRISPR/Cas9/guide RNA-based complex in vitro, or expressed in vivo, to generate an edited gene encoding a functional CAT protein. The edited products can be transformed into a host cell selecting for resistance to tetracycline and the ratio of cells conferring resistance to chloramphenicol to those conferring resistance to tetracycline compared to determine the efficiency of the editing process.

Mutagenized versions segments of DNA encoding components of the gene editing complex can be prepared and their effectiveness compared to complexes comprising unaltered components. Genes encoding nucleases, targeting proteins, and guide RNAs can be mutagenized and rapidly identified as being beneficial or not, if they increase the efficiency of conversion of an inactive truncated enzyme to a normal or extended version of an active enzyme, such as the CAT protein.

Similar types of assays can also be developed, based on genes encoding truncated or disrupted versions of NPT-II (conferring Kanamycin resistance), beta-lactamase (conferring resistance ampicillin resistance), and the tetracycline anti-porter (conferring resistance to tetracycline), and the lacZalpha polypeptide (which can complement an acceptor polypeptide in a host cell containing lacZΔM15 gene to generate a functional β-galactosidase protein).

Assays designed to determine the efficiency of small gene deletions can also be developed, where deletion of the stop codon and one or more additional codons in a truncated or disrupted gene can be performed, allowing expression of an active enzyme.

Assays can also designed to detect deletions or insertions of 1-bp or 2-bp insertions, by using a target sequence that has or is missing several nucleotides near a stop codon in a truncated gene, creating a frameshift leading to early termination of translation, and requiring one or more compensating insertions or deletions of several nucleotides upstream or downstream from that site to allow expression of an active enzyme.

It may be desirable in some cases to include the gene of interest being mutagenized on the same vector comprising the truncated, disrupted, or extended target gene. For example, a pACYC184-based vector comprising a cat gene with a stop codon near its 3′ end can also contain a gene encoding the Tn7 tnsD gene, along with a bacterial replicon and gene conferring resistance to tetracycline. Parts of the segment of DNA encoding the tnsD gene can be altered by mutagenesis, such as inserting a synthetic oligonucleotide containing one or more substitutions compared to the wild-type sequence, and the altered plasmid transformed into a cell comprising a helper plasmid (providing the products of the tnsA, B, and C genes, and a plasmid comprising a mini-Tn7 donor element. The cells can be grown on a series of plates containing tetracycline and different concentrations of chloramphenicol. Cells that are resistant to chloramphenicol should contain a transposon inserted into the mini-attTn7 target site downstream from the altered cat gene, if the product of the tnsD gene is functional. Direct selection for colonies that are resistant chloramphenicol under these conditions should allow the analysis of genes encoding products involved in transposition, including the left and right arms of the transposon and the ability of the product of the tnsD gene to bind to the target site and bind to one or more of the products of the tnsA, B, and C genes that direct insertion of the mini-transposon into its specific target site. Similar approaches can be used to mutagenize and test the effectiveness of one or more altered tnsA, B, and C genes carried on the altered target plasmid.

Vectors designed to test the efficiency and specificity of other types of gene editing complexes do not need to include mini-attTn7 based sequences located within or flanking the target genes, simplifying the design of the test vectors to some extent. CRISPR-Cas-based complexes, for example, can be tested using vectors encoding disrupted or truncated cat, NPT-II, bla, tet or lacZalpha genes, or almost any other type of gene encoding a selectable marker or reporter molecule. Vectors comprising a gene encoding an altered Cas protein, and the truncated or altered target site can be used in a program of directed evolution to select for genes encoding products that have one or more improved activities, such as ability to recognize the target site, with lower levels of off target nucleotide substitution, insertion, or deletion activities

### Statement Regarding Specific Aspects, Various Modifications, and Alternatives, are Meant to be Illustrative and not Limiting as to the Scope of the Invention

While specific aspects of the invention have been described in detail, it will be appreciated by those skilled in the art that various modifications and alternatives to those details could be developed in light of the overall teachings of the disclosure. Accordingly, the particular arrangements disclosed are meant to be illustrative only, and not limiting as to the scope of the invention, which is to be given the full breadth of the appended claims, and any equivalent, thereof.

It is recognized that a number of variations can be made to this invention as it is currently described but which do not depart from the scope and spirit of the invention without compromising any of its advantages. These include substitution of different genetic elements (e.g., drug resistance markers, transposable elements, promoters, heterologous genes, and/or replicons, etc.) on the donor plasmid, the helper plasmid, or the shuttle vector, particularly for improving the efficiency of transposition in E. coli or for optimizing the expression of the heterologous gene in the host cell. The helper functions or the donor cassette might also be moved to the attTn7 on the chromosome to improve the efficiency of transposition, by reducing the number of open attTn7 sites in a cell which compete as target sites for transposition in a cell harboring a shuttle vector containing an attTn7 site.

This invention is also directed to any substitution of analogous components. This includes, but is not restricted to, construction of bacterial-eukaryotic cell shuttle vectors using different eukaryotic viruses, use of bacteria other than E. coli as a host, use of replicons other than those specified to direct replication of the shuttle vector, the helper vector encoding one or more transposition genes, or the donor vector comprising the left and right arms of a transposon, each arm flanking a cargo DNA segment comprising one or more sequences of interest, use of selectable or differentiable genetic markers other than those specified, use of site-specific recombination elements other than those specified, and use of genetic elements for expression in eukaryotic cells other than those specified. It is intended that the scope of the present invention be determined by reference to the appended claims.

### BIBLIOGRAPHY

### Statement Regarding Incorporation by Reference of Journal Articles and Patent Documents

All references, patents, or applications cited herein are incorporated by reference in their entirety, as if written herein.

### PATENT DOCUMENTS

1. U.S. Pat. No. 5,348,886, issued 1994 Sep. 20, expired 2012-09-20, assigned to Monsanto Company.

### Journal Articles

1. Adrian W. Briggs, Xavier Rios, Raj Chari, Luhan Yang, Feng Zhang, Prashant Mali and George M. Church (2012) Iterative capped assembly: rapid and scalable synthesis of repeat-module DNA such as TAL effectors from individual monomers. Nucleic Acids Research, 2012, Vol. 40, No. 15 e117 doi:10.1093/nar/gks624].
2. Anderson, D., Harris, R., Polayes, D., Ciccarone, V., Donahue, R., Gerard, G., and Jessee, J. (1996) Rapid Generation of Recombinant Baculoviruses and Expression of Foreign Genes Using the Bac-To-Bac® Baculovirus Expression System. Focus 17, 53-58
3. Ausubel, F. M., Brent, R., Kingston, R. E., Moore, D. D., Seidman, J. G., Smith, J. A., and Struhl, K. (1994) Current Protocols in Molecular Biology, Greene Publishing Associates and Wiley-Interscience, New York
4. Ausubel, F. M., R. Brent, R. E. Kingston, D. D. Moore, J. G. Seidman, J. A. Smith, K. Struhl, P. Wang-Iverson, and S. G. Bonitz (ed.). 1989. Short Protocols in Molecular Biology: A Compendium of Methods from Current Protocols in Molecular Biology, p. 1-387. Greene Publishing Associates and Wiley-Interscience, New York.
5. Axe, D. D. (2000) Extreme functional sensitivity to conservative amino acid changes on enzyme exteriors. J. Mol. Biol. 301: 585-695.
6. Barany, F (1985) Two-codon insertion mutagenesis of plasmid genes by using single stranded hexameric oligonucleotides. Proc. Natl. Acad. Sci. USA 82: 4202-4206.
7. Barry, G. F. (1988) A Broad Host-Range Shuttle System for Gene Insertion into the Chromosomes of Gram-negative Bacteria. Gene 71: 75-84
8. Barry, G. F. 1986. Permanent insertion of foreign genes into the chromosomes of soil bacteria. Bio/Technology 4:446-449.
9. Barth P T, Datta N, Hedges R W, Grinter N J. (1976) Transposition of a deoxyribonucleic acid sequence encoding trimethoprim and streptomycin resistances from R483 to other replicons. J Bacteriol 25:800-10. [PubMed: 767328]
10. Bird, L. E., Rada, H., Flanagan, J., Diprose, J. M., Gilbert, R. J. C. and Owens, R. J. (2014). Application of In-Fusion™ cloning for the parallel construction of E. coli expression vectors. Methods Mol. Biol. Clifton N. J. 1116: 209-234;
11. Bochner, B. R., H. Huang, G. L. Schieven, and B. N. Ames. (1980) Positive selection for loss of tetracycline resistance. J. Bacteriol. 143:926-933.
12. Bryksin A. M. I., “Overlap extension PCR cloning: a simple and reliable way to create recombinant plasmids.” Biotechniques, 29(6): 997-1003, 2012]
13. C. Engler, R. Kandzia, and S. Marillonnet, “A one pot, one step, precision cloning method with high throughput capability.,” PLoS One, 3(11): p. e3647, January 2008.]
14. Carrington, J. C., and Dougherty, W. G. (1988) A Viral Cleavage Site Cassette: Identification of Amino Acid Sequences Required for Tobacco Etch Virus Polyprotein Processing. Proc. Natl. Acad. Sci. USA 85: 3391-3395.
15. Choi, K.-H. and Kim, K.-J. (2009) Applications of Transposon-Based Gene Delivery System in Bacteria. J. Microbiol. Biotechnol. 19(3): 217-228; doi: 10.4014/jmb.0811.669; First published online 23 Jan. 2009.
16. Ciccarone, V. C., Polayes, D., and Luckow, V. A. (1997) Generation of Recombinant Baculovirus DNA in E. coli Using Baculovirus Shuttle Vector. Methods in Molecular Medicine (Reischt, U., Ed.), 13, Humana Press Inc., Totowa, N.J.
17. Cole, C. N., and Stacy, T. P. (1985) Identification of Sequences in the Herpes Simplex Virus Thymidine Kinase Gene Required for Efficient Processing and Polyadenylation. Mol. Cell. Biol. 5: 2104-2113.
18. Craig, N. L. (1996) Transposition. In: Escherichia coli and Salmonella typhimurium: Cellular and Molecular Biology II (eds. Neidhardt, F. et al) American Society for Microbiology, Washington, D.C., pp. 2339-2362.
19. DeBoy, Robert T., Craig, Nancy L. (2000) Target Site Selection by Tn7:attTn7 Transcription and Target Activity. J. Bacteriol. 182(11): 3310-3313.
20. Deutscher, M. P. (ed) (1990) Guide to Protein Purification Vol. 182. Methods in Enzymology. Edited by Abelson, J. N., and Simon, M. I., Academic Press, San Diego, Calif.
21. Dougherty, W. G., Carrington, J. C., Cary, S. M., and Parks, T. D. (1988) Biochemical and Mutational Analysis of a Plant Virus Polyprotein Cleavage Site. EMBO J. 7: 1281-1287.
22. Durfee T, Nelson R, Baldwin S, Plunkett G 3rd, Burland V, Mau B, Petrosino J F, Qin X, Muzny D M, Ayele M, Gibbs R A, Csörgo B, Pósfai G, Weinstock G M, Blattner F R. (2008) The complete genome sequence of Escherichia coli DH10B: insights into the biology of a laboratory workhorse. J Bacteriol. 190(7): 2597-606. doi: 10.1128/JB.01695-07. Epub 2008 Feb. 1.
23. Fukasawa, T. and H. Nikaido. (1961) Galactose sensitive mutants of Salmonella. II. Bacteriolysis induced by galactose. Biochim. Biophys. Acta 48:470-483.
24. Gibson et al, (2008) “Complete chemical synthesis, assembly, and cloning of a Mycoplasma genitalium genome.” Science, 319:1215-1220.
25. Gibson et al, “Enzymatic assembly of DNA molecules up to several hundred kilobases.” Nat Meth, 6:343-5, 2009.
26. Gossen et al (1992) Application of galactose sensitive E. coli strains as selective hosts for LacZ-plasmids. Nucleic Acids Research 20(12): 3254.
27. Grant, S. G. N., J. Jessee, F. R. Bloom, and D. Hanahan. (1990) Differential plasmid rescue from transgenic mouse DNAs into Escherichia coli methylation restriction mutants. Proc. Natl. Acad. Sci. USA 87:4645-4669.
28. Griffith J K, Buckingham J M, Hanners J L, Hildebrand C E, Walters R A. (1982) Plasmid-conferred tetracycline resistance confers collateral cadmium sensitivity of E. coli cells. Plasmid 8: 86-88.
29. Gringauz, E. Orle, K. A., Waddell C. S., Craig N. L. (1988) Recognition of Escherichia coli attTn7 by transposon Tn7: lack of specific sequence requirements at the point of Tn7 insertion. J. Bacteriol. 170(6): 2832-2840.
30. Hall, New York, N.Y. Luckow, V. A. (1991) in Recombinant DNA Technology and Applications (Prokop, A., Bajpai, R. K., and Ho, C., eds), McGraw-Hill, New York.
31. Hamilton, C. M., M. Aldea, B. Washburn, P. Babitzke, and S. R. Kushner. 1989. New method for generating deletions and gene replacements in Escherichia coli. J. Bacteriol. 171:4617-4622.
32. Hanahan, D. (1983) Studies on Transformation of Escherichia coli with Plasmids. J. Mol. Biol. 166: 557-580.
33. Harris, R., and Polayes, D. (1997) A New Baculovirus Expression Vector for the Simultaneous Expression of Two Heterologous Proteins in the Same Insect Cell. Focus 19: 6-8.
34. Hecky, J., Muller, K. M. (2005) Structural perturbation and compensation by directed evolution at physiological temperature leads to thermostabilization of β-lactamase. Biochemistry 44: 12640-12654.
35. Hedges R W, Datta N, Fleming M P. (1972) R factors conferring resistance to trimethoprim but not sulphonamides. J. Gen. Microbiol. 73:573-5. [PubMed: 4571517].
36. Holton, T. A., Graham, M. W. (1991). A simple and efficient method for direct cloning of PCR products using ddT-tailed vectors. Nucleic Acids Research, 19(5): 1156.
37. In-Fusion® H D Cloning Kit User Manual, available from Takara Bio.
38. Janson, J. C., and Ryden, L. (1989) in Protein Purification: Principles, High Resolution Methods, and Applications, VCH Publishers, New York.
39. Juers et al (2012) LacZ β-galactosidase: Structure and function of an enzyme of historical and molecular biological importance. Protein Science 21:1792-1807.
40. Kertbundit, S., Greve, H. d., Deboeck, F., Montagu, M. V., and Hernalsteens, J. P. (1991) In vivo Random beta glucuronidase Gene Fusions in Arabidopsis thaliana. Proc. Natl. Acad. Sci. USA 88: 5212-5216.
41. King, L. A., and Possee, R. D. (1992) The Baculovirus Expression System: A Laboratory Guide, Chapman.
42. Knight, T. (2005) Idempotent Vector Design for Standard Assembly of BioBricks. MIT Synthetic Biology Working Group.
43. Levy et al (1999) Nomenclature for new tetracycline resistance determinants. Antimicrob. Agents Chemother. 43(6): 1523-1524.
44. Li, H., Yang, Y., Hong, W., Huang, M., Wu, M., and Zhao, X. (2020) Applications of genome editing technology in the targeted therapy of human diseases: mechanisms, advances and prospects. Signal Transduction and Targeted Therapy 5:1.
45. Luckow, V. A. (1991) Cloning and expression of heterologous genes in insect cells with baculovirus vectors., p. 97-152. In A. Prokop, R. K. Bajpai, and C. Ho (ed.), Recombinant DNA Technology and Applications.
46. Luckow, V. A., and M. D. Summers (1988a) Signals important for high-level expression of foreign genes in Autographa californica nuclear polyhedrosis virus expression vectors. Virology 167:56-71.
47. Luckow, V. A., and M. D. Summers (1988b) Trends in the development of baculovirus expression vectors. Bio/Technology 6:47-55.
48. Luckow, V. A., and M. D. Summers. 1989. High level expression of nonfused foreign genes with Autographa californica nuclear polyhedrosis virus expression vector. Virology 70:31-39.
49. Luckow, V. A., and Summers, M. D. (1988) Signals Important for High-Level Expression of Foreign Genes in Autographa californica Nuclear Polyhedrosis Virus Expression Vectors. Virology 167, 56-71.
50. Luckow, V. A., Lee, C. S., Barry, G. F., and Olins, P. O. (1993) Efficient Generation of Infectious Recombinant Baculoviruses by Site-Specific Transposon-Mediated Insertion of Foreign Genes into a Baculovirus Genome Propagated in Escherichia coli. J. Virol. 67: 4566-4579.
51. Lun et al (2011) Recent patents on the baculovirus systems. Recent Patents on Biotechnology 5:1-11.
52. Magota, K., Otsuji, N., Miki, T., Horiuchi, T., Tsunasawa, S., Kondo, J., Sakiyama, F., Amemura, M., Morita, T., Shinagawa, H. (1984) Nucleotide sequence of the phoS gene, the structural gene for the phosphate-binding protein of Escherichia coli. J. Bacteriol. 157(3): 909-917.
53. Maloy S R, Nunn W D. (1981) Selection for loss of tetracycline resistance by Escherichia coli. J. Bacteriol. 1981; 145:1110-1111.
54. Maniatis, T., E. F. Fritsch, and J. Sambrook (ed.). 1982. Molecular Cloning. Cold Spring Harbor, Cold Spring Harbor. McGraw-Hill, New York.
55. Matagne, A., Lamotte-Brasser, J., Frere, J.-M. (1998) Catalytic properties of Class A β-lactamases: efficiency and diversity. Biochem J. 330:581-598.
56. Mehalko, J. L., Esposito, D. (2016) Engineering the transposition-based baculovirus expression vector system for higher efficiency protein production from insect cells. J. Biotechnol. 238: 1-8.
57. Miller, J. H. 1972. Experiments in Molecular Genetics, p. 1-446. Cold Spring Harbor, Cold Spring Harbor, N.Y.
58. O'Reilly, D. R., Miller, L. K., and Luckow, V. A. (1992) Baculovirus Expression Vectors: A Laboratory Manual, W. H. Freeman and Company, New York, N.Y.
59. Parks, A. R., and Peters, J. E. (2007) Transposon Tn7 is widespread in diverse bacteria and forms genomic islands. J. Bacteriol. 189: 2170-2173.
60. Parks, A. R., and Peters, J. E. (2009) Tn7 elements: engendering diversity from chromosomes to episomes. Plasmid 61: 1-14.
61. Peters J. 2014. Tn7. Microbiol. Spectrum 2(5): MDNA3-0010-2014. doi:10.1128/microbiolspec.MDNA3-0010-2014.
62. Peters, J. E. (2014) Tn7. In Mobile DNA, 3^{rd} Edition. Craig Nancy, L., Rice, P., Lambowitz, A., Gellert, M., and Sandmeyer, S. B. (eds). Washington D. C.: ASM Press.
63. Podolsky T, Fong S T, Lee B T. (1996) Direct selection of tetracycline-sensitive Escherichia coli cells using nickel salts. Plasmid. 36:112-115.
64. Polayes, D., Harris, R., Anderson, D., and Ciccarone, V. (1996) New Baculovirus Expression Vectors for the Purification of Recombinant Proteins from Insect Cells. Focus 18, 10-13.
65. Possee et al (2019) Recent developments in the use of baculovirus expression vectors. Curr. Issues Mol. Biol. 34: 215-230.
66. Reddy (2004) Positive selection system for identification of recombinants using α-complementation plasmids. Biotechniques 37: 948-952.
67. Reiss, B., Sprengel, R. and Schaller, H. (1984) Protein fusions with the kanamycin resistance gene from transposon Tn5. EMBO J. 3(13): 3317-3322.
68. Reznikoff, W. S. (2008) Transposon Tn5. Ann. Rev. Genetics 42(1): 269-286.
69. Robben, J. Van der Schueren, J., and Volckaert G. (1993) Carboxyl terminus is essential for intracellular folding of chloramphenicol acetyltransferase. J. Biol, Chem. 268(33): 24555-24558.
70. Rohrmann, G. F. (2019) Baculovirus Molecular Biology [Internet]. 4th edition. Bethesda (Md.): National Center for Biotechnology Information (US); NBK543458.
71. Rose, R. E. (1988) The nucleotide sequence of pACYC184. Nucleic Acids. Res. 16: 355.
72. Roy, P. and Noad R. (2012) Use of bacterial artificial chromosomes in baculovirus research and recombinant protein expression: Current trends and future perspectives. ISRN Microbiology Article ID 628797, 11 pages.
73. Rubin and Levy (1991) J. Bacteriol. 173(14): 4503-4509].
74. Rubin, R. A. and Levy, S. B. (1990) J. Bacteriol. 172: 2303-2312]
75. Sambrook, J., Fritsch, E. F., and Maniatis, T. (1989) Molecular Cloning: A Laboratory Manual, Second Ed., Cold Spring Harbor Laboratory Press, Plainview, N.Y.
76. Saraceni-Richards and Levy (2000) Evidence for interactions between helices 5 and 8 and a role for interdomain loop in tetracycline resistance mediated by hybrid Tet proteins. J. Biol. Chem. 275(9): 6101-6106
77. Sigma Aldrich (2015) Topoisomerase I from Vaccinia Virus. Datasheet.
78. Skipper, K. A., Andersen, P. R., Sharma, N., and Mikkelsen, J. G. (2013) DNA transposition-based gene vehicles-scenes from an evolutionary drive. J. Biomedical Sci. 20(1): 92.
79. Stellwagen, A. E and Craig, N. L. (1997) Gain-of-function mutations in TnsC, an ATP-dependent transposition protein that activates the bacterial transposon Tn7. Genetics 145(3): 573-85.
80. Thermo Fisher (2015) TOPO Cloning Technology Brochure.
81. Urban, A. A. (1997) rapid and efficient method for site-directed mutagenesis using one-step overlap extension PCR. Nucleic Acids Res. 25(11): 2227-2228.
82. Van der Schueren, J., Robben, J. and Volckaert, G. (1998) Misfolding of chloramphenicol acetyl transferase due to carboxy-terminal truncation can be corrected by second site mutations. Protein Engineering 11(12): 1211-1217.
83. Walker, J. E., N. J. Gay, M. Saraste, and A. N. Eberle. (1984) DNA sequence around the Escherichia coli unc operon. Completion of the sequence of a 17 kilobase segment containing asnA, oriC, unc, glmS and phoS. Biochem. J. 224:799-815.
84. Waters et al (1983) The tetracycline resistance determinants of RP1 and Tn1721: nucleotide sequence analysis. Nucleic Acids Res. 11: 6089-6105.
85. Westwood, J. A., Jones, I. M., and Bishop, D. H. L. (1993) Analyses of Alternative Poly(A) Signals for Use in Baculovirus Expression Vectors. Virology 195: 90-93.
86. Wright and Tate (2015) Isolation and characterization of transport-defective substrate-binding mutants of the tetracycline antiporter TetA(B). Biochimica et Biophysica Acta 1848: 2261-2270.
87. Yao X-J, G P Kobinger, S Dandache, N Rougeau, E A Cohen (1999) HIV-1 Vpr-chloramphenicol acetyltransferase fusion proteins: sequence requirement for virion incorporation and analysis of antiviral effect. Gene Therapy 6: 1590-1599.
88. Zhu, B., Cai, G., Hall, E. O. and Freeman, G. J. (2007). In-fusion assembly: seamless engineering of multidomain fusion proteins, modular vectors, and mutations. BioTechniques 43: 354-359.

What is claimed is:

1. A nucleotide sequence comprising a target site for a site-specific transposon, wherein said target site comprises a target sequence comprising a transcriptionally or translationally fused marker sequence encoding a selectable marker sequence or a screenable marker sequence operably-linked to a sequence comprising a specific target sequence for recognition and insertion of a site-specific transposon, wherein said fused marker sequence encodes an inactive or an active polypeptide capable of conferring a selectable or screenable phenotype upon a cell comprising the fused marker sequence, wherein insertion of the site-specific transposon into the target sequence to create a composite target sequence changes the phenotype of a cell comprising the composite screenable or selectable marker sequence compared to a cell comprising just the selectable or screenable marker sequence.

2. The nucleotide sequence of claim 1, wherein said target site comprises a target sequence for a site-specific transposon comprising a translationally-fused selectable marker sequence or a screenable marker sequence operably-linked to a sequence comprising a specific target sequence for recognition and insertion of a site-specific transposon, wherein said fused marker sequence encodes an inactive or an active polypeptide capable of conferring a selectable or screenable phenotype upon a cell comprising the fused marker sequence, wherein insertion of the site-specific transposon into the target sequence to create a composite target sequence changes the phenotype of a cell comprising the composite screenable or selectable marker sequence compared to a cell comprising just the selectable or screenable marker sequence.

3. The nucleotide sequence of claim 2, wherein said sequence comprises a target site for a site-specific transposon comprising a translationally-fused selectable marker sequence operably-linked to a sequence comprising a specific target sequence for recognition and insertion of a site-specific transposon, wherein said fused marker sequence encodes an inactive polypeptide capable of conferring a selectable phenotype upon a cell comprising the fused marker sequence, wherein insertion of the site-specific transposon into the target sequence to create a composite target sequence changes the phenotype of a cell comprising the composite selectable marker sequence compared to a cell comprising just the selectable marker sequence.

4. The sequence of claim 3, wherein said wherein said fused marker sequence encodes a truncated or extended inactive polypeptide which is extended or truncated, respectively, after transposition to form a composite target sequence which encodes an active polypeptide conferring a selectable phenotype upon the cell.

5. The nucleotide sequence of claim 3, wherein said fused marker sequence encodes a truncated, inactive polypeptide which is extended after transposition to form a composite target sequence which encodes an active polypeptide conferring a selectable phenotype upon the cell.

6. The nucleotide sequence of claim 5, wherein the selectable marker sequence encodes an inactive bacterial chloramphenicol acetyl transferase (CAT) fusion protein.

7. The nucleotide sequence of claim 6, wherein the sequence encoding the inactive bacterial chloramphenicol acetyl transferase (CAT) fusion protein comprises in a 5′ to 3′ direction
(i) a sequence encoding an inactive bacterial chloramphenicol acetyl transferase (CAT) polypeptide;
(ii) a sequence comprising one or more stop codons;
(iii) a sequence comprising the attachment site for the site-specific transposon and encoding a synthetic polypeptide; and
(iv) a sequence comprising one or more in frame stop codons.

8. The nucleotide sequence of claim 5, wherein the composite selectable marker sequence encodes an active bacterial chloramphenicol acetyl transferase (CAT) fusion protein.

9. The nucleotide sequence of claim 8, wherein the sequence encoding the active bacterial chloramphenicol acetyl transferase (CAT) fusion protein comprises in a 5′ to 3′ direction
(i) a sequence encoding an inactive bacterial chloramphenicol acetyl transferase (CAT) polypeptide domain;
(ii) a sequence comprising one or more out of reading frame stop codons; and
(iii) a sequence comprising one end of the transposon and one or more in frame stop codons;
wherein the addition of polypeptides encoded by (ii) (iii) to the inactive CAT polypeptide domain restore CAT activity to the fusion protein.

10. The nucleotide sequence of claim 5, wherein said fused marker sequence encodes an extended, inactive polypeptide which is truncated after transposition to form a composite target sequence which encodes an active, polypeptide conferring a selectable phenotype upon the cell.

11. The nucleotide sequence of claim 10, wherein the selectable marker sequence encodes an inactive NPT-II fusion protein.

12. The nucleotide sequence of claim 11, wherein the sequence encoding the inactive NPT-II fusion protein comprises in a 5′ to 3′ direction
(i) a sequence encoding an inactive NPT-II polypeptide;
(ii) a sequence comprising one or more stop codons;
(iii) a sequence comprising the attachment site for the site-specific transposon and encoding a synthetic polypeptide; and
(iv) a sequence comprising one or more in frame stop codons.

13. The nucleotide sequence of claim 10, wherein the composite selectable marker sequence encodes an active NPT-II fusion protein.

14. The nucleotide sequence of claim 13, wherein the sequence encoding the active NPT-II fusion protein comprises in a 5′ to 3′ direction
(i) a sequence encoding an inactive NPT-II polypeptide domain;
(ii) a sequence comprising one or more out of reading frame stop codons; and
(iii) a sequence comprising one end of the transposon and one or more in frame stop codons;
wherein the removal of amino acids encoded by (ii) (iii) to the inactive NPT-II polypeptide domain restores NPT-II activity to the fusion protein.

15. The nucleotide sequence of claim 13, wherein the sequence encoding the active NPT-II fusion protein comprises in a 5′ to 3′ direction
(i) a sequence encoding an inactive NPT-II polypeptide domain;
(ii) a sequence comprising one or more out of reading frame stop codons; and
(iii) a sequence comprising one end of the transposon and one or more in frame stop codons;
wherein the addition of amino acids encoded by (ii) (iii) to the inactive NPT-II polypeptide domain restores NPT-II activity to the fusion protein.

16. A vector designated as a synthemid comprising the target sequence or composite target sequence of claim 1.

17. The vector of claim 16, wherein said vector propagates in bacteria.

18. The vector of claim 17, wherein said vector is a shuttle vector capable of propagating in bacteria and a non-bacterial host cell.

19. The vector of claim 18, wherein said vector is a baculovirus shuttle vector, capable of propagating in bacteria and in Lepidopteran insect cells susceptible to infection by the baculovirus.

20. The vector of claim 19, wherein said baculovirus shuttle vector is capable of propagating in Escherichia coli and insect cells selected from the group consisting of Spodoptera frugiperda, Trichoplusia ni cells, and Bombyx mori cells.
